SEQUENCE LISTING (127 sequences)

SEQ ID NO: 1 - DNA, 3900 bp, Homo sapiens
  CDS (138)...(3527)
cggcccatctctctgggtctctgtccctctctctctgggtctctgtccccgtctctctgggtctcggtccccgtctctctgggtctctgtccccgtctctctgggtctctgtccccctccctgtgtgccccgctcccatgtgtccacagttcctccggctctctgaccgaacggatccagctgcagtttatagtctggtcacacgcacatggggcttccgtgccccgaacctggtggtgtcagtgctggggggatcggggggccccgtcctccagacctggctgcaggacctgctgcgtcgtgggctggtgcgggctgcccagagcacaggagcctggattgtcactgggggtctgcacacgggcatcggccggcatgttggtgtggctgtacgggaccatcagatggccagcactgggggcaccaaggtggtggccatgggtgtggccccctggggtgtggtccggaatagagacaccctcatcaaccccaagggctcgttccctgcgaggtaccggtggcgcggtgacccggaggacggggtccagtttcccctggactacaactactcggccttcttcctggtggacgacggcacacacggctgcctggggggcgagaaccgcttccgcttgcgcctggagtcctacatctcacagcagaagacgggcgtgggagggactggaattgacatccctgtcctgctcctcctgattgatggtgatgagaagatgttgacgcgaatagagaacgccacccaggctcagctcccatgtctcctcgtggctggctcagggggagctgcggactgcctggcggagaccctggaagacactctggccccagggagtgggggagccaggcaaggcgaagcccgagatcgaatcaggcgtttctttcccaaaggggaccttgaggtcctgcaggcccaggtggagaggattatgacccggaaggagctcctgacagtctattcttctgaggatgggtctgaggaattcgagaccatagttttgaaggcccttgtgaaggcctgtgggagctcggaggcctcagcctacctggatgagctgcgtttggctgtggcttggaaccgcgtggacattgcacagagtgaactctttcggggggacatccaatggcggtccttccatctcgaagcttccctcatggacgccctgctgaatgaccggcctgagttcgtgcgcttgctcatttcccacggcctcagcctgggccacttcctgaccccgatgcgcctggcccaactctacagcgcggcgccctccaactcgctcatccgcaaccttttggaccaggcgtcccacagcgcaggcaccaaagccccagccctaaaagggggagctgcggagctccggccccctgacgtggggcatgtgctgaggatgctgctggggaagatgtgcgcgccgaggtacccctccgggggcgcctgggaccctcacccaggccagggcttcggggagagcatgtatctgctctcggacaaggccacctcgccgctctcgctggatgctggcctcgggcaggccccctggagcgacctgcttctttgggcactgttgctgaacagggcacagatggccatgtacttctgggagatgggttccaatgcagtttcctcagctcttggggcctgtttgctgctccgggtgatggcacgcctggagcctgacgctgaggaggcagcacggaggaaagacctggcgttcaagtttgaggggatgggcgttgacctctttggcgagtgctatcgcagcagtgaggtgagggctgcccgcctcctcctccgtcgctgcccgctctggggggatgccacttgcctccagctggccatgcaagctgacgcccgtgccttctttgcccaggatggggtacagtctctgctgacacagaagtggtggggagatatggccagcactacacccatctgggccctggttctcgccttcttttgccctccactcatctacacccgcctcatcaccttcaggaaatcagaagaggagcccacacgggaggagctagagtttgacatggatagtgtcattaatggggaagggcctgtcgggacggcggacccagccgagaagacgccgctgggggtcccgcgccagtcgggccgtccgggttgctgcgggggccgctgcggggggcgccggtgcctacgccgctggttccacttctggggcgcgccggtgaccatcttcatgggcaacgtggtcagctacctgctgttcctgctgcttttctcgcgggtgctgctcgtggatttccagccggcgccgcccggctccctggagctgctgctctatttctgggctttcacgctgctgtgcgaggaactgcgccagggcctgagcggaggcgggggcagcctcgccagcgggggccccgggcctggccatgcctcactgagccagcgcctgcgcctctacctcgccgacagctggaaccagtgcgacctagtggctctcacctgcttcctcctgggcgtgggctgccggctgaccccgggtttgtaccacctgggccgcactgtcctctgcatcgacttcatggttttcacggtgcggctgcttcacatcttcacggtcaacaaacagctggggcccaagatcgtcatcgtgagcaagatgatgaaggacgtgttcttcttcctcttcttcctcggcgtgtggctggtagcctatggcgtggccacggaggggctcctgaggccacgggacagtgacttcccaagtatcctgcgccgcgtcttctaccgtccctacctgcagatcttcgggcagattccccaggaggacatggacgtggccctcatggagcacagcaactgctcgtcggagcccggcttctgggcacaccctcctggggcccaggcgggcacctgcgtctcccagtatgccaactggctggtggtgctgctcctcgtcatcttcctgctcgtggccaacatcctgctggtcaacttgctcattgccatgttcagttacacattcggcaaagtacagggcaacagcgatctctactggaaggcgcagcgttaccgcctcatccgggaattccactctcggcccgcgctggccccgccctttatcgtcatctcccacttgcgcctcctgctcaggcaattgtgcaggcgaccccggagcccccagccgtcctccccggccctcgagcatttccgggtttacctttctaaggaagccgagcggaagctgctaacgtgggaatcggtgcataaggagaactttctgctggcacgcgctagggacaagcgggagagcgactccgagcgtctgaagcgcacgtcccagaaggtggacttggcactgaaacagctgggacacatccgcgagtacgaacagcgcctgaaagtgctggagcgggaggtccagcagtgtagccgcgtcctggggtgggtggccgaggccctgagccgctctgccttgctgcccccaggtgggccgccaccccctgacctgcctgggtccaaagactgagccctgctggcggacttcaaggagaagcccccacaggggattttgctcctagagtaaggctcatctgggcctcggcccccgcacctggtggccttgtccttgaggtgagccccatgtccatctgggccactgtcaggaccacctttgggagtgtcatccttacaaaccacagcatgcccggctcctcccagaaccagtcccagcctgggaggatcaaggcctggatcccgggccgttatccatctggaggctgcagggtccttggggtaacagggaccacagacccctcaccactcacagattcctcacactggggaaataaagccatttcagaggaaaaaaaaaaaaaaaaaarraaaaaaaaaaaaggcgg

SEQ ID NO: 2 - protein, 1129 aa, Homo sapiens
MCPQFLRLSDRTDPAAVYSLVTRTWGFRAPNLVVSVLGGSGGPVLQTWLQDLLRRGLVRAAQSTGAWIVTGGLHTGIGRHVGVAVRDHQMASTGGTKVVAMGVAPWGVVRNRDTLINPKGSFPARYRWRGDPEDGVQFPLDYNYSAFFLVDDGTHGCLGGENRFRLRLESYISQQKTGVGGTGIDIPVLLLLIDGDEKMLTRIENATQAQLPCLLVAGSGGAADCLAETLEDTLAPGSGGARQGEARDRIRRFFPKGDLEVLQAQVERIMTRKELLTVYSSEDGSEEFETIVLKALVKACGSSEASAYLDELRLAVAWNRVDIAQSELFRGDIQWRSFHLEASLMDALLNDRPEFVRLLISHGLSLGHFLTPMRLAQLYSAAPSNSLIRNLLDQASHSAGTKAPALKGGAAELRPPDVGHVLRMLLGKMCAPRYPSGGAWDPHPGQGFGESMYLLSDKATSPLSLDAGLGQAPWSDLLLWALLLNRAQMAMYFWEMGSNAVSSALGACLLLRVMARLEPDAEEAARRKDLAFKFEGMGVDLFGECYRSSEVRAARLLLRRCPLWGDATCLQLAMQADARAFFAQDGVQSLLTQKWWGDMASTTPIWALVLAFFCPPLIYTRLITFRKSEEEPTREELEFDMDSVINGEGPVGTADPAEKTPLGVPRQSGRPGCCGGRCGGRRCLRRWFHFWGAPVTIFMGNVVSYLLFLLLFSRVLLVDFQPAPPGSLELLLYFWAFTLLCEELRQGLSGGGGSLASGGPGPGHASLSQRLRLYLADSWNQCDLVALTCFLLGVGCRLTPGLYHLGRTVLCIDFMVFTVRLLHIFTVNKQLGPKIVIVSKMMKDVFFFLFFLGVWLVAYGVATEGLLRPRDSDFPSILRRVFYRPYLQIFGQIPQEDMDVALMEHSNCSSEPGFWAHPPGAQAGTCVSQYANWLVVLLLVIFLLVANILLVNLLIAMFSYTFGKVQGNSDLYWKAQRYRLIREFHSRPALAPPFIVISHLRLLLRQLCRRPRSPQPSSPALEHFRVYLSKEAERKLLTWESVHKENFLLARARDKRESDSERLKRTSQKVDLALKQLGHIREYEQRLKVLEREVQQCSRVLGWVAEALSRSALLPPGGPPPPDLPGSKD

SEQ ID NO: 3 - DNA, 3390 bp, Homo sapiens
  CDS (1)...(3390)
atgtgtccacagttcctccggctctctgaccgaacggatccagctgcagtttatagtctggtcacacgcacatggggcttccgtgccccgaacctggtggtgtcagtgctggggggatcggggggccccgtcctccagacctggctgcaggacctgctgcgtcgtgggctggtgcgggctgcccagagcacaggagcctggattgtcactgggggtctgcacacgggcatcggccggcatgttggtgtggctgtacgggaccatcagatggccagcactgggggcaccaaggtggtggccatgggtgtggccccctggggtgtggtccggaatagagacaccctcatcaaccccaagggctcgttccctgcgaggtaccggtggcgcggtgacccggaggacggggtccagtttcccctggactacaactactcggccttcttcctggtggacgacggcacacacggctgcctggggggcgagaaccgcttccgcttgcgcctggagtcctacatctcacagcagaagacgggcgtgggagggactggaattgacatccctgtcctgctcctcctgattgatggtgatgagaagatgttgacgcgaatagagaacgccacccaggctcagctcccatgtctcctcgtggctggctcagggggagctgcggactgcctggcggagaccctggaagacactctggccccagggagtgggggagccaggcaaggcgaagcccgagatcgaatcaggcgtttctttcccaaaggggaccttgaggtcctgcaggcccaggtggagaggattatgacccggaaggagctcctgacagtctattcttctgaggatgggtctgaggaattcgagaccatagttttgaaggcccttgtgaaggcctgtgggagctcggaggcctcagcctacctggatgagctgcgtttggctgtggcttggaaccgcgtggacattgcacagagtgaactctttcggggggacatccaatggcggtccttccatctcgaagcttccctcatggacgccctgctgaatgaccggcctgagttcgtgcgcttgctcatttcccacggcctcagcctgggccacttcctgaccccgatgcgcctggcccaactctacagcgcggcgccctccaactcgctcatccgcaaccttttggaccaggcgtcccacagcgcaggcaccaaagccccagccctaaaagggggagctgcggagctccggccccctgacgtggggcatgtgctgaggatgctgctggggaagatgtgcgcgccgaggtacccctccgggggcgcctgggaccctcacccaggccagggcttcggggagagcatgtatctgctctcggacaaggccacctcgccgctctcgctggatgctggcctcgggcaggccccctggagcgacctgcttctttgggcactgttgctgaacagggcacagatggccatgtacttctgggagatgggttccaatgcagtttcctcagctcttggggcctgtttgctgctccgggtgatggcacgcctggagcctgacgctgaggaggcagcacggaggaaagacctggcgttcaagtttgaggggatgggcgttgacctctttggcgagtgctatcgcagcagtgaggtgagggctgcccgcctcctcctccgtcgctgcccgctctggggggatgccacttgcctccagctggccatgcaagctgacgcccgtgccttctttgcccaggatggggtacagtctctgctgacacagaagtggtggggagatatggccagcactacacccatctgggccctggttctcgccttcttttgccctccactcatctacacccgcctcatcaccttcaggaaatcagaagaggagcccacacgggaggagctagagtttgacatggatagtgtcattaatggggaagggcctgtcgggacggcggacccagccgagaagacgccgctgggggtcccgcgccagtcgggccgtccgggttgctgcgggggccgctgcggggggcgccggtgcctacgccgctggttccacttctggggcgcgccggtgaccatcttcatgggcaacgtggtcagctacctgctgttcctgctgcttttctcgcgggtgctgctcgtggatttccagccggcgccgcccggctccctggagctgctgctctatttctgggctttcacgctgctgtgcgaggaactgcgccagggcctgagcggaggcgggggcagcctcgccagcgggggccccgggcctggccatgcctcactgagccagcgcctgcgcctctacctcgccgacagctggaaccagtgcgacctagtggctctcacctgcttcctcctgggcgtgggctgccggctgaccccgggtttgtaccacctgggccgcactgtcctctgcatcgacttcatggttttcacggtgcggctgcttcacatcttcacggtcaacaaacagctggggcccaagatcgtcatcgtgagcaagatgatgaaggacgtgttcttcttcctcttcttcctcggcgtgtggctggtagcctatggcgtggccacggaggggctcctgaggccacgggacagtgacttcccaagtatcctgcgccgcgtcttctaccgtccctacctgcagatcttcgggcagattccccaggaggacatggacgtggccctcatggagcacagcaactgctcgtcggagcccggcttctgggcacaccctcctggggcccaggcgggcacctgcgtctcccagtatgccaactggctggtggtgctgctcctcgtcatcttcctgctcgtggccaacatcctgctggtcaacttgctcattgccatgttcagttacacattcggcaaagtacagggcaacagcgatctctactggaaggcgcagcgttaccgcctcatccgggaattccactctcggcccgcgctggccccgccctttatcgtcatctcccacttgcgcctcctgctcaggcaattgtgcaggcgaccccggagcccccagccgtcctccccggccctcgagcatttccgggtttacctttctaaggaagccgagcggaagctgctaacgtgggaatcggtgcataaggagaactttctgctggcacgcgctagggacaagcgggagagcgactccgagcgtctgaagcgcacgtcccagaaggtggacttggcactgaaacagctgggacacatccgcgagtacgaacagcgcctgaaagtgctggagcgggaggtccagcagtgtagccgcgtcctggggtgggtggccgaggccctgagccgctctgccttgctgcccccaggtgggccgccaccccctgacctgcctgggtccaaagactga

SEQ ID NO: 4 - protein, 4 aa, Homo sapiens
  VARIANT (2)...(2): Xaa = Any amino acid except P
NXXX

SEQ ID NO: 5 - DNA, 2796 bp, Homo sapiens
  CDS (1)...(2796)
atgagccaaagcccggcgttcgggccccggaggggcatttctccccggggcgctgccggagccgctgcgcggcgcaacgagagccaggactatctgctcatggactcggagctgggagaagacggctgcccgcaagccccgctgccttgctacggctactacccctgcttccggggatctgacaacagactggctcaccggcggcagacagttctccgtgagaaggggagaaggttagctaatcgaggaccagcatacatgtttagtgatcgctccacaagcctatctatagaggaggaacgctttttggatgcagctgaatatggtaacatcccagtggtgcggaagatgttagaagaatgccactcactcaacgttaactgtgtggattacatgggccagaatgccctacagttggcagtggccaatgagcatctggaaattacagaacttcttctcaagaaagaaaacctctctcgaattggggatgctttgcttctagctattagtaaaggttatgttcggattgtggaagcaattctcagtcatccggcttttgctgaaggcaagaggttagcaaccagccctagccagtctgaactccagcaagatgatttttatgcctatgatgaagatgggacacggtcctcccatgatgtgactccaatcattctggctgcccactgccgggaatatgaaattgtgcataccctcctgcggaagggtgctaggattgaacggcctcatgattatttctgcaagtgcaatgactgcaaccagaaacagaagcatgactcgtttagccactccagatctaggattaatgcctataaaggcctggcaagtccggcttacctgtcattgtctagtgaagatccagtcatgacggctttagaacttagcaatgaactggcagttctggccaatattgagaaagagttcaagaatgactacaaaaaactgtcaatgcagtgcaaagactttgttgttggactccttgatctgtgcagaaacactgaagaagtcgaggccattctgaatggggatgttgaaacgctccagagtggtgatcacggtcgcccaaatctcagccgtttaaaacttgccattaaatatgaagtaaaaaaatttgtagctcatccaaactgccaacagcaacttctctccatttggtatgagaatctttctggtttacgacagcagacaatggcggtcaagttccttgtggcccttgctgttgccattggactgcccttcctggctctcatttactggtttgctccatgcagcaagatggggaagataatgcgtggaccattcatgaagtttgtagcacacgcagcctccttcaccatttttctgggactgctagtcatgaatgcagctgacagatttgaaggcacaaaactccttcctaatgaaaccagcacagataatgcaaaacagctgttcaggatgaaaacatcctgcttctcatggatggagatgctcattatatcctgggtaataggcatgatatgggctgaatgtaaagaaatctggactcagggccccaaggaatatttgtttgagttgtggaacatgcttgattttggtatgttagcaattttcccagcatcattcattgcgagattcatggcattttggcatgcttccaaagcccagagcatcattgacgcaaacgatactttgaaggacttgacgaaagtaacattgggagacaatgtgaaatactacaatttggccaggataaagtgggacccctctgatcctcaaataatatctgaaggtctttatgcaattgctgtagttttaagtttctctaggatagcttatattttaccagcaaatgaaagctttggacctctgcagatatcacttggaagaacagtcaaagacatcttcaagttcatggtcatattcattatggtgtttgtggcctttatgattggaatgttcaatctctactcctactacattggtgcaaaacaaaatgaagccttcacaacagttgaagagagttttaagacactgttctgggctatatttggactttctgaagtgaaatcagtggtcatcaactataaccacaaattcattgaaaacattggttacgttctttatggagtctataatgttacgatggtcattgttttgctaaatatgttaattgccatgatcaacagttcattccaggaaattgaggatgacgctgatgtggagtggaaatttgcaagggccaaactctggttttcctactttgaggagggcagaacacttcctgtacccttcaatctggtgccgagtccaaagtccctgttttatctcttactgaagcttaaaaaatggatttctgagctgttccagggccataaaaaaggtttccaggaagatgcagagatgaacaagataaatgaagaaaagaaacttggaattttaggaagtcatgaagacctttcaaaattatcacttgacaaaaaacaggttgggcacaataaacaaccaagtataaggagctcagaagatttccatctaaatagtttcaataatcctccaagacaatatcagaaaataatgaaaaggctcattaaaagatatgtactgcaggcccagatagataaggagagtgatgaagtgaacgaaggggaactgaaggaaattaagcaggacatctcaagtctccgctatgaactccttgaagaaaaatctcagaatacagaagacctagcagaacttattagagaacttggagagaaattatccatggaaccaaatcaagaggaaaccaatagataa

SEQ ID NO: 6 - protein, 931 aa, Homo sapiens
MSQSPAFGPRRGISPRGAAGAAARRNESQDYLLMDSELGEDGCPQAPLPCYGYYPCFRGSDNRLAHRRQTVLREKGRRLANRGPAYMFSDRSTSLSIEEERFLDAAEYGNIPVVRKMLEECHSLNVNCVDYMGQNALQLAVANEHLEITELLLKKENLSRIGDALLLAISKGYVRIVEAILSHPAFAEGKRLATSPSQSELQQDDFYAYDEDGTRSSHDVTPIILAAHCREYEIVHTLLRKGARIERPHDYFCKCNDCNQKQKHDSFSHSRSRINAYKGLASPAYLSLSSEDPVMTALELSNELAVLANIEKEFKNDYKKLSMQCKDFVVGLLDLCRNTEEVEAILNGDVETLQSGDHGRPNLSRLKLAIKYEVKKFVAHPNCQQQLLSIWYENLSGLRQQTMAVKFLVALAVAIGLPFLALIYWFAPCSKMGKIMRGPFMKFVAHAASFTIFLGLLVMNAADRFEGTKLLPNETSTDNAKQLFRMKTSCFSWMEMLIISWVIGMIWAECKEIWTQGPKEYLFELWNMLDFGMLAIFPASFIARFMAFWHASKAQSIIDANDTLKDLTKVTLGDNVKYYNLARIKWDPSDPQIISEGLYAIAVVLSFSRIAYILPANESFGPLQISLGRTVKDIFKFMVIFIMVFVAFMIGMFNLYSYYIGAKQNEAFTTVEESFKTLFWAIFGLSEVKSVVINYNHKFIENIGYVLYGVYNVTMVIVLLNMLIAMINSSFQEIEDDADVEWKFARAKLWFSYFEEGRTLPVPFNLVPSPKSLFYLLLKLKKWISELFQGHKKGFQEDAEMNKINEEKKLGILGSHEDLSKLSLDKKQVGHNKQPSIRSSEDFHLNSFNNPPRQYQKIMKRLIKRYVLQAQIDKESDEVNEGELKEIKQDISSLRYELLEEKSQNTEDLAELIRELGEKLSMEPNQEETNR

SEQ ID NO: 7 - DNA, 2796 bp, Homo sapiens; identical to SEQ ID NO: 5
  CDS (1)...(2796)

SEQ ID NO: 8 - protein, 35 aa, Artificial Sequence
  Amino Acid Consensus Sequence
DGRTPLHLAARNGHLEVVKLLLEXXAGADVNARDK

SEQ ID NO: 9 - protein, 33 aa, Artificial Sequence
  Amino Acid Consensus Sequence
DGRTPLHLAARNGHLEVVKLLLEAGADVNARDK

SEQ ID NO: 10 - protein, 33 aa, Artificial Sequence; identical to SEQ ID NO: 9
  Amino Acid Consensus Sequence

SEQ ID NO: 11 - protein, 250 aa, Artificial Sequence
  Amino Acid Consensus Sequence
ILFILDLLFVLLFLLEIVLKFIAYGLKSTSNIAAKYLKSIFNILDLLAILPLLLLLVLFLSGTEQVXKRLXXXXXXXXXXXXXXXXXXXXXXRERSLELSQYRILRFLRLLRLLRLLRLLRLLRRLETLFEFELGTLAWSLQSLGRALKSILRFLLLLLLLLIGFSVIGYLLFKGYEDLSENEVDGNSEFSSYFDAFYFLFVTLTTVGFGDLVPVXXXXXXXXXWLGIIFFVLFFIIVGLLLLNLLIAVI

SEQ ID NO: 12 - protein, 227 aa, Artificial Sequence
  Amino Acid Consensus Sequence
NVDESCEDRSWRSALLIAIENENLEIMELLLNHSVFDFRRVGDALLYAISKEYVRAVEALLSHPAKAEGKRLAPSPWEQELQQDDFYAYDEDGSEFTPDITPIILAAHTNNYEIIKLLLQKGVSIPRPHDVRCNCTECVSKQEVDSLRHSRSRLNIYKALASPSLIALSSEDPILTAFELSWELKELSKVENEFKNEYEELSRQCKQFAKDLLDQCRNSRELEVILN

SEQ ID NO: 13 - DNA, 2875 bp, Homo sapiens
  CDS (115)...(1869)
atgttgtccccctttctcactcccaccttgcaggccaccggagccgccagctgttggaactgagctactgcagaaagggaagtggagagtaagggccaggccccgtgggggcagatggccggcagaaggctgaatctgcgctgggcactgagtgtgctttgtgtgctgctaatggcggagacagtgtctgggactaggggctcgtctacaggagctcacattagcccccagtttccagcttcaggtgtgaaccagacccccgtggtagactgccgcaaggtgtgtggcctgaatgtctctgaccgctgtgacttcatccggaccaaccctgactgccacagtgatggggggtacctggactacctggaaggcatcttctgccacttccctcccagcctcctccctctggctgtcactctctacgtttcctggctgctctacctgtttctgattctgggagtcaccgcagccaagtttttctgccccaacttgtcggccatttctaccacactgaagctctcccacaacgtggcaggcgtcaccttcctggcatttgggaatggtgcacctgacatcttcagtgccctggtggccttctctgacccgcacacagccggcctggcccttggggcactgtttggcgctggcgtgctggttaccacagtggtggccggaggcattaccatcctacaccccttcatggctgcctccaggcccttcttcagggacatcgttttctacatggtggctgtgttcctgaccttcctcatgctcttccgtggcagggtcaccctggcatgggctctgggttacctgggcttgtatgtgttctatgtggtcactgtgattctctgcacctggatctaccaacggcaacggagaggatctctgttctgccccatgccagttactccagagatcctctcagactccgaggaggaccgggtatcttctaataccaacagctatgactacggtgatgagtaccggccgctgttcttctaccaggagaccacggctcagatcctggtccgggccctcaatcccctggattacatgaagtggagaaggaaatcagcatactggaaagccctcaaggtgttcaagctgcctgtggagttcctgctgctcctcacagtccccgtcgtggacccggacaaggatgaccagaactggaaacggcccctcaactgtctgcatctggttatcagccccctggttgtggtcctgaccctgcagtcggggacctatggtgtctatgagataggcggcctcgttcccgtctgggtcgtggtggtgatcgcaggcacagccttggcttcagtgaccttttttgccacatctgacagccagccccccaggcttcactggctctttgctttcctgggctttctgaccagcgccctgtggatcaacgcggccgccacagaggtggtgaacatcttgcggtccctgggtgtggtcttccggctgagcaacactgtgctggggctcacgctgctggcctgggggaacagcattggagatgccttctcggatttcacactggctcgccagggctacccacggatggcgttctccgcctgctttggcggcatcatcttcaacatcctcgtgggtgtggggctgggctgcctgctccagatctcccgaagccacacagaagtgaagctggagccagacggactgctggtgtgggtcctggcaggcgccctggggctcagcctcgtcttctccctggtctcagtcccattgcagtgcttccagctcagcagagtctatggcttctgcctgctcctcttctacctgaacttccttgtcgtggccctcctcactgaatttggagtgattcacctgaaaagcatgtgactgaagccgcttagtgctgtggcctcactgcaggcaggagccccgcccctcctgccgggggaggcccagggaccggagcatttctgcaaggcccttgtgggcacgagagtgcggcccttgctgctggagatctgaggtcactgctgtgagctgggagaactgctgtgtacctcttgctgccagcacccaacagccttgccgtggggaccttggaaacctggctttgctctggacaaagggttccagagagaagctagaagtcccccttgaatgacccccagagcccctctgagaagggctggagtttgggggaaggggatggctggatgtgctccaggccatgctggaggtacccccgaggcacaggcactgcccgttccccttgcctgggcttcaggccttctggcaccttctcaggacacaagtggctgcccaaccctgactcagagaatgagggtggcttggacccctgggaatcaggccgccgagggctgagctccagagccgcaccatctgccacaaacagaattcgagacatacttaattttgaatttctccttgccacgttaataaagccaaaagcagcgggtgctattcgtggcaacacacttcactgaacccacttgcttccaaaacgatgccagcccgaggcactgctacgccagcagctgccacatgggatggtggctcaggcgctccctccaggattctgcccctgcctgtccacagactcctttgtgctggaacctgggctcctccagctgccaggcaggagtcggtaggactgtgcctgtgcctccctcagcggggccctgggcggggttccaaggcctgcgagctgggaaaggacagatgaggggacctcgtgccttcttgctgtcatgcaatgaccccgccttatgttgccgaaataagcaactcttaggtttgcctgactgccttatgctggtaaagaaaagggattcaactgtctcttttccaaataaaaaaaaaagtcaaaatttca

SEQ ID NO: 14 - protein, 584 aa, Homo sapiens
MAGRRLNLRWALSVLCVLLMAETVSGTRGSSTGAHISPQFPASGVNQTPVVDCRKVCGLNVSDRCDFIRTNPDCHSDGGYLDYLEGIFCHFPPSLLPLAVTLYVSWLLYLFLILGVTAAKFFCPNLSAISTTLKLSHNVAGVTFLAFGNGAPDIFSALVAFSDPHTAGLALGALFGAGVLVTTVVAGGITILHPFMAASRPFFRDIVFYMVAVFLTFLMLFRGRVTLAWALGYLGLYVFYVVTVILCTWIYQRQRRGSLFCPMPVTPEILSDSEEDRVSSNTNSYDYGDEYRPLFFYQETTAQILVRALNPLDYMKWRRKSAYWKALKVFKLPVEFLLLLTVPVVDPDKDDQNWKRPLNCLHLVISPLVVVLTLQSGTYGVYEIGGLVPVWVVVVIAGTALASVTFFATSDSQPPRLHWLFAFLGFLTSALWINAAATEVVNILRSLGVVFRLSNTVLGLTLLAWGNSIGDAFSDFTLARQGYPRMAFSACFGGIIFNILVGVGLGCLLQISRSHTEVKLEPDGLLVWVLAGALGLSLVFSLVSVPLQCFQLSRVYGFCLLLFYLNFLVVALLTEFGVIHLKSM

SEQ ID NO: 15 - DNA, 1755 bp, Homo sapiens
  CDS (1)...(1755)
atggccggcagaaggctgaatctgcgctgggcactgagtgtgctttgtgtgctgctaatggcggagacagtgtctgggactaggggctcgtctacaggagctcacattagcccccagtttccagcttcaggtgtgaaccagacccccgtggtagactgccgcaaggtgtgtggcctgaatgtctctgaccgctgtgacttcatccggaccaaccctgactgccacagtgatggggggtacctggactacctggaaggcatcttctgccacttccctcccagcctcctccctctggctgtcactctctacgtttcctggctgctctacctgtttctgattctgggagtcaccgcagccaagtttttctgccccaacttgtcggccatttctaccacactgaagctctcccacaacgtggcaggcgtcaccttcctggcatttgggaatggtgcacctgacatcttcagtgccctggtggccttctctgacccgcacacagccggcctggcccttggggcactgtttggcgctggcgtgctggttaccacagtggtggccggaggcattaccatcctacaccccttcatggctgcctccaggcccttcttcagggacatcgttttctacatggtggctgtgttcctgaccttcctcatgctcttccgtggcagggtcaccctggcatgggctctgggttacctgggcttgtatgtgttctatgtggtcactgtgattctctgcacctggatctaccaacggcaacggagaggatctctgttctgccccatgccagttactccagagatcctctcagactccgaggaggaccgggtatcttctaataccaacagctatgactacggtgatgagtaccggccgctgttcttctaccaggagaccacggctcagatcctggtccgggccctcaatcccctggattacatgaagtggagaaggaaatcagcatactggaaagccctcaaggtgttcaagctgcctgtggagttcctgctgctcctcacagtccccgtcgtggacccggacaaggatgaccagaactggaaacggcccctcaactgtctgcatctggttatcagccccctggttgtggtcctgaccctgcagtcggggacctatggtgtctatgagataggcggcctcgttcccgtctgggtcgtggtggtgatcgcaggcacagccttggcttcagtgaccttttttgccacatctgacagccagccccccaggcttcactggctctttgctttcctgggctttctgaccagcgccctgtggatcaacgcggccgccacagaggtggtgaacatcttgcggtccctgggtgtggtcttccggctgagcaacactgtgctggggctcacgctgctggcctgggggaacagcattggagatgccttctcggatttcacactggctcgccagggctacccacggatggcgttctccgcctgctttggcggcatcatcttcaacatcctcgtgggtgtggggctgggctgcctgctccagatctcccgaagccacacagaagtgaagctggagccagacggactgctggtgtgggtcctggcaggcgccctggggctcagcctcgtcttctccctggtctcagtcccattgcagtgcttccagctcagcagagtctatggcttctgcctgctcctcttctacctgaacttccttgtcgtggccctcctcactgaatttggagtgattcacctgaaaagcatgtga

SEQ ID NO: 16 - protein, 152 aa, Artificial Sequence
  Amino Acid Consensus Sequence
ILIVLGADLFVDGASAIAEVLGISESVIGLTLVALGTSLPELFASLIAALKGQFQADIAIGNVIGSNIFNILLGLGIASLIAPLYHKAKGESFIVDPISLRRDVLFLLLVLLILIVFLLLGRSLIGRGDGVLLLILYILYLTFLVFSILLEV

SEQ ID NO: 17 - protein, 661 aa, Artificial Sequence
  Amino Acid Consensus Sequence
MDLQQSTTITSLEKWCLDESLSGCRRHYSVKKKLKLIRVLGLFMGLVAISTVSFSISAFSETDTQSTGEASVVSGPRVAQGYHQRTLLDLNDKILDYTPQPPLSKEGESENSTDHAQGDYPKDIFSLEERRKGAIILHVIGMIYMFIALAIVCDEFFVPSLTVITEKLGISDDVAGATFMAAGGSAPELFTSLIGVFIAHSNVGIGTIVGSAVFNILFVIGMCALFSREILNLTWWPLFRDVSFYIVDLIMLIIFFLDNVIMWWESLLLLTAYFCYVVFMKFNVQVEKWVKQMINRNKVVKVTAPEAQAKPSAARDKDEPTLPAKPRLQRGGSSASLHNSLMRNSIFQLMIHTLDPLAEELGSYGKLKYYDTMTEEGRFREKASILHKIAKKKCHVDENERQNGAANHVEKIELPNSTSTDVEMTPSSDASEPVQNGNLSHNIEGAEAQTADEEEDQPLSLAWPSETRKQVTFLIVFPIVFPLWITLPDVRKPSSRKFFPITFFGSITWIAVFSYLMVWWAHQVGETIGISEEIMGLTILAAGTSIPDLITSVIVARKGLGDMAVSSSVGSNIFDITVGLPLPWLLYTVIHRFQPVAVSSNGLFCAIVLLFIMLLFVILSIALCKWRMNKILGFIMFGLYFVFLVVSVLLEDRILTCPVSI

SEQ ID NO: 18 - DNA, 1408 bp, Homo sapiens
  CDS (84)...(1343)
ccacgcgtccgcggacgcgtgggtcggcaccggcgaggccgtgctggaacccgggcctcagccgcagccgcagcggggccgacatgacgacagctccccaggagccccccgcccggcccctccaggcgggcagtggagctggcccggcgcctgggcgcgccatgcgcagcaccacgctcctggccctgctggcgctggtcttgctttacttggtgtctggtgccctggtgttccgggccctggagcagccccacgagcagcaggcccagagggagctgggggaggtccgagagaagttcctgagggcccatccgtgtgtgagcgaccaggagctgggcctcctcatcaaggaggtggctgatgccctgggagggggtgcggacccagaaaccaactcgaccagcaacagcagccactcagcctgggacctgggcagcgccttctttttctcagggaccatcatcaccaccatcggctatggcaatgtggccctgcgcacagatgccgggcgcctcttctgcatcttttatgcgctggtggggattccgctgtttgggatcctactggcaggggtcggggaccggctgggctcctccctgcgccatggcatcggtcacattgaagccatcttcttgaagtggcacgtgccaccggagctagtaagagtgctgtcggcgatgcttttcctgctgatcggctgcctgctctttgtcctcacgcccacgttcgtgttctgctatatggaggactggagcaagctggaggccatctactttgtcatagtgacgcttaccaccgtgggctttggcgactatgtggccggcgcggaccccaggcaggactccccggcctatcagccgctggtgtggttctggatcctgctcggcctggcttacttcgcctcagtgctcaccaccatcgggaactggctgcgagtagtgtcccgccgcactcgggcagagatgggcggcctcacggctcaggctgccagctggactggcacagtgacagcgcgcgtgacccagcgagccgggcccgccgccccgccgccggagaaggagcagccactgctgcctccaccgccctgtccagcgcagccgctgggcaggccccgatccccttcgccccccgagaaggctcagccgccttccccgcccacggcctcggccctggattatcccagcgagaacctggccttcatcgacgagtcctcggatacgcagagcgagcgcggctgcccgctgccccgcgcgccgagaggtcgccgccgcccaaatccccccaggaagcccgtgcggccccgcggccccgggcgtccccgagacaaaggcgtgccggtgtaggggcaggatccctggccgggcctctcaagggcttcgtttctgctctccccggcatgcctggcttc

SEQ ID NO: 19 - protein, 419 aa, Homo sapiens
MTTAPQEPPARPLQAGSGAGPAPGRAMRSTTLLALLALVLLYLVSGALVFRALEQPHEQQAQRELGEVREKFLRAHPCVSDQELGLLIKEVADALGGGADPETNSTSNSSHSAWDLGSAFFFSGTIITTIGYGNVALRTDAGRLFCIFYALVGIPLFGILLAGVGDRLGSSLRHGIGHIEAIFLKWHVPPELVRVLSAMLFLLIGCLLFVLTPTFVFCYMEDWSKLEAIYFVIVTLTTVGFGDYVAGADPRQDSPAYQPLVWFWILLGLAYFASVLTTIGNWLRVVSRRTRAEMGGLTAQAASWTGTVTARVTQRAGPAAPPPEKEQPLLPPPPCPAQPLGRPRSPSPPEKAQPPSPPTASALDYPSENLAFIDESSDTQSERGCPLPRAPRGRRRPNPPRKPVRPRGPGRPRDKGVPV

SEQ ID NO: 20 - DNA, 1260 bp, Homo sapiens
  CDS (1)...(1260)
atgacgacagctccccaggagccccccgcccggcccctccaggcgggcagtggagctggcccggcgcctgggcgcgccatgcgcagcaccacgctcctggccctgctggcgctggtcttgctttacttggtgtctggtgccctggtgttccgggccctggagcagccccacgagcagcaggcccagagggagctgggggaggtccgagagaagttcctgagggcccatccgtgtgtgagcgaccaggagctgggcctcctcatcaaggaggtggctgatgccctgggagggggtgcggacccagaaaccaactcgaccagcaacagcagccactcagcctgggacctgggcagcgccttctttttctcagggaccatcatcaccaccatcggctatggcaatgtggccctgcgcacagatgccgggcgcctcttctgcatcttttatgcgctggtggggattccgctgtttgggatcctactggcaggggtcggggaccggctgggctcctccctgcgccatggcatcggtcacattgaagccatcttcttgaagtggcacgtgccaccggagctagtaagagtgctgtcggcgatgcttttcctgctgatcggctgcctgctctttgtcctcacgcccacgttcgtgttctgctatatggaggactggagcaagctggaggccatctactttgtcatagtgacgcttaccaccgtgggctttggcgactatgtggccggcgcggaccccaggcaggactccccggcctatcagccgctggtgtggttctggatcctgctcggcctggcttacttcgcctcagtgctcaccaccatcgggaactggctgcgagtagtgtcccgccgcactcgggcagagatgggcggcctcacggctcaggctgccagctggactggcacagtgacagcgcgcgtgacccagcgagccgggcccgccgccccgccgccggagaaggagcagccactgctgcctccaccgccctgtccagcgcagccgctgggcaggccccgatccccttcgccccccgagaaggctcagccgccttccccgcccacggcctcggccctggattatcccagcgagaacctggccttcatcgacgagtcctcggatacgcagagcgagcgcggctgcccgctgccccgcgcgccgagaggtcgccgccgcccaaatccccccaggaagcccgtgcggccccgcggccccgggcgtccccgagacaaaggcgtgccggtgtag

SEQ ID NO: 21 - DNA, 4586 bp, Homo sapiens
  CDS (146)...(2518)
ccacgcgtccgattccagccatccctctgcctgcaatgagagcttcccgccgcctcagccacagtcccacccgggggccttgggccccagacatgcggtgatctcagggcaagggttgccacgaccacccagaacctcaccagccatgaaagcccaccccaaggagatggtgcctctcatgggcaagagagttgctgcccccagtgggaaccctgccgtcctgccagagaagaggccggcggagatcacccccacaaagaagagtgcacacttcttcctggagatagaagggtttgaacccaaccccacagttgccaagacctctcctcctgtcttctccaagcccatggattccaacatccggcagtgcatctctggtaactgtgatgacatggactccccccagtctcctcaagatgatgtgacagagaccccatccaatcccaacagccccagtgcacagctggccaaggaagagcagaggaggaaaaagaggcggctgaagaagcgcatctttgcagccgtgtctgagggctgcgtggaggagttggtagagttgctggtggagctgcaggagctttgcaggcggcgccatgatgaggatgtgcctgacttcctcatgcacaagctgacggcctccgacacggggaagacctgcctgatgaaggccttgttaaacatcaaccccaacaccaaggagatcgtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgcccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgtgtttttgcttggatttggagtagccttggcctcgctgatcgagaagtgtcccaaagacaacaaggactgcagctcctacggcagcttcagtgacgcagtgctggaactcttcaagctcaccataggcctgggtgatctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaatggaagacgcacgtctccttccttaacgaagacccggggcctgtaagacgaacagatttcaacaaaatccaagattcttccaggaacaacagcaaaaccactctcaatgcatttgaagaagtcgaggaattcccggaaacctcggtgtagaagcggaacccagagctggtgtgcgcgtgcgctgtctggcgctgcaggcggagtcaccgactctgtgcagagaggctttgagggatgatggagtccggctctgctggcctagaagcagagtgcaccctcgtgctcagtgctcagtgggtgtctgaactgaggggcagttgtcaatttgtctgagtgggaaacatcctggattttgttacttggcaaacagctggtgtaaacctacagccagcagcagtctggagcctgggagcctcctgaagtcccgggtgaagcctctggttttaccaattgcaggtcggcttggctgggagagatggatggcgggaaaggggcagcagtcttgaggagcagggagaggagtctttcctcctgccagcttcccccgtcagccccaaccccagcccacacattgtaccatctcttctgctgtgactgggttgcctgaatttgtgggagacccgtgatcccatcccagagtgtgcgggggacggaggtaagctggatatcctgggggaggaggggaatgcgctctggaaacacccttccggaacccttcggggaaaaggagaccatccttggagtgaacgtcccctgacaccccaaggttcaaactgtctcaagctgagagatgtttttagtagcataattaacacagggttttaacttgcaatacggaaaagacatttcagttgagaatgaaaattactacaatgaagtttgtgattttaaaagtggagacagactgggggctttggggctggatgtaagtattatatatttggcctcagggtgcccagagcaagacaaaaagcttttcttcacacacacaaaagtctgcatgagacactccgggcaagtcctgctgggccgccgcgatctgggtgaaaggtcctggctcttttcctcgtcctgacctcacagtagcgcatgcctgtgtgctgggatcgtggctttcgctgaagcagaaatagcagctgctcgatcgatatcatcttggaactcagcagttagtcgcatacctcagtacgtctcagtgggggaatttaacaaaatgcctcaactgctttggtacgaagtattttttttttaattttaactgtgaattttgaagctgaaggggaagcttgtgagagaaaagcatttgccaagactttgagcttatttttaggtcctcgtcctctgatgttctctttctgaaatgacacggagtcagtctcgggggcagaggtgaagtggagacggaaggattttccaggtgactggggccgaaaccaccagaaaatccactctgccgccgttatctggtgaaaggattcatgtaaaaatgttcgaggtggaattataaaaatagtaaccataaatgttaatcttaaatggcagaaatagaaatttggccttcagataacatggcgatagataagttcatctggcttgaggcaaactgaagagtcggggcctagcattgcactctgggccagtttctctgccctgggccactctgtgtgccagactagctggacagatagagactttgtgcccctgatggggccgattggggagaggtgggctggggtgtgcaggcttcacaatccacagcagcccctgccctcccagctgacccagggagtaatcgcgtgctctaagccacagtggtcggggctgggcatgggcctctggagaagagaagatttgaggagaactgtcctagaggcaggaggagcagatgtgtttcagaatgggcagaattaggaaattgagaaagattttggctcaacagaatccagcaactgctccagatgttggagatgtttaagcagaagctggttgagcacttaatgaggaatgttgttgaaaatggtcattggaagaagtttaaggtcccttttagcctggagattgtacaaatcagcattccacatctggagttagctacccgcattaagcctgaacagacatcttggtctgaaaggaagtggtttggattcatgatgccaagctccacactatggagctgggaattccagaattgctttgactcagatattaatggagaaag

SEQ ID NO: 22 - protein, 790 aa, Homo sapiens
MKAHPKEMVPLMGKRVAAPSGNPAVLPEKRPAEITPTKKSAHFFLEIEGFEPNPTVAKTSPPVFSKPMDSNIRQCISGNCDDMDSPQSPQDDVTETPSNPNSPSAQLAKEEQRRKKRRLKKRIFAAVSEGCVEELVELLVELQELCRRRHDEDVPDFLMHKLTASDTGKTCLMKALLNINPNTKEIVRILLAFAEENDILGRFINAEYTEEAYEGQTALNIAIERRQGDIAALLIAAGADVNAHAKGAFFNPKYQHEGFYFGETPLALAACTNQPEIVQLLMEHEQTDITSRDSRGNNILHALVTVAEDFKTQNDFVKRMYDMILLRSGNWELETTRNNDGLTPLQLAAKMGKAEILKYILSREIKEKRLRSLSRKFTDWAYGPVSSSLYDLTNVDTTTDNSVLEITVYNTNIDNRHEMLTLEPLHTLLHMKWKKFAKHMFFLSFCFYFFYNITLTLVSYYRPREEEAIPHPLALTHKMGWLQLLGRMFVLIWAMCISVKEGIAIFLLRPSDLQSILSDAWFHFVFFIQAVLVILSVFLYLFAYKEYLACLVLAMALGWANMLYYTRGFQSMGMYSVMIQKVILHDVLKFLFVYIVFLLGFGVALASLIEKCPKDNKDCSSYGSFSDAVLELFKLTIGLGDLNIQQNSKYPILFLFLLITYVILTFVLLLNMLIALMGETVENVSKESERIWRLQRARTILEFEKMLPEWLRSRFRMGELCKVAEDDFRLCLRINEVKWTEWKTHVSFLNEDPGPVRRTDFNKIQDSSRNNSKTTLNAFEEVEEFPETSV

SEQ ID NO: 23 - DNA, 2373 bp, Homo sapiens
  CDS (1)...(2373)
atgaaagcccaccccaaggagatggtgcctctcatgggcaagagagttgctgcccccagtgggaaccctgccgtcctgccagagaagaggccggcggagatcacccccacaaagaagagtgcacacttcttcctggagatagaagggtttgaacccaaccccacagttgccaagacctctcctcctgtcttctccaagcccatggattccaacatccggcagtgcatctctggtaactgtgatgacatggactccccccagtctcctcaagatgatgtgacagagaccccatccaatcccaacagccccagtgcacagctggccaaggaagagcagaggaggaaaaagaggcggctgaagaagcgcatctttgcagccgtgtctgagggctgcgtggaggagttggtagagttgctggtggagctgcaggagctttgcaggcggcgccatgatgaggatgtgcctgacttcctcatgcacaagctgacggcctccgacacggggaagacctgcctgatgaaggccttgttaaacatcaaccccaacaccaaggagatcgtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgcccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgtgtttttgcttggatttggagtagccttggcctcgctgatcgagaagtgtcccaaagacaacaaggactgcagctcctacggcagcttcagtgacgcagtgctggaactcttcaagctcaccataggcctgggtgatctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaatggaagacgcacgtctccttccttaacgaagacccggggcctgtaagacgaacagatttcaacaaaatccaagattcttccaggaacaacagcaaaaccactctcaatgcatttgaagaagtcgaggaattcccggaaacctcggtgtag

SEQ ID NO: 24 - DNA, 3042 bp, Homo sapiens
  CDS (1)...(3042)
atggttggaggatgcaggtggacagaagacgtggagcctgcagaagtaaaggaaaagatgtcctttcgggcagccaggctcagcatgaggaacagaaggaatgacactctggacagcacccggaccctgtactccagcgcgtctcggagcacagacttgtcttacagtgaaagcgacttggtgaattttattcaagcaaattttaagaaacgagaatgtgtcttctttaccaaagattccaaggccacggagaatgtgtgcaagtgtggctatgcccagagccagcacatggaaggcacccagatcaaccaaagtgagaaatggaactacaagaaacacaccaaggaatttcctaccgacgcctttggggatattcagtttgagacactggggaagaaagggaagtatatacgtctgtcctgcgacacggacgcggaaatcctttacgagctgctgacccagcactggcacctgaaaacacccaacctggtcatttctgtgaccgggggcgccaagaacttcgccctgaagccgcgcatgcgcaagatcttcagccggctcatctacatcgcgcagtccaaaggtgcttggattctcacgggaggcacccattatggcctgatgaagtacctcggggaggtggtgagagataacaccatcagcaggagttcagaggagaatattgtggccattggcatagcagcttggggcatggtctccaaccgggacaccctcatcaggaattgcgatgctgagggctattttttagcccagtaccttatggatgacttcacaagagatccactgtatatcctggacaacaaccacacacatttgctgctcgtggacaatggctgtcatggacatcccactgtcgaagcaaagctccggaatcagctagagaagtatatctctgagcgcactattcaagattccaactatggtggcaagatccccattgtgtgttttgcccaaggaggtggaaaagagactttgaaagccatcaatacctccatcaaaaataaaattccttgtgtggtggtggaaggctcgggccagatcgctgatgtgatcgctagcctggtggaggtggaggatgccctgacatcttctgccgtcaaggagaagctggtgcgctttttaccccgcacggtgtcccggctgcctgaggaggagactgagagttggatcaaatggctcaaagaaattctcgaatgttctcacctattaacagttattaaaatggaagaagctggggatgaaattgtgagcaatgccatctcctacgctctatacaaagccttcagcaccagtgagcaagacaaggataactggaatgggcagctgaagcttctgctggagtggaaccagctggacttagccaatgatgagattttcaccaatgaccgccgatgggagaagagcaaaccgaggctcagagacacaataatccaggtcacatggctggaaaatggtagaatcaaggttgagagcaaagatgtgactgacggcaaagcctcttctcatatgctggtggttctcaagtctgctgaccttcaagaagtcatgtttacggctctcataaaggacagacccaagtttgtccgcctctttctggagaatggcttgaacctacggaagtttctcacccatgatgtcctcactgaactcttctccaaccacttcagcacgcttgtgtaccggaatctgcagatcgccaagaattcctataatgatgccctcctcacgtttgtctggaaactggttgcgaacttccgaagaggcttccggaaggaagacagaaatggccgggacgagatggacatagaactccacgacgtgtctcctattactcggcaccccctgcaagctctcttcatctgggccattcttcagaataagaaggaactctccaaagtcatttgggagcagaccaggggctgcactctggcagccctgggagccagcaagcttctgaagactctggccaaagtgaagaacgacatcaatgctgctggggagtccgaggagctggctaatgagtacctgacccgggctgttggtgagtccacagtgtggaatgctgtggtgggcgcggatctgccatgtggcacagacattgccagcggcactcatagaccagatggtggagagctgttcactgagtgttacagcagcgatgaagacttggcagaacagctgctggtctattcctgtgaagcttggggtggaagcaactgtctggagctggcggtggaggccacagaccagcatttcatcgcccagcctggggtccagaattttctttctaagcaatggtatggagagatttcccgagacaccaagaactggaagattatcctgtgtctgtttattatacccttggtgggctgtggctttgtatcatttaggaagaaacctgtcgacaagcacaagaagctgctttggtactatgtggcgttcttcacctcccccttcgtggtcttctcctggaatgtggtcttctacatcgccttcctcctgctgtttgcctacgtgctgctcatggatttccattcggtgccacacccccccgagctggtcctgtactcgctggtctttgtcctcttctgtgatgaagtgagacagggccggccggctgctcccagtgcggggcccgccaagcccacgcccacccggaactccatctggcccgcaagctccacacgcagccccggttcccgctcacgccactccttccacacttccctgcaagctgagggtgccagctctggccttggccagcccagaaaggggctcccacagtgcagcggtgggctgaagggctcctcaagtgccgccaaagtgggagcccaggcagaggaggtgccgagagcaagcgagggctgtgaggactgccagcacgctgtcacctctcagaagcgtaagacagcaatggaccaaacagacgaagatctcttcccctatggagcattctaccagttcctgatgatttccaggagctttcgaggagaggagatgagcatcggcaagcagcactaa

SEQ ID NO: 25 - protein, 1013 aa, Homo sapiens [sequence omitted]

SEQ ID NO: 26 - DNA, 3042 bp, Homo sapiens; identical to SEQ ID NO: 24
  CDS (1)...(3042)

SEQ ID NO: 27 - DNA, 3448 bp, Homo sapiens
  CDS (425)...(2971)
attaaccttctcttagtcttcaacctaagtacttgaatgtcaagtaccctccaaccctcaatgtcccaagacttttaagagcggaaggtaccgatgagttccatcctttactagggtcaccaaggaaggcatgggtatatggaaatttttattattattccatctgaatatcattttctagagaataggagcttttgttctgaagggctgccggcttccttctgggatctagcagccagggttagatcacaggtgtcactttcaggcgagtagttagcaacggtatcgctagcaactgagccgacccctgcagccagaggtttgcagtgggtagtgtgtattccagaaagggccctgacatgtgaaaggaaggaatgtgccctaatattctacagttgttttatcgttgctactgattaggtccatggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagtttgtggctcatcccaactgccagcagcagctcttgacgatctggtatgagaacctctcaggcctaagggagcagaccatagctatcaagtgtctcgttgtgctggtcgtggccctgggccttccattcctggccattggctactggatcgcaccttgcagcaggctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctctggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagattatatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatatcaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaaggcttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatgatgcagcaacctggatttggctttgactatagcacaaatgtgggcaataatatttctaagtatgaaatacttgaaaaactatgatgtaaatttttagtattaactacctttatcatgtgaacctttaaaagttagctcttaatggttttattgttttatcacatgaaaatgcattttatttgtctgctttgacattacagtggcataccattgtgttgaaaagcccaatattactatattattgaaacttttattcattttagagtaaactccacatctttgcactacctgtttgcctccaagagactatcagttccttggggacagggaccatgtcttattcatctttgtgtctccagcatctagtacagtgcctggtatatagtaggtgctcaataaatgttgaaaccaactgaactgccaacaaaataaaaataaaaagtcttcactatgtagcataaaaaaaaaaaaaaaaaaa

SEQ ID NO: 28 - protein, 848 aa, Homo sapiens
MEGSPSLRRMTVMREKGRRQAVRGPAFMFNDRGTSLTAEEERFLDAAEYGNIPVVRKMLEESKTLNVNCVDYMGQNALQLAVGNEHLEVTELLLKKENLARIGDALLLAISKGYVRIVEAILNHPGFAASKRLTLSPCEQELQDDDFYAYDEDGTRFSPDITPIILAAHCQKYEVVHMLLMKGARIERPHDYFCKCGDCMEKQRHDSFSHSRSRINAYKGLASPAYLSLSSEDPVLTALELSNELAKLANIEKEFKNDYRKLSMQCKDFVVGVLDLCRDSEEVEAILNGDLESAEPLEVHRHKASLSRVKLAIKYEVKKFVAHPNCQQQLLTIWYENLSGLREQTIAIKCLVVLVVALGLPFLAIGYWIAPCSRLGKILRSPFMKFVAHAASFIIFLGLLVFNASDRFEGITTLPNITVTDYPKQIFRVKTTQFTWTEMLIMVWVLGMMWSECKELWLEGPREYILQLWNVLDFGMLSIFIAAFTARFLAFLQATKAQQYVDSYVQESDLSEVTLPPEIQYFTYARDKWLPSDPQIISEGLYAIAVVLSFSRIAYILPANESFGPLQISLGRTVKDIFKFMVLFIMVFFAFMIGMFILYSYYLGAKVNAAFTTVEESFKTLFWSIFGLSEVTSVVLKYDHKFIENIGYVLYGIYNVTMVVVLLNMLIAMINSSYQEIEDDSDVEWKFARSKLWLSYFDDGKTLPPPFSLVPSPKSFVYFIMRIVNFPKCRRRRLQKDIEMGMGNSKSRLNLFTQSNSRVFESHSFNSILNQPTRYQQIMKRLIKRYVLKAQVDKENDEVNEGELKEIKQDISSLRYELLEDKSQATEELAILIHKLSEKLNPSMLRCE

SEQ ID NO: 29 - DNA, 2547 bp, Homo sapiens
  CDS (1)...(2547)
atggagggaagcccatccctgagacgcatgacagtgatgcgggagaagggccggcgccaggctgtcaggggcccggccttcatgttcaatgaccgcggcaccagcctcaccgccgaggaggagcgcttcctcgacgccgccgagtacggcaacatcccagtggtgcgcaagatgctggaggagtccaagacgctgaacgtcaactgcgtggactacatgggccagaacgcgctgcagctggctgtgggcaacgagcacctggaggtgaccgagctgctgctcaagaaggagaacctggcgcgcattggcgacgccctgctgctcgccatcagcaagggctacgtgcgcatcgtagaggccatcctcaaccaccctggcttcgcggccagcaagcgtctcactctgagcccctgtgagcaggagctgcaggacgacgacttctacgcttacgacgaggacggcacgcgcttctcgccggacatcacccccatcatcctggcggcgcactgccagaaatacgaagtggtgcacatgctgctgatgaagggtgccaggatcgagcggccgcacgactatttctgcaagtgcggggactgcatggagaagcagaggcacgactccttcagccactcacgctcgaggatcaatgcctacaaggggctggccagcccggcttacctctcattgtccagcgaggacccggtgcttacggccctagagctcagcaacgagctggccaagctggccaacatagagaaggagttcaagaatgactatcggaagctctccatgcaatgcaaagactttgtagtgggtgtgctggatctctgccgagactcagaagaggtagaagccattctgaatggagatctggaatcagcagagcctctggaggtacacaggcacaaagcttcattaagtcgtgtcaaacttgccattaagtatgaagtcaaaaagtttgtggctcatcccaactgccagcagcagctcttgacgatctggtatgagaacctctcaggcctaagggagcagaccatagctatcaagtgtctcgttgtgctggtcgtggccctgggccttccattcctggccattggctactggatcgcaccttgcagcaggctggggaaaattctgcgaagcccttttatgaagtttgtagcacatgcagcttctttcatcatcttcctgggtctgcttgtgttcaatgcctcagacaggttcgaaggcatcaccacgctgcccaatatcacagttactgactatcccaaacagatcttcagggtgaaaaccacccagtttacatggactgaaatgctaattatggtctgggttcttggaatgatgtggtctgaatgtaaagagctctggctggaaggacctagggaatacattttgcagttgtggaatgtgcttgactttgggatgctgtccatcttcattgctgctttcacagccagattcctagctttccttcaggcaacgaaggcacaacagtatgtggacagttacgtccaagagagtgacctcagtgaagtgacactcccaccagagatacagtatttcacttatgctagagataaatggctcccttctgaccctcagattatatctgaaggcctttatgccatagctgttgtgctcagcttctctcggattgcgtacatcctccctgcaaatgagagctttggccccctgcagatctctcttggaaggactgtaaaggacatattcaagttcatggtcctctttattatggtgttttttgcctttatgattggcatgttcatactttattcttactaccttggggctaaagttaatgctgcttttaccactgtagaagaaagtttcaagactttattttggtcaatatttgggttgtctgaagtgacttccgttgtgctcaaatatgatcacaaattcatagaaaatattggatacgttctttatggaatatacaatgtaactatggtggtcgttttactcaacatgctaattgctatgattaatagctcatatcaagaaattgaggatgacagtgatgtagaatggaagtttgctcgttcaaaactttggttatcctattttgatgatggaaaaacattacctccacctttcagtctagttcctagtccaaaatcatttgtttatttcatcatgcgaattgttaactttcccaaatgcagaaggagaaggcttcagaaggatatagaaatgggaatgggtaactcaaagtccaggttaaacctcttcactcagtctaactcaagagtttttgaatcacacagttttaacagcattctcaatcagccaacacgttatcagcagataatgaaaagacttataaagcggtatgttttgaaagcacaagtagacaaagaaaatgatgaagttaatgaaggtgaattaaaagaaatcaagcaagatatctccagccttcgttatgaacttttggaagacaagagccaagcaactgaggaattagccattctaattcataaacttagtgagaaactgaatcccagcatgctgagatgtgaatga

SEQ ID NO: 30 - protein, 250 aa, Artificial Sequence
  Amino Acid Consensus Sequence
ILFILDLLFVLLFLLEIVLKFIAYGLKSTSNIAAKYLKSIFNILDLLAILPLLLLLVLFLSGTEQVLXXXXXXXXXXXXXXXXXXXXXXRERXFXSLELSQYRILRFLRLLRLLRLLRLLRLLRRLETLFEFELGTLAWSLQSLGRALKSILRFLLLLLLLLIGFSVIGYLLFKGYEDLSENEVDGNSEFSSYFDAFYFLFVTLTTVGFGDLVPVXXXXXXXXXWLGIIFFVLFFIIVGLLLLNLLIAVI

SEQ ID NO: 31 - protein, 33 aa, Artificial Sequence; identical to SEQ ID NO: 9
  Amino Acid Consensus Sequence

SEQ ID NO: 32 - DNA, 3220 bp, Homo sapiens
  CDS (338)...(2476)
tcgggaagcgcgccattgtgttggtacccgggaattcgcggccgcgtcgacccccctgcggagttgtgttttctgggaatcagcaacaaattgcaaagaaatggctcaaaagcttcagctctttctgtgccctgggagctgagatgcacgtcagtggccttgccagcgtggccaattctctgctgactgccagaaaaaagaggccaggaagaaagaggaaagagaagagatcgctcagggtctgtggtgtgtggtccatcctcttgctgagcacattgaaaggaactggctatctttgatctcttcctccagatcagagtcaaggaatgtgtttataatggacacttcatccaaagaaaatatccagttgttctgcaaaacttcagtgcaacctgttggaaggccttcttttaaaacagaatatccctcctcagaagaaaagcaaccatgctgtggtgaactaaaggtgttcttgtgtgccttgtcttttgtttactttgccaaagcattggcagaaggctatctgaagagcaccatcactcagatagagagaaggtttgatatcccttcttcactggtgggagttattggtggtagttttgaaattgggaatctcttagttataacatttgttagctactttggagccaaacttcacaggccaaaaataattggagcagggtgtgtaatcatgggagttggaacactgctcattgcaatgcctcagttcttcatggagcagtacaaatatgagagatattctccttcctccaattctactctcagcatctctccgcgtctcctagagtcaagcagtcaattaccagtttcagttatggaaaaatcaaaatccaaaataagtaacgaatgtgaagtggacactagctcttccatgtggatttatgttttcctgggtaatcttcttcgtggaataggagaaactcccattcagcctttgggcattgcctacctggatgattttgccagtgaagacaatgcagctttctatattgggtgtgtgcagacggttgcaattataggaccaatctttggtttcctgttaggctcattatgtgccaaactatatgttgacattggctttgtaaacctagatcacataaccattacaccaaaagatccccagtgggtaggagcctggtggcttggctatctaatagcaggaatcataagtcttcttgcagctgtgcctttctggtatttaccaaagagtttaccaagatcccaaagtagagaggattctaattcttcctctgagaaatccaagtttattatagatgatcacacagactaccaaacaccccagggagaaaatgcaaaaataatggaaatggcaagagattttcttccatcactgaagaatctttttggaaacccagtatacttcctatatttatgtacaagcactgttcagttcaattctctgttcggcatggtgacgtacaaaccaaagtacattgagcagcagtatggacagtcatcctccagggccaactttgtgatcgggctcatcaacattccagcagtggcccttggaatattctctggggggatagttatgaaaaaattcagaatcagtgtgtgtggagctgcaaaactctacttgggatcatctgtctttggttacctcctatttctttccctgtttgcactgggctgtgaaaattctgatgtggcaggactaactgtctcctaccaaggaaccaaacctgtctcttatcatgaacgagctctcttttcagattgcaactcaagatgcaaatgttcagagacaaaatgggaacccatgtgcggtgaaaatggaatcacatatgtatcagcttgtcttgctggttgtcaaacctccaacaggagtggaaaaaatattatattttacaactgcacttgtgtgggaattgcagcttctaaatccggaaattcctcaggcatagtgggaagatgtcagaaagacaatggatgtccccaaatgtttctgtatttccttgtaatttcagtcatcacatcctatactttatccctaggtggcatacctggatacatattacttctgaggtgcattaagccacagcttaagtcttttgccttgggtatctacacattagcaataagagttcttgcaggaatcccagctccagtgtattttggagttttgattgatacttcatgcctcaaatggggatttaaaagatgtggaagtagaggatcatgcagattatatgattcaaatgtcttcagacatatatatctgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaacatataatggaagatgcagatgataaaactaattttgaactttttaatttatataaattattttatatcacttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtgtattttaataatgatcttaggatggagcagaacatggagaggaagatttcattttaagctcctccttttctttgaaatacaataatttatatagaaatgtgtagcagcaaattatattggggattagaattttgaattaatagctctcctactattaatttacatgtgctttttgtgtggcgctataagtgactatggttgtaaagtaataaaattgatgttaacatgcccaaaaaaaaaaaaaaaaaaaaccaaaaaaaaaaaaaaaaaagggcgggccgctagac

SEQ ID NO: 33 - protein, 712 aa, Homo sapiens
MDTSSKENIQLFCKTSVQPVGRPSFKTEYPSSEEKQPCCGELKVFLCALSFVYFAKALAEGYLKSTITQIERRFDIPSSLVGVIGGSFEIGNLLVITFVSYFGAKLHRPKIIGAGCVIMGVGTLLIAMPQFFMEQYKYERYSPSSNSTLSISPRLLESSSQLPVSVMEKSKSKISNECEVDTSSSMWIYVFLGNLLRGIGETPIQPLGIAYLDDFASEDNAAFYIGCVQTVAIIGPIFGFLLGSLCAKLYVDIGFVNLDHITITPKDPQWVGAWWLGYLIAGIISLLAAVPFWYLPKSLPRSQSREDSNSSSEKSKFIIDDHTDYQTPQGENAKIMEMARDFLPSLKNLFGNPVYFLYLCTSTVQFNSLFGMVTYKPKYIEQQYGQSSSRANFVIGLINIPAVALGIFSGGIVMKKFRISVCGAAKLYLGSSVFGYLLFLSLFALGCENSDVAGLTVSYQGTKPVSYHERALFSDCNSRCKCSETKWEPMCGENGITYVSACLAGCQTSNRSGKNIIFYNCTCVGIAASKSGNSSGIVGRCQKDNGCPQMFLYFLVISVITSYTLSLGGIPGYILLLRCIKPQLKSFALGIYTLAIRVLAGIPAPVYFGVLIDTSCLKWGFKRCGSRGSCRLYDSNVFRHIYLGLTVILGTVSILLSIAVLFILKKNYVSKHRSFITKRERTMVSTRFQKENYTTSDHLLQPNYWPGKETQL

SEQ ID NO: 34 - DNA, 2139 bp, Homo sapiens
  CDS (1)...(2139)
atggacacttcatccaaagaaaatatccagttgttctgcaaaacttcagtgcaacctgttggaaggccttcttttaaaacagaatatccctcctcagaagaaaagcaaccatgctgtggtgaactaaaggtgttcttgtgtgccttgtcttttgtttactttgccaaagcattggcagaaggctatctgaagagcaccatcactcagatagagagaaggtttgatatcccttcttcactggtgggagttattggtggtagttttgaaattgggaatctcttagttataacatttgttagctactttggagccaaacttcacaggccaaaaataattggagcagggtgtgtaatcatgggagttggaacactgctcattgcaatgcctcagttcttcatggagcagtacaaatatgagagatattctccttcctccaattctactctcagcatctctccgcgtctcctagagtcaagcagtcaattaccagtttcagttatggaaaaatcaaaatccaaaataagtaacgaatgtgaagtggacactagctcttccatgtggatttatgttttcctgggtaatcttcttcgtggaataggagaaactcccattcagcctttgggcattgcctacctggatgattttgccagtgaagacaatgcagctttctatattgggtgtgtgcagacggttgcaattataggaccaatctttggtttcctgttaggctcattatgtgccaaactatatgttgacattggctttgtaaacctagatcacataaccattacaccaaaagatccccagtgggtaggagcctggtggcttggctatctaatagcaggaatcataagtcttcttgcagctgtgcctttctggtatttaccaaagagtttaccaagatcccaaagtagagaggattctaattcttcctctgagaaatccaagtttattatagatgatcacacagactaccaaacaccccagggagaaaatgcaaaaataatggaaatggcaagagattttcttccatcactgaagaatctttttggaaacccagtatacttcctatatttatgtacaagcactgttcagttcaattctctgttcggcatggtgacgtacaaaccaaagtacattgagcagcagtatggacagtcatcctccagggccaactttgtgatcgggctcatcaacattccagcagtggcccttggaatattctctggggggatagttatgaaaaaattcagaatcagtgtgtgtggagctgcaaaactctacttgggatcatctgtctttggttacctcctatttctttccctgtttgcactgggctgtgaaaattctgatgtggcaggactaactgtctcctaccaaggaaccaaacctgtctcttatcatgaacgagctctcttttcagattgcaactcaagatgcaaatgttcagagacaaaatgggaacccatgtgcggtgaaaatggaatcacatatgtatcagcttgtcttgctggttgtcaaacctccaacaggagtggaaaaaatattatattttacaactgcacttgtgtgggaattgcagcttctaaatccggaaattcctcaggcatagtgggaagatgtcagaaagacaatggatgtccccaaatgtttctgtatttccttgtaatttcagtcatcacatcctatactttatccctaggtggcatacctggatacatattacttctgaggtgcattaagccacagcttaagtcttttgccttgggtatctacacattagcaataagagttcttgcaggaatcccagctccagtgtattttggagttttgattgatacttcatgcctcaaatggggatttaaaagatgtggaagtagaggatcatgcagattatatgattcaaatgtcttcagacatatatatctgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttag

SEQ ID NO: 35 - DNA, 2866 bp, Homo sapiens
  CDS (365)...(2026)
  misc_feature (1)...(2866): n = A,T,C or G
aagtgtcaccggtcacactttaattccagtctaaaattaaagtcttcagtctccacattccctactttccaaattcagctttcccgggaggtctggagcagctgcctctctggggagatgctggaggtctcggaatcacctcacacggcctcagggcccagttggagccaccccaagtgacaccagcaggcagatgaccagagagcctgagcctccggccccgagtctgtgaagcctagccgctgggctggagaagccactgtgggcaccaccgtgggggaaacaggcccgttgccctggcctctttgccctgggccagcctttgtgaagtgggcccctcttctgggccccttgagtaggttccatggcattttctgaactcctggacctcgtgggtggcctgggcaggttccaggttctccagacgatggctctgatggtctccatcatgtggctgtgtacccagagcatgctggagaacttctcggccgccgtgcccagccaccgctgctgggcacccctcctggacaacagcacggctcaggccagcatcctagggagcttgagtcctgaggccctcctggctatttccatcccgccgggccccaaccagaggccccaccagtgccgccgcttccgccagccacagtggcagctcttggaccccaatgccacggccaccagctggagcgaggccgacacggagccgtgtgtggatggctgggtctatgaccgcagcatcttcacctccacaatcgtggccaagtggaacctcgtgtgtgactctcatgctctgaagcccatggcccagtccatctacctggctgggattctggtgggagctgctgcgtgcggccctgcctcagacaggtttgggcgcaggctggtgctaacctggagctaccttcagatggctgtgatgggtacggcagctgccttcgcccctgccttccccgtgtactgnntgntccgcttcctgttggccttccacgcgtccggggctgaaccactcggtctccttgcagtgatggagtggacggcggcacgggcccgacccttggtgatgaccttgaactctctgggcttcagcttcggccatggcctgacagctgcagtggcctacggtgtgcgggactggacactgctgcagctggtggtctcggtccccttcttcctctgctttttgtactcctggtggctggcagagtcggcacgatggctcctcaccacaggcaggctggattggggcctgcaggagctgtggagggtggctcccatcaacggaaagggggcagtgcaggacaccctgacccctgaggtcttgctttcagccatgcgggaggagctgagcatgggccagcctcctgccagcctgggcaccctgctccgcatgcccggactgcgcttccggacctgtatctccacgttgtgctggttcgcctttggcttcaccttcttcggcctggccctggacctgcaggccctgggcagcaacatcttcctgctccaaatgttcattggtgtcgtggacatcccagccaagatgggcgccctgctgctgctgagccacctgggccgccgccccacgctggccgcatccctgttgctggcggggctctgcattctggccaacacgctggtgccccacgaaatgggggctctgcgctcagccttggccgtgctggggctgggcggggtgggggctgccttcacctgcatcaccatctacagcagcgagctcttccccactgtgctcaggatgacggcagtgggcttgggccagatggcagcccgtggaggagccatcctggggcctctggtccggctgctgggtgtccatggcccctggctgcccttgctggtgtatgggacggtgccagtgctgagtggcctggccgcactgcttctgcccgagacccagagcttgccgctgcccgacaccatccaagatgtgcagaaccaggcagtaaagaaggcaacacatggcacgctggggaactctgtcctaaaatccacacagttttagcctcctggggaacctgcgatgggacggtcagaggaagagacttcttctgttctctggagaaggcaggaggaaagcaaagacctccatttccagaggcccagaggctgccctctgaggtccccactctcccccagggctgcccctccaggtgagccctgcccctctcacagtccaaggggcccccttcaatactgaaggggaaaaggacagtttgattggcaggaggtgacccagtgcaccatcaccctgccctgccctcgtggcttcggagagcagaggggtcaggcccaggggaacgagctggccttgccaaccctctgcttgactccgcactgccacttgtccccccacacccgtccacctgcccagagctcagagctaaccaccatccatggtcaagacctctcctagctccacacaagcagtagagtctcagctccacagctttacccagaagccctgtaagcctggcccctggcccctccccatgtccctccaggcctcagccacctgcccaccacatcctctgcctgctgtccccttcccaccctcatccctgaccgactccacttaacccccaaacccagccccccttccaggggtccagggccagcctgagatgcccgtgaaactcctacccacagttacagccacaagcctgcctcctcccaccctgccagcctatgagttcccagagggttggggcagtcccatgaccccatgtcccagctccccacacagcgctgggccagagaggcattggtgcgagggattgaataaagaaacaaatgaaaaaaaaaaaaaaaaaaaagcattgcggccgcaagctttttcccttta

SEQ ID NO: 36 - protein, 553 aa, Homo sapiens
  VARIANT (200)...(202): Xaa = Any Amino Acid
MAFSELLDLVGGLGRFQVLQTMALMVSIMWLCTQSMLENFSAAVPSHRCWAPLLDNSTAQASILGSLSPEALLAISIPPGPNQRPHQCRRFRQPQWQLLDPNATATSWSEADTEPCVDGWVYDRSIFTSTIVAKWNLVCDSHALKPMAQSIYLAGILVGAAACGPASDRFGRRLVLTWSYLQMAVMGTAAAFAPAFPVYXXXRFLLAFHASGAEPLGLLAVMEWTAARARPLVMTLNSLGFSFGHGLTAAVAYGVRDWTLLQLVVSVPFFLCFLYSWWLAESARWLLTTGRLDWGLQELWRVAPINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQF

SEQ ID NO: 37 - DNA, 1662 bp, Homo sapiens
  CDS (1)...(1662)
  misc_feature (1)...(1662): n = A,T,C or G
atggcattttctgaactcctggacctcgtgggtggcctgggcaggttccaggttctccagacgatggctctgatggtctccatcatgtggctgtgtacccagagcatgctggagaacttctcggccgccgtgcccagccaccgctgctgggcacccctcctggacaacagcacggctcaggccagcatcctagggagcttgagtcctgaggccctcctggctatttccatcccgccgggccccaaccagaggccccaccagtgccgccgcttccgccagccacagtggcagctcttggaccccaatgccacggccaccagctggagcgaggccgacacggagccgtgtgtggatggctgggtctatgaccgcagcatcttcacctccacaatcgtggccaagtggaacctcgtgtgtgactctcatgctctgaagcccatggcccagtccatctacctggctgggattctggtgggagctgctgcgtgcggccctgcctcagacaggtttgggcgcaggctggtgctaacctggagctaccttcagatggctgtgatgggtacggcagctgccttcgcccctgccttccccgtgtactgnntgntccgcttcctgttggccttccacgcgtccggggctgaaccactcggtctccttgcagtgatggagtggacggcggcacgggcccgacccttggtgatgaccttgaactctctgggcttcagcttcggccatggcctgacagctgcagtggcctacggtgtgcgggactggacactgctgcagctggtggtctcggtccccttcttcctctgctttttgtactcctggtggctggcagagtcggcacgatggctcctcaccacaggcaggctggattggggcctgcaggagctgtggagggtggctcccatcaacggaaagggggcagtgcaggacaccctgacccctgaggtcttgctttcagccatgcgggaggagctgagcatgggccagcctcctgccagcctgggcaccctgctccgcatgcccggactgcgcttccggacctgtatctccacgttgtgctggttcgcctttggcttcaccttcttcggcctggccctggacctgcaggccctgggcagcaacatcttcctgctccaaatgttcattggtgtcgtggacatcccagccaagatgggcgccctgctgctgctgagccacctgggccgccgccccacgctggccgcatccctgttgctggcggggctctgcattctggccaacacgctggtgccccacgaaatgggggctctgcgctcagccttggccgtgctggggctgggcggggtgggggctgccttcacctgcatcaccatctacagcagcgagctcttccccactgtgctcaggatgacggcagtgggcttgggccagatggcagcccgtggaggagccatcctggggcctctggtccggctgctgggtgtccatggcccctggctgcccttgctggtgtatgggacggtgccagtgctgagtggcctggccgcactgcttctgcccgagacccagagcttgccgctgcccgacaccatccaagatgtgcagaaccaggcagtaaagaaggcaacacatggcacgctggggaactctgtcctaaaatccacacagttttag

SEQ ID NO: 38 - DNA, 2480 bp, Homo sapiens
  CDS (148)...(2307)
gtcgacccacgcgtccgggcggcaagagccccttgtggccaccgagtcctccgacgccctcgccaggctggcctttgggttggcccaggcaggacgggcagccgagagcactcgggccgcgtcgccaggagccgcccagggtgagccatgttcgtaggcgtcgcccggcactctgggagccaggatgaagtctcaaggggagtagagccgctggaggccgcgcgggcccagcctgctaaggacaggagggccaagggaaccccgaagtcctcgaagcccgggaaaaaacaccggtatctgagactacttccagaggccttgataaggttcggcggtttccgaaaaaggaaaaaagccaagtcctcagtttccaagaagccgggagaagtggatgacagtttggagcagccctgtggtttgggctgcttagtcagcacctgctgtgggtgttgcaataacattcgctgcttcatgattttctactgcatcctgctcatatgtcaaggtgtggtgtttggtcttatagatgtcagcattggcgattttcagaaggaatatcaactgaaaaccattgagaagttggcattggaaaagagttacgatatttcatctggcctggtagcaatatttatagcattctatggagacagaaaaaaagtaatatggtttgtagcttcctcctttttaataggacttggatcacttttatgtgcttttccatccattaatgaagaaaataaacaaagtaaggtaggaattgaagatatttgcgaagaaataaaggttgtcagtggttgccagagcagtggtatatcattccaatcaaaatacctgtctttcttcatccttgggcagactgtgcagggaatagcaggaatgcctctttatatccttggaataacctttattgatgagaatgttgctacacactcagctggtatctatttaggtattgcagaatgtacatcaatgattggatatgctctgggttatgtgctaggagcaccactagttaaagtccctgagaatactacttctgcaacaaacactacagtcaataatggtagtccagaatggctatggacttggtggattaattttctttttgccgctgtcgttgcatggtgtacattaataccattgtcatgctttccaaacaatatgccaggttcaacacggataaaagctaggaaacgtaaacagcttcatttttttgacagcagacttaaagatctgaaacttggaactaatatcaaggatttatgtgctgctctttggattctgatgaagaatccagtgctcatatgcctagctctgtcaaaagctacagaatatttagttattattggagcttctgaatttttgcctatatatttagaaaatcagtttatattaacacccactgtggcaactacacttgcaggacttgttttaattccaggaggtgcacttggccagcttctgggaggtgtcattgtttccacattagaaatgtcttgtaaagcccttatgagatttataatggttacatctgtgatatcacttatactgcttgtgtttattatttttgtacgctgtaatccagtgcaatttgctgggatcaatgaagattatgatggaacagggaagttgggaaacctcacggctccttgcaatgaaaaatgtagatgctcatcttcaatttattcttctatatgtggaagagatgatattgaatatttttctccctgctttgcagggtgtacatattctaaagcacaaaaccaaaaaaagatgtactacaattgttcttgcattaaagaaggattaataactgcagatgcagaaggtgattttattgatgccagacccgggaaatgtgatgcaaagtgctataagttacctttgttcattgcttttatcttttctacacttatattttctggtttttctggtgtaccaatcgtcttggccatgacgcgggttgtacctgacaaactgcgttctctggccttgggtgtaagctatgtgattttgagaatatttgggactattcctggaccatcaatctttaaaatgtcaggagaaacttcttgtattttacgggatgttaataaatgtggacacagaggacgttgttggatatataacaagacaaaaatggctttcttattggtaggaatatgttttctttgcaaactatgcactatcatcttcactactattgcatttttcatatacaaacgtcgtctaaatgagaacactgacttcccagatgtaactgtgaagaatccaaaagttaagaaaaaagaagaaactgacttgtaactggatcatcattgtattctccaagatttgtttctgtgcccaactttcagaagaggaaaatcacacattatgtttacataagtagcaaaaatatatttatggtgatctgcattttcataataaagtgtcctattgtgaaacaaaaaaaaaaaaaaaaaaaaaagggcggccgc

SEQ ID NO: 39 - protein, 719 aa, Homo sapiens
MFVGVARHSGSQDEVSRGVEPLEAARAQPAKDRRAKGTPKSSKPGKKHRYLRLLPEALIRFGGFRKRKKAKSSVSKKPGEVDDSLEQPCGLGCLVSTCCGCCNNIRCFMIFYCILLICQGVVFGLIDVSIGDFQKEYQLKTIEKLALEKSYDISSGLVAIFIAFYGDRKKVIWFVASSFLIGLGSLLCAFPSINEENKQSKVGIEDICEEIKVVSGCQSSGISFQSKYLSFFILGQTVQGIAGMPLYILGITFIDENVATHSAGIYLGIAECTSMIGYALGYVLGAPLVKVPENTTSATNTTVNNGSPEWLWTWWINFLFAAVVAWCTLIPLSCFPNNMPGSTRIKARKRKQLHFFDSRLKDLKLGTNIKDLCAALWILMKNPVLICLALSKATEYLVIIGASEFLPIYLENQFILTPTVATTLAGLVLIPGGALGQLLGGVIVSTLEMSCKALMRFIMVTSVISLILLVFIIFVRCNPVQFAGINEDYDGTGKLGNLTAPCNEKCRCSSSIYSSICGRDDIEYFSPCFAGCTYSKAQNQKKMYYNCSCIKEGLITADAEGDFIDARPGKCDAKCYKLPLFIAFIFSTLIFSGFSGVPIVLAMTRVVPDKLRSLALGVSYVILRIFGTIPGPSIFKMSGETSCILRDVNKCGHRGRCWIYNKTKMAFLLVGICFLCKLCTIIFTTIAFFIYKRRLNENTDFPDVTVKNPKVKKKEETDL

SEQ ID NO: 40 - DNA, 2160 bp, Homo sapiens
  CDS (1)...(2160)
atgttcgtaggcgtcgcccggcactctgggagccaggatgaagtctcaaggggagtagagccgctggaggccgcgcgggcccagcctgctaaggacaggagggccaagggaaccccgaagtcctcgaagcccgggaaaaaacaccggtatctgagactacttccagaggccttgataaggttcggcggtttccgaaaaaggaaaaaagccaagtcctcagtttccaagaagccgggagaagtggatgacagtttggagcagccctgtggtttgggctgcttagtcagcacctgctgtgggtgttgcaataacattcgctgcttcatgattttctactgcatcctgctcatatgtcaaggtgtggtgtttggtcttatagatgtcagcattggcgattttcagaaggaatatcaactgaaaaccattgagaagttggcattggaaaagagttacgatatttcatctggcctggtagcaatatttatagcattctatggagacagaaaaaaagtaatatggtttgtagcttcctcctttttaataggacttggatcacttttatgtgcttttccatccattaatgaagaaaataaacaaagtaaggtaggaattgaagatatttgcgaagaaataaaggttgtcagtggttgccagagcagtggtatatcattccaatcaaaatacctgtctttcttcatccttgggcagactgtgcagggaatagcaggaatgcctctttatatccttggaataacctttattgatgagaatgttgctacacactcagctggtatctatttaggtattgcagaatgtacatcaatgattggatatgctctgggttatgtgctaggagcaccactagttaaagtccctgagaatactacttctgcaacaaacactacagtcaataatggtagtccagaatggctatggacttggtggattaattttctttttgccgctgtcgttgcatggtgtacattaataccattgtcatgctttccaaacaatatgccaggttcaacacggataaaagctaggaaacgtaaacagcttcatttttttgacagcagacttaaagatctgaaacttggaactaatatcaaggatttatgtgctgctctttggattctgatgaagaatccagtgctcatatgcctagctctgtcaaaagctacagaatatttagttattattggagcttctgaatttttgcctatatatttagaaaatcagtttatattaacacccactgtggcaactacacttgcaggacttgttttaattccaggaggtgcacttggccagcttctgggaggtgtcattgtttccacattagaaatgtcttgtaaagcccttatgagatttataatggttacatctgtgatatcacttatactgcttgtgtttattatttttgtacgctgtaatccagtgcaatttgctgggatcaatgaagattatgatggaacagggaagttgggaaacctcacggctccttgcaatgaaaaatgtagatgctcatcttcaatttattcttctatatgtggaagagatgatattgaatatttttctccctgctttgcagggtgtacatattctaaagcacaaaaccaaaaaaagatgtactacaattgttcttgcattaaagaaggattaataactgcagatgcagaaggtgattttattgatgccagacccgggaaatgtgatgcaaagtgctataagttacctttgttcattgcttttatcttttctacacttatattttctggtttttctggtgtaccaatcgtcttggccatgacgcgggttgtacctgacaaactgcgttctctggccttgggtgtaagctatgtgattttgagaatatttgggactattcctggaccatcaatctttaaaatgtcaggagaaacttcttgtattttacgggatgttaataaatgtggacacagaggacgttgttggatatataacaagacaaaaatggctttcttattggtaggaatatgttttctttgcaaactatgcactatcatcttcactactattgcatttttcatatacaaacgtcgtctaaatgagaacactgacttcccagatgtaactgtgaagaatccaaaagttaagaaaaaagaagaaactgacttgtaa

SEQ ID NO: 41 - protein, 61 aa, Artificial Sequence
  Amino Acid Consensus Sequence
CSRYPNPTSKDGKLVACPREYDPVCGSDGVTYSNECELKKAACAENVEQGTNIEKKHDGPC

SEQ ID NO: 42 - protein, 58 aa, Artificial Sequence
  Amino Acid Consensus Sequence
DCSEYRSPTSGGLLACPREYDPVCGSDGVTYSNECELCAAACEAEQGKSIEVKHDGPC

SEQ ID NO: 43 - protein, 192 aa, Artificial Sequence
  Amino Acid Consensus Sequence
CNEQCSCETSTWHPVCGDDNGLAYYSPCHAGCSETNQSSGTGTNMVFTNCSCVQTSGNSSAVVKKCCKNPECQNKLQYFLILMIFGSFIYSLAAVPGYMVILRCVNPEEKSLALGLHWFCVRIFGTIPAPIIFGLLIDMSCLHWNKQCCGERGSCRMYDNDSLRNMYHGLTVALRVISLIPAFFVWWLMKKN

SEQ ID NO: 44 - protein, 712 aa, Homo sapiens
MDTSSKENIQLFCKTSVQPVGRPSFKTEYPSSEEKQPCCGELKVFLCALSFVYFAKALAEGYLKSTITQIERRFDIPSSLVGVIDGSFEIGNLLVITFVSYFGAKLHRPKIIGAGCVIMGVGTLLIAMPQFFMEQYKYERYSPSSNSTLSISPCLLESSSQLPVSVMEKSKSKISNECEVDTSSSMWIYVFLGNLLRGIGETPIQPLGIAYLDDFASEDNAAFYIGCVQTVAIIGPIFGFLLGSLCAKLYVDIGFVNLDHITITPKDPQWVGAWWLGYLIAGIISLLAAVPFWYLPKSLPRSQSREDSNSSSEKSKFIIDDHTDYQTPQGENAKIMEMARDFLPSLKNLFGNPVYFLYLCTSTVQFNSLFGMVTYKPKYIEQQYGQSSSRANFVIGLINIPAVALGIFSGGIVMKKFRISVCGAAKLYLGSSVFGYLLFLSLFALGCENSDVAGLTVSYQGTKPVSYHERALFSDCNSRCKCSETKWEPMCGENGITYVSACLAGCQTSNRSGKNIIFYNCTCVGIAASKSGNSSGIVGRCQKDNGCPQMFLYFLVISVITSYTLSLGGIPGYILLLRCIKPQLKSFALGIYTLAIRVLAGIPAPVYFGVLIDTSCLKWGFKRCGSRGSCRLYDSNVFRHIYLGLTVILGTVSILLSIAVLFILKKNYVSKHRSFITKRERTMVSTRFQKENYTTSDHLLQPNYWPGKETQL

SEQ ID NO: 45 - protein, 487 aa, Artificial Sequence
  Amino Acid Consensus Sequence
VALVAALGGGFLFGYDTGVIGGFLALIDFLFRFGLLTSSGALAELGYSTVLTGLVVSIFFLGRLIGSLFAGKLGDRFGRKKSLLIALVLFVIGALLSGAAPGYTTIGLWAFYLLIVGRVLVGLGVGGASVLVPMYISEIAPKALRGALGSLYQLAITIGILVAAIIGLGLNKTNNDSALNSWGWRIPLGLQLVPALLLLIGLLFLPESPRWLVEKGKLEEAREVLAKLRGVEDVDQEIQEIKAELEAGVEEEKAGKASWGELFRGRTRPKVRQRLLMGVMLQAFQQLTGINAIFYYSPTIFKSVGVSDSRASLLVTIIVGVVNFVFTLVALIFLVDRFGRRPLLLLGAAGMAICFLILGASIGVALLLLNKPKDPLSKAAGIVAIVFILLFIAFFALGWGPIPWVILSELFPTKVRSKALALATAANWLANFIIGFLFPYITGAIGLALGGYVFLVFAGLLVLFILFVFFFVPETKGRTLEEIEELF

SEQ ID NO: 46 - protein, 44 aa, Artificial Sequence
  Amino Acid Consensus Sequence
NGTEPNGSDPVTEPCSDGWVYDHSTFPSTIVTEWDLVCNHRALR

SEQ ID NO: 47 - protein, 550 aa, Homo sapiens
MAFSKLLEQAGGVGLFQTLQVLTFILPCLMIPSQMLLENFSAAIPGHRCWTHMLDNGSAVSTNMTPKALLTISIPPGPNQGPHQCRRFRQPQWQLLDPNATATSWSEADTEPCVDGWVYDRSVFTSTIVAKWDLVCSSQGLKPLSQSIFMSGILVGSFIWGLLSYRFGRKPMLSWCCLQLAVAGTSTIFAPTFVIYCGLRFVAAFGMAGIFLSSLTLMVEWTTTSRRAVTMTVVGCAFSAGQAALGGLAFALRDWRTLQLAASVPFFAISLISWWLPESARWLIIKGKPDQALQELRKVARINGHKEAKNLTIEVLMSSVKEEVASAKEPRSVLDLFCVPVLRWRSCAMLVVNFSLLISYYGLVFDLQSLGRDIFLLQALFGAVDFLGRATTALLLSFLGRRTIQAGSQAMAGLAILANMLVPQDLQTLRVVFAVLGKGCFGISLTCLTIYKAELFPTPVRMTADGILHTVGRLGAMMGPLILMSRQALPLLPPLLYGVISIASSLVVLFFLPETQGLPLPDTIQDLESQKSTAAQGNRQEAVTVESTSL

SEQ ID NO: 48 - protein, 553 aa, Mus musculus
MAFPELLDRVGGLGRFQLFQTVALVTPILWVTTQNMLENFSAAVPHHRCWVPLLDNSTSQASIPGDLGPDVLLAVSIPPGPDQQPHQCLRFRQPQWQLTESNATATNWSDAATEPCEDGWVYDHSTFRSTIVTTWDLVCNSQALRPMAQSIFLAGILVGAAVCGHASDRFGRRRVLTWSYLLVSVSGTAAAFMPTFPLYCLFRFLLASAVAGVMMNTASLLMEWTSAQGSPLVMTLNALGFSFGQVLTGSVAYGVRSWRMLQLAVSAPFFLFFVYSWWLPESARWLITVGKLDQGLQELQRVAAVNRRKAEGDTLTMEVLRSAMEEEPSRDKAGASLGTLLHTPGLRHRTIISMLCWFAFGFTFYGLALDLQALGSNIFLLQALIGIVDFPVKTGSLLLISRLGRRLCQVSFLVLPGLCILSNILVPHGMGVLRSALAVLGLGCLGGAFTCITIFSSELFPTVIRMTAVGLCQVAARGGAMLGPLVRLLGVYGSWMPLLVYGVVPVLSGLAALLLPETKNLPLPDTIQDIQKQSVKKVTHDTPDGSILMSTRL

SEQ ID NO: 49 - protein, 722 aa, Homo sapiens
MPLHQLGDKPLTFPSPNSAMENGLDHTPPSRRASPGTPLSPGSLRSAAHSPLDTSKQPLCQLWAEKHGARGTHEVRYVSAGQSVACGWWAFAPPCLQVLNTPKGILFFLCAAAFLQGMTVNGFINTVITSLERRYDLHSYQSGLIASSYDIAACLCLTFVSYFGGSGHKPRWLGWGVLLMGTGSLVFALPHFTAGRYEVELDAGVRTCPANPGAVCADSTSGLSRYQLVFMLGQFLHGVGATPLYTLGVTYLDENVKSSCSPVYIAIFYTAAILGPAAGYLIGGALLNIYTEMGRRTELTTESPLWVGAWWVGFLGSGAAAFFTAVPILGYPRQLPGSQRYAVMRAAEMHQLKDSSRGEASNPDFGKTIRDLPLSIWLLLKNPTFILLCLAGATEATLITGMSTFSPKFLESQFSLSASEAATLFGYLVVPAGGGGTFLGGFFVNKLRLRGSAVIKFCLFCTVVSLLGILVFSLHCPSVPMAGVTASYGGSLLPEGHLNLTAPCNAACSCQPEHYSPVCGSDGLMYFSLCHAGCPAATETNVDGQKVYRDCSCIPQNLSSGFGHATAGKCTSTCQRKPLLLVFIFVVIFFTFLSSIPALTATLRCVRDPQRSFALGIQWIVVRILGGIPGPIAFGWVIDKACLLWQDQCGQQGSCLVYQNSAMSRYILIMGLLYKVLGVLFFAIACFLYKPLSESSDGLETCLPSQSSAPDSATDSQLQSSV

SEQ ID NO: 50 - protein, 9 aa, Artificial Sequence
  Amino Acid Consensus Sequence
XXXXXXXXL

SEQ ID NO: 51 - protein, 18 aa, Artificial Sequence
  Amino Acid Consensus Sequence
XXXXXXXXGRXXXXXXXX

SEQ ID NO: 52 - protein, 26 aa, Artificial Sequence
  Amino Acid Consensus Sequence
XXGXXXGXXXXXXXXXXXXXXXXXXX

SEQ ID NO: 53 - DNA, 3227 bp, Homo sapiens
  CDS (345)...(2483)
ccctttctcgggaagcgcgccattgtgttggtacccgggaattcgcggccgcgtcgacccccctgcggagttgtgttttctgggaatcagcaacaaaytgcaaagaaatggctcaaaagcttcagctctttctgtgccctgggagctgagatgcacgtcagtggccttgccagcgtggccaattctctgctgactgccagaaaaaagaggccaggaagaaagaggaaagagaagagatcgctcagggtctgtggtgtgtggtccatcctcttgctgagcacattgaaaggaactggctatctttgatctcttcctccagatcagagtcaaggaatgtgtttataatggacacttcatccaaagaaaatatccagttgttctgcaaaacttcagtgcaacctgttggaaggccttcttttaaaacagaatatccctcctcagaagaaaagcaaccatgctgtggtgaactaaaggtgttcttgtgtgccttgtcttttgtttactttgccaaagcattggcagaaggctatctgaagagcaccatcactcagatagagagaaggtttgatatcccttcttcactggtgggagttattgatggtagttttgaaattgggaatctcttagttataacatttgttagctactttggagccaaacttcacaggccaaaaataattggagcagggtgtgtaatcatgggagttggaacactgctcattgcaatgcctcagttcttcatggagcagtacaaatatgagagatattctccttcctccaattctactctcagcatctctccgtgtctcctagagtcaagcagtcaattaccagtttcagttatggaaaaatcaaaatccaaaataagtaacgaatgtgaagtggacactagctcttccatgtggatttatgttttcctgggcaatcttcttcgtggaataggagaaactcccattcagcctttgggcattgcctacctggatgattttgccagtgaagacaatgcagctttctatattgggtgtgtgcagacggttgcaattataggaccaatctttggtttcctgttaggctcattatgtgccaaactatatgttgacattggctttgtaaacctagatcacataaccattaccccaaaagatccccagtgggtaggagcctggtggcttggctatctaatagcaggaatcataagtcttcttgcagctgtgcctttctggtatttaccaaagagtttaccaagatcccaaagtagagaggattctaattcttcctctgagaaatccaagtttattatagatgatcacacagactaccaaacaccccagggagaaaatgcaaaaataatggaaatggcaagagattttcttccatcactgaagaatctttttggaaacccagtatacttcctatatttatgtacaagcactgttcagttcaattctctgttcggcatggtgacgtacaaaccaaagtacattgagcagcagtatggacagtcatcctccagggccaactttgtgatcgggctcatcaacattccagcagtggcccttggaatattctctggggggatagttatgaaaaaattcagaatcagtgtgtgtggagctgcaaaactctacttgggatcatctgtctttggttacctcctatttctttccctgtttgcactgggctgtgaaaattctgatgtggcaggactaactgtctcctaccaaggaaccaaacctgtctcttatcatgaacgagctctcttttcagattgcaactcaagatgcaaatgttcagagacaaaatgggaacccatgtgcggtgaaaatggaatcacatatgtatcagcttgtcttgctggttgtcaaacctccaacaggagtggaaaaaatattatattttacaactgcacttgtgtgggaattgcagcttctaaatccggaaattcctcaggcatagtgggaagatgtcagaaagacaatggatgtccccaaatgtttctgtatttccttgtaatttcagtcatcacatcctatactttatccctaggtggcatacctggatacatattacttctgaggtgcattaagccacagcttaagtcttttgccttgggtatctacacattagcaataagagttcttgcaggaatcccagctccagtgtattttggagttttgattgatacttcatgcctcaaatggggatttaaaagatgtggaagtagaggatcatgcagattatatgattcaaatgtcttcagacatatatatctgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttagaaacatgatgactggaagtcatgtcttctaattggttgacattttgcaaacaaataaattgtaatcaaaagagctctaaatttgtaatttctttctcctttcaaaaaatgtctactttgttttggtcctaggcattaggtaatataactgataatatactgaaacatataatggaagatgcagatgataaaactaattttgaactttttaatttatataaattattttatatcacttacttatttcactttattttgctttgtgctcattgatatatattagctgtactcctagaagaacaattgtctctattgtcacacatggttatatttaaagtaatttctgaactgtgtaatgtgtctagagtaagcaaatactgctaacaattaactcataccttgggttccttcaagtattactcctatagtattttctcccatagctgtcttcatctgtgtattttaataatgatcttaggatggagcagaacatggagaggaagatttcattttaagctcctccttttctttgaaatacaataatttatatagaaatgtgtagcagcaaattatattggggattagaattttgaattaatagctctcctactattaatttacatgtgctttttgtgtggcgctataagtgactatggttgtaaagtaataaaattgatgttaacatgcccaaaaaaaaaaaaaaaaaaaaccaaaaaaaaaaaaaaaaaagggcgggccgctagac

SEQ ID NO: 54 - protein, 712 aa, Homo sapiens; identical to SEQ ID NO: 44

SEQ ID NO: 55 - DNA, 2139 bp, Homo sapiens
  CDS (1)...(2139)
atggacacttcatccaaagaaaatatccagttgttctgcaaaacttcagtgcaacctgttggaaggccttcttttaaaacagaatatccctcctcagaagaaaagcaaccatgctgtggtgaactaaaggtgttcttgtgtgccttgtcttttgtttactttgccaaagcattggcagaaggctatctgaagagcaccatcactcagatagagagaaggtttgatatcccttcttcactggtgggagttattgatggtagttttgaaattgggaatctcttagttataacatttgttagctactttggagccaaacttcacaggccaaaaataattggagcagggtgtgtaatcatgggagttggaacactgctcattgcaatgcctcagttcttcatggagcagtacaaatatgagagatattctccttcctccaattctactctcagcatctctccgtgtctcctagagtcaagcagtcaattaccagtttcagttatggaaaaatcaaaatccaaaataagtaacgaatgtgaagtggacactagctcttccatgtggatttatgttttcctgggcaatcttcttcgtggaataggagaaactcccattcagcctttgggcattgcctacctggatgattttgccagtgaagacaatgcagctttctatattgggtgtgtgcagacggttgcaattataggaccaatctttggtttcctgttaggctcattatgtgccaaactatatgttgacattggctttgtaaacctagatcacataaccattaccccaaaagatccccagtgggtaggagcctggtggcttggctatctaatagcaggaatcataagtcttcttgcagctgtgcctttctggtatttaccaaagagtttaccaagatcccaaagtagagaggattctaattcttcctctgagaaatccaagtttattatagatgatcacacagactaccaaacaccccagggagaaaatgcaaaaataatggaaatggcaagagattttcttccatcactgaagaatctttttggaaacccagtatacttcctatatttatgtacaagcactgttcagttcaattctctgttcggcatggtgacgtacaaaccaaagtacattgagcagcagtatggacagtcatcctccagggccaactttgtgatcgggctcatcaacattccagcagtggcccttggaatattctctggggggatagttatgaaaaaattcagaatcagtgtgtgtggagctgcaaaactctacttgggatcatctgtctttggttacctcctatttctttccctgtttgcactgggctgtgaaaattctgatgtggcaggactaactgtctcctaccaaggaaccaaacctgtctcttatcatgaacgagctctcttttcagattgcaactcaagatgcaaatgttcagagacaaaatgggaacccatgtgcggtgaaaatggaatcacatatgtatcagcttgtcttgctggttgtcaaacctccaacaggagtggaaaaaatattatattttacaactgcacttgtgtgggaattgcagcttctaaatccggaaattcctcaggcatagtgggaagatgtcagaaagacaatggatgtccccaaatgtttctgtatttccttgtaatttcagtcatcacatcctatactttatccctaggtggcatacctggatacatattacttctgaggtgcattaagccacagcttaagtcttttgccttgggtatctacacattagcaataagagttcttgcaggaatcccagctccagtgtattttggagttttgattgatacttcatgcctcaaatggggatttaaaagatgtggaagtagaggatcatgcagattatatgattcaaatgtcttcagacatatatatctgggactaactgtgatactgggcacagtgtcaattctcctaagcattgcagtacttttcattttaaagaaaaattatgtttcaaaacacagaagttttataaccaagagagaaagaacaatggtgtctacaagattccaaaaggaaaattacactacaagtgatcatctgctacaacccaactactggccaggcaaggaaactcaactttag

SEQ ID NO: 56 - DNA, 4197 bp, Homo sapiens
  CDS (1)...(3348)
atgaggagactgagtttgtggtggctgctgagcagggtctgtctgctgttgccgccgccctgcgcactggtgctggccggggtgcccagctcctcctcgcacccgcagccctgccagatcctcaagcgcatcgggcacgcggtgagggtgggcgcggtgcacttgcagccctggaccaccgccccccgcgcggccagccgcgctccggacgacagccgagcaggagcccagagggatgagccggagccagggactaggcggtccccggcgccctcgccgggcgcacgctggttggggagcaccctgcatggccgggggccgccgggctcccgtaagcccggggagggcgccagggcggaggccctgtggccacgggacgccctcctatttgccgtggacaacctgaaccgcgtggaagggctgctaccctacaacctgtctttggaagtagtgatggccatcgaggcaggcctgggcgatctgccacttttgcccttctcctcccctagttcgccatggagcagtgaccctttctccttcctgcaaagtgtgtgccataccgtggtggtgcaaggggtgtcggcgctgctcgccttcccccagagccagggcgaaatgatggagctcgacttggtcagcttagtcctgcacattccagtgatcagcatcgtgcgccacgagtttccgcgggagagtcagaatccccttcacctacaactgagtttagaaaattcattaagttctgatgctgatgtcactgtctcaatcctgaccatgaacaactggtacaattttagcttgttgctgtgccaggaagactggaacatcaccgacttcctcctccttacccagaataattccaagttccaccttggttctatcatcaacatcaccgctaacctcccctccacccaggacctcttgagcttcctacagatccagcttgagagtattaagaacagcacacccacagtggtgatgtttggctgcgacatggaaagtatccggcggattttcgaaattacaacccagtttggggtcatgccccctgaacttcgttgggtgctgggagattcccagaatatggaggaactgaggacagagggtctgcccttaggactcattgctcatggaaaaacaacacagtctgtctttgagcactacgtacaagatgctatggagctggtcgcaagagctgtagccacagccaccatgatccaaccagaacttgctctcattcccagcacgatgaactgcatggaggtggaaactacaaatctcacttcaggacaatatttatcaaggtttctagccaataccactttcagaggcctcagtggttccattagagtaaaaggttccaccatcgtcagctcagaaaacaactttttcatctggaatcttcaacatgaccccatgggaaagccaatgtggacccgcttgggcagctggcaggggagaaagattgtcatggactatggaatatggccagagcaggcccagagacacaaaacccacttccaacatccaagtaagctacacttgagagtggttaccctgattgagcatccttttgtcttcacaagggaggtagatgatgaaggcttgtgccctgctggccaactctgtctagaccccatgactaatgactcttccacactggacagcctttttagcagcctccatagcagtaatgatacagtgcccattaaattcaagaagtgctgctatggatattgcattgatctgctggaaaagatagcagaagacatgaactttgacttcgacctctatattgtaggggatggaaagtatggagcatggaaaaatgggcactggactgggctagtgggtgatctcctgagagggactgcccacatggcagtcacttcctttagcatcaatactgcacggagccaggtgatagacttcaccagccctttcttctccaccagcttgggcatcttagtgaggacccgagatacagcagctcccattggagccttcatgtggccactccactggacaatgtggctggggatttttgtggctctgcacatcactgccgtcttcctcactctgtatgaatggaagagtccatttggtttgactcccaaggggcgaaatagaagtaaagtcttctccttttcttcagccttgaacatctgttatgccctcttgtttggcagaacagtggccatcaaacctccaaaatgttggactggaaggtttctaatgaacctttgggccattttctgtatgttttgcctttccacatacacggcaaacttggctgctgtcatggtaggtgagaagatctatgaagagctttctggaatacatgaccccaagttacatcatccttcccaaggattccgctttggaactgtccgagaaagcagtgctgaagattatgtgagacaaagtttcccagagatgcatgaatatatgagaaggtacaatgttccagccacccctgatggagtggagtatctgaagaatgatccagagaaactagacgccttcatcatggacaaagcccttctggattatgaagtgtcaatagatgctgactgcaaacttctcactgtggggaagccatttgccatagaaggatacggcattggcctcccacccaactctccattgaccgccaacatatccgagctaatcagtcaatacaagtcacatgggtttatggatatgctccatgacaagtggtacagggtggttccctgtggcaagagaagttttgctgtcacggagactttgcaaatgggcatcaaacacttctctgggctctttgtgctgctgtgcattggatttggtctgtccattttgaccaccattggtgagcacatagtatacaggctgctgctaccacgaatcaaaaacaaatccaagctgcaatactggctccacaccagccagagattacacagagcaataaatacatcatttatagaggaaaagcagcagcatttcaagaccaaacgtgtggaaaagaggtctaatgtgggaccccgtcagcttaccgtatggaatacttccaatctgagtcatgacaaccgacggaaatacatctttagtgatgaggaaggacaaaaccagctgggcatccagatccaccaggacatccccctccctccaaggagaagagagctccctgccttgcggaccaccaatgggaaagcagactccctaaatgtatctcggaactcagtgatgcaggaactctcagagctcgagaagcagattcaggtgatccgtcaggagctgcagctggctgtgagcaggaaaacggagctggaggagtatcaaaggacaagtcggacttgtgagtcctaggtgaccacactgcttccctttctcagttcctgaccttcctctgagcccttgagacactttgtaatgctcttttgtaactatcgacaaaggtgtggggaagctgaggtctaggtcttcttaaaggtcaagtctgctctccctcgcctaaagtgcagcagcagctcctctcaagctcactctctaggtctccagggtaggagtgtttttctagcaagaatcttagtcaggagtaagctctgtgcgagagatctgtgaataaccagataaccccagctgccgttaaccttttcaccaggtgccacagtaatatttctggtttttagccctttctctgcactaccaacaagagataaaattgttactcacacttatgtcttactgggttgctggttttcatcgtaacacagaacgaggttatctagggttgtagcttttgatacaactccccgatctagatttattcctacattctgaatggggagcaggtaagagcagagcacctcccactgggggtggggtatttaaaaattaactcattagtatcataaacgtcaaggattgattggaccaggcaagagccatgtttttgagaaggttctggatctctgactccatcctgactgtttagtaagagcatgcttacaccctactgtgaaaaggggaggggatgtggtaagcagaaacagaagacaggcagcagaggcattaaaaatgcataccatgctttcagaacaaaagctctgggccagaaaggcaatttggctaaaaaatgaataagactacttctaatgtaactaagcatctccactatggtgtgtgccttttataaaggaaaaaaaaaaaaaaagg

SEQ ID NO: 57 - protein, 1115 aa, Homo sapiens
MRRLSLWWLLSRVCLLLPPPCALVLAGVPSSSSHPQPCQILKRIGHAVRVGAVHLQPWTTAPRAASRAPDDSRAGAQRDEPEPGTRRSPAPSPGARWLGSTLHGRGPPGSRKPGEGARAEALWPRDALLFAVDNLNRVEGLLPYNLSLEVVMAIEAGLGDLPLLPFSSPSSPWSSDPFSFLQSVCHTVVVQGVSALLAFPQSQGEMMELDLVSLVLHIPVISIVRHEFPRESQNPLHLQLSLENSLSSDADVTVSILTMNNWYNFSLLLCQEDWNITDFLLLTQNNSKFHLGSIINITANLPSTQDLLSFLQIQLESIKNSTPTVVMFGCDMESIRRIFEITTQFGVMPPELRWVLGDSQNMEELRTEGLPLGLIAHGKTTQSVFEHYVQDAMELVARAVATATMIQPELALIPSTMNCMEVETTNLTSGQYLSRFLANTTFRGLSGSIRVKGSTIVSSENNFFIWNLQHDPMGKPMWTRLGSWQGRKIVMDYGIWPEQAQRHKTHFQHPSKLHLRVVTLIEHPFVFTREVDDEGLCPAGQLCLDPMTNDSSTLDSLFSSLHSSNDTVPIKFKKCCYGYCIDLLEKIAEDMNFDFDLYIVGDGKYGAWKNGHWTGLVGDLLRGTAHMAVTSFSINTARSQVIDFTSPFFSTSLGILVRTRDTAAPIGAFMWPLHWTMWLGIFVALHITAVFLTLYEWKSPFGLTPKGRNRSKVFSFSSALNICYALLFGRTVAIKPPKCWTGRFLMNLWAIFCMFCLSTYTANLAAVMVGEKIYEELSGIHDPKLHHPSQGFRFGTVRESSAEDYVRQSFPEMHEYMRRYNVPATPDGVEYLKNDPEKLDAFIMDKALLDYEVSIDADCKLLTVGKPFAIEGYGIGLPPNSPLTANISELISQYKSHGFMDMLHDKWYRVVPCGKRSFAVTETLQMGIKHFSGLFVLLCIGFGLSILTTIGEHIVYRLLLPRIKNKSKLQYWLHTSQRLHRAINTSFIEEKQQHFKTKRVEKRSNVGPRQLTVWNTSNLSHDNRRKYIFSDEEGQNQLGIQIHQDIPLPPRRRELPALRTTNGKADSLNVSRNSVMQELSELEKQIQVIRQELQLAVSRKTELEEYQRTSRTCES

SEQ ID NO: 58 - DNA, 3348 bp, Homo sapiens
  CDS (1)...(3348)
atgaggagactgagtttgtggtggctgctgagcagggtctgtctgctgttgccgccgccctgcgcactggtgctggccggggtgcccagctcctcctcgcacccgcagccctgccagatcctcaagcgcatcgggcacgcggtgagggtgggcgcggtgcacttgcagccctggaccaccgccccccgcgcggccagccgcgctccggacgacagccgagcaggagcccagagggatgagccggagccagggactaggcggtccccggcgccctcgccgggcgcacgctggttggggagcaccctgcatggccgggggccgccgggctcccgtaagcccggggagggcgccagggcggaggccctgtggccacgggacgccctcctatttgccgtggacaacctgaaccgcgtggaagggctgctaccctacaacctgtctttggaagtagtgatggccatcgaggcaggcctgggcgatctgccacttttgcccttctcctcccctagttcgccatggagcagtgaccctttctccttcctgcaaagtgtgtgccataccgtggtggtgcaaggggtgtcggcgctgctcgccttcccccagagccagggcgaaatgatggagctcgacttggtcagcttagtcctgcacattccagtgatcagcatcgtgcgccacgagtttccgcgggagagtcagaatccccttcacctacaactgagtttagaaaattcattaagttctgatgctgatgtcactgtctcaatcctgaccatgaacaactggtacaattttagcttgttgctgtgccaggaagactggaacatcaccgacttcctcctccttacccagaataattccaagttccaccttggttctatcatcaacatcaccgctaacctcccctccacccaggacctcttgagcttcctacagatccagcttgagagtattaagaacagcacacccacagtggtgatgtttggctgcgacatggaaagtatccggcggattttcgaaattacaacccagtttggggtcatgccccctgaacttcgttgggtgctgggagattcccagaatatggaggaactgaggacagagggtctgcccttaggactcattgctcatggaaaaacaacacagtctgtctttgagcactacgtacaagatgctatggagctggtcgcaagagctgtagccacagccaccatgatccaaccagaacttgctctcattcccagcacgatgaactgcatggaggtggaaactacaaatctcacttcaggacaatatttatcaaggtttctagccaataccactttcagaggcctcagtggttccattagagtaaaaggttccaccatcgtcagctcagaaaacaactttttcatctggaatcttcaacatgaccccatgggaaagccaatgtggacccgcttgggcagctggcaggggagaaagattgtcatggactatggaatatggccagagcaggcccagagacacaaaacccacttccaacatccaagtaagctacacttgagagtggttaccctgattgagcatccttttgtcttcacaagggaggtagatgatgaaggcttgtgccctgctggccaactctgtctagaccccatgactaatgactcttccacactggacagcctttttagcagcctccatagcagtaatgatacagtgcccattaaattcaagaagtgctgctatggatattgcattgatctgctggaaaagatagcagaagacatgaactttgacttcgacctctatattgtaggggatggaaagtatggagcatggaaaaatgggcactggactgggctagtgggtgatctcctgagagggactgcccacatggcagtcacttcctttagcatcaatactgcacggagccaggtgatagacttcaccagccctttcttctccaccagcttgggcatcttagtgaggacccgagatacagcagctcccattggagccttcatgtggccactccactggacaatgtggctggggatttttgtggctctgcacatcactgccgtcttcctcactctgtatgaatggaagagtccatttggtttgactcccaaggggcgaaatagaagtaaagtcttctccttttcttcagccttgaacatctgttatgccctcttgtttggcagaacagtggccatcaaacctccaaaatgttggactggaaggtttctaatgaacctttgggccattttctgtatgttttgcctttccacatacacggcaaacttggctgctgtcatggtaggtgagaagatctatgaagagctttctggaatacatgaccccaagttacatcatccttcccaaggattccgctttggaactgtccgagaaagcagtgctgaagattatgtgagacaaagtttcccagagatgcatgaatatatgagaaggtacaatgttccagccacccctgatggagtggagtatctgaagaatgatccagagaaactagacgccttcatcatggacaaagcccttctggattatgaagtgtcaatagatgctgactgcaaacttctcactgtggggaagccatttgccatagaaggatacggcattggcctcccacccaactctccattgaccgccaacatatccgagctaatcagtcaatacaagtcacatgggtttatggatatgctccatgacaagtggtacagggtggttccctgtggcaagagaagttttgctgtcacggagactttgcaaatgggcatcaaacacttctctgggctctttgtgctgctgtgcattggatttggtctgtccattttgaccaccattggtgagcacatagtatacaggctgctgctaccacgaatcaaaaacaaatccaagctgcaatactggctccacaccagccagagattacacagagcaataaatacatcatttatagaggaaaagcagcagcatttcaagaccaaacgtgtggaaaagaggtctaatgtgggaccccgtcagcttaccgtatggaatacttccaatctgagtcatgacaaccgacggaaatacatctttagtgatgaggaaggacaaaaccagctgggcatccagatccaccaggacatccccctccctccaaggagaagagagctccctgccttgcggaccaccaatgggaaagcagactccctaaatgtatctcggaactcagtgatgcaggaactctcagagctcgagaagcagattcaggtgatccgtcaggagctgcagctggctgtgagcaggaaaacggagctggaggagtatcaaaggacaagtcggacttgtgagtcctag

SEQ ID NO: 59 - protein, 1115 aa, Rattus norvegicus
MRRLSLWWLLSRVCLLLPPPCALVLAGVPSSSSHPQPCQILKRIGHAVRVGAVHLQPWTTAPRAASRAQEGGRAGAQRDDPESGTWRPPAPSQGARWLGSALHGRGPPGSRKLGEGAGAETLWPRDALLFAVENLNRVEGLLPYNLSLEVVMAIEAGLGDLPLMPFSSPSSPWSSDPFSFLQSVCHTVVVQGVSALLAFPQSQGEMMELDLVSSVLHIPVLSIVRHEFPRESQNPLHLQLSLENSLSSDADVTVSILTMNNWYNFSLLLCQEDWNITDFLLLTENNSKFHLESVINITANLSSTKDLLSFLQVQMDNIRNSTPTMVMFGCDMDSIRQIFEMSTQFGLSPPELHWVLGDSQNVEELRTEGLPLGLIAHGKTTQSVFEYYVQDAMELVARAVATATMIQPELALLPSTMNCMDVKTTNLTSGQYLSRFLANTTFRGLSGSIKVKGSTIISSENNFFIWNLQHDPMGKPMWTRLGSWQGGRIVMDSGIWPEQAQRHKTHFQHPNKLHLRVVTLIEHPFVFTREVDDEGLCPAGQLCLDPMTNDSSMLDRLFSSLHSSNDTVPIKFKKCCYGYCIDLLEQLAEDMNFDFDLYIVGDGKYGAWKNGHWTGLVGDLLSGTANMAVTSFSINTARSQVIDFTSPFFSTSLGILVRTRDTAAPIGAFMWPLHWTMWLGIFVALHITAIFLTLYEWKSPFGMTPKGRNRNKVFSFSSALNVCYALLFGRTAAIKPPKCWTGRFLMNLWAIFCMFCLSTYTANLAAVMVGEKIYEELSGIHDPKLHHPSQGFRFGTVRESSAEDYVRQSFPEMHEYMRRYNVPATPDGVQYLKNDPEKLDAFIMDKALLDYEVSIDADCKLLTVGKPFAIEGYGIGLPPNSPLTSNISELISQYKSHGFMDVLHDKWYKVVPCGKRSFAVTETLQMGIKHFSGLFVLLCIGFGLSILTTIGEHIVHRLLLPRIKNKSKLQYWLHTSQRFHRALNTSFVEEKQPRSKTKRVEKRSNLGPQQLMVWNTSNLSHDNQRKYIFNDEEGQNQLGTQAHQDIPLPQRRRELPASLTTNGKADSLNVTRSSVIQELSELEKQIQVIRQELQLAVSRKTELEEYQKTNRTCES

SEQ ID NO: 60 - DNA, 2247 bp, Homo sapiens
  CDS (51)...(2048)
  misc_feature (1)...(2247): n = A,T,C or G
gcacgaggcaaaggacctccagaccagagccagccagcagcaaaaagagcatggagctgaggagtacagcagcccccagagctgagggctacagcaacgtgggcttccagaatgaagaaaactttcttgagaacgagaacacatcaggaaacaactcaataagaagcagagctgtgcaaagcagggagcacacaaacaccaaacaggatgaagaacaggtcacagttgagcaggattttccaagaaacagagaacacatggaggatgatgatgaggagatgcaacaaaaagggtgtttggaaaggaggtatgacacagtatgtggtttctgtaggaaacacaaaacaactcttcggcacatcatctggggcattttattagcaggttatctggttatggtgatttcggcctgtgtgctgaactttcacagagcccttcctctttttgtgatcaccgtggctgccatcttctttgttgtctgggatcacctgatggccaaatacgaacatcgaattgatgagatgctgtctcctggcagaaggcttctaaacagccattggttctggctgaagtgggtgatctggagctccctggtcctagcagttattttctggttggcctttgacactgccaaattgggtcaacagcagctggtgtccttcggtgggctcataatgtacattgtcctgttatttctattctccaagtacccaaccagagtttactggagacctgtcttatggggaatcgggctacagtttcttcttgggctcttgattctaaggactgaccctggatttatagcttttgattggttgggcagacaagttcaggtcctgccgatcgtggttttcttcagcactgtgatgtccatgctgtactacctgggactgatgcagtggattattagaaaggttggatggatcatgctagttactacgggatcatctcctattgaatctgtagttgcttctggcaatatatttgttggacaaacggagtctcctctgctggtccgaccatatttaccttacatcaccaagtctgaactccacgccatcatgaccgccgggttctctaccattgctggaagcgtgctaggtgcatacatttcttttggggttccatcctcccacttgttaacagcgtcagttatgtcagcacctgcgtcattggctgctgctaaactcttttggcctgagacagaaaaacctaaaataaccctcaagaatgccatgaaaatggaaagtggtgattcagggaatcttctagaagctgcaacacagggagcatcctcctccatctccctggtggccaacatcgctgtgaatctgattgccttcctggccctgctgtcttttatgaattcagccctgtcctggtttggaaacatgtttgactacccacagctgagttttgagctaatctgctcctacatcttcatgcccttttccttcatgatgggagtggaatggcaggacagctttatggttgccagactcataggttataagaccttcttcaatgaatttgtggcttatgagcacctctcaaaatggatccacttgaggaaagaaggtggacccaaatttgtaaacggtgtgcagcaatatatatcaattcgttctgagataatcgccacttacgctctctgtggttttgccaatatcgggtccctaggaatcgtgatcggcggactcacatccatggctccttccagaaagcgtgatatcgcctcgggggcagtgagagctctgattgcggggaccgtggcctgcttcatgacagcctgcatcgcaggcatactctccagcactcctgtggacatcaactgccatcacgttttagagaatgccttcaactccactttccctggaaacacaaccaaggtgatagcttgttgccaaagtctgttgagcagcactgttgccaagggtcctggtgaagtcatcccaggaggaaaccacagtctgtattctttgaagggctgctgcacattgttgaatccatcgacctttaactgcaatgggatctctaatacattttgaggtcagccacttctccagtggaactctgaagtacagatgctgaattttctgctttggaaagaaaaaaaaaagaagctattgtccacagattgatgcttccataatggaatcagctttaattgcaaggaatgaagaaaaacaagagtggaccttcaaagctacaacattttcctnctncccttccttcccaccagcccct

SEQ ID NO: 61 - protein, 665 aa, Homo sapiens
MELRSTAAPRAEGYSNVGFQNEENFLENENTSGNNSIRSRAVQSREHTNTKQDEEQVTVEQDFPRNREHMEDDDEEMQQKGCLERRYDTVCGFCRKHKTTLRHIIWGILLAGYLVMVISACVLNFHRALPLFVITVAAIFFVVWDHLMAKYEHRIDEMLSPGRRLLNSHWFWLKWVIWSSLVLAVIFWLAFDTAKLGQQQLVSFGGLIMYIVLLFLFSKYPTRVYWRPVLWGIGLQFLLGLLILRTDPGFIAFDWLGRQVQVLPIVVFFSTVMSMLYYLGLMQWIIRKVGWIMLVTTGSSPIESVVASGNIFVGQTESPLLVRPYLPYITKSELHAIMTAGFSTIAGSVLGAYISFGVPSSHLLTASVMSAPASLAAAKLFWPETEKPKITLKNAMKMESGDSGNLLEAATQGASSSISLVANIAVNLIAFLALLSFMNSALSWFGNMFDYPQLSFELICSYIFMPFSFMMGVEWQDSFMVARLIGYKTFFNEFVAYEHLSKWIHLRKEGGPKFVNGVQQYISIRSEIIATYALCGFANIGSLGIVIGGLTSMAPSRKRDIASGAVRALIAGTVACFMTACIAGILSSTPVDINCHHVLENAFNSTFPGNTTKVIACCQSLLSSTVAKGPGEVIPGGNHSLYSLKGCCTLLNPSTFNCNGISNTF

SEQ ID NO: 62 - DNA, 1998 bp, Homo sapiens
  CDS (1)...(1998)
atggagctgaggagtacagcagcccccagagctgagggctacagcaacgtgggcttccagaatgaagaaaactttcttgagaacgagaacacatcaggaaacaactcaataagaagcagagctgtgcaaagcagggagcacacaaacaccaaacaggatgaagaacaggtcacagttgagcaggattttccaagaaacagagaacacatggaggatgatgatgaggagatgcaacaaaaagggtgtttggaaaggaggtatgacacagtatgtggtttctgtaggaaacacaaaacaactcttcggcacatcatctggggcattttattagcaggttatctggttatggtgatttcggcctgtgtgctgaactttcacagagcccttcctctttttgtgatcaccgtggctgccatcttctttgttgtctgggatcacctgatggccaaatacgaacatcgaattgatgagatgctgtctcctggcagaaggcttctaaacagccattggttctggctgaagtgggtgatctggagctccctggtcctagcagttattttctggttggcctttgacactgccaaattgggtcaacagcagctggtgtccttcggtgggctcataatgtacattgtcctgttatttctattctccaagtacccaaccagagtttactggagacctgtcttatggggaatcgggctacagtttcttcttgggctcttgattctaaggactgaccctggatttatagcttttgattggttgggcagacaagttcaggtcctgccgatcgtggttttcttcagcactgtgatgtccatgctgtactacctgggactgatgcagtggattattagaaaggttggatggatcatgctagttactacgggatcatctcctattgaatctgtagttgcttctggcaatatatttgttggacaaacggagtctcctctgctggtccgaccatatttaccttacatcaccaagtctgaactccacgccatcatgaccgccgggttctctaccattgctggaagcgtgctaggtgcatacatttcttttggggttccatcctcccacttgttaacagcgtcagttatgtcagcacctgcgtcattggctgctgctaaactcttttggcctgagacagaaaaacctaaaataaccctcaagaatgccatgaaaatggaaagtggtgattcagggaatcttctagaagctgcaacacagggagcatcctcctccatctccctggtggccaacatcgctgtgaatctgattgccttcctggccctgctgtcttttatgaattcagccctgtcctggtttggaaacatgtttgactacccacagctgagttttgagctaatctgctcctacatcttcatgcccttttccttcatgatgggagtggaatggcaggacagctttatggttgccagactcataggttataagaccttcttcaatgaatttgtggcttatgagcacctctcaaaatggatccacttgaggaaagaaggtggacccaaatttgtaaacggtgtgcagcaatatatatcaattcgttctgagataatcgccacttacgctctctgtggttttgccaatatcgggtccctaggaatcgtgatcggcggactcacatccatggctccttccagaaagcgtgatatcgcctcgggggcagtgagagctctgattgcggggaccgtggcctgcttcatgacagcctgcatcgcaggcatactctccagcactcctgtggacatcaactgccatcacgttttagagaatgccttcaactccactttccctggaaacacaaccaaggtgatagcttgttgccaaagtctgttgagcagcactgttgccaagggtcctggtgaagtcatcccaggaggaaaccacagtctgtattctttgaagggctgctgcacattgttgaatccatcgacctttaactgcaatgggatctctaatacattttga

SEQ ID NO: 63 - protein, 427 aa, Artificial Sequence
  Amino Acid Consensus Sequence
MEVLISVLGLVVFIAIAFLFSSNKKKISWRTVISALVLQVVLGLIVLKTPVGRWVFGKLAEGVQKLLAYANEGINFVFGSLAGPDKIDDLGFVFAFKVLPIIIFFSALISILYYLGIMQVVIRKIGWALQKALGTSKLESLSAAANIFLGQTEAPLVIKPYLGKLTRSELFTVMTSGMASIAGSVLGAYAAMGVPPEYLLAASVLAAPGGLIFAKLIYPETEESQEKEEEILKLEEEEKKNFFEALANGALAGLKVALNVAAMLIAFVALIALINGILGGVGGLFGYEGLSFQSILGYLFSPLAFLMGVPDNWSDALLVGSLMGTKLAVNEFVAYLDLSKYLKTRLAGAEEWVEGEKQKLSPKTVAIITFALCGFANFSSIGIILGGLGGLAPGSRKSVISRLGLKALLAGTLVSLLSATIAGILFI

SEQ ID NO: 64 - protein, 322 aa, Artificial Sequence
  Amino Acid Consensus Sequence
VLPVIIFFSALISILYYLGIMQWVIRKIGWLLQKVLGTSKVESMSAAANIFLGQTEAPLVIRPYLEKMTQSELFAIMTSGMATVAGSVLGAYVSMGVPAEYLIAASVMSAPGGLIFAKLIYPETEESKEESDDSVKLEEEEEQNFIDAAANGAMAGLKIVLNIAAMLIAFVALIAFINGILSWIGGLFGYEGLTFQMIFGYIFAPLAFLMGVPWNDEAMLVGQLIGQKLVINEFVAYMNLSQYLQNNKAGVWVGKQWLSPRTEAIVTFALCGFANFSSIGILIGGLGGLAPERRSDVIARLGLRALIAGTLANFMSACIAGI

SEQ ID NO: 65 - protein, 61 aa, Artificial Sequence
  Amino Acid Consensus Sequence
LISIIGMVVFILIAFLFSSNRKKINWRTVITALVLQFLLGWIILRTPVGRWAFQWLGDGVQ

SEQ ID NO: 66 - protein, 77 aa, Artificial Sequence
  Amino Acid Consensus Sequence
SDTPVDINCHHVLENGFNSRFPGNTTEVIACCQNLFNSTVAKGPNDVIPGGNHSLYALKGCCNLLNPPTFNCNWIPN

SEQ ID NO: 67 - protein, 103 aa, Artificial Sequence
  Amino Acid Consensus Sequence
FCRKHKQLFRWIIWGLLCTGYLAFILAACILNFQRALALFVITCVVIFFLVWDFLKKKYGKKIDRCLKPGRHTLLNHWFWLKWVIWGAAILGLILWLALDTAQ

SEQ ID NO: 68 - protein, 91 aa, Artificial Sequence
  Amino Acid Consensus Sequence
MELRPTAAPRDEGYSNEGFQNEDNFLENQNTPGNNTIRNRAVQSGEHGHTKQDDRQITIEQDPPGNREHPEDDDEDEHQKGCLERRYDTIC

SEQ ID NO: 69 - DNA, 2473 bp, Homo sapiens
  CDS (88)...(2241)
gaaatgatggagtaagagactcttttctaagcaactcaagtttgcagtgattcaggcctacttctgaagagacagccttttatctcaatgaatgacacagaaaaaccagcagatactccctctgaggaagaggactttggtgatccaaggacatatgacccagatttcaaggggcctgttgccaacaggagttgtacagatgttctgtgctgtatgatcttcctactgtgtattattggctacattgttttaggacttgtggcctgggtacatggggaccccagaagagcagcctatcctacagacagccagggccacttttgtggccagaagggcactcccaatgagaacaagaccattttgttttactttaacctgttacgctgtaccagtccctccgtgttgctaaacctacagtgccctaccacacagatctgtgtctccaagtgcccagaaaaatttttaacctatgtggaaatgcaacttttgtacacaaaagacaaaagctactgggaagactaccgtcagttctgtaagaccactgctaagcctgtgaagtctctcacacagcttttactggatgatgattgtccaacagcgatttttcccagcaaaccttttctccagagatgtttccctgacttctctaccaaaaatggcactttaacaataggaagtaagatgatgtttcaagatggaaatggagggacaagaagtgttgtagaactcgggattgctgcaaatggtatcaataaacttcttgatgcaaagtcacttggattgaaagtgtttgaagactatgcaagaacttggtattggattcttattggcctgacgatcgccatggtccttagttggatatttttgatacttctgaggttcatagctggatgcctcttctgggtcttcatgattggtgtgattggaattataggttatggaatatggcactgttaccagcagtacaccaatcttcaggaacgcccaagttctgtattaactatctatgacatcgggattcagactaacataagcatgtactttgaactgcaacaaacatggttcacatttatgataatactctgcatcattgaagtgattgtcatcctcatgctgatcttcctcaggaatcgaatccgagtcgccattatcctgctgaaggaaggaagcaaagccattggatatgttcctagtacattagtctatccagctttaactttcattttgctctcaatctgcatttgctactgggtcgtgacagcagttttcttggcgacatcgggggtacctgtatacaaagtcatagctccaggggggcattgtatacatgaaaatcaaacctgtgacccagagatttttaatacaactgaaattgccaaagcttgccctggggctctgtgtaactttgctttctatggtggaaagagcttgtaccatcagtacatccctaccttccatgtatacaacttatttgtctttctctggcttataaacttcgtcattgcattaggtcagtgcgcccttgctggtgcattcgctacttattactgggccatgaaaaaacctgatgacatcccacgatatccactttttactgcatttggacgagccatacgatatcacacaggatccctagcatttggatctttaattattgcattaattcaaatgtttaaaattgtactagaatacttggaccaccgtcttaaacgtacccagaacacattgtctaaattcctacagtgctgcctgagatgctgcttctggtgtttggaaaatgcaataaagtttttaaacagaaatgcctatattatgattgcaatatatggcagaaacttctgcaggtcagcaaaagatgctttcaatctgctgatgagaaatgttttgaaagttgcagttacagatgaagttacatactttgtattattcctggggaaacttctagttgctggaagtataggtgttctggccttcctattcttcacacaaagactgccagtgattgcacaaggaccagcatctttaaattactactgggtacctttgctgacagtcatttttgggtcttacctgattgcacatgggttcttcagcgtctatgcaatgtgtgttgaaacaattttcatctgcttcttggaagatttagaaagaaatgatggttctactgcaagaccttattatgtgagtcaacctttgctgaagattttccaggaggaaaatccacaaactaggaagcagtagaagagcaaactggtcgtcctacagctgtgtgttaccttttctccatctgctgtgtctgtgcaacatttgtttcataagtgctttgtgtttagcaacactgtattcacgaccttgttggcttgcatttgcatgttttataccaaagcttatactgtactatgtgaagccatcagaagtcgcaagggaattgttaataacataaaacatttttatactaaaaaaaaaaaaaaaa

SEQ ID NO: 70 - protein, 717 aa, Homo sapiens
MNDTEKPADTPSEEEDFGDPRTYDPDFKGPVANRSCTDVLCCMIFLLCIIGYIVLGLVAWVHGDPRRAAYPTDSQGHFCGQKGTPNENKTILFYFNLLRCTSPSVLLNLQCPTTQICVSKCPEKFLTYVEMQLLYTKDKSYWEDYRQFCKTTAKPVKSLTQLLLDDDCPTAIFPSKPFLQRCFPDFSTKNGTLTIGSKMMFQDGNGGTRSVVELGIAANGINKLLDAKSLGLKVFEDYARTWYWILIGLTIAMVLSWIFLILLRFIAGCLFWVFMIGVIGIIGYGIWHCYQQYTNLQERPSSVLTIYDIGIQTNISMYFELQQTWFTFMIILCIIEVIVILMLIFLRNRIRVAIILLKEGSKAIGYVPSTLVYPALTFILLSICICYWVVTAVFLATSGVPVYKVIAPGGHCIHENQTCDPEIFNTTEIAKACPGALCNFAFYGGKSLYHQYIPTFHVYNLFVFLWLINFVIALGQCALAGAFATYYWAMKKPDDIPRYPLFTAFGRAIRYHTGSLAFGSLIIALIQMFKIVLEYLDHRLKRTQNTLSKFLQCCLRCCFWCLENAIKFLNRNAYIMIAIYGRNFCRSAKDAFNLLMRNVLKVAVTDEVTYFVLFLGKLLVAGSIGVLAFLFFTQRLPVIAQGPASLNYYWVPLLTVIFGSYLIAHGFFSVYAMCVETIFICFLEDLERNDGSTARPYYVSQPLLKIFQEENPQTRKQ

SEQ ID NO: 71 - DNA, 2154 bp, Homo sapiens
  CDS (1)...(2154)
atgaatgacacagaaaaaccagcagatactccctctgaggaagaggactttggtgatccaaggacatatgacccagatttcaaggggcctgttgccaacaggagttgtacagatgttctgtgctgtatgatcttcctactgtgtattattggctacattgttttaggacttgtggcctgggtacatggggaccccagaagagcagcctatcctacagacagccagggccacttttgtggccagaagggcactcccaatgagaacaagaccattttgttttactttaacctgttacgctgtaccagtccctccgtgttgctaaacctacagtgccctaccacacagatctgtgtctccaagtgcccagaaaaatttttaacctatgtggaaatgcaacttttgtacacaaaagacaaaagctactgggaagactaccgtcagttctgtaagaccactgctaagcctgtgaagtctctcacacagcttttactggatgatgattgtccaacagcgatttttcccagcaaaccttttctccagagatgtttccctgacttctctaccaaaaatggcactttaacaataggaagtaagatgatgtttcaagatggaaatggagggacaagaagtgttgtagaactcgggattgctgcaaatggtatcaataaacttcttgatgcaaagtcacttggattgaaagtgtttgaagactatgcaagaacttggtattggattcttattggcctgacgatcgccatggtccttagttggatatttttgatacttctgaggttcatagctggatgcctcttctgggtcttcatgattggtgtgattggaattataggttatggaatatggcactgttaccagcagtacaccaatcttcaggaacgcccaagttctgtattaactatctatgacatcgggattcagactaacataagcatgtactttgaactgcaacaaacatggttcacatttatgataatactctgcatcattgaagtgattgtcatcctcatgctgatcttcctcaggaatcgaatccgagtcgccattatcctgctgaaggaaggaagcaaagccattggatatgttcctagtacattagtctatccagctttaactttcattttgctctcaatctgcatttgctactgggtcgtgacagcagttttcttggcgacatcgggggtacctgtatacaaagtcatagctccaggggggcattgtatacatgaaaatcaaacctgtgacccagagatttttaatacaactgaaattgccaaagcttgccctggggctctgtgtaactttgctttctatggtggaaagagcttgtaccatcagtacatccctaccttccatgtatacaacttatttgtctttctctggcttataaacttcgtcattgcattaggtcagtgcgcccttgctggtgcattcgctacttattactgggccatgaaaaaacctgatgacatcccacgatatccactttttactgcatttggacgagccatacgatatcacacaggatccctagcatttggatctttaattattgcattaattcaaatgtttaaaattgtactagaatacttggaccaccgtcttaaacgtacccagaacacattgtctaaattcctacagtgctgcctgagatgctgcttctggtgtttggaaaatgcaataaagtttttaaacagaaatgcctatattatgattgcaatatatggcagaaacttctgcaggtcagcaaaagatgctttcaatctgctgatgagaaatgttttgaaagttgcagttacagatgaagttacatactttgtattattcctggggaaacttctagttgctggaagtataggtgttctggccttcctattcttcacacaaagactgccagtgattgcacaaggaccagcatctttaaattactactgggtacctttgctgacagtcatttttgggtcttacctgattgcacatgggttcttcagcgtctatgcaatgtgtgttgaaacaattttcatctgcttcttggaagatttagaaagaaatgatggttctactgcaagaccttattatgtgagtcaacctttgctgaagattttccaggaggaaaatccacaaactaggaagcagtag

SEQ ID NO: 72 - DNA, 2233 bp, Homo sapiens
  CDS (110)...(2071)
cccacgcgtccgccagccccggccccggccccggctcgcgggcgctgcgtctccgcgtacaggaggcggcggcggctcccagtcaccggcccccgccggcgagcgcacgatgcactgcctgggcgccgagtacctggtttctgcagaaggagcccctaggcaaagggagtggcgaccccagatttataggaaatgcacagatacggcatggttattcctgttctttctcttttggactggtttggtgtttatcatgggctactcggtggtggctggagccgcgggaagactcctctttggctatgacagctttggcaacatgtgtggcaagaagaactcccccgtggaaggggcccctctttcagggcaggacatgaccctaaaaaaacacgtgttctttatgaattcctgcaacctggaagtcaaaggtacgcagctcaaccgcatggccctctgtgtatccaactgccctgaagagcagcttgactccctggaagaggtccagttctttgcaaacaccagtgggtccttcctgtgtgtttatagtttgaattccttcaactatacccacagtccaaaagcagactcactgtgtcccaggctaccagttcctccaagcaagtcatttcccttatttaaccgatgtgtccctcaaacacctgagtgctactccctatttgcatctgttttgataaatgatgttgacaccctccaccgaattctaagtggaatcatgtcgggaagagatacaatccttggcctgtgtatcctcgcattagccttgtctttggccatgatgtttaccttcagattcatcaccacccttctggttcacattttcatttcattggttattttgggattgttgtttgtctgcggtgttttatggtggctgtattatgactataccaacgacctcagcatagaattggacacagaaagggaaaatatgaagtgcgtgctggggtttgctatcgtatccacaggcatcacggcagtgctgctcgtcttgatttttgttctcagaaagagaataaaattgacagttgagcttttccaaatcacaaataaagccatcagcagtgctcccttcctgctgttccagccactgtggacatttgccatcctcattttcttctgggtcctctgggtggctgtgctgctgagcctgggaactgcaggagctgcccaggttatggaaggcggccaagtggaatataagcccctttcgggcattcggtacatgtggtcgtaccatttaattggcctcatctggactagtgaattcatccttgcgtgccagcaaatgactatagctggggcagtggttacttgttatttcaacagaagtaaaaatgatcctcctgatcatcccatcctttcgtctctctccattctcttcttctaccatcaaggaaccattgtgaaagggtcatttttaatctctgtggtgaggattccgagaatcattgtcatgtacatgcaaaacgcactgaaagaacagcagcatggtgcattgtccaggtacctgttccgatgctgctactgctgtttctggtgtcttgacaaatacctgctccatctcaaccagaatgcatatactacaactgctattaatgggacagatttctgtacatcagcaaaagatgcattcaaaatcttgtccaagaactcaagtcactttacatctattaactgctttggagacttcataatttttctaggaaaggtgttagtggtgtgtttcactgtttttggaggactcatggcttttaactacaatcgggcattccaggtgtgggcagtccctctgttattggtagctttttttgcctacttagtagcccatagttttttatctgtgtttgaaactgtgctggatgcacttttcctgtgttttgctgttgatctggaaacaaatgatggatcgtcagaaaagccctactttatggatcaagaatttctgagtttcgtaaaaaggagcaacaaattaaacaatgcaagggcacagcaggacaagcactcattaaggaatgaggagggaacagaactccaggccattgtgagatagatacccatttaggtatctgtacctggaaaacatttccttctaagagccatttacagaatagaagatgagaccactagagaaaagttagtgaatttttttttaaaagacctaataaaccctattcttcctcaaaaaaaaaaaaaaaaaaaaaaaaaaaaaaaa

SEQ ID NO: 73 - protein, 653 aa, Homo sapiens [sequence omitted]

SEQ ID NO: 74 - DNA, 1962 bp, Homo sapiens
  CDS (1)...(1962)
atgcactgcctgggcgccgagtacctggtttctgcagaaggagcccctaggcaaagggagtggcgaccccagatttataggaaatgcacagatacggcatggttattcctgttctttctcttttggactggtttggtgtttatcatgggctactcggtggtggctggagccgcgggaagactcctctttggctatgacagctttggcaacatgtgtggcaagaagaactcccccgtggaaggggcccctctttcagggcaggacatgaccctaaaaaaacacgtgttctttatgaattcctgcaacctggaagtcaaaggtacgcagctcaaccgcatggccctctgtgtatccaactgccctgaagagcagcttgactccctggaagaggtccagttctttgcaaacaccagtgggtccttcctgtgtgtttatagtttgaattccttcaactatacccacagtccaaaagcagactcactgtgtcccaggctaccagttcctccaagcaagtcatttcccttatttaaccgatgtgtccctcaaacacctgagtgctactccctatttgcatctgttttgataaatgatgttgacaccctccaccgaattctaagtggaatcatgtcgggaagagatacaatccttggcctgtgtatcctcgcattagccttgtctttggccatgatgtttaccttcagattcatcaccacccttctggttcacattttcatttcattggttattttgggattgttgtttgtctgcggtgttttatggtggctgtattatgactataccaacgacctcagcatagaattggacacagaaagggaaaatatgaagtgcgtgctggggtttgctatcgtatccacaggcatcacggcagtgctgctcgtcttgatttttgttctcagaaagagaataaaattgacagttgagcttttccaaatcacaaataaagccatcagcagtgctcccttcctgctgttccagccactgtggacatttgccatcctcattttcttctgggtcctctgggtggctgtgctgctgagcctgggaactgcaggagctgcccaggttatggaaggcggccaagtggaatataagcccctttcgggcattcggtacatgtggtcgtaccatttaattggcctcatctggactagtgaattcatccttgcgtgccagcaaatgactatagctggggcagtggttacttgttatttcaacagaagtaaaaatgatcctcctgatcatcccatcctttcgtctctctccattctcttcttctaccatcaaggaaccattgtgaaagggtcatttttaatctctgtggtgaggattccgagaatcattgtcatgtacatgcaaaacgcactgaaagaacagcagcatggtgcattgtccaggtacctgttccgatgctgctactgctgtttctggtgtcttgacaaatacctgctccatctcaaccagaatgcatatactacaactgctattaatgggacagatttctgtacatcagcaaaagatgcattcaaaatcttgtccaagaactcaagtcactttacatctattaactgctttggagacttcataatttttctaggaaaggtgttagtggtgtgtttcactgtttttggaggactcatggcttttaactacaatcgggcattccaggtgtgggcagtccctctgttattggtagctttttttgcctacttagtagcccatagttttttatctgtgtttgaaactgtgctggatgcacttttcctgtgttttgctgttgatctggaaacaaatgatggatcgtcagaaaagccctactttatggatcaagaatttctgagtttcgtaaaaaggagcaacaaattaaacaatgcaagggcacagcaggacaagcactcattaaggaatgaggagggaacagaactccaggccattgtgagatag

SEQ ID NO: 75 - protein, 654 aa, Homo sapiens
MGCCSSASSAAQSSKREWKPLEDRSCTDIPWLLLFILFCIGMGFICGFSIATGAAARLVSGYDSYGNICGQKNTKLEAIPNSGMDHTQRKYVFFLDPCNLDLINRKIKSVALCVAACPRQELKTLSDVQKFAEINGSALCSYNLKPSEYTTSPKSSVLCPKLPVPASAPIPFFHRCAPVNISCYAKFAEALITFVSDNSVLHRLISGVMTSKEIILGLCLLSLVLSMILMVIIRYISRVLVWILTILVILGSLGGTGVLWWLYAKQRRSPKETVTPEQLQIAEDNLRALLIYAISATVFTVILFLIMLVMRKRVALTIALFHVAGKVFIHLPLLVFQPFWTFFALVLFWVYWIMTLLFLGTTGSPVQNEQGFVEFKISGPLQYMWWYHVVGLIWISEFILACQQMTVAGAVVTYYFTRDKRNLPFTPILASVNRLIRYHLGTVAKGSFIITLVKIPRMILMYIHSQLKGKENACARCVLKSCICCLWCLEKCLNYLNQNAYTATAINSTNFCTSAKDAFVILVENALRVATINTVGDFMLFLGKVLIVCSTGLAGIMLLNYQQDYTVWVLPLIIVCLFAFLVAHCFLSIYEMVVDVLFLCFAIDTKYNDGSPGREFYMDKVLMEFVENSRKAMKEAGKGGVADSRELKPMLKKR

SEQ ID NO: 76 - protein, 706 aa, Homo sapiens
MGDERPHYYGKHGTPQKYDPTFKGPIYNRGCTDIICCVFLLLAIVGYVAVGIIAWTHGDPRKVIYPTDSRGEFCGQKGTKNENKPYLFYFNIVKCASPLVLLEFQCPTPQICVEKCPDRYLTYLNARSSRDFEYYKQFCVPGFKNNKGVAEVLRDGDCPAVLIPSKPLARRCFPAIHAYKGVLMVGNETTYEDGHGSRKNITDLVEGAKKANGVLEARQLAMRIFEDYTVSWYWIIIGLVIAMAMSLLFIILLRFLAGIMVWVMIIMVILVLGYGIFHCYMEYSRLRGEAGSDVSLVDLGFQTDFRVYLHLRQTWLAFMIILSILEVIIILLLIFLRKRILIAIALIKEASRAVGYVMCSLLYPLVTFFLLCLCIAYWASTAVFLSTSNEAVYKIFDDSPCPFTAKTCNPETFPSSNESRQCPNARCQFAFYGGESGYHRALLGLQIFNAFMFFWLANFVLALGQVTLAGAFASYYWALRKPDDLPAFPLFSAFGRALRYHTGSLAFGALILAIVQIIRVILEYLDQRLKGAENKFAKCLMTCLKCCFWCLEKFIKFLNRNAYIMIAIYGTNFCTSARNAFFLLMRNIIRVAVLDKVTDFLFLLGKLLIVGSVGILAFFFFTHRIRIVQDTAPPLNYYWVPILTVIVGSYLIAHGFFSVYGMCVDTLFLCFLEDLERNDGSAERPYFMSSTLKKLLNKTNKKAAES

SEQ ID NO: 77 - protein, 653 aa, Rattus norvegicus
MGCCSSASAAQSSKREWKPLEDRSCTDIPWLLLFVLFCIGMGFICGFSVATGAAARLVSGYDSYGNICGQRNAKLEAIANSGLDHTHRKYVFFLDPCNLDLINRKIKSMALCVAACPRQELKTLSDVQKFAEINGSALCSYNIKPSEYTLTAKSSAFCPKLPVPASAPIPFFHRCAPVNISCYAKFAEALITFVSDNSVLHRLISGVMTSKEIILGLCLLSLVLSMILMVIIRYISRVLVWILTILVILGSLGGTGVLWWLYAKQRSSPKETVIPEQLQIAEDNLRALLIYAISATVFTVILFLIMLVMRKRVALTIALFHVAGKVFIHLPLLVFQPFWTFFALVLFWAYWIMTLLFLGTTGSAVQNEQGFVEYKISGPLQYMWWYHVVGLIWISEFILACQQMTVAGAVVTYYFTRDKRNLPFTPILASVNRLIRYHLGTVAKGSFIITLVKIPRMILMYIHSQLKGKENACARCMLKSCICCLWCLEKCLSYLNQNAYTATAINSTNFCTSAKDAFVILVENALRVAAINTVGDFMLFLGKVLIVCSTGLAGIMLLNYQQDYTVWVLPLIIVCLFAFLVAHCFLSIYEMVVDVLFLCFAIDTKYNDGSPGREFYMDKVLMEFVENSRKAMKEAGKGGAADARELKPMLRKR

SEQ ID NO: 78 - protein, 646 aa, Torpedo marmorata
MGCCGCGSEEGSVRQWKPLEQRSCTDVLWLLIFVLFCIGMAIICGFAIASGAAQRLVFGYDSYGNICGHKNTEIKDVTMSGLDHTDKKYVFFFEPCNWDMVHLKILSVALCVTKCPDMDLKTLEDVRNFAKYNGSRLCLYNLDPTQYTSKNSKSCPILPVKSSKPIPFFHRCVPMDSGCKINFKALTTFVSYNSVLQRVITGVMTSKEIIVGLCLMSLVLSILLMVIIRYISKVLVWILAILTIIGSIGGTAVLWWLYADHKKTLKLDPSQGDVAADNVTALLVCAIIATVITVILLLLMLIMRKRVALTIALFHVAGKVFIHIPFLIFQSLWTFLALAFFWIYWIAVLLLLATAGYPQKKDQGYVEFKVSGPLQYTWIYHLVGLIWISEFILACQQMTIAGAVVTYYFTRDKHNLPATPILASMCRLIKYHLGTVAKGSFIITLIKIPQMILVYIHSQLKGKENACAKCMLKACMCCLWCLEKCLLYLNRNAYIATSINGTSFCTSAKDAIVILVENAMRVAAINTVGDFVLFLGKLLIVLVTGFVGIILLNYQRDYTVWVLPLIIICLFAFFVSHCFLSIYEMVVDVLFLCFAVDCKHNDGSPGREYYMDKSLMEFMDESRKAMRSVTGSGAEMKSMASGSDNA

SEQ ID NO: 79 - protein, 52 aa, Artificial Sequence
  Amino Acid Consensus Sequence
XAGAXXXXYXXXXKXPXXPXXXXXXXXXXYHXGXXXXGXXXXXXXXXXXXXX

SEQ ID NO: 80 - protein, 49 aa, Artificial Sequence
  Amino Acid Consensus Sequence
LKXXXXXCCXWCLXXXXXXXXXNAYXXXXIXXXXFCXSAKDAXXXLXXN

SEQ ID NO: 81 - DNA, 4113 bp, Homo sapiens
  CDS (101)...(3691)
caaagagcggctcgcgcgctggcctgggctctaaccgaggagagatcccgggagaactccagagctccgggggagcgctcctcggaagaccggggccaacatgcctgtgcgcagggggcatgtggcaccacaaaatacatttctggggaccatcattcggaaatttgaagggcaaaataaaaaatttatcattgcaaatgccagagtgcagaactgtgccatcatttattgcaacgatgggttctgtgagatgactggtttctccaggccagatgtcatgcaaaagccatgcacctgcgactttctccatggacccgagaccaagaggcatgatattgcccaaattgcccaggcattgctggggtcagaagagaggaaagtggaggtcacctactatcacaaaaatgggtccacttttatttgtaacactcacataattccagtgaaaaaccaagagggcgtggctatgatgttcatcattaattttgaatatgtgacggataatgaaaacgctgccaccccagagagggtaaacccaatattaccaatcaaaactgtaaaccggaaattttttgggttcaaattccctggtctgagagttctcacttacagaaagcagtccttaccacaagaagaccccgatgtggtggtcatcgattcatctaaacacagtgatgattcagtagccatgaagcattttaagtctcctacaaaagaaagctgcagcccctctgaagcagatgacacaaaagctttgatacagcccagcaaatgttctcccttggtgaatatatccggacctcttgaccattcctctcccaaaaggcaatgggaccgactctaccctgacatgctgcagtcaagttcccagctgtcccattccagatcaagggaaagcttatgtagtatacggagagcatcttcggtccatgatatagaaggattcggcgtccaccccaagaacatatttagagaccgacatgccagcgaagacaatggtcgcaatgtcaaagggccttttaatcatatcaagtcaagcctcctgggatccacatcagattcaaacctcaacaaatacagcaccattaacaagattccacagctcactctgaatttttcagaggtcaaaactgagaaaaagaattcatcacctccttcttcagataaaaccattattgcacccaaggttaaagatcgaacacacaatgtgactgagaaagtgacccaggttctctctttaggagcagatgtcctacctgaatacaaactgcagacaccacgcatcaacaagtttacgatattgcactacagccctttcaaggcagtctgggactggcttatcctgctgttggtcatatacactgctatatttactccctactctgcagccttcctcctcaatgacagagaagaacagaagagacgagaatgtggctattcttgtagccctttgaatgtggtagacttgattgtggatattatgtttatcatagatattttaataaacttcagaacaacatatgttaatcagaatgaagaagtggtaagtgatcccgccaaaatagcaatacactacttcaaaggctggttcctgattgacatggttgcagcaattccttttgacttgctgatttttggatcaggttctgatgagacaacaacattaattggtcttttgaagactgcccgactcctccgtcttgtgcgcgtggccaggaaactggatcgatattcagaatatggcgctgctgttctaatgctctcaatgtgcatctttgccctgaatgcacactggctggcttgcatttggtatgcgattgggaatgtagaaaggccttacctgactgacaaaatcggatggttggattccttaggacagcaaattgggaaacgttacaatgacagtgactcaagttctggaccatccattaaagacaaatacgtcacagcactttattttaccttcagcagtttaaccagtgtaggattcgggaatgtgtctcctaacacgaattcggagaaaatcttttcaatttgtgtcatgttgattggctcactaatgtatgcaagcatttttgggaatgtatctgcaattatccaaagactatactcgggaactgccaggtaccacatgcagatgctgcgagtaaaagagttcattcgctttcaccaaatccccaaccctctgaggcaacgtcttgaagaatatttccagcacgcatggacttacaccaatggcattgacatgaacatggtcctaaagggtttcccagaatgcttacaagcagacatttgtctacatctcaaccagacattgctgcaaaactgcaaagcctttcggggggcaagtaaaggttgccttagagctttggcaatgaagttcaaaaccacccatgcactccaaggagacaccctcgttcactgtggggatgtcctcactgcactttatttcttatccagaggctccattgaaatctcaaagaatgacatggtggtggctattctgggaaaaaatgatatatttggagaaatggttcatctttatgccaaacctggaaagtctaatgcagatgtaagagccctcacatactgtgacttgcataagattcagcgagaagacttgttagaggttttggatatgtatcctgagttttctgatcactttctaacaaacctagagttgactttcaacctaaggcatgagagcgcaaaggctgatctcctacgatcacaatccatgaatgattcagaaggagacaactgtaaactaagaagaaggaaattgtcatttgaaagtgaaggagagaaagaaaacagtacaaatgatcctgaagactctgcagataccataagacattatcagagttccaagagacactttgaagagaaaaaaagcagatcctcatctttcatctcctccattgatgatgaacaaaagccgctcttctcaggaatagtagactcttctccaggaatagggaaagcatctgggctcgattttgaagaaacagtgcccacctcaggaagaatgcacatagataaaagaagtcactcttgcaaagatatcactgacatgcgaagctgggaacgagaaaatgcacatccccagcctgaagactccagtccatctgcacttcagcgagctgcctggggtatctctgaaaccgaaagcgacctcacctacggggaagtggaacaaagattagatctgctccaggagcaacttaacaggcttgaatcccaaatgaccactgacatccagaccatcttacagttgctgcagaaacaaaccactgtggtccccccagcctacagtatggtaacagcaggatcagaatatcagagacccatcatccagctgatgagaaccagtcaaccggaagcatccatcaaaactgaccgaagtttcagcccttcctcacaatgtcctgaatttctagaccttgaaaaatctaaacttaaatccaaagaatccctttcaagtggggtgcatctgaacacagcttcagaagacaacttgacttcacttttaaaacaagacagtgatctctctttagagcttcacctgcggcaaagaaaaacttacgttcatccaattaggcatccttctttgccagattcatccctaagcactgtaggaatcgtgggtcttcataggcatgtttctgatcctggtcttccagggaaataatcattttgtactatttactccacatacaatgtaagtgcttttaatggctgttttcctttttctatttaaatcctctctacttgactcaggggctcacaaggtaccattatatgcaaaagtactgtatattttcctaaattgaagcttgtaaggtaaaactgagcagttaggatgtaaatatacataagaacttttggttccaaatgttaaaactgccagcatctcacggcaccttattttttatttttattttttaaatcacatgcatgttaggaaactccaatttctcttgcatggagactcctatttactgcttttactaaaccagtacttcgttatgaaaatgccttccacgcaaataagaaaccaagggataaaactgttcatggatgcaactcaaattcagatgatcatcaaggcat

SEQ ID NO: 82 - protein, 1196 aa, Homo sapiens [sequence omitted]

SEQ ID NO: 83 - DNA, 3591 bp, Homo sapiens
  CDS (1)...(3591)
atgcctgtgcgcagggggcatgtggcaccacaaaatacatttctggggaccatcattcggaaatttgaagggcaaaataaaaaatttatcattgcaaatgccagagtgcagaactgtgccatcatttattgcaacgatgggttctgtgagatgactggtttctccaggccagatgtcatgcaaaagccatgcacctgcgactttctccatggacccgagaccaagaggcatgatattgcccaaattgcccaggcattgctggggtcagaagagaggaaagtggaggtcacctactatcacaaaaatgggtccacttttatttgtaacactcacataattccagtgaaaaaccaagagggcgtggctatgatgttcatcattaattttgaatatgtgacggataatgaaaacgctgccaccccagagagggtaaacccaatattaccaatcaaaactgtaaaccggaaattttttgggttcaaattccctggtctgagagttctcacttacagaaagcagtccttaccacaagaagaccccgatgtggtggtcatcgattcatctaaacacagtgatgattcagtagccatgaagcattttaagtctcctacaaaagaaagctgcagcccctctgaagcagatgacacaaaagctttgatacagcccagcaaatgttctcccttggtgaatatatccggacctcttgaccattcctctcccaaaaggcaatgggaccgactctaccctgacatgctgcagtcaagttcccagctgtcccattccagatcaagggaaagcttatgtagtatacggagagcatcttcggtccatgatatagaaggattcggcgtccaccccaagaacatatttagagaccgacatgccagcgaagacaatggtcgcaatgtcaaagggccttttaatcatatcaagtcaagcctcctgggatccacatcagattcaaacctcaacaaatacagcaccattaacaagattccacagctcactctgaatttttcagaggtcaaaactgagaaaaagaattcatcacctccttcttcagataaaaccattattgcacccaaggttaaagatcgaacacacaatgtgactgagaaagtgacccaggttctctctttaggagcagatgtcctacctgaatacaaactgcagacaccacgcatcaacaagtttacgatattgcactacagccctttcaaggcagtctgggactggcttatcctgctgttggtcatatacactgctatatttactccctactctgcagccttcctcctcaatgacagagaagaacagaagagacgagaatgtggctattcttgtagccctttgaatgtggtagacttgattgtggatattatgtttatcatagatattttaataaacttcagaacaacatatgttaatcagaatgaagaagtggtaagtgatcccgccaaaatagcaatacactacttcaaaggctggttcctgattgacatggttgcagcaattccttttgacttgctgatttttggatcaggttctgatgagacaacaacattaattggtcttttgaagactgcccgactcctccgtcttgtgcgcgtggccaggaaactggatcgatattcagaatatggcgctgctgttctaatgctctcaatgtgcatctttgccctgaatgcacactggctggcttgcatttggtatgcgattgggaatgtagaaaggccttacctgactgacaaaatcggatggttggattccttaggacagcaaattgggaaacgttacaatgacagtgactcaagttctggaccatccattaaagacaaatacgtcacagcactttattttaccttcagcagtttaaccagtgtaggattcgggaatgtgtctcctaacacgaattcggagaaaatcttttcaatttgtgtcatgttgattggctcactaatgtatgcaagcatttttgggaatgtatctgcaattatccaaagactatactcgggaactgccaggtaccacatgcagatgctgcgagtaaaagagttcattcgctttcaccaaatccccaaccctctgaggcaacgtcttgaagaatatttccagcacgcatggacttacaccaatggcattgacatgaacatggtcctaaagggtttcccagaatgcttacaagcagacatttgtctacatctcaaccagacattgctgcaaaactgcaaagcctttcggggggcaagtaaaggttgccttagagctttggcaatgaagttcaaaaccacccatgcactccaaggagacaccctcgttcactgtggggatgtcctcactgcactttatttcttatccagaggctccattgaaatctcaaagaatgacatggtggtggctattctgggaaaaaatgatatatttggagaaatggttcatctttatgccaaacctggaaagtctaatgcagatgtaagagccctcacatactgtgacttgcataagattcagcgagaagacttgttagaggttttggatatgtatcctgagttttctgatcactttctaacaaacctagagttgactttcaacctaaggcatgagagcgcaaaggctgatctcctacgatcacaatccatgaatgattcagaaggagacaactgtaaactaagaagaaggaaattgtcatttgaaagtgaaggagagaaagaaaacagtacaaatgatcctgaagactctgcagataccataagacattatcagagttccaagagacactttgaagagaaaaaaagcagatcctcatctttcatctcctccattgatgatgaacaaaagccgctcttctcaggaatagtagactcttctccaggaatagggaaagcatctgggctcgattttgaagaaacagtgcccacctcaggaagaatgcacatagataaaagaagtcactcttgcaaagatatcactgacatgcgaagctgggaacgagaaaatgcacatccccagcctgaagactccagtccatctgcacttcagcgagctgcctggggtatctctgaaaccgaaagcgacctcacctacggggaagtggaacaaagattagatctgctccaggagcaacttaacaggcttgaatcccaaatgaccactgacatccagaccatcttacagttgctgcagaaacaaaccactgtggtccccccagcctacagtatggtaacagcaggatcagaatatcagagacccatcatccagctgatgagaaccagtcaaccggaagcatccatcaaaactgaccgaagtttcagcccttcctcacaatgtcctgaatttctagaccttgaaaaatctaaacttaaatccaaagaatccctttcaagtggggtgcatctgaacacagcttcagaagacaacttgacttcacttttaaaacaagacagtgatctctctttagagcttcacctgcggcaaagaaaaacttacgttcatccaattaggcatccttctttgccagattcatccctaagcactgtaggaatcgtgggtcttcataggcatgtttctgatcctggtcttccagggaaataa

SEQ ID NO: 84 - protein, 215 aa, Artificial Sequence
  Amino Acid Consensus Sequence
ILFILDLLFVLLFLLEIVLKFIAYGLKSTSNAKYLKSIFNILDLLAILPLLLLLVLFLSGTEQVAKKRLRERFSLELSQWYYRILRFLRLLRLLRLLRLLRLLRRLETLFEFELGTLAWSLQSLGLKSILRFLLLLLLLLIGFSVIGYLLFKGYEDLSEVDGNSEFSSYFDAFYFLFVTLTTVGFGDLVPVGIIFFVLFFIIVGLLLLNLLIAVI

SEQ ID NO: 85 - protein, 20 aa, Artificial Sequence
  Amino Acid Consensus Sequence
ILYWNAAAEELTGLSREEVI

SEQ ID NO: 86 - protein, 28 aa, Artificial Sequence
  Amino Acid Consensus Sequence
TLEYRNLRKDGSLIWVLVSASPIRDEDG

SEQ ID NO: 87 - protein, 120 aa, Artificial Sequence
  Amino Acid Consensus Sequence
ALEERSYPAGEVIIRQGDPGDSFYIVLSGEVEVYKLTEDGARTPEVSQKQDTREQVVATLGPGDFFGELALLTNDGNKNAVLPSLDQGAPRTATVRALTDSELLRLDREDFRRLLQKYPE

SEQ ID NO: 88 - protein, 31 aa, Artificial Sequence
  Coiled-coil segment
ALELSNELAVLANIEKEFKNDYKKLSMQCKD

SEQ ID NO: 89 - protein, 46 aa, Artificial Sequence
  Coiled-coil segment
EVNEGELKEIKQDISSLRYELLEEKSQNTEDLAELIRELGEKLSME

SEQ ID NO: 90 - protein, 22 aa, Artificial Sequence
  Leucine zipper pattern
LVPSPKSLFYLLLKLKKWISEL

SEQ ID NO: 91 - protein, 4 aa, Artificial Sequence
  Casein kinase II phosphorylation site
SIEE

SEQ ID NO: 92 - protein, 4 aa, Artificial Sequence
  Casein kinase II phosphorylation site
SQSE

SEQ ID NO: 93 - protein, 4 aa, Artificial Sequence
  Casein kinase II phosphorylation site
SSHD

SEQ ID NO: 94 - protein, 4 aa, Artificial Sequence
  Casein kinase II phosphorylation site
SSED

SEQ ID NO: 95 - protein, 4 aa, Artificial Sequence
  Casein kinase II phosphorylation site
TALE

SEQ ID NO: 96 - protein, 4 aa, Artificial Sequence
  Casein kinase II phosphorylation site
TSTD

SEQ ID NO: 97 - protein, 4 aa, Artificial Sequence
  Casein kinase II phosphorylation site
SWME

SEQ ID NO: 98 - protein, 4 aa, Artificial Sequence
  Casein kinase II phosphorylation site
SIID

SEQ ID NO: 99 - protein, 4 aa, Artificial Sequence
  Casein kinase II phosphorylation site
TLKD

SEQ ID NO: 100 - protein, 4 aa, Artificial Sequence
  Casein kinase II phosphorylation site
TLGD

SEQ ID NO: 101 - protein, 4 aa, Artificial Sequence
  Casein kinase II phosphorylation site
TVKD

SEQ ID NO: 102 - protein, 4 aa, Artificial Sequence
  Casein kinase II phosphorylation site
TTVE

SEQ ID NO: 103 - protein, 4 aa, Artificial Sequence
  Casein kinase II phosphorylation site
SFQE

SEQ ID NO: 104 - protein, 4 aa, Artificial Sequence
  Casein kinase II phosphorylation site
SYFE

SEQ ID NO: 105 - protein, 4 aa, Artificial Sequence
  Casein kinase II phosphorylation site
SHED

SEQ ID NO: 106 - protein, 4 aa, Artificial Sequence
  Casein kinase II phosphorylation site
SSED

SEQ ID NO: 107 - protein, 4 aa, Artificial Sequence
  cAMP/cGMP-dependent protein kinase phosphorylation site
RRQT

SEQ ID NO: 108 - protein, 4 aa, Artificial Sequence
  cAMP/cGMP-dependent protein kinase phosphorylation site
KKLS

SEQ ID NO: 109 - protein, 8 aa, Artificial Sequence
  Tyrosine phosphorylation site
RRNESQDY

SEQ ID NO: 110 - protein, 8 aa, Artificial Sequence
  Tyrosine phosphorylation site
RFLDAAEY

SEQ ID NO: 111 - protein, 8 aa, Artificial Sequence
  Tyrosine phosphorylation site
KFIENIGY

SEQ ID NO: 112 - protein, 4 aa, Artificial Sequence
  Amidation site
KGRR

SEQ ID NO: 113 - protein, 4 aa, Artificial Sequence
  Amidation site
EGKR

SEQ ID NO: 114 - protein, 4 aa, Artificial Sequence
  N-glycosylation site
NESQ

SEQ ID NO: 115 - protein, 4 aa, Artificial Sequence
  N-glycosylation site
NLSR

SEQ ID NO: 116 - protein, 4 aa, Artificial Sequence
  N-glycosylation site
NLSR

SEQ ID NO: 117 - protein, 4 aa, Artificial Sequence
  N-glycosylation site
NLSG

SEQ ID NO: 118 - protein, 4 aa, Artificial Sequence
  N-glycosylation site
NETS

SEQ ID NO: 119 - protein, 4 aa, Artificial Sequence
  N-glycosylation site
NDTL

SEQ ID NO: 120 - protein, 4 aa, Artificial Sequence
  N-glycosylation site
NESF

SEQ ID NO: 121 - protein, 4 aa, Artificial Sequence
  N-glycosylation site
NVTM

SEQ ID NO: 122 - protein, 4 aa, Artificial Sequence
  N-glycosylation site
NSSF

SEQ ID NO: 123 - protein, 6 aa, Artificial Sequence
  N-myristoylation site
GAAGAA

SEQ ID NO: 124 - protein, 6 aa, Artificial Sequence
  N-myristoylation site
GTRSSH

SEQ ID NO: 125 - protein, 6 aa, Artificial Sequence
  N-myristoylation site
GMIWAE

SEQ ID NO: 126 - protein, 6 aa, Artificial Sequence
  N-myristoylation site
GAKQNE

SEQ ID NO: 127 - protein, 6 aa, Artificial Sequence
  N-myristoylation site
GILGSH

(19) United States
(12) Patent Application Publication
(10) Pub. No.: US 2009/0226448 A1
(43) Pub. Date: Sep. 10, 2009

(54) Novel 18607, 15603, 69318, 12303, 48000, 52920, 5433, 38554, 57301, 58324, 55063, 52991, 59914, 59921 and 33751 molecules and uses therefor

(75) Inventors: Maria A. Glucksmann, Lexington, MA (US); Rory A.J. Curtis, Ashland, MA (US); Jose M. Lora, Mountain View, CA (US); Katherine M. Galvin, Jamaica Plain, MA (US); Inmaculada Silos-Santiago, Del Mar, CA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc., Cambridge, MA (US)

(21) Appl. No.: 12/381,747

(22) Filed: Mar. 16, 2009

Related U.S. Application Data
(63) Continuation of application No. 10/391,399, filed on Mar. 18, 2003.
Application No. 10/391,399 is a continuation-in-part of application No. 09/789,481, filed on Feb. 20, 2001, now abandoned.
Application No. 09/789,481 is a continuation-in-part of application No. 09/634,669, filed on Aug. 8, 2000, now abandoned.
Application No. 09/634,669 is a continuation-in-part of application No. 09/583,373, filed on May 31, 2000, now abandoned.
Application No. 09/583,373 is a continuation-in-part of application No. 09/510,706, filed on Feb. 22, 2000, now abandoned.
Application No. 10/391,399 is a continuation-in-part of application No. 10/309,804, filed on Dec. 4, 2002, now abandoned.
Application No. 10/391,399 is a continuation-in-part of application No. 10/094,214, filed on Mar. 8, 2002, now abandoned.
Application No. 10/391,399 is a continuation-in-part of application No. 09/828,035, filed on Apr. 6, 2001, now abandoned.
Application No. 10/391,399 is a continuation-in-part of application No. 09/891,762, filed on Jun. 26, 2001, now abandoned.
Application No. 10/391,399 is a continuation-in-part of application No. 10/245,121, filed on Sep. 17, 2002, now abandoned.
Application No. 10/391,399 is a continuation-in-part of application No. 10/095,139, filed on Mar. 11, 2002, now abandoned.
Application No. 10/391,399 is a continuation-in-part of application No. 09/957,683, filed on Sep. 19, 2001, now abandoned.
Application No. 10/391,399 is a continuation-in-part of application No. 09/942,447, filed on Aug. 29, 2001, now abandoned.
Application No. 10/391,399 is a continuation-in-part of application No. 10/062,937, filed on Jan. 31, 2002, now abandoned.
Application No. 10/391,399 is a continuation-in-part of application No. 10/255,532, filed on Sep. 26, 2002, now abandoned.
(60) Provisional application No. 60/336,936, filed on Dec. 4, 2001.
(60) Provisional application No. 60/275,078, filed on Mar. 12, 2001.
(60) Provisional application No. 60/195,734, filed on Apr. 7, 2000.
(60) Provisional application No. 60/214,176, filed on Jun. 26, 2000.
(60) Provisional application No. 60/322,983, filed on Sep. 17, 2001.
(60) Provisional application No. 60/275,172, filed on Mar. 12, 2001.
(60) Provisional application No. 60/233,537, filed on Sep. 19, 2000.
(60) Provisional application No. 60/229,036, filed on Aug. 31, 2000.
(60) Provisional application No. 60/267,076, filed on Feb. 1, 2001.
(60) Provisional application No. 60/325,854, filed on Sep. 27, 2001.

(51) Int. Cl. A61K 39/395; C12N 15/12; C12N 5/10; C07K 14/47; C07K 16/18; C12P 21/02; C12Q 1/68; G01N 33/566; A61K 31/7105; A61K 38/48; A61K 38/17; A61P 35/00; A61P 43/00

(52) U.S. Cl. 424/139.1; 536/23.5; 435/325; 530/350; 530/387.9; 435/69.1; 435/6; 436/501; 435/375; 514/44 A; 514/44 R; 424/94.63; 514/12

(57) ABSTRACT

The invention provides isolated nucleic acids molecules, designated 18607, 15603, 69318, 12303, 48000, 52920, 5433, 38554, 57301, 58324, 55063, 52991, 59914, 59921 and 33751 nucleic acid molecules. The invention also provides antisense nucleic acid molecules, recombinant expression vectors containing 18607, 15603, 69318, 12303, 48000, 52920, 5433, 38554, 57301, 58324, 55063, 52991, 59914, 59921 and 33751 nucleic acid molecules, host cells into which the expression vectors have been introduced, and nonhuman transgenic animals in which a 18607, 15603, 69318, 12303, 48000, 52920, 5433, 38554, 57301, 58324, 55063, 52991, 59914, 59921 or 33751 gene has been introduced or disrupted. The invention still further provides isolated 18607, 15603, 69318, 12303, 48000, 52920, 5433, 38554, 57301, 58324, 55063, 52991, 59914, 59921 or 33751 proteins, fusion proteins, antigenic peptides and anti-18607, 15603, 69318, 12303, 48000, 52920, 5433, 38554, 57301, 58324, 55063, 52991, 59914, 59921 or 33751 antibodies. Diagnostic and therapeutic methods utilizing compositions of the invention are also provided.

### RELATED APPLICATIONS

The present application is a continuation of U.S. patent application Ser. No. 10/391,399, filed Mar. 18, 2003 (pending), which is a continuation-in-part of U.S. patent application Ser. No. 09/789,481, filed Feb. 20, 2001 (abandoned), which is a continuation-in-part of U.S. patent application Ser. No. 09/634,669, filed Aug. 8, 2000 (abandoned), which is a continuation-in-part of U.S. patent application Ser. No. 09/583,373, filed May 31, 2000 (abandoned), which is a continuation-in-part of U.S. patent application Ser. No. 09/510,706, filed Feb. 22, 2000 (abandoned). U.S. patent application Ser. No. 10/391,399 is also a continuation-in-part of U.S. patent application Ser. No. 10/309,804, filed Dec. 4, 2002 (abandoned), which claims the benefit of U.S. Provisional Application Ser. No. 60/336,936, filed Dec. 4, 2001 (abandoned). U.S. patent application Ser. No. 10/391,399 is also a continuation-in-part of U.S. patent application Ser. No. 10/094,214, filed Mar. 8, 2002 (abandoned), which claims the benefit of U.S. Provisional Application Ser. No. 60/275,078, filed Mar. 12, 2001 (abandoned). U.S. patent application Ser. No. 10/391,399 is also a continuation-in-part of U.S. patent application Ser. No. 09/828,035, filed Apr. 6, 2001 (abandoned), which claims the benefit of U.S. Provisional Application Ser. No. 60/195,734, filed Apr. 7, 2000 (abandoned). U.S. patent application Ser. No. 10/391,399 is also a continuation-in-part of U.S. patent application Ser. No. 09/891,762, filed Jun. 26, 2001 (abandoned), which claims the benefit of U.S. Provisional Application Ser. No. 60/214,176, filed Jun. 26, 2000 (abandoned). U.S. patent application Ser. No. 10/391,399 is also a continuation-in-part of U.S. patent application Ser. No. 10/245,121, filed Sep. 17, 2002 (abandoned), which claims the benefit of U.S. Provisional Application Ser. No. 60/322,983, filed Sep. 17, 2001 (abandoned). U.S. patent application Ser. No. 10/391,399 is also a continuation-in-part of U.S. patent application Ser. No. 10/095,139, filed Mar. 11, 2002 (abandoned), which claims the benefit of U.S. Provisional Application Ser. No. 60/275,172, filed Mar. 12, 2001 (abandoned). U.S. patent application Ser. No. 10/391,399 is also a continuation-in-part of U.S. patent application Ser. No. 09/957,683, filed Sep. 19, 2001 (abandoned), which claims the benefit of U.S. Provisional Application Ser. No. 60/233,537, filed on Sep. 19, 2000 (abandoned). U.S. patent application Ser. No. 10/391,399 is also a continuation-in-part of U.S. patent application Ser. No. 09/942,447, filed Aug. 29, 2001 (abandoned), which claims the benefit of U.S. Provisional Application Ser. No. 60/229,036, filed Aug. 31, 2000 (abandoned). U.S. patent application Ser. No. 10/391,399 is also a continuation-in-part of U.S. patent application Ser. No. 10/062,937, filed Jan. 31, 2002 (abandoned), which claims the benefit of U.S. Provisional Application Ser. No. 60/267,076, filed Feb. 1, 2001 (abandoned). U.S. patent application Ser. No. 10/391,399 is also a continuation-in-part of U.S. patent application Ser. No. 10/255,532, filed Sep. 26, 2002 (abandoned), which claims the benefit of U.S. Provisional Application Ser. No. 60/325,854, filed Sep. 27, 2001 (abandoned). The entire contents of each of the above-referenced patent applications are incorporated herein by this reference.

### BACKGROUND OF THE INVENTION

Many intercellular and intracellular events depend on the regulation of the concentration of certain ions such as calcium, sodium, potassium, and chloride within the cell. Ion channels have been found which regulate the flow of anions or cations through a membrane based on voltage (voltage-gated channels), pH (mechanically gated channels), phosphorylation, and ligand binding (ligand-gated channels). Typically, an ion channel consists of multiple transmembrane domains which form a channel through which the ions pass from one side of the membrane to the other. These ion channels may play important roles in how a cell responds to hormones or neurotransmitters with increased activity of enzymes such as phospholipase C and a subsequent rise in the concentration of intracellular free calcium (Ca⁺²). The increase in intracellular calcium concentration may occur as a result of the release of calcium from intracellular stores as well as an influx of calcium through the plasma membrane.

Examples of ion channels include, for example, calcium channel, calcium/sodium antiporters, potassium channel, organic ion transporter, and choline transporters. Such ion channels have the ability, for example: 1) to modulate membrane excitability; 2) to influence the resting potential of membranes; 3) to modulate wave forms and frequencies of action potentials; 4) to modulate thresholds of excitation; 5) to modulate neurite outgrowth and synaptogenesis; 6) to modulate signal transduction, 7) to bind a second messenger; 8) to bind diacylglycerol; 9) to regulate the flow of cations through a membrane; 10) to transport a substrate or target molecule, e.g., an ion (e.g., a calcium ion) across a membrane; 11) to transport a second substrate or target molecule, e.g., another ion (e.g., a sodium ion) across a membrane; 12) to transport a third substrate or target molecule, e.g., another ion (e.g., a potassium ion) across a membrane; 13) to interact with and/or modulate the activity of a second non-transporter protein; 14) to modulate cellular signaling and/or gene transcription (e.g., either directly or indirectly; 15) to interact with a non-TWIK protein molecule; 16) to activate a TWIK-dependent signal transduction pathway; 17) to modulate the release of neurotransmitters; 18) to protect cells and/or tissues from organic ions; 19) to modulate intracellular Ca²⁺ concentration; 20) to bind a ligand, e.g., L-glutamate, and/or glycine; 21) to modulate (e.g., promote, catalyze, regulate, initiate, facilitate or inhibit) the manufacture of choline metabolites and/or compounds of which choline is a component or precursor, e.g., phospholipids (e.g., phosphatidylcholine (lecithin), sphingomyelin, sphingophosphorylcholine, and platelet activating factor), acetylcholine, very low density lipoproteins (VLDLs), and betaine, e.g., by transporting choline into or out of cells; 22) to modulate (e.g., promote, catalyze, regulate, initiate, facilitate or inhibit) transport of choline, its metabolites, and/or compounds of which choline is a component or precursor across membranes (e.g., plasma membranes), e.g., from an extracellular medium into a cell, or vice versa; 23) to modulate (e.g., promote, catalyze, regulate, initiate, facilitate or inhibit) transport of choline, its metabolites, and/or compounds of which choline is a component or precursor across barriers between tissues (e.g., the blood-brain barrier); as well as many others. Accordingly, there exists a need to identify additional ion channels, for example, for use as disease markers and as targets for identifying various therapeutic modulators.

### SUMMARY OF THE INVENTION

The present invention is based, at least in part, on the discovery of novel nucleic acid molecules and proteins encoded by such nucleic acid molecules, referred to herein as “18607, 15603, 69318, 12303, 48000, 52920, 5433, 38554, 57301, 58324, 55063, 52991, 59914, 59921 or 33751”. The 18607, 15603, 69318, 12303, 48000, 52920, 5433, 38554, 57301, 58324, 55063, 52991, 59914, 59921 or 33751 nucleic acid and protein molecules of the present invention are useful as modulating agents in regulating a variety of cellular processes, e.g., including but not limited to, modulating cellular response to hormones or neurotransmitters. In particular, these nucleic acid molecules will be advantageous in the regulation of any cellular function, uncontrolled proliferation and differentiation, such as in cases of pain. Accordingly, in one aspect, this invention provides isolated nucleic acid molecules encoding 18607, 15603, 69318, 12303, 48000, 52920, 5433, 38554, 57301, 58324, 55063, 52991, 59914, 59921 or 33751 proteins or biologically active portions thereof, as well as nucleic acid fragments suitable as primers or hybridization probes for the detection of 18607, 15603, 69318, 12303, 48000, 52920, 5433, 38554, 57301, 58324, 55063, 52991, 59914, 59921 or 33751-encoding nucleic acids.

The nucleotide sequence of the cDNA encoding 18607, 15603, 69318, 12303, 48000, 52920, 5433, 38554, 57301, 58324, 55063, 52991, 59914, 59921 or 33751, and the amino acid sequence of 18607, 15603, 69318, 12303, 48000, 52920, 5433, 38554, 57301, 58324, 55063, 52991, 59914, 59921 or 33751 polypeptides are depicted in Table 1.

TABLE 1
Sequences of the invention

| Gene | | | |
|---|---|---|---|
| Name | cDNA | Protein | Coding Region |
| 18607 | SEQ ID NO: 1 | SEQ ID NO: 2 | SEQ ID NO: 3 |
| 15603 | SEQ ID NO: 5 | SEQ ID NO: 6 | SEQ ID NO: 7 |
| 69318 | SEQ ID NO: 13 | SEQ ID NO: 14 | SEQ ID NO: 15 |
| 12303 | SEQ ID NO: 18 | SEQ ID NO: 19 | SEQ ID NO: 20 |
| 48000 | SEQ ID NO: 21 | SEQ ID NO: 22 | SEQ ID NO: 23 |
| 52920 | SEQ ID NO: 24 | SEQ ID NO: 25 | SEQ ID NO: 26 |
| 5433 | SEQ ID NO: 27 | SEQ ID NO: 28 | SEQ ID NO: 29 |
| 38554 | SEQ ID NO: 32 | SEQ ID NO: 33 | SEQ ID NO: 34 |
| | and 53 | and 54 | and 55 |
| 57301 | SEQ ID NO: 35 | SEQ ID NO: 36 | SEQ ID NO: 37 |
| 58324 | SEQ ID NO: 38 | SEQ ID NO: 39 | SEQ ID NO: 40 |
| 55063 | SEQ ID NO: 56 | SEQ ID NO: 57 | SEQ ID NO: 58 |
| 52991 | SEQ ID NO: 60 | SEQ ID NO: 61 | SEQ ID NO: 62 |
| 59914 | SEQ ID NO: 69 | SEQ ID NO: 70 | SEQ ID NO: 71 |
| 59921 | SEQ ID NO: 72 | SEQ ID NO: 73 | SEQ ID NO: 74 |
| 33751 | SEQ ID NO: 81 | SEQ ID NO: 82 | SEQ ID NO: 83 |

Accordingly, in one aspect, the invention features a nucleic acid molecule which encodes a 18607, 15603, 69318, 12303, 48000, 52920, 5433, 38554, 57301, 58324, 55063, 52991, 59914, 59921 or 33751 protein or polypeptide, e.g., a biologically active portion of the 18607, 15603, 69318, 12303, 48000, 52920, 5433, 38554, 57301, 58324, 55063, 52991, 59914, 59921 or 33751 protein. In a preferred embodiment, the isolated nucleic acid molecule encodes a polypeptide having the amino acid sequence of SEQ ID NO:2, 6, 14, 19, 22, 25, 28, 33, 36, 39, 54, 57, 61, 70, 73 or 82. In other embodiments, the invention provides isolated 18607, 15603, 69318, 12303, 48000, 52920, 5433, 38554, 57301, 58324, 55063, 52991, 59914, 59921 or 33751 nucleic acid molecules having the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83. In still other embodiments, the invention provides nucleic acid molecules that are substantially identical (e.g., naturally occurring allelic variants) to the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83. In other embodiments, the invention provides a nucleic acid molecule which hybridizes under a stringent hybridization condition as described herein to a nucleic acid molecule comprising the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, wherein the nucleic acid encodes a full length 18607, 15603, 69318, 12303, 48000, 52920, 5433, 38554, 57301, 58324, 55063, 52991, 59914, 59921 or 33751 protein or an active fragment thereof.

In a related aspect, the invention further provides nucleic acid constructs which include a 18607, 15603, 69318, 12303, 48000, 52920, 5433, 38554, 57301, 58324, 55063, 52991, 59914, 59921 or 33751 nucleic acid molecule described herein. In certain embodiments, the nucleic acid molecules of the invention are operatively linked to native or heterologous regulatory sequences. Also included are vectors and host cells containing the 18607, 15603, 69318, 12303, 48000, 52920, 5433, 38554, 57301, 58324, 55063, 52991, 59914, 59921 or 33751 nucleic acid molecules of the invention e.g., vectors and host cells suitable for producing polypeptides.

In another related aspect, the invention provides nucleic acid fragments suitable as primers or hybridization probes for the detection of 18607, 15603, 69318, 12303, 48000, 52920, 5433, 38554, 57301, 58324, 55063, 52991, 59914, 59921 or 33751-encoding nucleic acids.

In still another related aspect, isolated nucleic acid molecules that are antisense to a 18607, 15603, 69318, 12303, 48000, 52920, 5433, 38554, 57301, 58324, 55063, 52991, 59914, 59921 or 33751 encoding nucleic acid molecule are provided.

In another aspect, the invention features 18607, 15603, 69318, 12303, 48000, 52920, 5433, 38554, 57301, 58324, 55063, 52991, 59914, 59921 or 33751 polypeptides, and biologically active or antigenic fragments thereof that are useful, e.g., as reagents or targets in assays applicable to treatment and diagnosis of 18607, 15603, 69318, 12303, 48000, 52920, 5433, 38554, 57301, 58324, 55063, 52991, 59914, 59921 or 33751-associated disorders. In another embodiment, the invention provides 18607, 15603, 69318, 12303, 48000, 52920, 5433, 38554, 57301, 58324, 55063, 52991, 59914, 59921 or 33751 polypeptides having a 18607, 15603, 69318, 12303, 48000, 52920, 5433, 38554, 57301, 58324, 55063, 52991, 59914, 59921 or 33751 activity.

In other embodiments, the invention provides 18607, 15603, 69318, 12303, 48000, 52920, 5433, 38554, 57301, 58324, 55063, 52991, 59914, 59921 or 33751 polypeptides, e.g., a 18607, 15603, 69318, 12303, 48000, 52920, 5433, 38554, 57301, 58324, 55063, 52991, 59914, 59921 or 33751 polypeptide having the amino acid sequence shown in SEQ ID NO:2, 6, 14, 19, 22, 25, 28, 33, 36, 39, 54, 57, 61, 70, 73 or 82; an amino acid sequence that is substantially identical to the amino acid sequence shown in SEQ ID NO:2, 6, 14, 19, 22, 25, 28, 33, 36, 39, 54, 57, 61, 70, 73 or 82; or an amino acid sequence encoded by a nucleic acid molecule having a nucleotide sequence which hybridizes under a stringent hybridization condition as described herein to a nucleic acid molecule comprising the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, wherein the nucleic acid encodes a full length 18607, 15603, 69318, 12303, 48000, 52920, 5433, 38554, 57301, 58324, 55063, 52991, 59914, 59921 or 33751 protein or an active fragment thereof.

In a related aspect, the invention further provides nucleic acid constructs which include a 18607, 15603, 69318, 12303, 48000, 52920, 5433, 38554, 57301, 58324, 55063, 52991, 59914, 59921 or 33751 nucleic acid molecule described herein.

In a related aspect, the invention provides 18607, 15603, 69318, 12303, 48000, 52920, 5433, 38554, 57301, 58324, 55063, 52991, 59914, 59921 or 33751 polypeptides or fragments operatively linked to non-18607, 15603, 69318, 12303, 48000, 52920, 5433, 38554, 57301, 58324, 55063, 52991, 59914, 59921 or 33751 polypeptides to form fusion proteins.

In another aspect, the invention features antibodies and antigen-binding fragments thereof, that react with, or more preferably specifically or selectively bind 18607, 15603, 69318, 12303, 48000, 52920, 5433, 38554, 57301, 58324, 55063, 52991, 59914, 59921 or 33751 polypeptides.

In another aspect, the invention provides methods of screening for compounds that modulate the expression or activity of the 18607, 15603, 69318, 12303, 48000, 52920, 5433, 38554, 57301, 58324, 55063, 52991, 59914, 59921 or 33751 polypeptides or nucleic acids.

In still another aspect, the invention provides a process for modulating 18607, 15603, 69318, 12303, 48000, 52920, 5433, 38554, 57301, 58324, 55063, 52991, 59914, 59921 or 33751 polypeptide or nucleic acid expression or activity, e.g., using the compounds identified in the screens described herein. In certain embodiments, the methods involve treatment of conditions related to aberrant activity or expression of the 18607, 15603, 69318, 12303, 48000, 52920, 5433, 38554, 57301, 58324, 55063, 52991, 59914, 59921 or 33751 polypeptides or nucleic acids, such as conditions or disorders involving aberrant or deficient 18607, 15603, 69318, 12303, 48000, 52920, 5433, 38554, 57301, 58324, 55063, 52991, 59914, 59921 or 33751 expression. Examples of such disorders include, but are not limited to cellular proliferative and/or differentiative disorders, brain disorders, blood vessel disorders, platelet disorders, breast disorders, colon disorders, kidney disorders, lung disorders, ovarian disorders, prostate disorders, pancreatic disorders, skeletal muscle disorders, testicular disorders, eye disorders, hormonal disorders, disorders associated with bone metabolism, immune e.g., inflammatory, disorders, cardiovascular disorders, endothelial cell disorders, liver disorders, viral diseases, pain, metabolic disorders, neurological disorders, neurodegenerative disorders or angiogenic disorders.

The invention also provides assays for determining the activity of or the presence or absence of 18607, 15603, 69318, 12303, 48000, 52920, 5433, 38554, 57301, 58324, 55063, 52991, 59914, 59921 or 33751 polypeptides or nucleic acid molecules in a biological sample, including for disease diagnosis.

In a further aspect, the invention provides assays for determining the presence or absence of a genetic alteration in a 18607, 15603, 69318, 12303, 48000, 52920, 5433, 38554, 57301, 58324, 55063, 52991, 59914, 59921 or 33751 polypeptide or nucleic acid molecule, including for disease diagnosis.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a 18607, 15603, 69318, 12303, 48000, 52920, 5433, 38554, 57301, 58324, 55063, 52991, 59914, 59921 or 33751 molecule. In one embodiment, the capture probe is a nucleic acid, e.g., a probe complementary to a 18607, 15603, 69318, 12303, 48000, 52920, 5433, 38554, 57301, 58324, 55063, 52991, 59914, 59921 or 33751 nucleic acid sequence. In another embodiment, the capture probe is a polypeptide, e.g., an antibody specific for 18607, 15603, 69318, 12303, 48000, 52920, 5433, 38554, 57301, 58324, 55063, 52991, 59914, 59921 or 33751 polypeptides. Also featured is a method of analyzing a sample by contacting the sample to the aforementioned array and detecting binding of the sample to the array.

Other features and advantages of the invention will be apparent from the following detailed description, and from the claims.

### DETAILED DESCRIPTION OF THE INVENTION

### Human 18607 (TLCC)

The present invention is based, at least in part, on the discovery of novel molecules, referred to herein as “TRP-like calcium channel”, “TLCC” or “18607” nucleic acid and protein molecules, which are novel members of the calcium channel family. These novel molecules are capable of, for example, modulating a calcium channel mediated activity in a cell, e.g., a neuronal, muscle (e.g., cardiac muscle), or liver cell. The present invention is further based, at least in part, on the discovery that TLCC genes are up-regulated in stellate cells (the main effectors of liver fibrosis) as compared to their expression in normal hepatic cells, and, thus, may be associated with a hepatic disorder. Accordingly, the present invention further provides methods and compositions for the diagnosis and treatment of a hepatic disorder, including but not limited to, liver fibrosis, hepatitis, liver tumors, cirrhosis of the liver, hemochromatosis, liver parasite induced disorders, alpha-1 antitrypsin deficiency, and autoimmune hepatitis.

The human TLCC or 18607 sequence (SEQ ID NO:1), which is approximately 3900 nucleotides long including untranslated regions, contains a predicted methionine-initiated coding sequence of about 3387 nucleotides (nucleotides 138-3524 of SEQ ID NO:1; nucleotides 1-3387 of SEQ ID NO:3), not including the terminal codon. The coding sequence encodes a 1129 amino acid protein (SEQ ID NO:2).

A BLASTN 2.0 search against the NRN database, using a score of 100 and a word length of 12 (Altschul et al. (1990) J. Mol. Biol. 215:403) of the nucleotide sequence of human TLCC revealed that human TLCC is 97% identical to human STS WI-30695, sequence tagged site (Accession Number G22461) over nucleotides 3874-3605. This search further revealed that human TLCC is homologous to human chromosome 11p15.5 PAC clone pDJ915f1 containing KvLQT1 gene, complete sequence (Accession Number AC003693).

A BLASTN 2.0 search against the dbEST database, using a score of 100 and a word length of 12 (Altschul et al. (1990) J. Mol. Biol. 215:403) of the nucleotide sequence of human TLCC revealed that human TLCC is 98% identical to nf99c01.s1 NCI_CGAP_Co3 Homo sapiens cDNA clone IMAGE:928032 (Accession Number AA551759) over nucleotides 3865-3369 of SEQ ID NO:1. This search further revealed that human TLCC is 100% identical to tg78b06.x1Soares_NhHMPu_S1 Homo sapiens cDNA clone IMAGE:2114867 (Accession Number AI417040) over nucleotides 3866-3391 of SEQ ID NO:1. This search further revealed that human TLCC is 97% identical to nq58f08.s1 NCI_CGAP_Co9 Homo sapiens cDNA clone IMAGE:1148103 (Accession Number AA633315) over nucleotides 3868-3428 of SEQ ID NO:1. This search further revealed that human TLCC is 98% identical to qp09f02.x1 NCI_CGAP_Kid5 Homo sapiens cDNA clone IMAGE:1917531 3′, mRNA sequence (Accession Number AI344661) over nucleotides 3866-3437 of SEQ ID NO:1. This search further revealed that human TLCC is 97% identical to ah33h08.s1 Soares testis NHT Homo sapiens cDNA clone 1276383 3′ (Accession Number AA694490) over nucleotides 3863-3418 of SEQ ID NO:1.

A BLASTN 2.0 search against the PATENT_{—}2/gsnuc database, using a score of 100 and a wordlength of 12, of the nucleotide sequence of human TLCC revealed that human TLCC is 98% identical to human PS112 consensus DNA fragment from gene specific clones (Accession Number V26656) over nucleotides 1509-3900 of SEQ ID NO:1. This search further revealed that human TLCC is 99% identical to full-length cDNA sequence of prostate tumor clone J1-17 (Accession Number V61200) over nucleotides 2360-3881 of SEQ ID NO:1. This search further revealed that human TLCC is 99% identical to prostate tumour specific gene clone J1-17 (Accession Number V58585) over nucleotides 2360-3881 of SEQ ID NO:1. This search further revealed that human TLCC is 99% identical to human PS112 5′-EST DNA fragment (Accession Number V26657) over nucleotides 2614-3900 of SEQ ID NO:1. This search further revealed that human TLCC is 94% identical to 3′ cDNA sequence of prostate tumor clone J1-17 (Accession Number V61142) over nucleotides 3204-3755 of SEQ ID NO:1. This search further found that human TLCC is 94% identical to 3′ fragment of prostate tumour specific gene J1-17 (Accession Number V58485) over nucleotides 3204-3755 of SEQ ID NO:1. A CLUSTAL W (1.74) alignment of the human TLCC nucleotide sequence with the top hit in this search confirms the similarity of the sequences.

A BLASTN 2.0 search against the PATENT_{—}2/Patent DbPreviewNuc database, using a score of 100 and a wordlength of 12, of the nucleotide sequence of human TLCC revealed that human TLCC is 99% identical to human nucleic acid (Accession Number AC31503 (WO99/46374)) over nucleotides 2339-3886 of SEQ ID NO:1, and 56% identical over nucleotides 3778-3895 of SEQ ID NO:1. This search further revealed that human TLCC is 99% identical to human nucleic acid (Accession Number AC31066 (WO99/46374)) over nucleotides 2621-3170 of SEQ ID NO:1. This search further revealed that human TLCC is 62% identical to 36 secreted proteins (Accession Number AC28066 (WO99/35158)) over nucleotides 2261-3173 of SEQ ID NO:1. This search further revealed that human TLCC is 64% identical to 36 secreted proteins (Accession Number AC28051 (WO99/35158)) over nucleotides 2421-3173 of SEQ ID NO:1.

A BLASTX 2.0 search against the NRP/protot database, using a wordlength of 3, a score of 100, and a BLOSUM62 matrix, of the translated nucleotide sequence of human TLCC revealed that human TLCC is 35% identical to the amino acid sequence of C. elegans hypothetical protein CET01H8.1, CEC05C12.3, CEF54D1.5 similar to trp and trp-like proteins [Homo sapiens] (Accession Number AB001535) over translated nucleic acid residues 147 to 2018 of SEQ ID NO:1, and 41% identical over translated nucleic acid residues 2205-3470 of SEQ ID NO:1. This search further found that human TLCC is 32% identical to the amino acid sequence of Accession Number Z83117, similarity with Drosophila transient-reporter-potential protein (Swiss Prot accession number P19334); cDNA EST EMBL: D27562 comes from this gene, cDNA EST yk219f12.5 comes from this gene [Caenorhabditis elegans] over translated nucleic acid residues 84-1418 of SEQ ID NO:1, 27% identical over translated nucleic acid residues 2190-3368 of SEQ ID NO:1, 30% identical over translated nucleic acid residues 1470-2063 of SEQ ID NO:1, 28% identical over translated nucleic acid residues 3076-3213 of SEQ ID NO:1, 46% identical over translated nucleic acid residues 1613-1651 of SEQ ID NO:1, and 32% identical over translated nucleic acid residues 3705-3839 of SEQ ID NO:1. This search further found that human TLCC is 33% identical to Homo sapiens melastatin I (Accession Number AF071787) over translated nucleic acid residues 2205-3401 of SEQ ID NO:1, 33% identical over translated nucleic acid residues 150-1142 of SEQ ID NO:1, 27% identical over translated nucleic acid residues 1548 to 2405 of SEQ ID NO:1, 48% identical over translated nucleic acid residues 1155-1298, 34% identical over translated nucleic acid residues 3801-3896 of SEQ ID NO:1, 30% identical over translated nucleic acid residues 1261-1380 of SEQ ID NO:1, and 36% identical over translated nucleic acid residues 2451-2516 of SEQ ID NO:1. This search further found that human TLCC is 31% identical to cDNA EST yk308e9.3 comes from this gene; cDNA EST yk308e9.5 comes from this gene; cDNA EST yk318f4.3 comes from this gene; cDNA EST yk318f4.5 comes from this gene; cDNA EST yk398a12.3 comes from this gene, cDNA EST yk398a12.5 comes from this gene (Accession Number Z68333) over translated nucleic acid residues 147-1328 of SEQ ID NO:1, is 23% identical over translated nucleic acid residues 2190-3422 of SEQ ID NO:1, is 31% identical over translated nucleic acid residues 1554-2099 of SEQ ID NO:1, is 34% identical over translated nucleic acid residues 1355-1468 of SEQ ID NO:1, and is 32% identical over translated nucleic acid residues 3225-3338 of SEQ ID NO:1. This search further found that human TLCC is 29% identical to similarity to Worm protein C05C12.3; cDNA EST yk224b10.3 comes from this gene; cDNA EST yk224b10.5 comes from this gene; cDNA EST yk301f12.3 comes from this gene; cDNA EST yk301f12.5 comes from this gene; cDNA EST yk405b7.3 comes from this gene over translated nucleic acid residues 147-2069 of SEQ ID NO:1, is 26% identical over translated nucleic acid residues 2193-2978 of SEQ ID NO:1, and is 34% identical over translated nucleic acid residues 2895-3257 of SEQ ID NO:1. This search further found that human TLCC is 34% identical to Mus musculus melastatin (Accession Number AF047714) over translated nucleic acid residues 150-1142 of SEQ ID NO:1, is 48% identical over translated nucleic acid residues 1155-1298 of SEQ ID NO:1, and is 36% identical over translated nucleic acid residues 2427-2516 of SEQ ID NO:1. A CLUSTAL W (1.74) alignment of the translated human TLCC sequence with the top three hits in this search confirms the similarity of the sequences.

A BLASTX 2.0 search against the PATENT_{—}2/gsprot database, using a score of 100, a wordlength of 3 and a BLOSUM62 matrix, of the translated nucleotide sequence of human TLCC revealed that human TLCC is 95% identical to human PS112 protein sequence from gene-specific clones (Accession Number W54425) over translated nucleic acid residues 1509-3524 of SEQ ID NO:1. This search further revealed that human TLCC is 100% identical to amino acid encoded by prostate tumour clone J1-17 (Accession Number W71868) over translated nucleic acid residues 2580-3524 of SEQ ID NO:1. This search further revealed that human TLCC is 100% identical to prostate tumour specific gene clone J1-17 protein (Accession Number W69384) over translated nucleic acid residues 2580-3524 of SEQ ID NO:1. This search further revealed that human TLCC is 34% identical to prostate-tumour derived antigen #4 (Accession Number Y00931) over translated nucleic acid residues 147-1310 of SEQ ID NO:1, 37% identical over translated nucleic acid residues 2457-3401 of SEQ ID NO:1, 36% identical over translated nucleic acid residues 1554-2018 of SEQ ID NO:1, 46% identical over translated nucleic acid residues 2196-2390 of SEQ ID NO:1, and 38% identical over translated nucleic acid residues 2931-2993 of SEQ ID NO:1. A ClustalW (1.74) alignment of the translated cDNA sequence of human TLCC with the top four hits of this search confirms the similarity of the sequences.

A search was performed against the Memsat database and correlated with an analysis of the hydrophilicity and surface probability of human TLCC, resulting in the identification of six transmembrane domains in the amino acid sequence of human TLCC (SEQ ID NO:2) at about residues 599-619, residues 690-712, residues 784-803, residues 811-831, residues 845-862, and residues 933-957.

A search was also performed against the Prosite database, and resulted in the identification of an N-glycosylation site at residues 143-146, at residues 205-208, and at residues 907-910 of SEQ ID NO:2.

A search was also performed against the ProDom database resulting in the identification of a “transmembrane calcium channel” domain in human TLCC (SEQ ID NO:2) at about residues 783-845. This search further identified significant sequence similarity between the amino acid sequence of human TLCC and human melastatin (Accession Number AAC80000). An alignment (using the GAP program in the GCG software package (Blosum 62 matrix), a gap weight of 12, and a length weight of 4) of the amino acid sequence of human TLCC with human melastatin (Accession Number AAC80000), revealed that human TLCC is 31.739% identical to human melastatin.

As used herein, a “calcium channel” includes a protein or polypeptide which is involved in receiving, conducting, and transmitting signals in an electrically excitable cell, e.g., a neuronal or muscle cell. Calcium channels are calcium ion selective, and can determine membrane excitability (the ability of, for example, a neuronal cell to respond to a stimulus and to convert it into a sensory impulse). Calcium channels can also influence the resting potential of membranes, waveforms and frequencies of action potentials, and thresholds of excitation. Calcium channels are typically expressed in electrically excitable cells, e.g., neuronal cells, and may form heteromultimeric structures (e.g., composed of more than one type of subunit). Calcium channels may also be found in nonexcitable cells (e.g., adipose cells or liver cells), where they may play a role in, e.g., signal transduction. Examples of calcium channels include the low-voltage-gated channels and the high-voltage-gated channels. Calcium channels are described in, for example, Davila et al. (1999) Annals New York Academy of Sciences 868:102-17 and McEnery, M. W. et al. (1998) J. Bioenergetics and Biomembranes 30(4): 409-418, the contents of which are incorporated herein by reference.

Calcium signaling has been implicated in the regulation of a variety of cellular responses, such as growth and differentiation. There are two general methods by which intracellular concentrations of calcium ions may be increased: calcium ions may be brought into the cell from the extracellular milieu through the use of specific channels in the cellular membrane, or calcium ions may be freed from intracellular stores, again being transported by specific membrane channels in the storage organelle. In the situation in which the intracellular stores of calcium have been depleted, a specific type of calcium channel, termed a ‘capacitative calcium channel’ or a ‘store-operated calcium channel’ (SOC), is activated in the plasma membrane to import calcium ions from the extracellular environment to the cytosol (for review, see Putney and McKay (1999) BioEssays 21:38-46).

Members of the capacitative calcium channel family include the calcium release-activated calcium current (CRAC) (Hoth and Penner (1992) Nature 355: 353-355), calcium release-activated nonselective cation current (CRANC) (Krause et al. (1996) J. Biol. Chem. 271: 32523-32528), and the transient receptor potential (TRP) proteins. There is no single electrophysiological profile characteristic of the family; rather, a wide array of single channel conductances, cation selectivity, and current properties have been observed for different specific channels. Further, in several instances it has been demonstrated that homo- or heteropolymerization of the channel molecule may occur, further changing the channel properties from that of the single molecule. In general, though, these channels function similarly, in that they are calcium ion-permeable cation channels which become activated upon stimulation of phospholipase C_{β} by a G protein-coupled receptor. Depletion of intracellular calcium stores activate these channels by a mechanism which is as yet undefined, but which has been demonstrated to involve a diffusible factor using studies in which calcium stores were artificially depleted (e.g., by the introduction of chelators into the cell, by activating phospholipase C_{γ}, or by inhibiting the those enzymes responsible for pumping calcium ions into the stores or those enzymes responsible for maintaining resting intracellular calcium ion concentrations) (Putney, J. W., (1986) Cell Calcium 7: 1-12; Putney, J. W. (1990) Cell Calcium 11:611-624).

The TRP channel family is one of the best characterized of the capacitative calcium channel group. These channels include transient receptor potential protein and homologues thereof (to date, seven homologs and splice variants have been identified in a variety of organisms), the vanilloid receptor subtype I (also known as the capsaicin receptor), stretch-inhibitable non-selective cation channel (SIC), olfactory, mechanosensitive channel, insulin-like growth factor I-regulated calcium channel, and vitamin D-responsive apical, epithelial calcium channel (ECaC) (see, e.g., Montell and Rubin (1989) Neuron 2:1313-1323; Caterina et al. (1997) Nature 389: 816-824; Suzuki et al. (1999) J. Biol. Chem. 274: 6330-6335; Kiselyov et al. (1998) Nature 396: 478-482; and Hoenderop et al. (1999) J. Biol. Chem. 274: 8375-8378). Each of these molecules is 700 or more amino acids (TRP and TRP homologs have 1300 or more amino acid residues), and shares certain conserved structural features. Predominant among these structural features are six transmembrane domains, with an additional hydrophobic loop present between the fifth and sixth transmembrane domains. It is believed that this loop is integral to the activity of the pore of the channel formed upon membrane insertion (Hardie and Minke (1993) Trends Neurosci 16: 371-376). TRP channel proteins also include one or more ankyrin domains and frequently display a proline-rich region at the N-terminus. Although found in disparate tissues and organisms, members of the TRP channel protein family all serve to transduce signals by means of calcium entry into cells, particularly pain (see, e.g., McClesky and Gold (1999) Annu. Rev. Physiol. 61: 835-856), light (Hardie and Minke, supra), or olfactory signals (Colbert et al. (1997) J. Neurosci 17(21): 8259-8269). Thus, this family of molecules may play important roles in sensory signal transduction in general.

Calcium signaling may play a role in liver disease. Ca²⁺ influx has been shown to be essential for the contractile phenotype of activated stellate cells, being the phenotype considered responsible for the high portal hypertension associated with hepatic fibrosis. Hepatic stellate cells, a scarce liver cell type, have been proposed as the main effector of the fibrotic process. Once stimulated, stellate cells acquire the activated phenotype, proliferate, and become fibrogenic. Activated stellate cells contribute to the build-up of extracellular matrix (ECM) via overproduction of ECM components (e.g., collagen), and inhibition of their breakdown. The stimuli for stellate cell activation are not yet clear, although inflammatory cells (e.g., T-lymphocytes) and their mediators (e.g., growth factors, cytokines, and chemokines) interacting with their specific receptors (e.g., GPCRs), have all been postulated to play a role. In addition, PDGF-mediated stellate cell proliferation (a key phenotype of activated stellate cells) depends on Ca²⁺ influx.

As the TLCC molecules of the present invention may modulate calcium channel mediated activities, they may be useful for developing novel diagnostic and therapeutic agents for calcium channel associated disorders.

As used herein, a “calcium channel associated disorder” includes a disorder, disease or condition which is characterized by a misregulation of calcium channel mediated activity. Calcium channel associated disorders include cardiovascular disease and hepatic disorders. A cardiovascular disease or disorder also includes an endothelial cell and/or smooth muscle cell disorder.

Calcium channel disorders may also include CNS disorders and pain disorders. Pain disorders include those that affect pain signaling mechanisms.

Calcium channel disorders also include cellular proliferation, growth, differentiation, or migration disorders. The TLCC molecules of the present invention are involved in signal transduction mechanisms, which are known to be involved in cellular growth, differentiation, and migration processes. Thus, the TLCC molecules may modulate cellular growth, differentiation, or migration, and may play a role in disorders characterized by aberrantly regulated growth, differentiation, or migration.

As used herein, a “calcium channel mediated activity” includes an activity which involves a calcium channel, e.g., a calcium channel in a neuronal cell, a muscular cell, a vascular cell, or a liver cell, associated with receiving, conducting, and transmitting signals, in, for example, the nervous system. Calcium channel mediated activities include release of neurotransmitters or second messenger molecules (e.g., dopamine or norepinephrine), from cells, e.g., neuronal cells; modulation of resting potential of membranes, wave forms and frequencies of action potentials, and thresholds of excitation; participation in signal transduction pathways, and modulation of processes such as integration of sub-threshold synaptic responses and the conductance of back-propagating action potentials in, for example, neuronal cells (e.g., changes in those action potentials resulting in a morphological or differentiative response in the cell).

The term “family” when referring to the protein and nucleic acid molecules of the invention is intended to mean two or more proteins or nucleic acid molecules having a common structural domain or motif and having sufficient amino acid or nucleotide sequence homology as defined herein. For example, the family of TLCC proteins comprises at least one “transmembrane domain” and preferably six transmembrane domains. As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neurosci. 19: 235-263, the contents of which are incorporated herein by reference. Amino acid residues 599-619, 690-712, 784-803, 811-831, 845-862, and 933-957 of the TLCC protein (SEQ ID NO:2) comprise transmembrane domains. Accordingly, TLCC proteins having at least 50-60% homology, preferably about 60-70%, more preferably about 70-80%, or about 80-90% homology with a transmembrane domain of human TLCC are within the scope of the invention.

In another embodiment, a TLCC molecule of the present invention is identified based on the presence of at least one pore domain between the fifth and sixth transmembrane domains. As used herein, the term “pore domain” includes an overall hydrophobic amino acid sequence which is located between two transmembrane domains of a calcium channel protein, preferably transmembrane domains 5 and 6, and which is believed to be a major determinant of ion selectivity and channel activity in calcium channels. Pore domains are described, for example in Vannier et al. (1998) J. Biol. Chem. 273: 8675-8679 and Phillips, A. M. et al. (1992) Neuron 8, 631-642, the contents of which are incorporated herein by reference. Amino acid residues 880-900 of the TLCC protein (SEQ ID NO:2) comprise a pore domain.

In another embodiment, a TLCC molecule of the present invention is identified based on the presence of at least one N-glycosylation site. As used herein, the term “N-glycosylation site” includes an amino acid sequence of about 4 amino acid residues in length which serves as a glycosylation site. More preferably, an N-glycosylation site has the consensus sequence Asn-Xaa-Ser/Thr-Xaa (where Xaa may be any amino acid except proline) (SEQ ID NO:4). N-glycosylation sites are described in, for example, Prosite PDOC00001. Amino acid residues 143-146, 205-208, and 907-910 of the TLCC protein (SEQ ID NO:2) comprise N-glycosylation sites. Accordingly, TLCC proteins having at least one N-glycosylation site are within the scope of the invention.

In another embodiment, a TLCC molecule of the present invention is identified based on the presence of a “transmembrane calcium channel domain” in the protein or corresponding nucleic acid molecule. As used herein, the term “transmembrane calcium channel domain” includes a protein domain having an amino acid sequence of about 40-100 amino acid residues and having a bit score for the alignment of the sequence to the transmembrane calcium channel domain of at about 50-100. Preferably, a transmembrane calcium channel domain includes at least about 60-80, or more preferably about 63 amino acid residues, and has a bit score for the alignment of the sequence to the transmembrane calcium channel domain of at least 20, 25, 30, 35, 40, 45, 50, 55, 60, 65, 70 or higher. The transmembrane calcium channel domain has been assigned ProDom entry 2328. To identify the presence of a transmembrane calcium channel domain in a TLCC protein, and make the determination that a protein of interest has a particular profile, the amino acid sequence of the protein is searched against a database of known protein domains (e.g., the ProDom database) using the default parameters. A search was performed against the ProDom database resulting in the identification of a transmembrane calcium channel domain in the amino acid sequence of human TLCC (SEQ ID NO: 2) at about residues 783-845 of SEQ ID NO: 2.

Isolated proteins of the present invention, preferably TLCC proteins, have an amino acid sequence sufficiently identical to the amino acid sequence of SEQ ID NO:2 or are encoded by a nucleotide sequence sufficiently identical to SEQ ID NO:1 or 3. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably, 70-80%, or 90-95% homology and share a common functional activity are defined herein as sufficiently identical.

As used interchangeably herein, an “TLCC activity”, “biological activity of TLCC” or “functional activity of TLCC”, refers to an activity exerted by a TLCC protein, polypeptide or nucleic acid molecule on a TLCC responsive cell or tissue, or on a TLCC protein substrate, as determined in vivo, or in vitro, according to standard techniques. In one embodiment, a TLCC activity is a direct activity, such as an association with a TLCC-target molecule. As used herein, a “target molecule” or “binding partner” is a molecule with which a TLCC protein binds or interacts in nature, such that TLCC-mediated function is achieved. A TLCC target molecule can be a non-TLCC molecule or a TLCC protein or polypeptide of the present invention. In an exemplary embodiment, a TLCC target molecule is a TLCC ligand, e.g., a calcium channel ligand. Alternatively, a TLCC activity is an indirect activity, such as a cellular signaling activity mediated by interaction of the TLCC protein with a TLCC ligand. The biological activities of TLCC are described herein. For example, the TLCC proteins of the present invention can have one or more of the following activities: (1) modulate membrane excitability, (2) influence the resting potential of membranes, (3) modulate wave forms and frequencies of action potentials, (4) modulate thresholds of excitation, (5) modulate neurite outgrowth and synaptogenesis, (6) modulate signal transduction, (7) participate in nociception, (8) modulate hepatic disorders, (9) modulate angiogenesis, (10) modulate endothelial cell proliferation, and (11) modulate vascular tone.

Accordingly, another embodiment of the invention features isolated TLCC proteins and polypeptides having a TLCC activity. Preferred proteins are TLCC proteins having at least one transmembrane domain, and, preferably, a TLCC activity. Other preferred proteins are TLCC proteins having an N-glycosylation site and, preferably, a TLCC activity. Yet other preferred proteins are TLCC proteins having at least one transmembrane calcium channel domain and, preferably, a TLCC activity. Yet other preferred proteins are TLCC proteins having at least one transmembrane domain, at least one N-glycosylation site, and a transmembrane calcium channel domain and, preferably, a TLCC activity.

Additional preferred proteins have at least one transmembrane domain, and one or more of the following domains: at least one N-glycosylation site, and a transmembrane calcium channel domain, and are, preferably, encoded by a nucleic acid molecule having a nucleotide sequence which hybridizes under stringent hybridization conditions to a nucleic acid molecule comprising the nucleotide sequence of SEQ ID NO:1 or 3.

Tissue Distribution of TLCC mRNA

This example describes the tissue distribution of TLCC mRNA, as was qualitatively determined by Polymerase Chain Reaction (PCR), and quantitatively measured using the Taqman™ procedure.

Using PCR techniques, the human TLCC gene was determined to be predominantly expressed in osteoblasts, with some expression also seen in brain, adipose tissue, breast, colon, all fetal tissues, liver, pituitary, melanocyte, prostate, cervix, muscle, small intestine, megakaryocytes, and aorta, as well as in lymphoma and colon to liver metastases.

Using the Taqman™ procedure, it was determined that TLCC mRNA was expressed at low levels in normal human heart, kidney, lung, and liver. A very marked upregulation was detected in passaged human stellate cells, as well as in human fibrotic livers, although expression was low in quiescent stellate cells. TLCC mRNA was upregulated in human dermal and lung fibroblasts cultured in the presence of TGF-β.

It was also determined that the rat orthologue of TLCC was highly increased in all bile duct ligation-induced fibrotic livers tested as compared to control animals. Upregulation was detected in all carbon tetrachloride-induced fibrotic livers as compared to controls. However, there was no significant regulation in the serum-induced fibrotic livers as compared to controls, and no regulation in the cultured rat stellate cells. These data reveal that TLCC is highly regulated in activated stellate cells and in fibrotic livers, being expressed only at low levels in other organs and cell types. These observations suggest that TLCC may play an important role in Ca²⁺-dependent phenomena (e.g., hepatic cell contractility and proliferation). The functional linkage of TRP channels to inositol triphosphate further suggests that TLCC might be related to key signaling events during stellate cell activation.

### Expression of TLCC in Blood Vessels

Reverse Transcriptase PCR (RT-PCR) was performed using the Taqman procedure to detect the presence of RNA transcripts corresponding to human TLCC in mRNA prepared from isolated human vessels or cells cultured from the endothelial vasculature. Significant TLCC expression was detected in vascular smooth muscle cells cultured from human aorta as well as in endothelial cells cultured from lung microvasculature or umbilical vein. Expression of TLCC was downregulated when cultured umbilical vein endothelial cells were treated with human recombinant IL-1β for six hours. Expression of TLCC in several isolated human vessels exceeded the expression level of TLCC in human adipose tissue which was included as a control.

### Expression of TLCC in Endothelial Cells During Laminar Shear Stress

Human umbilical vein endothelial cells (HUVECs) were cultured in vitro under standard conditions, described in, for example, U.S. Pat. No. 5,882,925. Experimental cultures were then exposed to laminar shear stress (LSS) conditions.

Cultured HUVEC monolayers were exposed to laminar sheer stress by culturing the cells in a specialized apparatus containing liquid culture medium. Static cultures grown in the same medium served as controls. The in vitro LSS treatment at 10 dyns/cm² was performed for 24 hours and was designed to simulate the shear stress generated by blood flow in a straight, healthy artery.

The effect of LSS on TLCC expression in endothelial cells was assessed from total RNA prepared from the cells and used to probe clones arrayed on nylon filters. A TLCC clone showed a higher signal when probed with two of the three LSS samples when compared to their static controls, indicating that expression of TLCC is upregulated by laminar shear stress.

### Human 15603

The present invention relates to a human TRP6, 15603. Drosophila transient receptor potential (TRP) proteins and some mammalian homologues (TRPC proteins) are thought to mediate capacitative Ca⁺² entry (Hofmann et al., Nature 397:259-263 (1999)). Seven mammalian homologous genes (TRPC 1 to TRPC 7) have been cloned and characterized. TRPC6, together with TRPC 3 have been identified as the first members of a new functional family of second-messenger-operated cation channels, which are activated by diacylglycerol independently of protein kinase C (Hofmann et al., supra). It has recently been demonstrated that TRPC6 is likely to be the essential component of the α1-adrenoceptor-activated nonselective cation channel (α1-AR-NSCC), which may serve as a store-depletion-independent Ca²⁺ entry pathway during increased sympathetic activity. The α1-androgen receptor (α1-AR) is expressed widely in the vascular system. α1-AR stimulation leads to activation of G protein-coupled phospholipase Cβ, which catalyzes formation from phosphoinositide of 1,4,5-triphosphate (IP3) and diacylglycerol, which leads to the release of stored Ca²⁺ and sustained Ca²⁺ entry.

The human 15603 sequence (SEQ ID NO:5), which is approximately 2796 nucleotides long including untranslated regions, contains a predicted methionine-initiated coding sequence of about 2793 nucleotides (nucleotides 1-2793 of both SEQ ID NO:5 and SEQ ID NO:7) not including the terminal codon. The coding sequence encodes a 931 amino acid protein (SEQ ID NO:6).

The human 15603 protein of SEQ ID NO:6 includes an amino-terminal hydrophobic amino acid sequence, consistent with a signal sequence, of about 26 amino acids (from amino acid 1 to about amino acid 26 of SEQ ID NO:6, PSORT, Nakai and Kanehisa (1992) Genomics 14:897-911), which upon cleavage results in the production of a mature protein form. This mature protein form is approximately 905 amino acid residues in length (from about amino acid 27 to amino acid 931 of SEQ ID NO:6).

Human 15603 contains the following regions or other structural features (for general information regarding PFAM identifiers, PS prefix and PF prefix domain identification numbers, refer to Sonnhammer et al. (1997) Protein 28:405-420: an ank domain (PFAM Accession Number PF0023) located at about amino acid residues 97 to 131 of SEQ ID NO:6; an ank domain (PFAM Accession Number PF0023) located at about amino acid residues 132 to 163 of SEQ ID NO:6; an ank domain (PFAM Accession Number PF0023) located at about amino acid residues 218 to 250 of SEQ ID NO:6; an ion transport protein domain (PFAM Accession Number PF00520) located at about amino acid residues 493 to 727 of SEQ ID NO:6; a Receptor Channel Potential Transient Repeat Variant Calcium Entry Ionic domain (ProDom No. PD140156) located at about amino acid residues 1 to 39 of SEQ ID NO:6; a Channel Receptor Transient Potential Calcium Repeat Variant Entry Ion domain (ProDom No. PD323618) located at about amino acid residues 40 to 71 of SEQ ID NO:6; a Channel Receptor Calcium Potential Capacitative Entry Trp-Related Transient Repeat Variant domain (ProDom No. PD186301) located at about amino acid residues 93 to 133 of SEQ ID NO:6; a Repeat Channel Ionic Receptor Trp Vision Transient Potential Ank domain (ProDom No. PD140149) located at about amino acid residues 95 to 154 of SEQ ID NO:6; a Repeat Ankyrin Channel Gene ORF Family Receptor Ankyrin-Like Factor (ProDom No. PD007334) located at about amino acid residues 102 to 166 of SEQ ID NO:6; a Ankyrin Repeat Kinase Domain UNC-44 Ankyrin-Related Alternative Glycoprotein EGF-like domain (ProDom No. PD000041) located at about amino acid residues 105 to 188 of SEQ ID NO:6; a Channel Receptor Transient Repeat Calcium Potential Ion Ank Transport Transmembrane domain (ProDom No. PD004194) located at about amino acid residues 123 to 347 of SEQ ID NO:6; a Receptor Channel Potential Transient NOMPC TRP2 Y71A12B.4 Y71A12B.E 2-Beta 2-Alpha domain (ProDom No. PD296552) located at about amino acid residues 299 to 509 of SEQ ID NO:6; a Transmembrane Fis Receptor MTR1 domain (ProDom No. PD039592) located at about amino acid residues 308 to 916 of SEQ ID NO:6; a Channel Receptor Calcium Repeat Transient Potential Ion Transmembrane Ionic Transport domain (ProDom No. PD328255) located at about amino acid residues 362 to 509 of SEQ ID NO:6; a Channel Receptor Calcium Transient Potential Repeat Vanilloid Transmembrane Ion Transport domain (ProDom No. PD003230) located at about amino acid residues 409 to 748 of SEQ ID NO:6; a Channel Receptor Calcium Potential Repeat Transient Ion Ionic Variant Ank domain (ProDom No. PD238062) located at about amino acid residues 510 to 597 of SEQ ID NO:6; a Channel Receptor Calcium Repeat Potential Transient Capacitative Entry Ion Transport domain (ProDom No. PD342728) located at about amino acid residues 744 to 895 of SEQ ID NO:6; a Channel Receptor Transient Potential Repeat Calcium Transport Transmembrane Ank Ionic domain (ProDom No. PD004174) located at about amino acid residues 749 to 900 of SEQ ID NO:6; a Channel Repeat Calcium Ionic Entry Receptor Transient Ank Transport domain (ProDom No. PD266294) located at about amino acid residues 786 to 854 of SEQ ID NO:6; a Gelsolin-Related domain (ProDom No. PD202783) located at about amino acid residues 792 to 931 of SEQ ID NO:6; a Coiled Coil Myosin Repeat Chain Heavy Filament Heptad Pattern Muscle domain (ProDom No. PD000002) located at about amino acid residues 796 to 928 of SEQ ID NO:6; a Channel Receptor Transient Potential Calcium Repeat Variant Entry Ion (ProDom No. PD137340) located at about amino acid residues 901 to 931 of SEQ ID NO:6; a transmembrane domain (predicted by MEMSAT, Jones et al. (1994) Biochemistry 33:3038-3049) at about amino acids 407 to 426 of SEQ ID NO:6; a transmembrane domain (predicted by MEMSAT, Jones et al. (1994) Biochemistry 33:3038-3049) at about amino acids 443 to 459 of SEQ ID NO:6; a transmembrane domain (predicted by MEMSAT, Jones et al. (1994) Biochemistry 33:3038-3049) at about amino acids 490 to 507 of SEQ ID NO:6; a transmembrane domain (predicted by MEMSAT, Jones et al. (1994) Biochemistry 33:3038-3049) at about amino acids 598 to 614 of SEQ ID NO:6; a transmembrane domain (predicted by MEMSAT, Jones et al. (1994) Biochemistry 33:3038-3049) at about amino acids 637 to 653 of SEQ ID NO:6; a transmembrane domain (predicted by MEMSAT, Jones et al. (1994) Biochemistry 33:3038-3049) at about amino acids 703 to 727 of SEQ ID NO:6; two coiled coil segments located at about amino acids 297 to 327 (ALELSNELAVLANIEKEFKNDYKKLSMQCKD; SEQ ID NO:88) and 878 to 923 (EVNEGELKEIKQDISSLRYELLEEKSQNTEDLAELIRELGEKLSME; SEQ ID NO:89) of SEQ ID NO:6; a leucine zipper pattern (Prosite PS00029) located at about amino acids 766 to 787 (LVPSPKSLFYLLLKLKKWISEL; SEQ ID NO:90) of SEQ ID NO:6; nine protein kinase C phosphorylation sites (Prosite PS00005) located at about amino acids 14 to 16 (SPR), 89 to 91 (SDR), 563 to 565 (TLK), 630 to 632 (TVK), 674 to 676 (SFK), 769 to 771 (SPK), 836 to 838 (SIR), 893 to 895 (SLR), and 929 to 931 (TNR) of SEQ ID NO:6; sixteen casein kinase II phosphorylation sites (Prosite PS00006) located at about amino acids 96 to 99 (SIEE; SEQ ID NO:91), 197 to 200 (SQSE; SEQ ID NO:92), 216 to 219 (SSHD; SEQ ID NO:93), 289 to 292 (SSED; SEQ ID NO:94), 296 to 299 (TALE; SEQ ID NO:95), 475 to 478 (TSTD; SEQ ID NO:96), 492 to 495 (SWME; SEQ ID NO:97), 556 to 559 (SIID; SEQ ID NO:98), 563 to 566 (TLKD; SEQ ID NO:99), 571 to 574 (TLGD; SEQ ID NO:100), 630 to 633 (TVKD; SEQ ID NO:101), 669 to 672 (TTVE; SEQ ID NO:102), 730 to 733 (SFQE; SEQ ID NO:103), 752 to 755 (SYFE; SEQ ID NO:104), 815 to 818 (SHED; SEQ ID NO:105), and 839 to 842 (SSED; SEQ ID NO:106) of SEQ ID NO:6; two cAMP/cGMP-dependent protein kinase phosphorylation sites (Prosite PS00004) located at about amino acids 67 to 70 (RRQT; SEQ ID NO:107) and 319 to 322 (KKLS; SEQ ID NO:108) of SEQ ID NO:6; three tyrosine phosphorylation sites (Prosite PS00007) located at about amino acids 24 to 31 (RRNESQDY; SEQ ID NO:109), 101 to 108 (RFLDAAEY; SEQ ID NO:110), and 698 to 705 (KFIENIGY; SEQ ID NO:111) of SEQ ID NO:6; two amidation sites (Prosite PS00009) located at about amino acids 75 to 78 (KGRR; SEQ ID NO:112) and 188 to 191 (EGKR; SEQ ID NO:113) of SEQ ID NO:6; nine N-glycosylation sites (Prosite PS00001) located at about amino acids 26 to 29 (NESQ; SEQ ID NO:114), 157 to 160 (NLSR; SEQ ID NO:115), 362 to 365 (NLSR; SEQ ID NO:116), 394 to 397 (NLSG; SEQ ID NO:117), 473 to 476 (NETS; SEQ ID NO:118), 561 to 564 (NDTL; SEQ ID NO:119), 617 to 620 (NESF; SEQ ID NO:120) 712 to 715 (NVTM; SEQ ID NO:121) and 728 to 731 (NSSF; SEQ ID NO:122) of SEQ ID NO:6; and five N-myristoylation sites (Prosite PS00008) located at about amino acids 17 to 22 (GAAGAA; SEQ ID NO:123), 213 to 218 (GTRSSH; SEQ ID NO:124), 504 to 509 (GMIWAE; SEQ ID NO:125), 661 to 666 (GAKQNE; SEQ ID NO:126), and 811 to 816 (GILGSH; SEQ ID NO:127) of SEQ ID NO:6.

A hydropathy plot of human 15603 was performed. Polypeptides of the invention include fragments which include: all or part of a hydrophobic sequence, e.g., the sequence from about amino acid 162 to 172, from about amino acid 215 to 225, from about amino acid 325 to 335, from about amino acid 401 to 431, from about amino acid 441 to 461, from about amino acid 486 to 506, from about amino acid 521 to 551, from about amino acid 591 to 616, from about amino acid 631 to 656, from about amino acid 665 to 675, from about amino acid 701 to 731, and from about amino acid 770 to 780 of SEQ ID NO:6; all or part of a hydrophilic sequence, e.g., the sequence from about amino acid 20 to 32, from about amino acid 55 to 85, from about amino acid 181 to 221, from about amino acid 241 to 281, from about amino acid 306 to 321, from about amino acid 331 to 340, from about amino acid 461 to 486, from about amino acid 780 to 805, from about amino acid 810 to 841, from about amino acid 841 to 851, from about amino acid 865 to 890, from about amino acid 895 to 905, and from about amino acid 915 to 931 of SEQ ID NO:6; a sequence which includes a Cys, or a glycosylation site.

The 15603 protein contains a significant number of structural characteristics in common with members of the ion channel family and the ank repeat family. As used herein, the term “ion channel” includes a protein or polypeptide which is capable of regulating the flow of ions such as calcium cations through a channel. The channel may regulate the flow of a particular cation or anion or may be less discriminating and allow multiple types of cations or anions to flow through it. The flow of ions may be regulated by the presence or absence of a bound ligand or may be regulated by the phosphorylation state of the channel or another protein associated with the channel. The ion channel protein may undergo a conformational change based on the binding of a ligand, the phosphorylation of a particular residue, or the binding of another protein or biomolecule.

Members of an ion channel family of proteins are characterized by transmembrane domains, cytoplasmic domains, extracellular domains, and may be homodimers, homotrimers, homomultimers, heterodimeric etc. The pores of ion channels are typically formed by multiple transmembrane proteins encoded by the same or different genes. Typically, a hydrophilic transmembrane channel is formed that allows passage of ions from one side of the plasma membrane to the other. Clusters of charged amino acids at the mouth of the channel may increase the selectivity for a particular type of ion, e.g., clusters of negatively charged amino acid residues at the mouth of the channel may exclude negative ions from cation channels.

All ion channel family proteins form water-filled pores across membranes. Ion channel proteins are located in the plasma membrane of animal and plant cells and are further characterized by small highly selective pores that participate in ion transport. Typically, more than 10⁶ ions can pass through such a channel each second. Many ion channels allow specific ions, such as Na⁺, K⁺, Ca²⁺, or Cl⁻, to diffuse down their electrochemical gradients across the lipid bilayer. The ion channel proteins show ion selectivity, permitting some ions to pass but not others. Another feature of ion channel proteins is that they are not continuously open, in contrast to simple aqueous pores. Instead, they have “gates,” which open briefly and then close again. This opening and closing is usually in response to a specific perturbation of the membrane, such as a change in voltage across the membrane (voltage-gated channels), mechanical stimulation (mechanically-gated channels), or the binding of a signaling molecule (ligand-gated channels). In the case of ligand-gated channels, the signaling ligand can be either an extracellular mediator, such as a neurotransmitter (transmitter-gated channels), an intracellular mediator, such as an ion (ion-gated channels), a nucleotide (nucleotide-gated channels), or a GTP-binding regulatory protein (G-protein-gated channels).

Ion channels are responsible for the electrical excitability of nerve and muscle cells and mediate most forms of electrical signaling in the nervous system. A single nerve cell typically contains more than five kinds of ion channels. However, these ion channels are not restricted to electrically excitable cells. Ion channels are present in all animal cells and are found in plant cells and microorganisms. For example, ion channel proteins propagate the leaf-closing response of the mimosa plant and allow the single-celled paramecium to reverse direction after collision. 15603, also known as TRPC6, is a non-selective cation channel that is activated by diacylglycerol in a membrane-delimited fashion, independently of protein kinase C

A 15603 polypeptide can include a “ion channel domain” or regions homologous with a “ion channel domain”. A 15603 polypeptide can further include a “ank domain” or regions homologous with a “ank domain”.

As used herein, the term “ion transport protein domain” includes an amino acid sequence of about 200 to 250 amino acid residues in length and having a bit score for the alignment of the sequence to the ion transport protein domain (HMM) of at least 75. Preferably, an ion transport domain mediates the flow of ions through a membrane. Preferably, an ion transport protein domain includes at least about 200 to 300 amino acids, more preferably about 200 to 250 amino acid residues, or about 210 to 240 amino acids and has a bit score for the alignment of the sequence to the ion transport protein domain (HMM) of at least 50, 75, 80, or greater. The ion transport domain can include transmembrane domains. The ion transport protein domain (HMM) has been assigned the PFAM Accession Number PF00520. The ion transport protein domain (amino acids 493 to 727 of SEQ ID NO:6) of human 15603 aligns with the Pfam ion transport protein consensus amino acid sequence (SEQ ID NO:11) derived from a hidden Markov model.

As used herein, the term “ank repeat domain” includes an amino acid sequence of about 30 to 35 amino acid residues in length and having a bit score for the alignment of the sequence to the ank repeat domain (HMM) of at least 15. Preferably, an ank repeat domain includes at least about 20 to 40 amino acids, more preferably about 25 to 35 amino acid residues, or about 30 to 35 amino acids and has a bit score for the alignment of the sequence to the ank repeat domain (HMM) of at least 10, 15, 20, or greater. The ank repeat domain (HMM) has been assigned the PFAM Accession Number PF0023. Additionally, the ank repeat domain (HMM) has been assigned the SMART identifier ANK_{—}2a. The ank repeat domains (amino acids 97 to 131, 132 to 163, and 218 to 250 of SEQ ID NO:6) of human 15603 align with the Pfam ank repeat domain consensus amino acid sequences (SEQ ID NO:8-10) derived from a hidden Markov model. The ank repeat domains (amino acids 97 to 126, 132 to 160, and 218 to 247 of SEQ ID NO:6) of human 15603 align with the SMART ANK_{—}2a domain consensus amino acid sequences (SEQ ID NO:8-10) derived from a hidden Markov model.

In a preferred embodiment, a 15603 polypeptide or protein has an “ion transport protein domain” or a region which includes at least about 200 to 300 more preferably about 200 to 250 or 210 to 240 amino acid residues and has at least about 60%, 70% 80% 90% 95%, 99%, or 100% homology with a “ion transport protein domain,” e.g., the ion transport protein domain of human 15603 (e.g., residues 493 to 727 of SEQ ID NO:6).

In a preferred embodiment, a 15603 polypeptide or protein has an “ank repeat domain” or a region which includes at least about 25 to 40 more preferably about 25 to 35 or 30 to 35 amino acid residues and has at least about 60%, 70% 80% 90% 95%, 99%, or 100% homology with an “ank repeat domain,” e.g., an ank repeat domain of human 15603 (e.g., residues 97 to 131, 132 to 163, or 218 to 250 of SEQ ID NO:6).

To identify the presence of a “ion transport protein” domain or an “ank repeat” domain in a 15603 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of an “ion transport protein” domain in the amino acid sequence of human 15603 at about residues 493 to 727 of SEQ ID NO:6. A search was performed against the HMM database resulting in the identification of three “ank repeat” domains in the amino acid sequence of human 15603 at about residues 97 to 131, 132 to 163, and 218 to 250 of SEQ ID NO:6.

An additional method to identify the presence of an “ank repeat” domain in a 15603 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (2000) Nucl. Acids Res 28:231. The database contains domains identified by profiling with the hidden Markov models of the HMMer2 search program (Durbin et al. (1998) Biological sequence analysis: probabilistic models of proteins and nucleic acids. Cambridge University Press). The database also is extensively annotated and monitored by experts to enhance accuracy. A search was performed against the HMM database resulting in the identification of three “ank 2a” domains in the amino acid sequence of human 15603 at about residues 97 to 126, 132 to 160, and 218 to 247 of SEQ ID NO:6.

For further identification of domains in a 15603 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the HMM database resulting in the identification of a “channel receptor transient repeat calcium potential ion ank transport transmembrane” domain in the amino acid sequence of human 15603 at about residues 123 to 347 of SEQ ID NO:6. A BLAST alignment of the human 15603 ion channel domain with a consensus amino acid sequence of a domain derived from the ProDomain database (“Channel Receptor Transient Repeat Calcium Potential Ion Ank Transport Transmembrane;” No. PD004194; ProDomain Release 2001.1) showed the amino acid residues 52 to 278 of the 278 amino acid PD004194 consensus sequence (SEQ ID NO:12) aligned with the ion channel domain of human 15603, amino acid residues 123 to 347 of SEQ ID NO:6.

A 15603 molecule can further include a transmembrane domain or an ank repeat domain.

A 15603 polypeptide can include at least one, two, three, four, five, preferably six “transmembrane domains” or regions homologous with “transmembrane domains”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta et al., (1996) Annual Rev. Neurosci. 19:235-263, the contents of which are incorporated herein by reference. The transmembrane domains of human 15603 are located at about residues 407 to 426, 443 to 459, 490 to 507, 598 to 614, 637 to 653, and 703 to 727 of SEQ ID NO:6.

In a preferred embodiment, a 15603 polypeptide or protein has at least one, two, three, four, five, preferably six “transmembrane domain” or a region which includes at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 15603 (e.g., residues 407 to 426, 443 to 459, 490 to 507, 598 to 614, 637 to 653, or 703 to 727 of SEQ ID NO:6). The transmembrane domain of human 15603 can be visualized in a hydropathy plot as regions of about 15 to 25 amino acids where the hydropathy trace is mostly above the horizontal line.

To identify the presence of a “transmembrane” domain in a 15603 protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 15603 polypeptide can include at least one, two, three, four, five, six, preferably seven “non-transmembrane regions.” As used herein, the term “non-transmembrane region” includes an amino acid sequence not identified as a transmembrane domain. The non-transmembrane regions in 15603 are located at about amino acids 1 to 406, 427 to 442, 460 to 489, 508 to 597, 615 to 636, 654 to 702, and 727 to 931 of SEQ ID NO:6. Non-transmembrane domains can be cytoplasmic or extracellular.

The non-transmembrane regions of 15603 include at least one, two, three, preferably four cytoplasmic regions. When located at the N-terminus, the cytoplasmic region is referred to herein as the “N-terminal cytoplasmic domain.” As used herein, an “N-terminal cytoplasmic domain” includes an amino acid sequence having about 1 to 500, preferably about 1 to 450, more preferably about 1 to 425, or even more preferably about 1 to 410 amino acid residues in length, is located inside of a cell or within the cytoplasm of a cell. The C-terminal amino acid residue of an “N-terminal cytoplasmic domain” is adjacent to an N-terminal amino acid residue of a transmembrane domain in a 15603 protein. For example, an N-terminal cytoplasmic domain is located at about amino acid residues 1 to 406 of SEQ ID NO:6.

In a preferred embodiment, a 15603 polypeptide or protein has an N-terminal cytoplasmic domain or a region which includes about 1 to 450, preferably about 1 to 425, and more preferably about 1 to 410 amino acid residues and has at least about 60%, 70% 80% 90% 95%, 99%, or 100% homology with an “N-terminal cytoplasmic domain,” e.g., the N-terminal cytoplasmic domain of human 15603 (e.g., residues 1 to 406 of SEQ ID NO:6).

In another embodiment, a 15603 cytoplasmic region includes at least one, preferably two cytoplasmic loops. As used herein, the term “loop” includes an amino acid sequence which is not included within a phospholipid membrane, having a length of at least about 4, preferably about 5 to 95, more preferably about 6 to 35 amino acid residues, and has an amino acid sequence that connects two transmembrane domains within a protein or polypeptide. Accordingly, the N-terminal amino acid of a loop is adjacent to a C-terminal amino acid of a transmembrane domain in a 15603 molecule, and the C-terminal amino acid of a loop is adjacent to an N-terminal amino acid of a transmembrane domain in a 15603 molecule. As used herein, a “cytoplasmic loop” includes a loop located inside of a cell or within the cytoplasm of a cell. For example, a “cytoplasmic loop” can be found at about amino acid residues 460 to 489 and 615 to 636 of SEQ ID NO:6.

In a preferred embodiment, a 15603 polypeptide or protein has a cytoplasmic loop or a region which includes at least about 4, preferably about 5 to 40, and more preferably about 6 to 35 amino acid residues and has at least about 60%, 70% 80% 90% 95%, 99%, or 100% homology with a cytoplasmic loop, e.g., a cytoplasmic loop of human 15603 (e.g., residues 460 to 489 and 615 to 636 of SEQ ID NO:6).

In another embodiment, a 15603 non-transmembrane region includes at least one, two, preferably three non-cytoplasmic loops. As used herein, a “non-cytoplasmic loop” includes a loop located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 427 to 442, 508 to 597, and 654 to 702 of SEQ ID NO:6.

In a preferred embodiment, a 15603 polypeptide or protein has at least one non-cytoplasmic loop or a region which includes at least about 4, preferably about 5 to 100, more preferably about 6 to 90 amino acid residues and has at least about 60%, 70% 80% 90% 95%, 99%, or 100% homology with a “non-cytoplasmic loop,” e.g., at least one non-cytoplasmic loop of human 15603 (e.g., residues 427 to 442, 508 to 597, and 654 to 702 of SEQ ID NO:6).

In another embodiment, a cytoplasmic region of a 15603 protein can include the C-terminus and can be a “C-terminal cytoplasmic domain,” also referred to herein as a “C-terminal cytoplasmic tail.” As used herein, a “C-terminal cytoplasmic domain” includes an amino acid sequence having a length of at least about 200, preferably about 150 to 250, more preferably about 175 to 225 amino acid residues, is located inside of a cell or within the cytoplasm of a cell. The N-terminal amino acid residue of a “C-terminal cytoplasmic domain” is adjacent to a C-terminal amino acid residue of a transmembrane domain in a 15603 protein. For example, a C-terminal cytoplasmic domain is located at about amino acid residues 728 to 931 of SEQ ID NO:6.

In a preferred embodiment, a 15603 polypeptide or protein has a C-terminal cytoplasmic domain or a region which includes at least about 200, preferably about 150 to 250, and more preferably about 175 to 225 amino acid residues and has at least about 60%, 70% 80% 90% 95%, 99%, or 100% homology with a C-terminal cytoplasmic domain, e.g., the C-terminal cytoplasmic domain of human 15603 (e.g., residues 728 to 931 of SEQ ID NO:6).

A human 15603 protein can further include a leucine zipper or coiled coil structure. Preferably a leucine zipper domain has at least about 60%, 70%, 80%, 90%, 95%, 99%, or 100% homology with the leucine zipper domain of human 15603 (e.g., residues 766 to 787 of SEQ ID NO:2). Preferably a coiled coil domain has at least about 60%, 70%, 80%, 90%, 95%, 99%, or 100% homology with the coiled coil domain of human 15603 (e.g., residues 296 to 907 of SEQ ID NO:6). A human 15603 protein can further include N-glycosylation sites, cAMP and cGMP-dependent protein kinase phosphorylation sites, protein kinase C phosphorylation sites, casein kinase II phosphorylation site, tyrosine kinase phosphorylation sites, N-myristoylation sites, and amidation sites.

A 15603 family member can include at least one ion channel protein domain; and at least one, two, preferably three ank repeat domains or transmembrane or non-transmembrane domains. A 15603 family member can include at least one leucine zipper structure or at least one coiled coil structure. Furthermore, a 15603 family member can include at least one, two, three, four, five, six, seven, eight, preferably nine protein kinase C phosphorylation sites (Prosite PS00005); at least one, two, three, four, five, six, seven, eight, nine, ten, eleven, twelve, thirteen, fourteen, fifteen, and preferably sixteen casein kinase II phosphorylation sites (Prosite PS00006); at least one, two, three, four, five, six, seven, eight, preferably nine N-glycosylation sites (Prosite PS00001); at least one, preferably two cAMP/cGMP protein kinase phosphorylation sites (Prosite PS00004); at least one, preferably two amidation sites (Prosite PS00009); and at least one, two, three, four, and preferably five N-myristoylation sites (Prosite PS00008).

As the 15603 polypeptides of the invention can modulate 15603-mediated activities, they can be useful for developing novel diagnostic and therapeutic agents for ion channel-associated or other 15603-associated disorders, as described below.

As used herein, a “ion channel-associated activity” includes an activity which involves the regulation of the flow of ions across a membrane. The flow of ions may be controlled by a second-messenger such as diacylglycerol. Members of the family can play a role in cardiovascular disease such as abnormal angiogenesis.

As used herein, a “15603 activity”, “biological activity of 15603” or “functional activity of 15603”, refers to an activity exerted by a 15603 protein, polypeptide or nucleic acid molecule on e.g., a 15603-responsive cell or on a 15603 substrate, e.g., a protein substrate, as determined in vivo or in vitro. In one embodiment, a 15603 activity is a direct activity, such as an association with a 15603 target molecule. A “target molecule” or “binding partner” is a molecule with which a 15603 protein binds or interacts in nature. In an exemplary embodiment, 15603 is a ion channel, e.g., a non-selective cation channel that is activated by diacylglycerol, and thus interacts in nature with a molecule such as a second messenger (e.g., diacylglycerol) to regulate the flow of cations through a membrane.

A 15603 activity can also be an indirect activity, e.g., a cellular signaling activity mediated by interaction of the 15603 protein with a 15603 receptor. Based on the above-described sequence structures and similarities to molecules of known function, the 15603 molecules of the present invention can have similar biological activities as ion channel family members. For example, the 15603 proteins of the present invention can have one or more of the following activities: (1) the ability to bind a second messenger; (2) the ability to bind diacylglycerol; (3) the ability to regulate the flow of cations through a membrane; (4) the ability to regulate angiogenesis; and (5) the ability to regulate intracellular calcium levels.

The 15603 molecules of the invention can modulate the activities of cells in tissues where they are expressed. For example, using TaqMan analysis, 15603 mRNA is expressed in moderate to high levels in hemangiomas, megakaryocytes, Wilm's tumor, vascular smooth muscle cells, proliferative endothelial cells, uterine, and normal brain cortex. Accordingly, the 15603 molecules of the invention can act as therapeutic or diagnostic agents for cardiovascular, angiogenic, or neoproliferative disorders.

Thus, the 15603 molecules can act as novel diagnostic targets and therapeutic agents for controlling one or more cardiovascular, angiogenic, neoproliferative, or other ion channel disorders. As used herein, “ion channel disorders” are diseases or disorders whose pathogenesis is caused by, is related to, or is associated with aberrant or deficient ion channel protein function or expression. Examples of such disorders, e.g., ion channel-associated or other 15603-associated disorders, include but are not limited to, cellular proliferative and/or differentiative disorders, immune e.g., inflammatory, disorders, cardiovascular disorders, endothelial cell disorders, or renal disorders.

The 15603 molecules can be used to treat cardiovascular, immune system, or proliferative disorders in part because family members are found in the hemangiomas, blood vessels, and megakaryocytes.

The 15603 molecules of the invention can be used to monitor, treat and/or diagnose a variety of proliferative disorders. Such disorders include hematopoietic neoplastic disorders.

Aberrant expression and/or activity of 15603 molecules can mediate disorders associated with bone metabolism.

The 15603 nucleic acid and protein of the invention can be used to treat and/or diagnose a variety of immune, e.g., inflammatory, (e.g. respiratory inflammatory) disorders

Additionally, 15603 molecules can play an important role in the etiology of certain viral diseases, including but not limited to Hepatitis B, Hepatitis C and Herpes Simplex Virus (HSV). Modulators of 15603 activity could be used to control viral diseases. The modulators can be used in the treatment and/or diagnosis of viral infected tissue or virus-associated tissue fibrosis, especially liver and liver fibrosis. Also, 15603 modulators can be used in the treatment and/or diagnosis of virus-associated carcinoma, especially hepatocellular cancer.

Additionally, 15603 can play an important role in the regulation of metabolism or pain disorders.

### Gene Expression Analysis of 15603

Human 15603 expression was measured by TaqMan® quantitative PCR (Perkin Elmer Applied Biosystems) in cDNA prepared from a variety of normal and diseased (e.g., cancerous) human tissues or cell lines.

The results indicate significant 15603 expression in hemangioma, vascular smooth muscle, megakaryocytes, Wilm's tumor; at medium levels in normal brain cortex, uterine, and proliferative endothelial cells; and at low levels in adipose tissue, normal human internal mammary artery, diseased human aorta, diseased human artery, diseased human vein, and normal human vein, normal heart, CHF heart, normal kidney, skeletal muscle, pancreas, primary osteoblasts, brain hypothalamus, normal breast, normal ovary, normal prostate, prostate tumor, salivary glands, normal colon, colon tumor, normal lung, lung tumor, lung COPD, colon IBD, normal spleen, normal tonsil, normal small intestine, skin decubitus, synovium, glioblastomas, fetal adrenal, fetal kidney, and fetal heart.

TABLE 2
Phase 1.5.1 Expression of 15603 w/beta 2

| Tissue Type | Expression |
|---|---|
| Artery normal | 0.6556 |
| Aorta diseased | 0.3871 |
| Vein normal | 0.1012 |
| Coronary SMC | 0 |
| HUVEC | 0 |
| Hemangioma | 28.1641 |
| Heart normal | 0.2302 |
| Heart CHF | 0.0759 |
| Kidney | 0.618 |
| Skeletal Muscle | 0.1668 |
| Adipose normal | 0.0932 |
| Pancreas | 0.172 |
| Primary osteoblasts | 0.1037 |
| Osteoclasts (diff) | 0 |
| Skin normal | 0 |
| Spinal cord normal | 0 |
| Brain Cortex normal | 2.2203 |
| Brain-Hypothalamus normal | 0.4603 |
| Nerve | 0 |
| DRG (Dorsal Root Ganglion) | 0 |
| Breast normal | 0.3587 |
| Breast tumor | 0 |
| Ovary normal | 0.7609 |
| Ovary Tumor | 0 |
| Prostate Normal | 0.0355 |
| Prostate Tumor | 0.1041 |
| Salivary glands | 0.0261 |
| Colon normal | 0.0164 |
| Colon Tumor | 0.0566 |
| Lung normal | 0.6053 |
| Lung tumor | 0.9175 |
| Lung COPD | 0.6978 |
| Colon IBD | 0.006 |
| Liver normal | 0 |
| Liver fibrosis | 0 |
| Spleen normal | 0.0303 |
| Tonsil normal | 0.0084 |
| Lymph node normal | 0 |
| Small intestine normal | 0.0894 |
| Skin-Decubitus | 0.3123 |
| Synovium | 0.0708 |
| BM-MNC | 0 |
| Activated PBMC | 0 |
| Neutrophils | 0 |
| Megakaryocytes | 17.3972 |
| Erythroid | 0 |

TABLE 3
15603 Expression in Vessel Panel 2.1

| Tissue Type | Expression |
|---|---|
| Coronary SMC | 0 |
| Huvec NS | 0 |
| Huvec Shear/static pooled | 0 |
| H/Adipose/PIT 695 | 0.1151 |
| H/Internal Mam Artery/Normal/AMC 263 | 0 |
| H/Internal Mam Artery/Normal/AMC 347 | 0.3221 |
| H/Internal Mam Artery/Normal/AMC350 | 0 |
| H/Internal Mam Artery/Normal/AMC 352 | 0.2863 |
| H/Artery/Normal/PIT 1180 | 0.0511 |
| H/Artery/normal/AMC 150 | 0 |
| H/Artery/normal/PIT 912 | 0.0765 |
| H/Artery/normal/NDR 352 | 0.045 |
| H/Aorta/Diseased/PIT 710 | 0 |
| H/Aorta/Diseased/PIT 712 | 0 |
| H/Aorta/Diseased/PIT 732 | 0.1909 |
| H/Artery/Diseased/iliac/NDR 753 | 0 |
| H/Artery/Diseased/Tibial/PIT 679 | 0.1238 |
| H/Vein/Normal/PIT 1010 | 0.3255 |
| H/Vein/Normal/NDR 239 | 0.0477 |
| H/Vein/Normal/AMC 130 | 0.2581 |
| H/Vein/Normal/AMC 131 | 0 |
| H/Vein/Normal/AMC 137 | 0 |
| H/Vein/Normal/AMC 153 | 0.0661 |
| H/Vein/Normal/AMC 176 | 0.0345 |
| H/Vein/Normal/AMC 177 | 0 |
| H/Vein/Normal/AMC 178 | 0.1002 |
| H/Vein/Normal/AMC 182 | 0.0323 |
| H/Vein/Normal/AMC 190 | 0 |
| H/Vein/Normal/AMC 192 | 0 |
| H/Vein/Normal/AMC 195 | 0 |
| H/Vein/Normal/AMC 211 | 0 |
| M/Aorta/Normal/PRI 286 | 0 |
| M/Vein/Normal/PRI 328 | 0 |
| M/Vein/Normal/PRI 230 | 0 |
| M/Aorta/Diseased/CAR 1216 | 0 |
| M/Aorta/Diseased/CAR 1237 | 0 |
| M/Aorta/Diseased/CAR 1192 | 0 |
| M/Aorta/Diseased/CAR 1196 | 0 |
| M/Artery/Diseased/CAR 1174 | 6.3899 |
| M/Artery/Diseased/CAR 1175 | 0 |

TABLE 4
Expression of 15063

| Tissue Type | Value |
|---|---|
| HUVECs Stat | 0. |
| HUVECs LSS | 0. |
| HUVECs Stat | 0. |
| LSS | 0. |
| HUVECs Prolif | 0. |
| HUVECs Conf | 0. |
| HUVECs -GF | 0. |
| HUVECs IL-1 | 0. |
| HMVEC-Cardiac Prolif | 0. |
| HMVEC-Cardiac Conf | 0. |
| HMVEC-Cardiac Prolif | 0. |
| HMVEC-Cardiac Conf | 0. |
| HMVEC-Lung Prolif | 0. |
| HMVEC-Lung Conf | 0. |
| HMVEC-Lung -GF | 0. |
| HMVEC-Lung Prolif | 1. |
| HMVEC-Lung Conf | 0. |
| Aortic C4h | 0. |
| Aortic TNF | 0. |
| Aortic C14 | 0. |
| Aortic TNF | 0. |
| 293 | 0. |
| HUVEC Stat | 0. |
| HUVEC LSS | 0. |
| HUVEC Stat | 0. |
| HUVEC LSS | 0. |
| HUVEC T6 | 0. |
| HUVEC RDC | 0. |
| HUVEC RDH | 0. |
| HUVEC HuC | 0. |
| HUVEC HuH | 0. |
| Hemangioma | 12 |
| Hemangioma | 7. |

TABLE 5
Expression of 15603 with β2 in the Angiogenesis Panel

| Tissue Type | Expression |
|---|---|
| ONC 101 Hemangioma | 14.9885 |
| ONC 102 Hemangioma | 4.6615 |
| ONC 103 Hemangioma | 5.1902 |
| CHT 1273 Glioblastoma | 0.1909 |
| CHT 216 Glioblastoma | 0.0318 |
| CHT 501 Glioblastoma | 0.1578 |
| NDR 203 Normal Kidney | 0.2082 |
| PIT 213 Renal Cell Carcinoma | 0 |
| CHT 732 Wilms Tumor | 1.4853 |
| CHT 765 Wilms Tumor | 4.4871 |
| NDR 295 Skin | 0 |
| CHT 1424 Uterine Adenocarcinoma | 1.0911 |
| CHT 1238 Neuroblastoma | 0 |
| BWH 78 Fetal Adrenal | 0.0346 |
| BWH 74 Fetal Kidney | 0.2635 |
| BWH 4 Fetal Heart | 0.1023 |
| MPI 849 Normal Heart | 0 |
| CLN 796 Spinal Cord | 0 |

### Human 69318

There are over 30 families of secondary transporters, also known as solute carriers or SLC (reviewed by Berger, et al. (2000) in The Kidney: Physiology and Pathophysiology, eds. Seldin D W and Giebisch G., Lippincott, Williams & Wilkins, Philadelphia 1:107-138). The SLC families are classified according to the pair of molecules they move. The SLC8 and SLC24 families transport calcium. The SLC8 family members are calcium/sodium antiporters, while the SLC24 family members couple potassium with calcium in exchange for sodium. 69318 is a member of the SLC8 and SLC24 families.

The human 69318 sequence (SEQ ID NO:13), which is approximately 2875 nucleotides long including untranslated regions, contains a predicted methionine-initiated coding sequence of about 1752 nucleotides, not including the termination codon (nucleotides 115-1866 of SEQ ID NO:13; 1-1752 SEQ ID NO:15). The coding sequence encodes a 584 amino acid protein (SEQ ID NO:14).

Human 69318 contains the following regions or other structural features (for general information regarding PFAM identifiers, PS prefix and PF prefix domain identification numbers, refer to Sonnhammer et al. (1997) Protein 28:405-420: two sodium/calcium exchanger domains (SEQ ID NO:16, PFAM PF01699) located at about amino acid residues 113 to 252 and 431 to 576 of SEQ ID NO:14; twelve transmembrane domains (predicted by MEMSAT, Jones et al. (1994) Biochemistry 33:3038-3049) at about amino acids 10 to 26, 95 to 119, 139 to 161, 168 to 192, 205 to 221, 228 to 251, 384 to 408, 419 to 437, 447 to 465, 486 to 509, 525 to 547, and 555 to 573 of SEQ ID NO:14; two protein kinase C phosphorylation sites (Prosite PS00005) at about amino acids 62 to 64 and 132 to 134 of SEQ ID NO:14; four casein kinase II phosphorylation sites (Prosite PS00006) located at about amino acids 70 to 73, 271 to 274, 468 to 471, and 514 to 517 of SEQ ID NO:14; two cAMP/cGMP-dependent protein kinase phosphorylation sites (Prosite PS00004) located at about amino acids 255 to 258 and 318 to 321 of SEQ ID NO:14; two N-glycosylation sites (Prosite PS00001) from about amino acids 60 to 63, and 125 to 128 of SEQ ID NO:14; one amidation site (Prosite PS00009) from about amino acids 2 to 5 of SEQ ID NO:14; and nine N-myristoylation sites (Prosite PS00008) from about amino acids 26 to 31, 58 to 63, 115 to 120, 168 to 173, 178 to 183, 398 to 403, 466 to 471, 494 to 499, and 502 to 507 of SEQ ID NO:14.

A hydropathy plot of human 69318 was performed. Polypeptides of the invention include fragments which include: all or part of a hydrophobic sequence, e.g., the sequence from about amino acid 10 to 26, from about 168 to 192, and from about 486 to 509 of SEQ ID NO:14; all or part of a hydrophilic sequence, e.g., the sequence from about amino acid 251 to 258, from about 271 to 281, and from about 346 to 357 of SEQ ID NO:14; a sequence which includes a Cys, or a glycosylation site.

The 69318 protein contains a significant number of structural characteristics in common with transporters, more specifically, with members of the sodium/calcium exchanger families, SLC8 and SLC24. The term “family” when referring to the protein and nucleic acid molecules of the invention means two or more proteins or nucleic acid molecules having a common structural domain or motif and having sufficient amino acid or nucleotide sequence homology as defined herein. As used herein, the term “transporter” or “sodium/calcium exchanger” refers to secondary active transport proteins. Secondary active transporters typically couple the active transport of one molecule, e.g., an ion, e.g., a calcium ion against its concentration gradient to the energy gained by concomitant transport of a second molecule, e.g., another ion (e.g., a sodium ion) with its concentration gradient. Human sodium/calcium exchangers have been grouped into two families, named SLC8 and SLC24. In the SLC8 family, the calcium transport is coupled with sodium exchange. In the SLC24 family, the sodium exchange energy is supplemented with additional energy derived from exchange of potassium down its gradient. Thus, in the SLC24 family, calcium moves against its concentration gradient in the same direction as potassium, which moves with its concentration gradient, both at the same time as the opposite movement of sodium with its concentration gradient.

Typically, sodium/calcium exchangers or SLC8 or SLC24 family members are integral membrane proteins having at least one, two, three, four, five, six, seven, eight, nine, ten, eleven and preferably twelve transmembrane domains. These transmembrane domains can be divided into two homologous groups, one encompassing transmembrane domains 2 to 6 and the other encompassing transmembrane domains 8 to 12. Each group is named a sodium/calcium exchanger domain and is involved in the actual cross-membrane ion transfer. The loops before and between the sodium/calcium exchanger domains are hypervariable and involved in the tissue and ion specificity, ion binding and transporter regulation. The first hypervariable loop is extracellular and the second hypervariable loop is cytoplasmic (Prinsen et al. (2000) J. Neuroscience 20:1424-34). In SLC8, the cytoplasmic hypervariable loop is at least 500 amino acids long (Berger et al. supra). SLC24 family members typically have shorter cytoplasmic loops. A GAP alignment of 69318 with human NCKX2, an SLC24 family member (Accession number 6650379 in GenPept, corresponding to AF097366 in Genbank, SEQ ID NO:17 found a 24% identity (as determined using a matrix made by matblas from blosum62.iij).

A 69318 polypeptide can include at least one, preferably two “sodium/calcium exchanger domains” or regions homologous with a “sodium/calcium exchanger domain”.

As used herein, the term “sodium/calcium exchanger domain” includes an amino acid sequence of about 50 to 250 amino acid residues in length and having a bit score for the alignment of the sequence to the sodium/calcium exchanger domain (HMM) of at least 50. Preferably, a sodium/calcium exchanger domain mediates transport of an ion e.g. a sodium, calcium or potassium ion from one side of a membrane to the opposite side of the membrane. Preferably, a sodium/calcium exchanger domain includes at least about 80 to 200 amino acids, more preferably about 110 to 175 amino acid residues, or about 135 to 150 amino acids and has a bit score for the alignment of the sequence to the sodium/calcium exchanger domain (HMM) of at least 60, 70, 80 or greater. The sodium/calcium exchanger domain (HMM) has been assigned the PFAM Accession Number PF01699. An alignment of the first sodium/calcium exchanger domain (amino acids 113 to 252 of SEQ ID NO:14) of human 69318 with a consensus amino acid sequence (SEQ ID NO:16) derived from a hidden Markov model yields a bit score of 85.2. An alignment of the second sodium/calcium exchanger domain (amino acids 431 to 576 of SEQ ID NO:14) of human 69318 with a consensus amino acid sequence (SEQ ID NO:16) derived from a hidden Markov model yields a bit score of 92.1.

In a preferred embodiment, a 69318 polypeptide or protein has a “sodium/calcium exchanger domain” or a region which includes at least about 80 to 200, more preferably about 110 to 175 or 135 to 150 amino acid residues and has at least about 60%, 70% 80% 90% 95%, 99%, or 100% homology with a “sodium/calcium exchanger domain,” e.g., the sodium/calcium exchanger domain of human 69318 (e.g., residues 113 to 252 and 431 to 576 of SEQ ID NO:14).

To identify the presence of a “sodium/calcium exchanger” domain in a 69318 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “sodium/calcium exchanger domain” domain in the amino acid sequence of human 69318 at about residues 113 to 252 and 431 to 576 of SEQ ID NO:14.

A 69318 polypeptide can include at least one, two, three, four, five, six, seven, eight, nine, ten, eleven and preferably twelve transmembrane domains or regions homologous with a “transmembrane domain”. As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta, W. N. et al., (1996) Annual Rev. Neurosci. 19:235-263, the contents of which are incorporated herein by reference.

In a preferred embodiment, a 69318 polypeptide or protein has at least one, two, three, four, five, six, seven, eight, nine, ten, eleven and preferably twelve “transmembrane domains” or regions which include at least about 12 to 35 more preferably about 14 to 30 or 15 to 25 amino acid residues and has at least about 60%, 70% 80% 90% 95%, 99%, or 100% homology with a “transmembrane domain,” e.g., the transmembrane domains of human 69318 (e.g., residues 10 to 26, 95 to 119, 139 to 161, 168 to 192, 205 to 221, 228 to 251, 384 to 408, 419 to 437, 447 to 465, 486 to 509, 525 to 547, and 555 to 573 of SEQ ID NO:14). The transmembrane domains of human 69318 can be visualized in a hydropathy plot as regions of about 15 to 25 amino acids where the hydropathy trace is mostly above the horizontal line.

To identify the presence of a “transmembrane” domain in a 69318 protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 69318 polypeptide can include at least one, two, three, four, five, six, seven, eight, nine, ten, eleven, twelve and preferably thirteen “non-transmembrane regions.” As used herein, the term “non-transmembrane region” includes an amino acid sequence not identified as a transmembrane domain. The non-transmembrane regions in 69318 are located at about amino acids 1 to 9, 27 to 94, 120 to 138, 162 to 167, 193 to 204, 222 to 227, 252 to 383, 409 to 418, 438 to 446, 466 to 485, 510 to 524, 548 to 554 and 574 to 584 of SEQ ID NO:14.

In one embodiment, a 69318 protein includes at least one, two, three, four, preferably five cytoplasmic loops. As used herein, the term “loop” includes an amino acid sequence that resides outside of a phospholipid membrane, having a length of at least about 4, preferably about 5 to 600, and more preferably about 5 to 150 amino acid residues, and has an amino acid sequence that connects two transmembrane domains within a protein or polypeptide. As used herein, a “cytoplasmic loop” includes an amino acid sequence having about 1 to 600, preferably about 1 to 400, preferably about 1 to 300, more preferably about 1 to 200, more preferably about 1 to 150, or even more preferably about 1 to 135 amino acid residues in length and is located inside of a cell or intracellularly. The C-terminal amino acid residue of a “cytoplasmic loop” is adjacent to an N-terminal amino acid residue of a transmembrane domain in a 69318 protein. For example, a cytoplasmic loop is located at about amino acid residues 120 to 138, 193 to 204, 252 to 383, 438 to 446, or 510 to 524 of SEQ ID NO:14.

In a preferred embodiment, a 69318 polypeptide or protein has at least one cytoplasmic loop or a region which includes at least about 5, preferably about 7 to 300, and more preferably about 9 to 150 amino acid residues and has at least about 60%, 70% 80% 90% 95%, 99%, or 100% homology with a “cytoplasmic loop,” e.g., at least one cytoplasmic loop of human 69318 (e.g., residues 252 to 383 of SEQ ID NO:14).

In another embodiment, a 69318 protein includes at least one, two, three, four, five, preferably six non-cytoplasmic loops. As used herein, a “non-cytoplasmic loop” includes an amino acid sequence located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. Accordingly, the N-terminal amino acid of a non-cytoplasmic loop is adjacent to a C-terminal amino acid of a transmembrane domain in a 69318 molecule, and the C-terminal amino acid of a non-cytoplasmic loop is adjacent to an N-terminal amino acid of a transmembrane domain in a 69318 molecule. For example, a “non-cytoplasmic loop” can be found at about amino acids 27 to 94, 162 to 167, 222 to 227, 409 to 418, 466 to 485, and 548 to 554 of SEQ ID NO:14.

In a preferred embodiment, a 69318 polypeptide or protein has at least one non-cytoplasmic loop or a region which includes at least about 4, preferably about 5 to 40, preferably about 6 to 60, and more preferably about 6 to 70 amino acid residues and has at least about 60%, 70% 80% 90% 95%, 99%, or 100% homology with an “non-cytoplasmic loop,” e.g., at least one non-cytoplasmic loop of human 69318 (e.g., residues 27 to 94 of SEQ ID NO:14).

A 69318 family member can include at least one, preferably two sodium/calcium exchanger domains; and at least one, two, three, four, five, six, seven, eight, nine, ten, eleven and preferably twelve transmembrane domains; at least one cytoplasmic loop; at least one non-cytoplasmic loop; or at least one, two, three, four, five, six, seven, eight, nine, ten, eleven, twelve and preferably thirteen non-transmembrane regions. Furthermore, a 69318 family member can include at least one, preferably two protein kinase C phosphorylation sites (PS00005); at least one, two, three, and preferably four casein kinase II phosphorylation sites (PS00006); at least one, preferably two N-glycosylation sites (PS00001); at least one, preferably two cAMP- and cGMP-dependent protein kinase phosphorylation sites (PS00004); at least one amidation site (PS00009); and at least one, three, five, seven and preferably nine N-myristoylation sites (PS00008).

As the 69318 polypeptides of the invention can modulate 69318-mediated activities, they can be useful for developing novel diagnostic and therapeutic agents for sodium/calcium exchanger-associated or other 69318-associated disorders, as described below.

Sodium/calcium exchangers actively transport calcium and participate in calcium homeostasis. These transporters can be found in the heart, muscle, brain, retina and kidney, especially in the excitable cells, where they participate in the many physiological processes which require management of intracellular calcium levels. These transporters dynamically coordinate with calcium channels and calcium binding proteins to control the availability of these ions for calcium-dependent cellular responses. For example, in an excitable or contractile cell, a stimulus activates calcium channels to allow rapid cytosolic influx of calcium and induce a response to the stimulus. After the stimulus is removed, the sodium/calcium exchangers transport the calcium back out of the cytoplasm to restore the potential function of the cell.

Alterations in the dynamic interplay of calcium influx and efflux processes play roles in many diseases. These diseases can be associated with an intracellular calcium overload, such as in MELAS, an encephalomyopathy (Moudy et al. (1995) Proc. Natl. Acad. Sci. USA 92:729-33), in retinal degeneration (Edward et al. (1991) Arch. Opthalmol. 109:554-62), or in myocardial reoxygenation/reperfusion injury (Mochizuki and Jiang (1998) Jpn. Heart J. 39:707-14); an intracellular potassium overload in a variant of Bartter's syndrome (Peleg et al. (1997) Hypertension 30:1338-41); or an intracellular calcium deficiency in stimulated platelets of some diabetic patients (Yamaguchi et al. (1991) Diabetes Res. 18:89-94).

As used herein, a “69318 activity”, “biological activity of 69318” or “functional activity of 69318”, refers to an activity exerted by a 69318 protein, polypeptide or nucleic acid molecule on e.g., a 69318-responsive cell or on a 69318 substrate, e.g., a protein substrate, as determined in vivo or in vitro. In one embodiment, a 69318 activity is a direct activity, such as an association with a 69318 target molecule. A “target molecule” or “binding partner” is a molecule with which a 69318 protein binds or interacts in nature. In an exemplary embodiment, 69318 is a transporter, e.g., an SLC8 family sodium/calcium exchanger or SLC24 family sodium/calcium/potassium exchanger, and thus binds to or interacts in nature with a molecule, e.g., an ion, (e.g., a calcium ion), a second molecule, e.g., an ion, (e.g., a sodium ion), and/or a third molecule, e.g., an ion, (e.g., a potassium ion).

A 69318 activity can also be an indirect activity, e.g., a cellular signaling activity mediated by interaction of the 69318 protein with a 69318 receptor. Based on the above-described sequence structures and similarities to molecules of known function, the 69318 molecules of the present invention have similar biological activities as sodium/calcium exchanger family members. For example, the 69318 proteins of the present invention can have one or more of the following activities: (1) the ability to reside within a membrane, e.g., a cell or organelle membrane; (2) the ability to interact with, e.g., bind to, a substrate or target molecule; (3) the ability to transport a substrate or target molecule, e.g., an ion (e.g., a calcium ion) across a membrane; (4) the ability to transport a second substrate or target molecule, e.g., another ion (e.g., a sodium ion) across a membrane; (5) the ability to transport a third substrate or target molecule, e.g., another ion (e.g., a potassium ion) across a membrane; (6) the ability to interact with and/or modulate the activity of a second non-transporter protein; (7) the ability to modulate cellular signaling and/or gene transcription (e.g., either directly or indirectly); (8) the ability to modulate calcium homeostasis; (9) the ability to modulate muscle contraction; (10) the ability to modulate responses to stimuli or (11) the ability to modulate vision.

The 69318 molecules of the invention can modulate the activities of cells in tissues where they are expressed. TaqMan analysis shows 69318 mRNA is expressed in normal artery, human umbilical vein endothelial cells (HUVEC), kidney, pancreas, normal brain cortex, breast tumor, normal ovary, and lung tumor. Accordingly, the 69318 molecules of the invention can act as therapeutic or diagnostic agents for cardiovascular disorders, including endothelial cell disorders, renal disorders, pancreatic disorders, neurological disorders, cellular proliferative and/or differentiative disorders and ovarian disorders.

The 69318 molecules can be used to treat cardiovascular disorders in part because the 69318 mRNA is expressed in normal artery, and human umbilical vein endothelial cells. A cardiovascular disease or disorder also includes an endothelial cell disorder.

The 69318 molecules can be used to treat renal disorders in part because the 69318 mRNA is expressed in the kidney.

The 69318 molecules can be used to treat pancreatic disorders in part because the 69318 mRNA is expressed in the pancreas.

The 69318 molecules can be used to treat neurological disorders in part because the 69318 mRNA is expressed in the brain cortex

The 69318 molecules can be used to treat cellular proliferative and/or differentiative disorders in part because the 69318 mRNA is expressed in lung tumor and breast tumor but not in normal lung or normal breast.

The 69318 molecules can be used to treat ovarian disorders in part because the 69318 mRNA is expressed in normal ovary.

Thus, the 69318 molecules can act as novel diagnostic targets and therapeutic agents for controlling one or more cardiovascular disorders, including endothelial cell disorders, renal disorders, pancreatic disorders, neurological disorders, and cellular proliferative and differentiative disorders and other transporter, e.g., sodium/calcium exchanger or sodium/calcium/potassium exchanger disorders. Examples of such disorders, e.g., sodium/calcium exchanger-associated or other 69318-associated disorders, include but are not limited to, eye and vision disorders, immune and inflammatory disorders, hematopoietic disorders, pain disorders, or metabolic disorders.

The 69318 nucleic acid and protein of the invention can be used to treat and/or diagnose a variety of immune, e.g., inflammatory disorders.

The 69318 molecules of the invention can be used to monitor, treat and/or diagnose a variety of proliferative disorders. Such disorders include hematopoietic neoplastic disorders.

Additionally, 69318 can play an important role in the regulation of metabolism or pain disorders.

### Gene Expression Analysis of 69318

Human 69318 expression was measured by TaqMan® quantitative PCR (Perkin Elmer Applied Biosystems) in cDNA prepared from a variety of normal and diseased (e.g., cancerous) human tissues or cell lines.

The results indicate significant 69318 expression in normal artery, human umbilical vein endothelial cells (HUVEC), kidney, pancreas, normal brain cortex, breast tumor, normal ovary, and lung tumor.

### Human 12303 (TWIK-8)

The present invention is based, at least in part, on the discovery of novel molecules, referred to herein as “TWIK-8” or “12303” nucleic acid and protein molecules, which are novel members of the TWIK (for Tandem of P domains in a Weak Inward rectifying K⁺ channel)-like family of potassium channels. These novel molecules are capable of, for example, modulating a potassium channel mediated activity in a cell, e.g., a neuronal cell, or a muscle cell.

Potassium (K⁺) channels are ubiquitous proteins which are involved in the setting of the resting membrane potential as well as in the modulation of the electrical activity of cells. In excitable cells, K⁺ channels influence action potential waveforms, firing frequency, and neurotransmitter secretion (Rudy, B. (1988) Neuroscience, 25, 729-749; Hille, B. (1992) Ionic Channels of Excitable Membranes, 2nd Ed.). In non-excitable cells, they are involved in hormone secretion, cell volume regulation and potentially in cell proliferation and differentiation (Lewis et al. (1995) Annu. Rev. Immunol., 13, 623-653). Developments in electrophysiology have allowed the identification and the characterization of an astonishing variety of K⁺ channels that differ in their biophysical properties, pharmacology, regulation and tissue distribution (Rudy, B. (1988) Neuroscience, 25, 729-749; Hille, B. (1992) Ionic Channels of Excitable Membranes, 2nd Ed.). More recently, cloning efforts have shed considerable light on the mechanisms that determine this functional diversity. Furthermore, analyses of structure-function relationships have provided an important set of data concerning the molecular basis of the biophysical properties (selectivity, gating, assembly) and the pharmacological properties of cloned K⁺ channels.

Functional diversity of K⁺ channels arises mainly from the existence of a great number of genes coding for pore-forming subunits, as well as for other associated regulatory subunits. Two main structural families of pore-forming subunits have been identified. The first one consists of subunits with a conserved hydrophobic core containing six transmembrane domains (TMDs). These K⁺ channel α subunits participate in the formation of outward rectifier voltage-gated (Kv) and Ca²⁺-dependent K⁺ channels. The fourth TMD contains repeated positive charges involved in the voltage gating of these channels and hence in their outward rectification (Logothetis et al. (1992) Neuron, 8, 531-540; Bezanilla et al. (1994) Biophys. J. 66, 1011-1021).

The second family of pore-forming subunits have only two TMDs. They are essential subunits of inward-rectifying (IRK), G-protein-coupled (GIRK) and ATP-sensitive (K_{ATP}) K⁺ channels. The inward rectification results from a voltage-dependent block by cytoplasmic Mg²⁺ and polyamines (Matsuda, H. (1991) Annu. Rev. Physiol., 53, 289-298). A conserved domain, called the P domain, is present in all members of both families (Pongs, O. (1993) J. Membr. Biol., 136, 1-8; Heginbotham et al. (1994) Biophys. J. 66, 1061-1067; Mackinnon, R. (1995) Neuron, 14, 889-892; Pascual et al., (1995) Neuron., and 14, 1055-1063). This domain is an essential element of the aqueous K⁺-selective pore. In both groups, the assembly of four subunits is necessary to form a functional K⁺ channel (Mackinnon, R. (1991) Nature, 350, 232-235; Yang et al., (1995) Neuron, 15, 1441-1447.

In both six TMD and two TMD pore-forming subunit families, different subunits coded by different genes can associate to form heterotetramers with new channel properties (Isacoff et al., (1990) Nature, 345, 530-534). A selective formation of heteropolymeric channels may allow each cell to develop the best K⁺ current repertoire suited to its function. Pore-forming α subunits of Kv channels are classified into different subfamilies according to their sequence similarity (Chandy et al. (1993) Trends Pharmacol. Sci., 14: 434). Tetramerization is believed to occur preferentially between members of each subgroup (Covarrubias et al. (1991) Neuron, 7, 763-773). The domain responsible for this selective association is localized in the N-terminal region and is conserved between members of the same subgroup. This domain is necessary for hetero- but not homo-multimeric assembly within a subfamily and prevents co-assembly between subfamilies. Recently, pore-forming subunits with two TMDs were also shown to co-assemble to form heteropolymers (Duprat et al. (1995) Biocheni. Biophys. Res. Commun., 212, 657-663. This heteropolymerization seems necessary to give functional GIRKs. IRKs are active as homopolymers but also form heteropolymers.

New structural types of K⁺ channels were identified recently in both humans and yeast. These channels have two P domains in their functional subunit instead of only one (Ketchum et al. (1995) Nature, 376, 690-695; Lesage et al. (1996) J. Biol. Chem., 271, 4183-4187; Lesage et al. (1996) EMBO J., 15, 1004-1011; Reid et al. (1996) Receptors Channels 4, 51-62). The human channel called TWIK-1, has four TMDs. TWIK-1 is expressed widely in human tissues and is particularly abundant in the heart and the brain. TWIK-1 currents are time independent and inwardly rectifying. These properties suggest that TWIK-1 channels are involved in the control of the background K⁺ membrane conductance (Lesage et al. (1996) EMBO J., 15, 1004-1011).

The human TWIK-8 or 12303 sequence (SEQ ID NO:18), which is approximately 1408 nucleotides long including untranslated regions, contains a predicted methionine-initiated coding sequence of about 1257 nucleotides, not including the termination codon (nucleotides 84-1340 of SEQ ID NO:18; 1-1257 SEQ ID NO:20). The coding sequence encodes a 419 amino acid protein (SEQ ID NO:19).

The amino acid sequence of human TWIK-8 was analyzed using the program PSORT to predict the localization of the proteins within the cell. This program assesses the presence of different targeting and localization amino acid sequences within the query sequence. The results of the analysis show that human TWIK-8 (SEQ ID NO:19) may be localized to the endoplasmic reticulum or to the mitochondrion.

An analysis of the amino acid sequence of human TWIK-8 using the Signal P program (Henrik, et al. (1997) Protein Engineering 10:1-6), identified the presence of a signal peptide from amino acids 1-46 of SEQ ID NO:19.

A search was performed against the Memsat database resulting in the identification of six transmembrane domains in the amino acid sequence of the native human TWIK-8 (SEQ ID NO: 19) at about residues 32-50, 116-137, 144-165, 195-219, 226-242, and 260-283. This search further identified five transmembrane domains in the amino acid sequence of the predicted mature form of this protein, at about residues 70-91, 98-119, 149-173, 180-196, and 214-237 of SEQ ID NO:19.

A search was performed against the HMM database, resulting in the identification of a “seven-transmembrane receptor domain” from about residues 25-244, and a “cyclic nucleotide-gated channel domain” from about residues 27-204 in the amino acid sequence of human TWIK-8 (SEQ ID NO:19).

A search was also performed against the ProDom database, resulting in the identification of “TRAAK potassium channel domains” from about residues 50-104 (score=175), 175-199 (score=115), and 288-382 (score=135) of SEQ ID NO:19; a “potassium channel protein domain” from about residues 99-153 (score=101) of SEQ ID NO:19; a “voltage-gated potassium channel domain” from about residues 102-168 (score=115) of SEQ ID NO:19; an “outward-rectifier TOK1 potassium channel domain” from about residues 215-270 (score=70) of SEQ ID NO:19; and a “potassium channel subunit domain” from about residues 216-287 (score=156) in the amino acid sequence of human TWIK-8 (SEQ ID NO:19).

A BLASTX 2.0 search against the NRP/protot database, using a score of 100, a wordlength of 3, and a Blosum 62 matrix (Altschul et al. (1990) J. Mol. Biol. 215:403), of the translated nucleotide sequence of human TWIK-8 revealed that human TWIK-8 has limited sequence homology to Mus musculus TRAAK K+ channel subunit mRNA (Accession Number AF056492), to Homo sapiens TREK-1 potassium channel (KCNK2) mRNA (Accession Number AF129399), to Mus musculus TREK-1 K+ channel subunit mRNA (Accession Number U73488), and to Homo sapiens two-pore potassium channel TPKC1 mRNA (Accession number AF004711).

As used herein, a “potassium channel” includes a protein or polypeptide which is involved in receiving, conducting, and transmitting signals in an electrically excitable or a non-electrically excitable cell, e.g., a neuronal cell, or a muscle cell (e.g., a cardiac muscle cell). Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming α and cytoplasmic β subunits. Potassium channels may also be found in non-excitable cells (e.g., spleen cells or prostate cells), where they may play a role in, e.g., signal transduction. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R. et al., Principles of Neural Science, second edition, (Elsevier Science Publishing Co., Inc., N.Y. (1985)), the contents of which are incorporated herein by reference. As the TWIK-like proteins of the present invention may modulate potassium channel mediated activities, they may be useful for developing novel diagnostic and therapeutic agents for potassium channel associated disorders.

As used herein, a “potassium channel associated disorder” includes a disorder, disease or condition which is characterized by a misregulation of a potassium channel mediated activity. Potassium channel associated disorders can detrimentally affect conveyance of sensory impulses from the periphery to the brain and/or conductance of motor impulses from the brain to the periphery; integration of reflexes; interpretation of sensory impulses; and emotional, intellectual (e.g., learning and memory), or motor processes.

Examples of potassium channel associated disorders include CNS disorders such as cognitive and neurodegenerative disorders.

Further examples of potassium channel associated disorders include cardiac-related disorders. TWIK-8-mediated or related disorders also include disorders of the musculoskeletal system such as paralysis and muscle weakness, e.g., ataxia, myotonia, and myokymia.

Other examples of potassium channel-associated disorders include pain disorders. Pain disorders include those disorders that affect pain signaling mechanisms. The TWIK-8 molecules of the present invention may be present on these sensory neurons and, thus, may be involved in detecting these noxious chemical, mechanical, or thermal stimuli and transducing this information into membrane depolarization events. Thus, the TWIK-8 molecules by participating in pain signaling mechanisms, may modulate pain elicitation and act as targets for developing novel diagnostic targets and therapeutic agents to control pain.

Potassium channel disorders also include cellular proliferation, growth, differentiation, or migration disorders. Cellular proliferation, growth, differentiation, or migration disorders include those disorders that affect cell proliferation, growth, differentiation, or migration processes.

The TWIK-8 molecules of the present invention are involved in signal transduction mechanisms, which are known to be involved in cellular growth, differentiation, and migration processes. Thus, the TWIK-8 molecules may modulate cellular growth, differentiation, or migration, and may play a role in disorders characterized by aberrantly regulated growth, differentiation, or migration. Such disorders include cancer, e.g., carcinoma, sarcoma, or leukemia; tumor angiogenesis and metastasis; skeletal dysplasia; neuronal deficiencies resulting from impaired neural induction and patterning; and hematopoietic and/or myeloproliferative disorders.

TWIK-8-associated or related disorders also include disorders of tissues in which TWIK-8 protein is expressed, e.g., brain cortex, hypothalamus and dorsal root ganglia.

As used herein, a “potassium channel mediated activity” includes an activity which involves a potassium channel, e.g., a potassium channel in a neuronal cell, or a muscle cell (e.g., a cardiac muscle cell), associated with receiving, conducting, and transmitting signals in, for example, the nervous system. Potassium channel mediated activities include release of neurotransmitters, e.g., dopamine or norepinephrine, from cells, e.g., neuronal cells; modulation of resting potential of membranes, wave forms and frequencies of action potentials, and thresholds of excitation; and modulation of processes such as integration of sub-threshold synaptic responses, the conductance of back-propagating action potentials in, for example, neuronal cells or muscle cells, participation in signal transduction pathways, and participation in nociception.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., monkey proteins. Members of a family may also have common functional characteristics.

For example, the family of TWIK-8 proteins comprises at least one “transmembrane domain” and preferably six transmembrane domains. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al. (1996) Annual Rev. Neurosci. 19: 235-263, the contents of which are incorporated herein by reference. Amino acid residues 32-50, 116-137, 144-165, 195-219, 226-242, and 260-283 of the native TWIK-8 protein (SEQ ID NO:19), and amino acid residues 70-91, 98-119, 149-173, 180-196 and 214-237 of the mature TWIK-8 protein are predicted to comprise transmembrane domains. Accordingly, TWIK-8 proteins having at least 50-60% homology, preferably about 60-70%, more preferably about 70-80%, or about 80-90% homology with a transmembrane domain of human TWIK-8 are within the scope of the invention.

In another embodiment, a TWIK-8 molecule of the present invention is identified based on the presence of a Pore loop (P-loop). As used herein, the term “Pore loop” or “P-loop” includes an amino acid sequence of about 15-45 amino acid residues in length, preferably about 15-35 amino acid residues in length, and most preferably about 15-25 amino acid residues in length, which is involved in lining the potassium channel pore. A P-loop is typically found between transmembrane domains of potassium channels and is believed to be a major determinant of ion selectivity in potassium channels. Preferably, P-loops contain a G-[HYDROPHOBIC AMINO ACID]-G sequence, e.g., a GYG, GLG, or GFG sequence. P-loops are described in, for example, Warmke et al. (1991) Science 252:1560-1562; Zagotta W. N. et al., (1996) Annual Rev. Neuronsci. 19:235-63 (Pongs, O. (1993) J. Membr. Biol., 136, 1-8; Heginbotham et al. (1994) Biophys. J. 66, 1061-1067; Mackinnon, R. (1995) Neuron, and 14, 889-892; Pascual et al., (1995) Neuron., 14, 1055-1063), the contents of which are incorporated herein by reference. Amino acid residues 243-259 (SEQ ID NO:19) of the native human TWIK-8 protein, and residues 197-213 of the predicted mature human TWIK-8 protein comprise a P-loop.

In a preferred embodiment, the TWIK-8 molecules of the invention include at least one and, preferably, six transmembrane domains and at least one P-loop domain.

In another embodiment, a TWIK-8 molecule of the present invention is identified based on the presence of a “seven-transmembrane receptor domain” in the protein or corresponding nucleic acid molecule. Seven-transmembrane receptor domains are described, for example, in Hamann et al. (1996) Genomics 32: 144-147. As used herein, the term “seven-transmembrane receptor domain” includes a protein domain having an amino acid sequence of about 150-320 amino acid residues. Preferably, a seven-transmembrane receptor domain includes at least about 200-250, or more preferably about 220 amino acid residues. To identify the presence of a seven-transmembrane receptor domain in a TWIK-8 protein, and to make the determination that a protein of interest has a particular profile, the amino acid sequence of the protein is searched against a database of known protein domains (e.g., the HMM database). The seven-transmembrane receptor domain (HMM) has been assigned the PFAM Accession PF00002. A search was performed against the HMM database resulting in the identification of a seven-transmembrane receptor domain in the amino acid sequence of human TWIK-8 at about residues 25-244 of SEQ ID NO: 19.

In another embodiment, a TWIK-8 molecule of the present invention is identified based on the presence of a “cyclic nucleotide-gated channel domain” in the protein or corresponding nucleic acid molecule. Cyclic nucleotide-gated channel domains are described, for example, in Yau (1994) Proc. Natl. Acad. Sci. USA 91: 3481-3483. As used herein, the term “cyclic nucleotide-gated channel domain” includes a protein domain having an amino acid sequence of about 100-225 amino acid residues. Preferably, a cyclic nucleotide-gated channel domain includes at least about 150-200, or more preferably about 178 amino acid residues. To identify the presence of a cyclic nucleotide-gated channel domain in a TWIK-8 protein, and to make the determination that a protein of interest has a particular profile, the amino acid sequence of the protein is searched against a database of known protein domains (e.g., the HMM database). The cyclic nucleotide-gated channel domain (HMM) has been assigned the PFAM Accession PF00914. A search was performed against the HMM database resulting in the identification of a cyclic nucleotide-gated channel domain in the amino acid sequence of human TWIK-8 at about residues 27-204 of SEQ ID NO:19.

In another embodiment, a TWIK-8 molecule of the present invention is identified based on the presence of a “TRAAK potassium channel domain” in the protein or corresponding nucleic acid molecule. As used herein, the term “TRAAK potassium channel domain” includes a protein domain having an amino acid sequence of about 20-150 amino acid residues and having a bit score for the alignment of the sequence to the TRAAK potassium channel domain of at least 115-175. Preferably, a TRAAK potassium channel domain includes at least about 23-100, or more preferably about 25, 55, or 95 amino acid residues, and has a bit score for the alignment of the sequence to the TRAAK potassium channel domain of at least 20, 30, 40, 50, 60, 70, 80, 90, 100, 110 or higher. The TRAAK potassium channel domain has been assigned ProDom entries 73512, 98483, and 105542. To identify the presence of a TRAAK potassium channel domain in a TWIK-8 protein, and to make the determination that a protein of interest has a particular profile, the amino acid sequence of the protein is searched against a database of known protein domains (e.g., the ProDom database) using the default parameters. A search was performed against the ProDom database resulting in the identification of a TRAAK potassium channel domains in the amino acid sequence of human TWIK-8 at about residues 50-104, 175-199, and 288-382 of SEQ ID NO:19.

In another embodiment, a TWIK-8 molecule of the present invention is identified based on the presence of a “potassium channel protein domain” in the protein or corresponding nucleic acid molecule. As used herein, the term “potassium channel protein domain” includes a protein domain having an amino acid sequence of about 20-100 amino acid residues and having a bit score for the alignment of the sequence to the potassium channel protein domain of at least 101. Preferably, a potassium channel protein domain includes at least about 40-75, or more preferably about 55 amino acid residues, and has a bit score for the alignment of the sequence to the potassium channel protein domain of at least 20, 30, 40, 50, 60, 70, 80, 90, 100, or higher. The potassium channel protein domain has been assigned ProDom entry 129403. To identify the presence of a potassium channel protein domain in a TWIK-8 protein, and to make the determination that a protein of interest has a particular profile, the amino acid sequence of the protein is searched against a database of known protein domains (e.g., the ProDom database) using the default parameters. A search was performed against the ProDom database resulting in the identification of a potassium channel protein domain in the amino acid sequence of human TWIK-8 at about residues 99-153 of SEQ ID NO:19.

In another embodiment, a TWIK-8 molecule of the present invention is identified based on the presence of a “voltage-gated potassium channel domain” in the protein or corresponding nucleic acid molecule. As used herein, the term “voltage-gated potassium channel domain” includes a protein domain having an amino acid sequence of about 20-100 amino acid residues and having a bit score for the alignment of the sequence to the potassium channel protein domain of at least 115. Preferably, a voltage-gated potassium channel domain includes at least about 40-75, or more preferably about 55 amino acid residues, and has a bit score for the alignment of the sequence to the voltage-gated potassium channel domain of at least 20, 30, 40, 50, 60, 70, 80, 90, 100, 110 or higher. The voltage-gated potassium channel domain has been assigned ProDom entry 36. To identify the presence of a voltage-gated potassium channel domain in a TWIK-8 protein, and to make the determination that a protein of interest has a particular profile, the amino acid sequence of the protein is searched against a database of known protein domains (e.g., the ProDom database) using the default parameters. A search was performed against the ProDom database resulting in the identification of a voltage-gated potassium channel domain in the amino acid sequence of human TWIK-8 at about residues 102-168 of SEQ ID NO:19.

In another embodiment, a TWIK-8 molecule of the present invention is identified based on the presence of an “outward-rectifier TOK1 potassium channel domain” in the protein or corresponding nucleic acid molecule. As used herein, the term “outward-rectifier TOK1 potassium channel domain” includes a protein domain having an amino acid sequence of about 25-100 amino acid residues and having a bit score for the alignment of the sequence to the outward-rectifier TOK1 potassium channel domain of at least 70. Preferably, an outward-rectifier TOK1 potassium channel domain includes at least about 40-75, or more preferably about 56 amino acid residues, and has a bit score for the alignment of the sequence to the outward-rectifier TOK1 potassium channel domain of at least 20, 30, 40, 50, 60, or higher. The outward-rectifier TOK1 potassium channel domain has been assigned ProDom entry 32818. To identify the presence of an outward-rectifier TOK1 potassium channel domain in a TWIK-8 protein, and to make the determination that a protein of interest has a particular profile, the amino acid sequence of the protein is searched against a database of known protein domains (e.g., the ProDom database) using the default parameters. A search was performed against the ProDom database resulting in the identification of an outward-rectifier TOK1 potassium channel domain in the amino acid sequence of human TWIK-8 at about residues 215-270 of SEQ ID NO:19.

In another embodiment, a TWIK-8 molecule of the present invention is identified based on the presence of a “potassium channel subunit domain” in the protein or corresponding nucleic acid molecule. As used herein, the term “potassium channel subunit domain” includes a protein domain having an amino acid sequence of about 25-125 amino acid residues and having a bit score for the alignment of the sequence to the potassium channel subunit domain of at least 156. Preferably, a potassium channel subunit domain includes at least about 40-100, or more preferably about 72 amino acid residues, and has a bit score for the alignment of the sequence to the potassium channel subunit domain of at least 20, 30, 40, 50, 60, or higher. The potassium channel subunit domain has been assigned ProDom entry 1641. To identify the presence of a potassium channel subunit domain in a TWIK-8 protein, and to make the determination that a protein of interest has a particular profile, the amino acid sequence of the protein is searched against a database of known protein domains (e.g., the ProDom database) using the default parameters. A search was performed against the ProDom database resulting in the identification of a potassium channel subunit domain in the amino acid sequence of human TWIK-8 at about residues 216-287 of SEQ ID NO:19.

Isolated proteins of the present invention, preferably TWIK-8 proteins, have an amino acid sequence sufficiently identical to the amino acid sequence of SEQ ID NO:19 or are encoded by a nucleotide sequence sufficiently identical to SEQ ID NO:18 or 20. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably, 70-80%, or 90-95% homology and share a common functional activity are defined herein as sufficiently identical.

As used interchangeably herein, an “TWIK-8 activity”, “biological activity of TWIK-8” or “functional activity of TWIK-8”, refers to an activity exerted by a TWIK-8 protein, polypeptide or nucleic acid molecule on a TWIK-8 responsive cell or tissue, or on a TWIK-8 protein substrate, as determined in vivo, or in vitro, according to standard techniques. In one embodiment, a TWIK-8 activity is a direct activity, such as an association with a TWIK-8-target molecule. As used herein, a “target molecule” or “binding partner” is a molecule with which a TWIK-8 protein binds or interacts in nature, such that TWIK-8-mediated function is achieved. A TWIK-8 target molecule can be a non-TWIK-8 molecule or a TWIK-8 protein or polypeptide of the present invention. In an exemplary embodiment, a TWIK-8 target molecule is a TWIK-8 ligand, e.g., a potassium channel pore-forming subunit or a potassium channel ligand. Alternatively, a TWIK-8 activity is an indirect activity, such as a cellular signaling activity mediated by interaction of the TWIK-8 protein with a TWIK-8 ligand. The biological activities of TWIK-8 are described herein. For example, the TWIK-8 proteins of the present invention can have one or more of the following activities: (1) interacting with a non-TWIK protein molecule; (2) activating a TWIK-dependent signal transduction pathway; (3) modulating the release of neurotransmitters; (4) modulating membrane excitability; (5) influencing the resting potential of membranes, wave forms and frequencies of action potentials, and thresholds of excitation, (6) modulating processes which underlie learning and memory, such as integration of sub-threshold synaptic responses and the conductance of back-propagating action potentials, and (7) mediating nociception.

Accordingly, another embodiment of the invention features isolated TWIK-8 proteins and polypeptides having a TWIK-8 activity. Preferred proteins are TWIK-8 proteins having at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and, preferably, a TWIK-8 activity.

Additional preferred proteins have at least one or more of the following domains: a transmembrane domain, a pore loop domain, a seven-transmembrane receptor domain, a cyclic nucleotide-gated channel domain, a TRAAK potassium channel domain, a potassium channel protein domain, a voltage-gated potassium channel domain, a potassium channel subunit domain, and an outward-rectifier TOK1 potassium channel domain, and are, preferably, encoded by a nucleic acid molecule having a nucleotide sequence which hybridizes under stringent hybridization conditions to a nucleic acid molecule comprising the nucleotide sequence of SEQ ID NO:18 or 20.

Tissue Distribution of Human TWIK-8 mRNA Using Taqman™ Analysis

The tissue distribution of human TWIK-8 mRNA in a variety of cells and tissues was determined using the TaqMan™ procedure.

Highest expression of TWIK-8 mRNA was detected in brain cortex, followed by dorsal root ganglia, and hypothalamus. Weak expression was also detected in erythroid tissue, followed by HUVEC, spinal cord, hemangioma, kidney, normal ovary and ovary tumor, megakaryocytes, normal prostate and prostate tumor. Weak expression was also detected in breast tumor although no expression was detected in normal breast tissue.

### Human 48000 (TLCC-4) and 52920 (TLCC-5)

The present invention is based, at least in part, on the discovery of novel molecules, referred to herein as “TRP-like calcium channel-4” and “TRP-like calcium channel-5” or “TLCC-4” or “48000” and “TLCC-5” or “52920” nucleic acid and polypeptide molecules, which are novel members of the ion channel, e.g., calcium channel and/or vanilloid receptor, family. These novel molecules are capable of, for example, modulating an ion-channel mediated activity (e.g., a calcium channel- and/or vanilloid receptor-mediated activity) in a cell, e.g., a neuronal, skin, muscle (e.g., cardiac muscle), or liver cell.

Calcium signaling has been implicated in the regulation of a variety of cellular responses, such as growth and differentiation. There are two general methods by which intracellular concentrations of calcium ions may be increased: calcium ions may be freed from intracellular stores, transported by specific membrane channels in the storage organelle, or calcium ions may be brought into the cell from the extracellular milieu through the use of specific channels in the cellular membrane. In the situation in which the intracellular stores of calcium have been depleted, a specific type of calcium channel, termed a ‘capacitative calcium channel’ or a ‘store-operated calcium channel’ (SOC), is activated in the plasma membrane to import calcium ions from the extracellular environment to the cytosol (see Putney and McKay (1999) BioEssays 21:38-46). Calcium may also enter the cell via receptor-stimulated cation channels (see Hofmann et al. (2000) J. Mol. Med. 78:14-25).

There is no single electrophysiological profile characteristic of the calcium channel family; rather, a wide array of single channel conductances, cation selectivity, and current properties have been observed for different channels. Further, in several instances it has been demonstrated that homo- or hetero-polymerization of the channel molecule may occur, further changing the channel properties from those of the single molecule. In general, though, these channels function similarly, in that they are calcium ion-permeable cation channels which become activated after agonist binding to a G protein-coupled receptor.

Members of the capacitative calcium channel family include the calcium release-activated calcium current (CRAC) (Hoth and Penner (1992) Nature 355: 353-355), calcium release-activated non-selective cation current (CRANC) (Krause et al. (1996) J. Biol. Chem. 271: 32523-32528), and the transient receptor potential (TRP) proteins TRP1, TRP2, TRP4, and TRP5. Depletion of intracellular calcium stores activate these channels by a mechanism which is yet undefined, but which has been demonstrated to involve a diffusible factor using studies in which calcium stores were artificially depleted (e.g., by the introduction of chelators into the cell, by activating phospholipase C_{γ}, or by inhibiting those enzymes responsible for pumping calcium ions into the stores or those enzymes responsible for maintaining resting intracellular calcium ion concentrations) (Putney, J. W., (1986) Cell Calcium 7:1-12; Putney, J. W. (1990) Cell Calcium 11:611-624).

Recently, it has been elucidated that three TRP family members, TRP3, TRP6, and a mouse homologue, TRP7, form a sub-family of receptors that are activated in a calcium store-depletion independent manner. TRP3 and TRP6 are activated by diacylglycerols in a membrane delimited manner (Hofmann et al. (1999) Nature 397:259-263). Similarly, murine TRP7 is activated via diacylglycerol stimulation by G_{q} protein coupled receptors (Okada et al. (1999) J. Biol. Chem. 274:27359-27370).

The TRP channel family is one of the best characterized calcium channel protein families. These channels include transient receptor potential protein and homologues thereof (to date, seven TRP homologues and splice variants have been identified in a variety of organisms), the vanilloid receptor subtype I (also known as the capsaicin receptor); the stretch-inhibitable non-selective cation channel (SIC); the olfactory, mechanosensitive channel; the insulin-like growth factor I-regulated calcium channel; the vitamin D-responsive apical, epithelial calcium channel (ECaC); melastatin; and the polycystic kidney disease protein family (see, e.g., Montell and Rubin (1989) Neuron 2:1313-1323; Caterina et al. (1997) Nature 389: 816-824; Suzuki et al. (1999) J. Biol. Chem. 274: 6330-6335; Kiselyov et al. (1998) Nature 396: 478-482; Hoenderop et al. (1999) J. Biol. Chem. 274: 8375-8378; and Chen et al. (1999) Nature 401(6751): 383-6). Each of these molecules is 700 or more amino acids in length, and shares certain conserved structural features. Predominant among these structural features are six transmembrane domains, with an additional hydrophobic loop present between the fifth and sixth transmembrane domains. It is believed that this loop is integral to the activity of the pore of the channel formed upon membrane insertion (Hardie and Minke (1993) Trends Neurosci 16: 371-376). TRP channel proteins also include one or more ankyrin domains. Although found in disparate tissues and organisms, members of the TRP channel protein family all serve to transduce signals by means of calcium entry into cells, particularly pain signals (see, e.g., McClesky and Gold (1999) Annu. Rev. Physiol. 61: 835-856), light signals (Hardie and Minke, supra), or olfactory signals (Colbert et al. (1997) J. Neurosci 17(21): 8259-8269). Thus, this family of molecules may play important roles in sensory signal transduction in general.

Vanilloid receptors (VRs) are cation channels that are structurally related to members of the TRP family of ion channels. VRs share several physical characteristics including an N-terminal cytoplasmic domain which contains three ankyrin repeats, six transmembrane domains, a pore-loop region located between transmembrane domains 5 and 6, and several kinase consensus sequences. These receptors have been proposed to mediate the entry of extracellular calcium into cells in response to the depletion of intracellular calcium stores. VRs are expressed in nociceptive neurons, as well as other cells types, and are activated by a variety of stimuli including noxious heat and protons. A well-known agonist of VR1 is capsaicin, which induces pain behavior in humans and rodents. VR1 knockout mice have been shown to be impaired in their detection of painful heat, to exhibit no vanilloid-evoked pain behavior, and to show little thermal hypersensitivity after inflammation (Szallasi and Blumberg (1999) Pharmacol. Rev. 51:159-211).

The human TLCC-4 or 48000 sequence (SEQ ID NO:21), which is approximately 4586 nucleotides long including untranslated regions, contains a predicted methionine-initiated coding sequence of about 2370 nucleotides, not including the termination codon (nucleotides 146-2515 of SEQ ID NO:21; 1-2370 of SEQ ID NO:23). The coding sequence encodes a 790 amino acid protein (SEQ ID NO:22).

The human TLCC-5 or 52920 sequence (SEQ ID NO:24), which is approximately 3042 nucleotides long including untranslated regions, contains a predicted methionine-initiated coding sequence of about 3039 nucleotides, not including the termination codon (nucleotides 1-3039 of SEQ ID NO:24; 1-3039 SEQ ID NO:26). The coding sequence encodes a 1013 amino acid protein (SEQ ID NO:25).

The human TLCC-5 amino acid sequence was aligned with the amino acid sequence of transient receptor potential polypeptide 7 (TRP7) and melastatin from Homo sapiens using the CLUSTAL W (1.74) multiple sequence alignment program. That alignment shows the homology of the proteins.

A search was performed against the HMM database in PFAM resulting in the identification of four ankyrin repeat domains at about residues 167-202 (score=1.6), 214-246 (score=30.6), 261-294 (score=27.9), and 340-372 (score=18.6), and an ion transport protein domain at about residues 510-677 (score=34.5) in the amino acid sequence of human TLCC-4 (SEQ ID NO:22).

A search was also performed against the MEMSAT database resulting in the identification of six transmembrane domains in the amino acid sequence of human TLCC-4 at about residues 440-461, 488-508, 520-540, 547-565, 590-609, and 652-676 of SEQ ID NO:22.

A search was further performed against the HMM database resulting in the identification of two transient receptor domains at about residues 720-778 (score=21.7) and 820-876 (score=1.5), in the amino acid sequence of human TLCC-5 (SEQ ID NO:25).

A search was also performed against the MEMSAT database resulting in the identification of two transmembrane domains in the amino acid sequence of human TLCC-5 at about residues 786-803 and 826-848 of SEQ ID NO:25.

A search in the Prosite database further resulted in the identification of eight protein kinase C phosphorylation sites in the amino acid sequence of human TLCC-4 (SEQ ID NO:22) at about residues 37-39, 167-169, 290-292, 335-337, 374-376, 476-478, 498-500, and 688-690; two N-glycosylation sites in the amino acid sequence of human TLCC-4 (SEQ ID NO:22) at about residues 452-455 and 683-686; a cAMP- and cGMP-dependent protein kinase phosphorylation site in the amino acid sequence of human TLCC-4 (SEQ ID NO:22) at about residues 375-378; fourteen casein kinase II phosphorylation sites in the amino acid sequence of human TLCC-4 (SEQ ID NO:22) at about residues 88-91, 163-166, 290-293, 305-308, 312-315, 388-391, 393-396, 397-400, 402-405, 411-414, 498-501, 607-610, 624-627, and 699-702; three tyrosine kinase phosphorylation sites in the amino acid sequence of human TLCC-4 (SEQ ID NO:22) at about residues 253-260, 375-382, and 614-622; two N-myristoylation sites in the amino acid sequence of human TLCC-4 (SEQ ID NO:22) at about residues 238-243 and 602-607; an amidation site in the amino acid sequence of human TLCC-4 (SEQ ID NO:22) at about residues 12-15; and a leucine zipper site in the amino acid sequence of human TLCC-4 (SEQ ID NO:22) at about residues 584-605.

A search performed in the Prosite database further resulted in the identification of thirteen protein kinase C phosphorylation sites in the amino acid sequence of human TLCC-5 (SEQ ID NO:25) at about residues 21-23, 28-30, 39-41, 105-107, 240-242, 305-307, 331-333, 338-340, 711-713, 802-804, 901-903, 972-974, and 1001-1003; twelve casein kinase II phosphorylation sites in the amino acid sequence of human TLCC-5 (SEQ ID NO:25) at about residues 54-57, 143-146, 223-226, 240-243, 308-311, 360-363, 436-439, 487-490, 576-579, 725-728, 977-980, and 982-985; and three tyrosine kinase phosphorylation sites in the amino acid sequence of human TLCC-5 (SEQ ID NO:25) at about residues 49-55, 247-254, and 307-314.

Further domain motifs were identified by using the amino acid sequence of TLCC-4 (SEQ ID NO:22) to search through the ProDom database. Numerous matches against protein domains described as “receptor vanilloid channel activated receptor-related receptor-like type OTRPC4”, “channel vanilloid receptor activated receptor-related receptor-like OTRPC4 2B ion”, “repeat ankyrin kinase nuclear factor channel”, “ankyrin repeat kinase domain UNC-44 alternative glycoprotein EGF-like”, “ankyrin”, “channel osmotically receptor-related vanilloid cation”, “receptor vanilloid channel activated receptor-related receptor-like calcium type”, “calcium epithelial channel transporter homolog CAT2”, “channel protein receptor calcium transient potential transmembrane ion transport”, and “receptor vanilloid channel activated osmotically”, and the like were identified.

Further domain motifs were identified by using the amino acid sequence of TLCC-5 (SEQ ID NO:25) to search through the ProDom database. Numerous matches against protein domains described as “channel protein calcium entry capacitative ionic transmembrane ion transport transient” and the like were identified.

As used herein, an “ion channel” includes a protein or polypeptide which is involved in receiving, conducting, and transmitting signals in an electrically excitable cell, e.g., a neuronal or muscle cell. Ion channels include calcium channels, potassium channels, and sodium channels. As used herein, a “calcium channel” includes a protein or polypeptide which is involved in receiving, conducting, and transmitting calcium ion-based signals in an electrically excitable cell. Calcium channels are calcium ion selective, and can determine membrane excitability (the ability of, for example, a neuronal cell to respond to a stimulus and to convert it into a sensory impulse). Calcium channels can also influence the resting potential of membranes, wave forms and frequencies of action potentials, and thresholds of excitation. Calcium channels are typically expressed in electrically excitable cells, e.g., neuronal cells, and may form heteromultimeric structures (e.g., composed of more than one type of subunit). Calcium channels may also be found in non-excitable cells (e.g., adipose cells or liver cells), where they may play a role in, e.g., signal transduction. Examples of calcium channels include the low-voltage-gated channels and the high-voltage-gated channels. Calcium channels are described in, for example, Davila et al. (1999) Annals New York Academy of Sciences 868:102-17 and McEnery, M. W. et al. (1998) J. Bioenergetics and Biomembranes 30(4): 409-418, the contents of which are incorporated herein by reference. As the TLCC-4 and TLCC-5 molecules of the present invention are calcium channels modulating ion channel mediated activities (e.g., calcium channel- and/or vanilloid receptor-mediated activities), they may be useful for developing novel diagnostic and therapeutic agents for ion channel associated disorders (e.g., calcium channel and/or vanilloid receptor associated disorders).

As used herein, an “ion channel associated disorder” includes a disorder, disease or condition which is characterized by a misregulation of an ion channel mediated activity. For example, a “calcium channel associated disorder” includes a disorder, disease or condition which is characterized by a misregulation of a calcium channel mediated activity. Ion channel associated disorders, e.g., calcium channel associated disorders, include CNS disorders, such as cognitive and neurodegenerative disorders.

Ion channel associated disorders, e.g., vanilloid receptor associated disorders also include pain disorders. As used herein, the term “pain disorders” includes those disorders, diseases or conditions that affect pain signaling mechanisms.

Thus, the TLCC-4 or TLCC-5 molecules by participating in pain signaling mechanisms, may modulate pain elicitation and act as targets for developing novel diagnostic targets and therapeutic agents to control pain.

Ion channel associated disorders, e.g., calcium channel and/or vanilloid receptor disorders, also include cellular proliferation, growth, differentiation, or migration disorders.

As used herein, an “ion channel mediated activity” includes an activity which involves an ion channel, e.g., an ion channel and/or a vanilloid receptor, in a neuronal cell, a muscular cell, a skin cell or a liver cell, associated with receiving, conducting, and transmitting signals. Ion channel mediated activities (e.g., calcium channel and/or vanilloid receptor mediated activities) include release of neurotransmitters or second messenger molecules (e.g., dopamine or norepinephrine), from cells, e.g., neuronal cells; modulation of resting potential of membranes, wave forms and frequencies of action potentials, and thresholds of excitation; participation in signal transduction pathways, and modulation of processes such as integration of sub-threshold synaptic responses and the conductance of back-propagating action potentials in, for example, neuronal cells (e.g., changes in those action potentials resulting in a morphological or differentiative response in the cell).

The term “family” when referring to the polypeptide and nucleic acid molecules of the invention is intended to mean two or more polypeptides or nucleic acid molecules having a common structural domain or motif and having sufficient amino acid or nucleotide sequence homology as defined herein. For example, the family of TLCC-4 and TLCC-5 polypeptides comprise at least one “transmembrane domain.” As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al, (1996) Annual Rev. Neurosci. 19: 235-263, the contents of which are incorporated herein by reference. Amino acid residues 440-461, 488-508, 520-540, 547-565, 590-609, and 652-676 of the human TLCC-4 polypeptide (SEQ ID NO:22) comprise transmembrane domains. Amino acid residues 786-803 and 826-848 of the human TLCC-5 polypeptide (SEQ ID NO:25) comprise transmembrane domains. Accordingly, TLCC-4 and/or TLCC-5 polypeptides having at least 50-60% homology, preferably about 60-70%, more preferably about 70-80%, or about 80-90% homology with a transmembrane domain of human TLCC-4 and/or TLCC-5 are within the scope of the invention.

In another embodiment, a TLCC-4 molecule of the present invention is identified based on the presence of at least one “pore domain” between the fifth and sixth transmembrane domains. As used herein, the term “pore domain” includes an overall hydrophobic amino acid sequence which is located between two transmembrane domains of a calcium channel protein, preferably transmembrane domains 5 and 6, and which is believed to be a major determinant of ion selectivity and channel activity in calcium channels. Pore domains are described in, for example, Vannier et al. (1998) J. Biol. Chem. 273: 8675-8679 and Phillips, A. M. et al. (1992) Neuron 8, 631-642, the contents of which are incorporated herein by reference. TLCC-4 molecules having at least one pore domain are within the scope of the invention. Amino acid residues 620-640 of the human TLCC-4 sequence (SEQ ID NO:22) comprise a pore domain.

In another embodiment, a TLCC-4 molecule of the present invention is identified based on the presence of at least one “ankyrin repeat domain.” As used herein, the term “ankyrin repeat domain” includes an amino acid sequence of about 10-110 amino acid residues which serves as an ankyrin repeat. Preferably, an ankyrin repeat domain includes at least about 30 amino acid residues. To identify the presence of an ion transport domain in a TLCC-4 protein, and make the determination that a protein of interest has a particular profile, the amino acid sequence of the protein may be searched against a database of known protein domains (e.g., the HMM database). The ankyrin repeat domain (HMM) has been assigned the PFAM Accession PF00023. A search was performed against the HMM database resulting in the identification of ankyrin repeat domains in the amino acid sequence of human TLCC-4 at about residues 167-202, 214-246, 261-294, and 340-372 of SEQ ID NO:22.

In another embodiment, a TLCC-4 molecule of the present invention is identified based on the presence of at least one “ion transport protein domain.” As used herein, the term “ion transport protein domain” includes a protein domain having an amino acid sequence of about 100-200 amino acid residues which serves to transportions. Preferably, an ion transport protein domain includes at least about 160 amino acid residues. To identify the presence of an ion transport protein domain in a TLCC-4 protein, and make the determination that a protein of interest has a particular profile, the amino acid sequence of the protein may be searched against a database of known protein domains (e.g., the HMM database). The ion transport domain (HMM) has been assigned the PFAM Accession PF00520. A search was performed against the HMM database resulting in the identification of an ion transport protein domain in the amino acid sequence of human TLCC-4 at about residues 510-677 of SEQ ID NO:22.

In another embodiment, a TLCC-5 molecule of the present invention is identified based on the presence of at least one “transient receptor domain.” As used herein, the term “transient receptor domain” includes a protein domain having an amino acid sequence of about 100-200 amino acid residues which is found in transient receptor potential (Trp) proteins and related ion channel proteins. Preferably, a transient receptor domain includes at least about 56-58 amino acid residues. To identify the presence of a transient receptor domain in a TLCC-5 protein, and make the determination that a protein of interest has a particular profile, the amino acid sequence of the protein may be searched against a database of known protein domains (e.g., the HMM database). The transient receptor domain (HMM) has been assigned the PFAM Accession PF02164. A search was performed against the HMM database resulting in the identification of transient receptor domains in the amino acid sequence of human TLCC-5 at about residues 720-778 and 820-876 of SEQ ID NO:25.

A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

In a preferred embodiment, the TLCC-4 or TLCC-5 molecules of the invention include at least one transmembrane domain, at least one ankyrin repeat domain, at least one pore domain, at least one transient receptor domain, and/or at least one ion transport protein domain.

In a preferred embodiment, a TLCC-4 or TLCC-5 polypeptide includes at least one or more of the following domains: an ankyrin repeat domain, and/or a transmembrane domain, and/or a pore domain, and/or a transient receptor domain, and/or an ion transport protein domain, and has an amino acid sequence at least about 50%, 55%, 60%, 65%, 70%, 75%, 80%, 85%, 90%, 95%, 96%, 97%, 98%, 99% or more homologous or identical to the amino acid sequence of SEQ ID NO:22 or 25. In yet another preferred embodiment, a TLCC-4 or TLCC-5 polypeptide includes at least one or more of the following domains: an ankyrin repeat domain, and/or a transmembrane domain, and/or a pore domain, and/or a transient receptor domain, and/or an ion transport protein domain, and is encoded by a nucleic acid molecule having a nucleotide sequence which hybridizes under stringent hybridization conditions to a complement of a nucleic acid molecule comprising the nucleotide sequence of SEQ ID NO:21, 23, 24, or 26. In another preferred embodiment, a TLCC-4 or TLCC-5 polypeptide includes at least one or more of the following domains: an ankyrin repeat domain, and/or a transmembrane domain, and/or a pore domain, and/or a transient receptor domain, and/or an ion transport protein domain, and has a TLCC-4 or TLCC-5 activity.

As used interchangeably herein, a “TLCC-4 or TLCC-5 activity”, “biological activity of TLCC-4 or TLCC-5” or “functional activity of TLCC-4 or TLCC-5”, refers to an activity exerted by a TLCC-4 or TLCC-5 polypeptide or nucleic acid molecule on a TLCC-4 or TLCC-5 responsive cell or tissue, or on a TLCC-4 or TLCC-5 polypeptide substrate, as determined in vivo, or in vitro, according to standard techniques. In one embodiment, a TLCC-4 or TLCC-5 activity is a direct activity, such as an association with a TLCC-4-target molecule or TLCC-5-target molecule. As used herein, a “target molecule” or “binding partner” is a molecule with which a TLCC-4 or TLCC-5 polypeptide binds or interacts in nature, such that TLCC-4-mediated or TLCC-5-mediated function is achieved. A TLCC-4 or TLCC-5 target molecule can be a non-TLCC-4 or non-TLCC-5 molecule or a TLCC-4 or TLCC-5 polypeptide or polypeptide of the present invention. In an exemplary embodiment, a TLCC-4 or TLCC-5 target molecule is a TLCC-4 or TLCC-5 ligand, e.g., a calcium channel ligand such as calcium. Alternatively, a TLCC-4 or TLCC-5 activity is an indirect activity, such as a cellular signaling activity mediated by interaction of the TLCC-4 or TLCC-5 polypeptide with a TLCC-4 or TLCC-5 ligand. The biological activities of TLCC-4 or TLCC-5 are described herein. For example, the TLCC-4 or TLCC-5 polypeptides of the present invention can have one or more of the following activities: (1) modulate membrane excitability, (2) influence the resting potential of membranes, (3) modulate wave forms and frequencies of action potentials, (4) modulate thresholds of excitation, (5) modulate neurite outgrowth and synaptogenesis, (6) modulate signal transduction, and (7) participate in nociception.

Accordingly, another embodiment of the invention features isolated TLCC-4 or TLCC-5 polypeptides and polypeptides having a TLCC-4 or TLCC-5 activity. Preferred polypeptides are TLCC-4 or TLCC-5 polypeptides having at least one or more of the following domains: an ankyrin repeat domain, and/or a transmembrane domain, and/or a pore domain, and/or a transient receptor domain, and/or an ion transport protein domain and, preferably, a TLCC-4 or TLCC-5 activity.

Additional preferred polypeptides have one or more of the following domains: an ankyrin repeat domain, a transmembrane domain, a pore domain, a transient receptor domain, and/or an ion transport protein domain, and are, preferably, encoded by a nucleic acid molecule having a nucleotide sequence which hybridizes under stringent hybridization conditions to a nucleic acid molecule comprising the nucleotide sequence of SEQ ID NO:21, 23, 24, or 26.

Tissue Distribution of TLCC-4 mRNA

The tissue distribution of TLCC-4 mRNA was determined by RT-PCR using various cDNA libraries and a human TLCC-4-specific probe. From this analysis it was determined that TLCC-4 mRNA was expressed predominantly in the hypothalamus and skin. TLCC-4 mRNA was found in moderate levels in adipose and testes, and in low levels in skeletal muscle and brain.

In situ hybridization experiments were performed using a human TLCC-4-specific probe indicating TLCC-4 expression in monkey brain (cortex, thalamus, caudate, and hippocampus), spinal cord, DRG and SRG neurons, and in hair follicles. In situ hybridization with rat pain models indicated that TLCC-4 mRNA was down-regulated after chronic constriction injury, which causes persistent, spontaneous firing of neurons and results in pain. TLCC-4 mRNA was also down-regulated after treatment with clofibric acid, a selective muscle toxin which produces muscle pain and inflammation.

### Gene Expression of TLCC-4

The tissue distribution of human TLCC-4 mRNA was also determined using the TaqMan procedure on a variety of cells and tissues.

Strong expression of TLCC-4 was detected in human brain (hypothalamus) and skin tissues. In addition, TLCC-4 expression was detected at moderate levels in adipose and testis tissues, and at low levels in the fetal heart, skeletal muscle, brain, and colon tissues. Pain human panel phase I and MP Phase 1.3.3 libraries were also analyzed and it was determined that TLCC-4 was expressed at high levels in the brain, cortex, and testis, at moderate levels in the spinal cord, dorsal root ganglion (DRG), and the hypothalamus, and at low levels in the skin, placenta, small intestine, ovary, prostate epithelial cells, liver, skin (decubitus), colon tumor cells, and breast tumor cells. Monkey libraries were also analyzed indicating that TLCC-4 was expressed at high levels in the monkey cortex and hairy skin, and at low levels in the monkey spinal cord. Metabolic libraries were also analyzed demonstrating that TLCC-4 was expressed at high levels in adipose and brain tissues, and at low levels in differentiated adipocytes and pre-adipocytes, as well as in the hypothalamus, colon, small intestine, skeletal muscle, and liver tissues.

### Regulation of Calcium Influx Through TLCC-4

The regulation of calcium influx though TLCC-4 in 911 cells was determined by Fluorometric Imaging Plate Reader experiments (FLIPR) (Molecular Devices Corp., Sunnyvale, Calif.).

The FLIPR is a screening tool for cell-based fluorescent assays which allows the simultaneous stimulation and measurement of separate cell populations in a high throughput format. Therefore, using this system, it is possible to quantify transient signals, such as the release of intracellular calcium, from cell populations, in parallel and in real time. The FLIPR contains chambers in which to hold the test plate and plates containing antagonists or agonists to be added to the test plate. The FLIPR utilizes an argon laser that provides discrete spectral lines spaced from approximately 350 to 530 nm. For use with fluorescent Ca²⁺ dyes, the 88-nm line of the laser is employed. The laser simultaneously illuminates the wells in a test plate. The image of each well in the plate is captured by a cooled charge coupled device (CCD) camera, which updates images once per second, if required, for the measurement of rapid calcium responses. Because both excitation and emission are read via the bottom of the plate, black-walled, transparent bottomed 96-well plates are used. Data captured by the CCD camera is converted to digital data and then transferred to a computer.

Briefly, a calcium indicator (e.g., fluo-3/AM or Calcium Green-1/AM) was transferred to the culture medium. Because the FLIPR collects fluorescence from the bottom of the well. Suspension cells require centrifugation to the base of the well following dye loading. Viable 911 cells were resuspended in loading medium and incubated for one hour. The cells were then centrifuged and resuspended with wash buffer. The cell suspension containing the dye was then aliquoted into each well of the black-walled, transparent bottomed 96-well plate and the plate was centrifuged. The FLIPR assay was then carried out and the results analyzed. (If adherent cells are used, they may be plated at an appropriate density in the 96-well plates and cultured overnight. Dye may then be loaded and incubated).

Results show a constitutive calcium influx through TLCC-4 in 911 cells that were incubated with NMDG/0 Ca²⁺ and stimulated afterwards with 5 mM Ca²⁺.

### Human 5433

Non-voltage gated cation currents, which are activated following stimulation of phospholipase C (PLC), appear to be major modes for Ca⁺⁺ and Na⁺ entry in cells. The TRP channel family may mediate some of these conductances since their expression in vitro leads to PLC-dependent calcium influx. Members of this family have been proposed to mediate the entry of extracellular calcium into cells in response to the depletion of intracellular calcium stores. The influx of Ca⁺⁺ and Na⁺ is essential in the nervous system for propagation of action potential, synaptic transmission etc. Furthermore, one member of the channel family, TRPC3 or 5433, has been shown to contribute to a PLC-dependent calcium influx induced by brain-derived neurotrophic factor (BDNF), a known mediator of neuropathic pain. Thus, this family of molecules may play important roles in sensory signal transduction in general.

The human 5433 sequence (SEQ ID NO:27), which is approximately 3448 nucleotides long including untranslated regions, contains a predicted methionine-initiated coding sequence of about 2544 nucleotides (nucleotides 425-2968 of SEQ ID NO:27; 1-2544 of SEQ ID NO:29), not including the termination codon. The coding sequence encodes a 848 amino acid protein (SEQ ID NO:28).

Human 5433 contains the following regions or other structural features: one predicted calcium channel domain (ion transport protein domain, PFAM Accession Number PF00520) located at about amino acid residues 436 to 670 of SEQ ID NO:28; two predicted ank repeat domains (PFAM Accession Number PF00023) located at about amino acids 73 to 105, and 159 to 191 of SEQ ID NO:28; six predicted transmembrane segments located at about amino acids 386 to 402, 434 to 450, 474 to 492, 541 to 557, 580 to 603, and 646 to 670 of SEQ ID NO:28; one predicted N-terminal cytoplasmic domain located at about amino acids 1 to 385 of SEQ ID NO:28; one predicted C-terminal cytoplasmic domain located at about amino acids 671 to 848 of SEQ ID NO:28; two predicted cytoplasmic loops located at about amino acids 451 to 473, and 558 to 579 of SEQ ID NO:28; three predicted non-cytoplasmic loops located at about amino acids 403 to 433, 493 to 540, and 604 to 645 of SEQ ID NO:28; six predicted N-glycosylation sites (PS00001) located at about amino acids 337 to 340, 403 to 406, 416 to 419, 560 to 563, 655 to 658, and 671 to 674 of SEQ ID NO:28; three predicted cAMP- and cGMP-dependent protein kinase phosphorylation sites (PS00004) located at about amino acids 8 to 11, 131 to 134, and 260 to 263 of SEQ ID NO:28; nine predicted Protein Kinase C phosphorylation sites (PS00005) located at about amino acids 6 to 8, 130 to 132, 405 to 407, 485 to 487, 573 to 575, 617 to 619, 712 to 714, 813 to 815, and 837 to 839 of SEQ ID NO:28; eleven predicted Casein Kinase II phosphorylation sites (PS00006) located at about amino acids 37 to 40, 136 to 139, 230 to 233, 237 to 240, 418 to 421, 435 to 438, 573 to 576, 612 to 615, 673 to 676, 681 to 684, and 695 to 698 of SEQ ID NO:28; two predicted Tyrosine kinase phosphorylation sites (PS00007) located at about amino acids 42 to 49, and 641 to 648 of SEQ ID NO:28; four predicted N-myristylation sites (PS00008) located at about amino acids 33 to 38, 447 to 452, 604 to 609, and 741 to 746 of SEQ ID NO:28; and one predicted Amidation site (PS00009) located at about amino acids 16 to 19 of SEQ ID NO:28.

For general information regarding PFAM identifiers, PS prefix and PF prefix domain identification numbers, refer to Sonnhammer et al. (1997) Protein 28:405-420.

TABLE 6
Summary of transmembrane domains.

| Transmembrane | | Extracellular |
|---|---|---|
| segment | | sequence |
| located in | Cytoplasmic sequence | located in |
| SEQ ID NO: 28 | located in SEQ ID NO: 28 | SEQ ID NO: 28 |
| about 386 to 402 | about 1 to 385 (N-terminal) | |
| about 434 to 450 | | about 403 to 433 |
| about 474 to 492 | about 451 to 473 | |
| about 541 to 557 | | about 493 to 540 |
| about 580 to 603 | about 558 to 579 | |
| about 646 to 670 | | about 604 to 645 |
| | about 671 to 848 (C-terminal) | |

A hydropathy plot of human 5433 was performed. Polypeptides of the invention include fragments which include: all or part of a hydrophobic sequence, e.g., the sequence from about amino acid 265 to 275, from about 345 to 380, and from about 702 to 720 of SEQ ID NO:28; all or part of a hydrophilic sequence, e.g., the sequence of from about amino acid 194 to 220, from about 245 to 262, and from about 720 to 741 of SEQ ID NO:28; a sequence which includes a Cys, or a glycosylation site.

The 5433 protein contains a significant number of structural characteristics in common with members of the calcium channel family. In particular, the 5433 protein shows homology to the transient receptor potential (TRP) proteins. The term “family” when referring to the protein and nucleic acid molecules of the invention means two or more proteins or nucleic acid molecules having a common structural domain or motif and having sufficient amino acid or nucleotide sequence homology as defined herein.

As used herein, a “calcium channel” includes a protein or polypeptide that is involved in receiving, conducting, and transmitting signals in an electrically excitable cell, e.g., a neuronal or muscular cell. Calcium channels are calcium ion selective, and can determine membrane excitability (the ability of, for example, a muscle cell to respond to a stimulus and to convert it into an impulse resulting in a contraction). Calcium channels can also influence the resting potential of membranes, wave forms and frequencies of action potentials, and thresholds of excitation. Calcium channels are typically expressed in electrically excitable cells, e.g., neuronal or muscle cells, and may form heteromultimeric structures (e.g., composed of more than one type of subunit). Calcium channels are described in, for example, Davila et al. (1999) Annals New York Academy of Sciences 868: 102-17 and McEnery, M. W. et al. (1998) J. Bioenergetics and Biomembranes 30(4): 409-418, the contents of which are incorporated herein by reference.

Calcium signaling has been implicated in the regulation of a variety of cellular responses, such as neuronal development and maintenance, and cell growth and differentiation. There are two general methods by which intracellular concentrations of calcium ions may be increased: calcium ions may be brought into the cell from the extracellular milieu through the use of specific channels in the cellular membrane, or calcium ions may be freed from intracellular stores, again being transported by specific membrane channels in the storage organelle. In the situation in which the intracellular stores of calcium have been depleted, a specific type of calcium channel, termed a “capacitative calcium channel” or a “store-operated calcium channel” (SOC), is activated in the plasma membrane to import calcium ions from the extracellular environment to the cytosol (for review, see Putney and McKay (1999) BioEssays 21: 38-46). Members of the capacitative calcium channel family include the calcium release-activated calcium current (Hoth and Penner (1992) Nature 355: 353-355), calcium release-activated nonselective cation current (Krause et al. (1996) J. Biol. Chem. 271: 32523-32528), and the transient receptor potential (TRP) proteins. See, e.g., Putney, J. W., (1986) Cell Calcium 7: 1-12; Putney, J. W. (1990) Cell Calcium 11: 611-624.

The TRP channel family is one of the best characterized of the capacitative calcium channel group. These channels include transient receptor potential protein and homologues thereof, the vanilloid receptor subtype I, stretch-inhibitable non-selective cation channel, olfactory, mechanosensitive channel, insulin-like growth factor I-regulated calcium channel, and vitamin D-responsive apical, epithelial calcium channel (ECaC) (see, e.g., Montell and Rubin (1989) Neuron 2:1313-1323; Caterina et al. (1997) Nature 389: 816-824; Suzuki et al. (1999) J. Biol. Chem. 274: 6330-6335; Kiselyov et al. (1998) Nature 396: 478-482; and Hoenderop et al. (1999) J. Biol. Chem. 274: 8375-8378). Each of these molecules is at least 700 amino acids, and shares certain conserved structural features. Predominant among these structural features are six transmembrane domains, with an additional hydrophobic loop present between the fifth and sixth transmembrane domains. It is believed that this loop is integral to the activity of the pore of the channel formed upon membrane insertion (Hardie and Minke (1993) Trends Neurosci 16: 371-376). TRP channel proteins also include one or more ankyrin domains and frequently display a proline-rich region at the N-terminus. See, e.g., McClesky and Gold (1999) Annu. Rev. Physiol. 61: 835-856; and Colbert et al. (1997) J. Neurosci 17(21): 8259-8269.

Non-voltage gated cation currents, which are activated following stimulation of phospholipase C (PLC), appear to be major modes for Ca⁺⁺ and Na⁺ entry in cells. The TRP channel family may mediate some of these conductances since their expression in vitro leads to PLC-dependent calcium influx. Members of this family have been proposed to mediate the entry of extracellular calcium into cells in response to the depletion of intracellular calcium stores. The influx of Ca⁺⁺ and Na⁺ is essential in the nervous system for propagation of action potential, synaptic transmission etc. Furthermore, one member of the channel family, TRPC3, has been shown to contribute to a PLC-dependent calcium influx induced by brain-derived neurotrophic factor (BDNF), a known mediator of neuropathic pain. Thus, this family of molecules may play important roles in sensory signal transduction in general.

The 5433 protein shows homology to transient receptor potential (TRP) proteins. The term “transient receptor potential” protein refers to a membrane-spanning, glycoprotein cation (calcium or sodium) channels. A limited sequence similarity to voltage-gated calcium channel al subunits lead to the prediction of six transmembrane segments flanked by intracellular N and C termini and a putative pore region between the transmembrane segments 5 and 6. Generally, the first hydrophobic region rather than being a transmembrane segment is intracellular and available for protein-protein interactions. See, e.g., Vannier B. et al. (1998) J. Biol. Chem. 273: 8675-8679. Preferably, the TRP protein includes six endogenous glycosylation sites (Zhu X. et al. (1996) Cell 85: 661-671).

The 5433 polypeptides contain structural features similar to calcium channel protein family members, in particular, to the TRP proteins. For example, the 5433 polypeptide has seven predicted hydrophobic regions present at about amino acids 350 to 369, 386 to 402, 434 to 450, 474 to 492, 541 to 557, 580 to 603, and 646 to 670 of SEQ ID NO:28, and the first region rather than being a transmembrane segment is intracellular domain. Accordingly, the 5433 polypeptide has six transmembrane segments flanked by intracellular N and C termini. The 5433 polypeptide also has six predicted N-glycosylation sites (PS00001) located at about amino acids 337 to 340, 403 to 406, 416 to 419, 560 to 563, 655 to 658, and 671 to 674 of SEQ ID NO:28.

Non-voltage gated cation currents, which are activated following stimulation of phospholipase C, appear to be major modes for calcium and sodium entry in cells. TRP family members are believed to mediate some of these conductances since their expression in vitro leads to phospholipase C (PLC)-dependent calcium influx. Members of this family have been proposed to mediate entry of extracellular calcium into cells in response to the depletion of intracellular stores. The influx of calcium and sodium is essential in the nervous system for the propagation of action potentials, synaptic transmission, etc. Furthermore, TRPs have been shown to contribute to PLC-dependent calcium influx induced by BDNF, a known mediator of neuropathic pain. Accordingly, 5433-activity may be involved in neurological processes, including PLC-mediated conductances associated with the propagation of action potentials, synaptic transmission, nociceptive responses, and neuropathic pain.

A 5433 polypeptide can include an “ion channel domain” or a “calcium channel domain” (or “ion transport protein domain”), or regions homologous with a “calcium channel domain.”

As used herein, the term “calcium channel domain” includes an amino acid sequence of about 150 to 450 amino acid residues in length and having a bit score for the alignment of the sequence to the calcium channel domain profile (PFAM HMM) of at least 50. Preferably, a calcium channel domain includes at least about 200 to 300 amino acids, more preferably about 210 to 250 amino acid residues, or about 234 amino acids and has a bit score for the alignment of the sequence to the calcium channel domain (HMM) of at least 60, preferable 70, 75 or greater. The calcium channel domain (HMM) has been assigned the PFAM Accession Number PF00520. The calcium channel domain (amino acids 436 to 670 of SEQ ID NO:28) of human 5433 aligns with a consensus amino acid sequence (SEQ ID NO:30) derived from a hidden Markov model.

In a preferred embodiment 5433 polypeptide or protein has a “calcium channel domain” or a region which includes at least about 150 to 450, more preferably about 200 to 300 amino acids, more preferably about 210 to 250 amino acid residues and has at least about 70% 80% 90% 95%, 99%, or 100% homology with a “calcium channel domain,” e.g., the calcium channel domain of human 5433 (e.g., residues 436 to 670 of SEQ ID NO:28).

To identify the presence of a “calcium channel” domain in a 5433 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3): 405-420 and a detailed description of HMMs can be found, for example, in Gribskov et al. (1990) Meth. Enzymol. 183: 146-159; Gribskov et al. (1987) Proc. Natl. Acad. Sci. USA 84: 4355-4358; Krogh et al. (1994) J. Mol. Biol. 235: 1501-1531; and Stultz et al. (1993) Protein Sci. 2: 305-314, the contents of which are incorporated herein by reference. A search was performed against the HMM database resulting in the identification of a “calcium channel” domain in the amino acid sequence of human 5433 at about residues 436 to 670 of SEQ ID NO:28.

A 5433 molecule can further include at least one, or two ank repeat domains. An ank repeat domain is characterized by a common fold, of about 30 amino acids, characterized by a helix-beta-turn-helix core. See, for example, Kalus W. et al. (1997) FEBS Lett 401(2-3): 127-32.

As used herein, the term “ank repeat domain” (or “ankyrin domain”) includes an amino acid sequence of about 10 to 50 amino acid residues in length and having a bit score for the alignment of the sequence to the ank repeat domain (HMM) of at least 5. Preferably, an ank domain includes at least about 20 to 40 amino acids, more preferably about 30 to 35 amino acids, or about 32 amino acids, and has a bit score for the alignment of the sequence to the ank repeat domain (HMM) of at least 10, preferably 14, or more preferably 15 or greater. The ank repeat domain (HMM) has been assigned the PFAM Accession Number PF00023. The ank repeat domain (amino acids 73 to 105, or 159 to 191 of SEQ ID NO:28) of human 5433 aligns with a consensus amino acid sequence (SEQ ID NO:31) derived from a hidden Markov model.

In a preferred embodiment 5433 polypeptide or protein has an “ank repeat domain” or a region which includes at least about 10 to 50 more preferably about 20 to 40, or about 32 amino acid residues and has at least about 70% 80% 90% 95%, 99%, or 100% homology with an “ank repeat domain,” e.g., the ank repeat domain of human 5433 (e.g., amino acids 73 to 105, or 159 to 191 of SEQ ID NO:28).

To identify the presence of an “ank repeat” domain in a 5433 protein sequence, and make the determination that a polypeptide or protein of interest has a particular profile, the amino acid sequence of the protein can be searched against a database of HMMs as described above. A search was performed against the HMM database resulting in the identification of an “ank repeat domain” domain in the amino acid sequence of human 5433 at about residues acids 73 to 105, or 159 to 191 of SEQ ID NO:28.

A 5433 protein further includes a predicted N-terminal cytoplasmic domain located at about amino acids 1-385 of SEQ ID NO:28. As used herein, a “N-terminal cytoplasmic domain” includes an amino acid sequence having about 1-500, preferably about 1-400, or even more preferably about 1-390 amino acid residues in length and is located inside of a cell or intracellularly. The C-terminal amino acid residue of a “N-terminal cytoplasmic domain” is adjacent to a N-terminal amino acid residue of a transmembrane domain in a 5433 protein. For example, a N-terminal cytoplasmic domain is located at about amino acid residues 1-385 of SEQ ID NO:28.

In a preferred embodiment 5433 polypeptide or protein has an “N-terminal cytoplasmic domain” or a region which includes at least about 1-600, preferably about 100-400, and even more preferably about 385 amino acid residues and has at least about 60%, 70% 80% 90% 95%, 99%, or 100% homology with an “N-terminal cytoplasmic domain,” e.g., the N-terminal cytoplasmic domain of human 5433 (e.g., residues 1-385 of SEQ ID NO:28).

In another embodiment, a 5433 protein includes a “C-terminal cytoplasmic domain,” also referred to herein as a C-terminal cytoplasmic tail, in the sequence of the protein. As used herein, a “C-terminal cytoplasmic domain” includes an amino acid sequence having a length of at least about 100, more preferably 150 or more amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring 5433 protein. For example, a C-terminal cytoplasmic domain is found at about amino acid residues 671 to 848 of SEQ ID NO:28.

In a preferred embodiment, a 5433 polypeptide or protein has a C-terminal cytoplasmic domain or a region which includes at least about 100, more preferably 150 or more amino acid residues and has at least about 60%, 70% 80% 90% 95%, 99%, or 100% homology with an “C-terminal cytoplasmic domain,” e.g., the C-terminal cytoplasmic domain of human 5433 (e.g., residues 671 to 848 of SEQ ID NO:28).

5433 proteins can further include at least one, two, three, four, five, and preferably six transmembrane domains. As used herein, the term “transmembrane domain” includes an amino acid sequence of about 10 to 45, preferably 12 to 30, and most preferably 15 to 25, amino acid residues in length that spans the plasma membrane. More preferably, a transmembrane domain includes about at least 17, 19, 24, or 25 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neurosci. 19: 235-263, the contents of which are incorporated herein by reference. Amino acid residues 386 to 402, 434 to 450, 474 to 492, 541 to 557, 580 to 603, and 646 to 670 of SEQ ID NO:28 are transmembrane domains. Accordingly, proteins having at least 50-60% homology, preferably about 60-70%, more preferably about 70-80%, about 80-90%, or about 90-100% homology with amino acids 386 to 402, 434 to 450, 474 to 492, 541 to 557, 580 to 603, and 646 to 670 of SEQ ID NO:28 are within the scope of the invention.

In another embodiment, a 5433 protein includes at least one, or two cytoplasmic loop, also referred to herein as a cytoplasmic domain. As used herein, a “cytoplasmic loop” includes an amino acid sequence having a length of at least about 10, preferably about 20, amino acid residues located within a cell or within the cytoplasm of a cell. For example, a cytoplasmic loop is found at about amino acids 451 to 473, or 558 to 579 of SEQ ID NO:28.

In a preferred embodiment 5433 polypeptide or protein has at least one cytoplasmic loop or a region which includes at least about 10, preferably about 20 amino acid residues and has at least about 60%, 70% 80% 90% 95%, 99%, or 100% homology with an “cytoplasmic loop,” e.g., at least one cytoplasmic loop of human 5433 (e.g., residues 451 to 473, or 558 to 579 of SEQ ID NO:28).

In another embodiment, a 5433 protein include at least one, two, or three non-cytoplasmic (extracellular) loop. As defined herein, the term “loop” includes an amino acid sequence that resides outside of a phospholipid membrane, having a length of at least about 20 to 70, and preferably about 30 to 50 amino acid residues, and has an amino acid sequence that connects two transmembrane domains within a protein or polypeptide. Extracellular domains are located outside of the cell. Accordingly, the N-terminal amino acid of a non-cytoplasmic loop is adjacent to a C-terminal amino acid of a transmembrane domain in a 5433 protein, and the C-terminal amino acid of a non-cytoplasmic loop is adjacent to an N-terminal amino acid of a transmembrane domain in a 5433 protein. For example, an “extracellular loop” can be found at about amino acids 403 to 433, 493 to 540, and 604 to 645 of SEQ ID NO:28.

In a preferred embodiment, a 5433 polypeptide or protein has at least one, two, or three non-cytoplasmic loops or regions which include at least about 20 to 70, and preferably about 30 to 50 amino acid residues and has at least about 70% 80% 90% 95%, 99%, or 100% homology with an “extracellular loop,” e.g., at least one non-cytoplasmic loop of human 5433 (e.g., residues 403 to 433, 493 to 540, and 604 to 645 of SEQ ID NO:28).

Accordingly, in one embodiment of the invention, a 5433 includes at least one, two, three, four, five, preferably six, transmembrane domains, at least one, or two cytoplasmic loops, and/or at least one, two, or three extracellular loops. In another embodiment, the 5433 further includes an N-terminal and a C-terminal cytoplasmic domains.

A 5433 family member can include at least one predicted calcium channel domain; and at least one, preferably two predicted ank repeat domains. Furthermore, a 5433 family member can include at least one, two, three, four, five, preferably six predicted N-glycosylation sites (PS00001); at least one, two, preferably three predicted cAMP- and cGMP-dependent protein kinase phosphorylation sites (PS00004); at least one, two, three, four, five, six, seven, eight, preferably nine predicted protein kinase C phosphorylation sites (PS00005); at least one, two, three, four, five, six, seven, eight, nine, ten, and preferably eleven predicted casein kinase II phosphorylation sites (PS00006); at least one, preferably two Tyrosine kinase phosphorylation sites (PS00007); at least one, two, three, and preferably four predicted N-myristylation sites (PS00008); and at least one predicted Amidation site (PS00009).

As the 5433 polypeptides of the invention may modulate 5433-mediated activities, they may be useful as of for developing novel diagnostic and therapeutic agents for 5433-mediated or related disorders, as described below.

As used herein, a “5433 activity,” “biological activity of 5433,” or “functional activity of 5433,” refers to an activity exerted by a 5433 protein, polypeptide or nucleic acid molecule on e.g., a 5433-responsive cell or on a 5433 substrate, e.g., a protein substrate, as determined in vivo or in vitro. In one embodiment, a 5433 activity is a direct activity, such as an association with a 5433 target molecule. A “target molecule” “substrate” or “binding partner” is a molecule with which a 5433 protein binds or interacts in nature. A 5433 activity can also be an indirect activity, e.g., a cellular signaling activity mediated by interaction of the 5433 protein with a 5433 binding partner. In an exemplary embodiment, 5433 is controlling neurotransmitter release from neurons.

Based on the above-described sequence similarities and the tissue distribution described below, the 5433 molecules of the present invention are predicted to have similar biological activities as calcium channel family members. Thus, in accordance with the invention, a 5433 calcium channel or subsequence or variant polypeptide may have one or more domains and, therefore, one or more activities or functions characteristic of a calcium channel family member, including, but not limited to, (1) controlling neurotransmitter release from neurons; (2) regulating nociceptive responses; (3) regulating synaptic transmission; (3) modulating cation, e.g., calcium or sodium, entry into a cell, e.g., a neuronal cell; or (5) modulating pain or inflammation response. Thus, the 5433 molecules can act as novel diagnostic targets and therapeutic agents for controlling ion (e.g., calcium) channel-associated disorders.

Nociceptive responses in general are heterogeneous processes involving intracellular signaling mediated by phospholipase C, a known activator of TRP receptor channels. TRP receptor channels has been shown to be regulated by brain-derived neurotrophic factor (BDNF), a known mediator of neuropathic pain. Thus, 5433 calcium channel protein or subsequence or variant having calcium channel activity is capable of, e.g., modulating nociceptive responses, and also pain responses.

Expression of 5433 mRNA is detected in human and rat brain, spinal cord, and dorsal root ganglia (DRG) (Tables 7-11). Expression was low in any other normal human and rat tissues. In situ hybridization experiments with the human probe showed expression in monkey and rat cortex, spinal cord, and DRG neurons. Hence, 5433 is likely a neuro-related calcium channel protein involved in neurological response, e.g., nociceptive and pain responses.

Animal models indicate a role for the 5433 molecule in pain response. Examples of animal models of pain response that can be tested include, but are not limited to, axotomy, the cutting or severing of an axon; chronic constriction injury (CCI), a model of neuropathic pain which involves ligation of the sciatic nerve in rodents, e.g., rats; or intraplantar Freund's adjuvant injection as a model of arthritic pain. Other animal models of pain response are described in, e.g., ILAR Journal (1999) Volume 40, Number 3 (entire issue). Taqman experiments in rat animal models show no regulation in DRGs. However, 5433 is up-regulated in the spinal cord after CCI axotomy, and after CFA intraplantar injection.

Therefore, 5433 associated disorders can detrimentally affect regulation and modulation of the pain response; and vasoconstriction response and pain therefrom. Examples of 5433 associated disorders in which the 5433 molecules of the invention may be directly or indirectly involved include pain, pain syndromes, and inflammatory disorders, including inflammatory pain. Accordingly, the 5433 molecules can act as novel diagnostic targets and therapeutic agents controlling neurological, e.g., neurodegenerative, disorders and pain disorders.

Agents that modulate 5433 polypeptide or nucleic acid activity or expression can be used to treat pain elicited by any medical condition. A subject receiving the treatment can be additionally treated with a second agent, e.g., an anti-inflammatory agent, an antibiotic, or a chemotherapeutic agent, to further ameliorate the condition.

The 5433 molecules can also act as novel diagnostic targets and therapeutic agents for brain or neurological disorders.

5433 RNA expression is also detected in a panel of cardiovascular (CV) organ and vessel tissues (Tables 12-13). In Table 12, Taqman with CV organ panel shows expression of 5433 in the fetal heart. In Table 13, Taqman with CV vessel panel shows expression of 5433 blood vessels, e.g., artery, Huvec, aorta, and vein. These data show that 5433 calcium channel protein is capable of modulating cardiovascular-related disorders. A cardiovascular disease or disorder also can include an endothelial cell disorder.

Tissue Distribution of 5433 mRNA by TaqMan Analysis

Taqman experiments using a panel of human normal and tumor tissues, are described in Table 7. The expression of 5433 mRNA using additional human tissues is depicted in Table 8. Taqman experiments using rat panels are depicted in Tables 9-11.

In humans, 5433 mRNA was highly expressed in the normal brain cortex, hypothalamus, dorsal root ganglion (DRG), and prostate/testis. Further analysis shows expression of 5433 mRNA to be observed in brain, followed by testis, spinal cord, and DRG.

In rat, expression of 5433 mRNA was observed in the brain, DRG, spinal cord, SGC, and optic nerve. Expression was very low in other normal human and rat tissues tested. Taqman experiments in rat models, respectively, showed no significant regulation of 5433 mRNA in the DRGs. However, 5433 mRNA is upregulated in the spinal cord after CCI, axotomy and after CFA intraplantar injection. The relative tissue distribution of 5433 mRNA is depicted in tabular form in Tables 9-11.

TABLE 7

| Tissue Type | Mean | β 2 Mean | ∂∂ Ct | Expression |
|---|---|---|---|---|
| Artery normal | 37.24 | 22.67 | 12.78 | 0 |
| Aorta diseased | 40 | 22.98 | 15.22 | 0 |
| Vein normal | 40 | 21.34 | 16.86 | 0 |
| Coronary SMC | 40 | 22.23 | 15.98 | 0 |
| HUVEC | 38.54 | 22.68 | 14.07 | 0 |
| Hemangioma | 37.06 | 21.36 | 13.89 | 0 |
| Heart normal | 37.23 | 21.41 | 14.03 | 0 |
| Heart CHF | 38.94 | 21.68 | 15.47 | 0 |
| Kidney | 37.41 | 21.16 | 14.46 | 0 |
| Skeletal Muscle | 40 | 23.43 | 14.77 | 0 |
| Adipose normal | 40 | 22.7 | 15.5 | 0 |
| Pancreas | 40 | 23.6 | 14.6 | 0 |
| primary osteoblasts | 40 | 21.3 | 16.91 | 0 |
| Osteoclasts (diff) | 40 | 18.26 | 19.94 | 0 |
| Skin normal | 40 | 23.28 | 14.92 | 0 |
| Spinal cord normal | 39.56 | 22.29 | 15.47 | 0 |
| Brain Cortex normal | 30.3 | 23.42 | 5.08 | 29.5643 |
| Brain Hypothalamus normal | 34.9 | 24 | 9.11 | 1.816 |
| Nerve | 37.1 | 22.51 | 12.79 | 0 |
| DRG (Dorsal Root Ganglion) | 32.52 | 22 | 8.71 | 2.3797 |
| Breast normal | 40 | 22.11 | 16.09 | 0 |
| Breast tumor | 40 | 21.95 | 16.25 | 0 |
| Ovary normal | 39.95 | 21.27 | 16.87 | 0 |
| Ovary Tumor | 39.67 | 20.1 | 17.77 | 0 |
| Prostate Normal | 36.98 | 20.2 | 14.98 | 0 |
| Prostate Tumor | 38.45 | 21.12 | 15.53 | 0 |
| Salivary glands | 39.95 | 20.36 | 17.79 | 0 |
| Colon normal | 40 | 20.18 | 18.02 | 0 |
| Colon Tumor | 40 | 22.68 | 15.53 | 0 |
| Lung normal | 35.23 | 19.74 | 13.69 | 0 |
| Lung tumor | 36.56 | 21.1 | 13.66 | 0 |
| Lung COPD | 35.32 | 19.41 | 14.11 | 0 |
| Colon IBD | 40 | 18.72 | 19.48 | 0 |
| Liver normal | 40 | 21.3 | 16.9 | 0 |
| Liver fibrosis | 40 | 22.15 | 16.06 | 0 |
| Spleen normal | 39.81 | 19.96 | 18.05 | 0 |
| Tonsil normal | 37.44 | 19.16 | 16.48 | 0 |
| Lymph node normal | 39.39 | 21.02 | 16.57 | 0 |
| Small intestine normal | 35.09 | 21.15 | 12.15 | 0 |
| Macrophages | 40 | 18.15 | 20.05 | 0 |
| Synovium | 40 | 21.27 | 16.93 | 0 |
| BM-MNC | 40 | 20.42 | 17.78 | 0 |
| Activated PBMC | 36.02 | 18.59 | 15.64 | 0 |
| Neutrophils | 40 | 18.86 | 19.34 | 0 |
| Megakaryocytes | 40 | 19.68 | 18.52 | 0 |
| Erythroid | 40 | 22.02 | 16.18 | 0 |
| positive control | 30.72 | 21.56 | 7.36 | 6.0872 |

TABLE 8

| Tissue Type | 5433 | β2.803 | ∂Ct | Expression |
|---|---|---|---|---|
| Adrenal Gland | 40.00 | 19.75 | 20.26 | 0.00 |
| Brain | 29.73 | 21.21 | 8.52 | 2.73 |
| Heart | 39.69 | 20.31 | 19.39 | 0.00 |
| Kidney | 38.68 | 20.31 | 18.37 | 0.00 |
| Liver | 39.43 | 20.46 | 18.97 | 0.00 |
| Lung | 34.02 | 18.86 | 15.17 | 0.03 |
| Mammary Gland | 40.00 | 21.84 | 18.17 | 0.00 |
| Pancreas | 40.00 | 21.75 | 18.26 | 0.00 |
| Placenta | 35.69 | 21.23 | 14.46 | 0.04 |
| Prostate | 36.85 | 19.06 | 17.80 | 0.00 |
| Salivary Gland | 38.51 | 20.69 | 17.83 | 0.00 |
| Muscle | 40.00 | 22.91 | 17.10 | 0.01 |
| Sm. Intestine | 36.65 | 20.26 | 16.40 | 0.01 |
| Spleen | 37.69 | 18.55 | 19.14 | 0.00 |
| Stomach | 34.63 | 19.33 | 15.31 | 0.02 |
| Teste | 32.56 | 21.64 | 10.92 | 0.52 |
| Thymus | 40.00 | 26.20 | 13.80 | 0.07 |
| Trachea | 40.00 | 22.37 | 17.63 | 0.00 |
| Uterus | 35.09 | 20.23 | 14.86 | 0.03 |
| Spinal Cord | 32.51 | 19.30 | 13.21 | 0.11 |
| DRG | 34.33 | 20.24 | 14.09 | 0.06 |
| Skin | 39.24 | 19.96 | 19.28 | 0.00 |

TABLE 9

| Tissue | r5433 | 18S | ∂Ct | Expression |
|---|---|---|---|---|
| Brain | 27.14 | 11.12 | 16.02 | 0.015 |
| Spinal Cord | 29.76 | 11.42 | 18.34 | 0.003 |
| DRG | 28.13 | 11.51 | 16.62 | 0.010 |
| SCG | 30.19 | 11.78 | 18.42 | 0.003 |
| Optic Nerve | 29.94 | 10.90 | 19.05 | 0.002 |
| Hairy Skin | 33.83 | 12.21 | 21.62 | 0.000 |
| Gastro Muscle | 32.77 | 11.37 | 21.40 | 0.000 |
| Heart | 31.47 | 11.00 | 20.47 | 0.001 |
| Kidney | 34.01 | 12.53 | 21.48 | 0.000 |
| Liver | 39.22 | 12.09 | 27.13 | 0.000 |
| Lung | 32.37 | 12.28 | 20.09 | 0.001 |
| Spleen | 33.76 | 11.98 | 21.78 | 0.000 |
| Aorta | 31.94 | 11.61 | 20.34 | 0.001 |
| Adrenal Gland | 32.90 | 11.40 | 21.50 | 0.000 |
| Salivary Gland | 34.50 | 11.21 | 23.29 | 0.000 |
| Thyroid | 31.62 | 11.33 | 20.29 | 0.001 |
| Prostate | 38.23 | 11.35 | 26.88 | 0.000 |
| Thymus | 34.43 | 11.17 | 23.26 | 0.000 |
| Trachea | 32.69 | 11.92 | 20.77 | 0.001 |
| Esophagus | 32.85 | 11.52 | 21.33 | 0.000 |
| Duodenum | 33.95 | 12.37 | 21.59 | 0.000 |
| Diaphragm | 33.21 | 12.38 | 20.83 | 0.001 |
| Colon | 34.05 | 12.49 | 21.56 | 0.000 |

TABLE 10

| Tissue | r5433 | 18S | ∂Ct | Expression |
|---|---|---|---|---|
| Naïve DRG | 28.38 | 11.75 | 16.64 | 0.010 |
| I DRG CCI 3 | 28.06 | 11.59 | 16.48 | 0.011 |
| I DRG CCI 7 | 28.13 | 11.75 | 16.38 | 0.012 |
| I DRG CCI 10 | 28.33 | 11.77 | 16.56 | 0.010 |
| I DRG CCI 28 | 27.97 | 11.95 | 16.02 | 0.015 |
| Naïve DRG | 28.38 | 11.68 | 16.71 | 0.009 |
| I DRG CFA 1 | 28.91 | 12.01 | 16.90 | 0.008 |
| I DRG CFA 3 | 28.41 | 11.98 | 16.44 | 0.011 |
| I DRG CFA 7 | 28.01 | 11.94 | 16.08 | 0.014 |
| I DRG CFA 10 | 28.20 | 11.96 | 16.24 | 0.013 |
| I DRG CFA 14 | 27.99 | 11.55 | 16.44 | 0.011 |
| I DRG CFA 28 | 28.07 | 11.61 | 16.46 | 0.011 |
| Naïve DRG | 28.18 | 11.83 | 16.35 | 0.012 |
| I DRG AXT 1 | 28.04 | 11.56 | 16.48 | 0.011 |
| I DRG AXT 3 | 28.27 | 11.74 | 16.54 | 0.011 |
| I DRG AXT 7 | 28.44 | 11.59 | 16.86 | 0.008 |
| I DRG AXT 14 | 28.23 | 11.74 | 16.49 | 0.011 |

TABLE 11

| Tissue | r5433 | 18S | ∂Ct | Expression |
|---|---|---|---|---|
| Naïve SC | 27.21 | 11.60 | 15.62 | 0.12 |
| I SC CCI 3 | 26.02 | 11.78 | 14.24 | 0.30 |
| I SC CCI 7 | 25.89 | 12.12 | 13.77 | 0.41 |
| I SC CCI 10 | 26.30 | 12.52 | 13.78 | 0.41 |
| I SC CCI 14 | 26.09 | 11.76 | 14.33 | 0.28 |
| I SC CCI 28 | 26.41 | 12.28 | 14.14 | 0.32 |
| Naïve SC | 27.45 | 11.65 | 15.81 | 0.10 |
| I SC CFA 1 | 26.06 | 12.19 | 13.87 | 0.39 |
| I SC CFA 3 | 25.63 | 11.66 | 13.97 | 0.36 |
| I SC CFA 7 | 25.99 | 11.63 | 14.36 | 0.28 |
| I SC CFA 10 | 25.93 | 11.74 | 14.19 | 0.31 |
| I SC CFA 14 | 26.84 | 12.30 | 14.54 | 0.24 |
| I SC CFA 28 | 26.27 | 12.18 | 14.09 | 0.33 |
| Naïve SC | 27.08 | 11.57 | 15.51 | 0.12 |
| I SC AXT 1 | 25.87 | 11.89 | 13.98 | 0.36 |
| I SC AXT 3 | 26.33 | 11.88 | 14.45 | 0.26 |
| I SC AXT 7 | 26.12 | 11.82 | 14.30 | 0.29 |
| I SC AXT 14 | 25.94 | 12.43 | 13.52 | 0.50 |

The expression of 5433 mRNA in a panel of cardiovascular (CV) organ and vessel tissues are shown below as Tables 12-13. Tables 12 and 13 show results from Taqman studies with a cardiovascular organ panel showing expression of 5433 mRNA in fetal heart, and in the artery, followed by human vascular endothelial cells (Huvec shear), aorta, and vein.

TABLE 12

| Tissue Type | Mean | β 2 Mean | ∂∂ Ct | Expression |
|---|---|---|---|---|
| H/Fetal Heart/normal/BWH 4 | 29.34 | 22.34 | 7 | 7.8125 |
| H/Heart/Normal/Atrium/ | 35.19 | 21.08 | 14.11 | 0 |
| MPI 1097 | | | | |
| H/Heart/Normal/Ventricle/ | 35.98 | 18.22 | 17.75 | 0 |
| PIT 272 | | | | |
| H/Heart/Normal/Ventricle/ | 36.7 | 21.24 | 15.47 | 0 |
| PIT 206 | | | | |
| H/Heart/Normal/Ventricle/ | 37.98 | 18.54 | 19.44 | 0 |
| PIT 204 | | | | |
| H/Heart/Normal/Ventricle/ | 33.91 | 18.66 | 15.24 | 0.0258 |
| PIT 205 | | | | |
| H/Heart/Diseased/Ventricle/ | 32.47 | 17.04 | 15.44 | 0.0226 |
| ELI 5 | | | | |
| H/Heart/Diseased/Ventricle/ | 33.12 | 18.43 | 14.7 | 0.0377 |
| PIT 16 | | | | |
| H/Heart/Diseased/Ventricle/ | 38.58 | 22.33 | 16.25 | 0 |
| PIT 1 | | | | |
| H/Heart/Diseased/Ventricle/ | 37.53 | 20.43 | 17.11 | 0 |
| PIT 14 | | | | |
| H/Kidney/normal/NDR 171 | 40 | 21.48 | 18.52 | 0 |
| H/Kidney/normal/NDR 179 | 34.98 | 20.59 | 14.39 | 0.0466 |
| H/Kidney/normal/PIT 289 | 39.51 | 24.04 | 15.47 | 0 |
| H/Kidney/normal/PIT 351 | 35.9 | 19.61 | 16.3 | 0 |
| H/Kidney/normal/PIT 353 | 37.23 | 19.62 | 17.61 | 0 |
| H/Kidney/HT/NDR 233 | 37.13 | 18.77 | 18.36 | 0 |
| H/Kidney/HT/NDR 224 | 38.86 | 19.81 | 19.05 | 0 |
| H/Kidney/HT/CHT 1176 | 39.17 | 23.31 | 15.87 | 0 |
| H/Kidney/HT/NDR 252 | 38.6 | 23.38 | 15.23 | 0 |
| H/Kidney/HT/CHT 762 | 38.72 | 22.11 | 16.61 | 0 |
| H/Skeletal Muscle/Normal/ | 37.59 | 22.68 | 14.9 | 0 |
| PIT 915 | | | | |
| H/Skeletal Muscle/Normal/ | 40 | 23.98 | 16.02 | 0 |
| PIT 685 | | | | |
| H/Skeletal Muscle/Normal/ | 40 | 24.3 | 15.7 | 0 |
| PIT 428 | | | | |
| H/Liver/Normal/MPI 146 | 33.08 | 18.02 | 15.05 | 0.0295 |
| H/Liver/Normal/CHT 339 | 40 | 22.25 | 17.75 | 0 |
| H/Liver/Normal/CHT 1237 | 38.8 | 22.14 | 16.66 | 0 |

TABLE 13

| Tissue Type | Mean | β 2 Mean | ∂∂ Ct | Expression |
|---|---|---|---|---|
| Coronary SMC | 39.65 | 21.25 | 18.4 | 0 |
| Huvec NS | 35.55 | 20.52 | 15.03 | 0 |
| Huvec Shear/static pooled | 32.94 | 21.2 | 11.73 | 0.2934 |
| H/Adipose/PIT 695 | 33.87 | 19.64 | 14.23 | 0.052 |
| H/Internal Mam Artery/ | 35.85 | 21.23 | 14.62 | 0 |
| Normal/AMC 263 | | | | |
| H/Internal Mam Artery/ | 37.19 | 21.99 | 15.2 | 0 |
| Normal/AMC 347 | | | | |
| H/Internal Mam Artery/ | 36.63 | 22.11 | 14.53 | 0 |
| Normal/AMC350 | | | | |
| H/Internal Mam Artery/ | 35.27 | 22.08 | 13.2 | 0 |
| Normal/AMC 352 | | | | |
| H/Artery/Normal/PIT 1180 | 33.95 | 19.48 | 14.47 | 0.0442 |
| H/Artery/normal/AMC 150 | 34.58 | 23.97 | 10.61 | 0.6421 |
| H/Artery/normal/PIT 912 | 35.84 | 21.54 | 14.31 | 0 |
| H/Artery/normal/NDR 352 | 33.17 | 20.82 | 12.35 | 0.1922 |
| H/Aorta/Diseased/PIT 710 | 36.95 | 21.34 | 15.62 | 0 |
| H/Aorta/Diseased/PIT 712 | 33.8 | 20.91 | 12.89 | 0.1322 |
| H/Aorta/Diseased/PIT 732 | 36.96 | 21.7 | 15.27 | 0 |
| H/Artery/Diseased/iliac/ | 34.99 | 21.02 | 13.97 | 0.0623 |
| NDR 753 | | | | |
| H/Artery/Diseased/Tibial/ | 34.01 | 20.22 | 13.79 | 0.0706 |
| PIT 679 | | | | |
| H/Vein/Normal/PIT 1010 | 35.17 | 21 | 14.16 | 0 |
| H/Vein/Normal/NDR 239 | 37.19 | 21.04 | 16.15 | 0 |
| H/Vein/Normal/AMC 130 | 36.7 | 20.97 | 15.73 | 0 |
| H/Vein/Normal/AMC 131 | 39.55 | 21.52 | 18.02 | 0 |
| H/Vein/Normal/AMC 137 | 35.9 | 20.51 | 15.39 | 0 |
| H/Vein/Normal/AMC 153 | 36.05 | 20.52 | 15.53 | 0 |
| H/Vein/Normal/AMC 176 | 35.41 | 20.31 | 15.1 | 0 |
| H/Vein/Normal/AMC 177 | 36.9 | 21.48 | 15.42 | 0 |
| H/Vein/Normal/AMC 178 | 36.22 | 21.16 | 15.05 | 0 |
| H/Vein/Normal/AMC 182 | 35.52 | 19.84 | 15.68 | 0 |
| H/Vein/Normal/AMC 190 | 34.98 | 21.12 | 13.86 | 0.0675 |
| H/Vein/Normal/AMC 192 | 36.6 | 22.39 | 14.21 | 0 |
| H/Vein/Normal/AMC 195 | 36.33 | 21.4 | 14.93 | 0 |
| H/Vein/Normal/AMC 211 | 36.53 | 21.11 | 15.43 | 0 |

### Human 38554, 57301 and 58324

There are over 30 families of secondary transporters, also known as solute carriers or SLC (reviewed by Berger, et al. (2000) in The Kidney: Physiology and Pathophysiology, eds. Seldin D W and Giebisch G., Lippincott, Williams & Wilkins, Philadelphia 1:107-138. The SLC families are classified according to the pair of molecules they move, for instance, the SLC21 and 22 families transport organic ions. The 38554, 57301 and 58324 molecules of the invention are members of the SLC21 and SLC22 families.

### Human 38554

Human 38554 is represented by two sequences containing amino acid substitutions at several residues. The human 38554 sequences (SEQ ID NO:32 or SEQ ID NO:53), which are approximately 3220 and 3227 nucleotides long, respectively, including untranslated regions, contain a predicted methionine-initiated coding sequence of about 2136 nucleotides, not including the termination codon (nucleotides 338-2473 of SEQ ID NO:32; 1-2136 of SEQ ID NO:34 or 345-2480 of SEQ ID NO:53; 1-2136 of SEQ ID NO:55). The coding sequences encode 712 amino acid proteins (SEQ ID NO:33 or SEQ ID NO:54).

Human 38554 contains the following regions or other structural features (for general information regarding PFAM identifiers, PS prefix and PF prefix domain identification numbers, refer to Sonnhammer et al. (1997) Protein 28:405-420: twelve transmembrane domains, thirteen non-transmembrane regions, a kazal domain (PFAM Accession Number PF00050, SEQ ID NO:41) located at about amino acid residues 476 to 523 of SEQ ID NO:33 or SEQ ID NO:54; and one peroxisomal targeting signal (PSORT PTS2, SEQ ID NO:50) at about amino acids 154 to 162 of SEQ ID NO:33 (not in SEQ ID NO:54). The transmembrane domains (predicted by MEMSAT, Jones et al., (1994) Biochemistry 33:3038-3049) are located at about amino acids 42 to 58, 80 to 102, 111 to 128, 190 to 212, 221 to 245, 274 to 295, 354 to 373, 393 to 414, 427 to 446, 553 to 577, 588 to 612, and 641 to 664 of SEQ ID NO:33 or SEQ ID NO:54; and the non-transmembrane regions are located at about amino acids 1 to 41, 59 to 79, 103 to 110, 129 to 189, 213 to 220, 246 to 273, 296 to 353, 374 to 392, 415 to 426, 447 to 552, 578 to 587, 613 to 640, and 665 to 712 of SEQ ID NO:33 or SEQ ID NO:54.

Human 38554 also contains the following regions or other structural features: one tyrosine kinase phosphorylation site (Prosite PS00007) at about amino acids 378 to 384 of SEQ ID NO:33 or SEQ ID NO:54; thirteen protein kinase C phosphorylation sites (Prosite PS00005) at about amino acids 4 to 6, 24 to 26, 152 to 154, 264 to 266, 312 to 314, 345 to 347, 374 to 376, 388 390, 509 to 511, 512 to 514, 629 to 631, 677 to 679 and 685 to 687 of SEQ ID NO:33 or twelve protein kinase C phosphorylation sites (Prosite PS00005) at about amino acids 4 to 6, 24 to 26, 264 to 266, 312 to 314, 345 to 347, 374 to 376, 388 390, 509 to 511, 512 to 514, 629 to 631, 677 to 679 and 685 to 687 of SEQ ID NO:54; eleven casein kinase II phosphorylation sites (Prosite PS00006) located at about amino acids 4 to 7, 31 to 34, 68 to 71, 165 to 168, 264 to 267, 304 to 307, 310 to 313, 466 to 469, 485 to 488, 677 to 680, and 694 to 697 of SEQ ID NO:33 or SEQ ID NO:54; six N-glycosylation sites (Prosite PS00001) from about amino acids 146 to 149, 309 to 312, 510 to 513, 520 to 523, 533 to 536, and 692 to 695 of SEQ ID NO:33 or SEQ ID NO:54; and twelve N-myristoylation sites (Prosite PS00008) from about amino acids 82 to 87, 226 to 231, 243 to 248, 385 to 390, 406 to 411, 446 to 451, 454 to 459, 505 to 510, 525 to 530, 537 to 537, 568 to 573, and 625 to 630 of SEQ ID NO:33 or SEQ ID NO:54.

A hydropathy plot of human 38554 was performed. Polypeptides of the invention include fragments which include: all or part of a hydrophobic sequence, e.g., the sequence from about amino acid 111 to 128, from about 274 to 295, and from about 641 to 664 of SEQ ID NO:33 or SEQ ID NO:54; all or part of a hydrophilic sequence, e.g., the sequence of from about amino acid 28 to 36, from about 134 to 142, and from about 301 to 316 of SEQ ID NO:33 or SEQ ID NO:54; a sequence which includes a Cys, or a glycosylation site.

### Human 57301

The human 57301 sequence (SEQ ID NO:35), which is approximately 2866 nucleotides long including untranslated regions, contains a predicted methionine-initiated coding sequence of about 1659 nucleotides, not including the termination codon (nucleotides 365-2023 of SEQ ID NO:35; 1-1659 of SEQ ID NO:37). The coding sequence encodes a 553 amino acid protein (SEQ ID NO:36).

Human 57301 contains the following regions or other structural features: twelve transmembrane domains, thirteen non-transmembrane regions, and a sugar (and other) transporter domain (PFAM Accession Number PF00083) located at about amino acid residues 106 to 530 of SEQ ID NO:36. The transmembrane domains (predicted by MEMSAT, Jones et al., (1994) Biochemistry 33:3038-3049) are located at about amino acids 21 to 37, 151 to 167, 174 to 196, 204 to 222, 232 to 255, 263 to 279, 352 to 369, 378 to 400, 409 to 426, 436 to 455, 466 to 486 and 495 to 515 of SEQ ID NO:36; and the non-transmembrane regions at about amino acids 1 to 20, 38 to 150, 168 to 173, 197 to 203, 223 to 231, 256 to 262, 280 to 351, 370 to 377, 401 to 408, 427 to 435, 456 to 465, 487 to 494, and 516 to 553 of SEQ ID NO:36.

Human 57301 also contains the following regions or other structural features: four protein kinase C phosphorylation sites (Prosite PS00005) at about amino acids 46 to 48, 167 to 169, 282 to 284, and 289 to 291 of SEQ ID NO:36; four casein kinase II phosphorylation sites (Prosite PS00006) located at about amino acids 35 to 38, 107 to 110, 211 to 214, and 526 to 529 of SEQ ID NO:36; two cAMP/cGMP-dependent protein kinase phosphorylation sites (Prosite PS00004) located at about amino acids 405 to 408 and 536 to 539 of SEQ ID NO:36; three N-glycosylation sites (Prosite PS00001) from about amino acids 39 to 42, 56 to 59, and 102 to 105 of SEQ ID NO:36; two amidation sites (Prosite PS00009) from about amino acids 170 to 173 and 403 to 406 of SEQ ID NO:36; and eight N-myristoylation sites (Prosite PS00008) from about amino acids 155 to 160, 187 to 192, 246 to 251, 331 to 336, 431 to 436, 443 to 448, 472 to 477 and 541 to 546 of SEQ ID NO:36.

A hydropathy plot of human 57301 was performed. Polypeptides of the invention include fragments which include: all or part of a hydrophobic sequence, e.g., the sequence from about amino acid 151 to 167, from about 263 to 279, and from about 352 to 369 of SEQ ID NO:36; all or part of a hydrophilic sequence, e.g., the sequence of from about amino acid 82 to 95, from about 325 to 332, and from about 528 to 537 of SEQ ID NO:36; a sequence which includes a Cys, or a glycosylation site.

### Human 58324

The human 58324 sequence (SEQ ID NO:38), which is approximately 2480 nucleotides long including untranslated regions, contains a predicted methionine-initiated coding sequence of about 2157 nucleotides, not including the termination codon (nucleotides 148-2304 of SEQ ID NO:38; 1-2157 of SEQ ID NO:40). The coding sequence encodes a 719 amino acid protein (SEQ ID NO:39).

Human 58324 contains the following regions or other structural features: twelve transmembrane domains, thirteen non-transmembrane regions, and a kazal domain (PFAM Accession Number PF00050) located at about amino acid residues 502 to 549 of SEQ ID NO:39. The transmembrane domains (predicted by MEMSAT, Jones et al., (1994) Biochemistry 33:3038-3049) are located at about amino acids 107 to 126, 150 to 166, 173 to 193, 231 to 254, 265 to 289, 314 to 335, 372 to 391, 420 to 444, 457 to 475, 580 to 603, 614 to 635, and 667 to 691 of SEQ ID NO:39; and the non-transmembrane regions are located at about amino acids 1 to 106, 127 to 149, 167 to 172, 194 to 230, 255 to 264, 290 to 313, 336 to 371, 392 to 419, 445 to 456, 476 to 579, 604 to 613, 636 to 666, and 692 to 719 of SEQ ID NO:39.

Human 58324 also contains the following regions or other structural features: seven protein kinase C phosphorylation sites (Prosite PS00005) at about amino acids 38 to 40, 41 to 43, 75 to 77, 342 to 344, 450 to 452, 492 to 494, and 705 to 707 of SEQ ID NO:39; five casein kinase II phosphorylation sites (Prosite PS00006) located at about amino acids 11 to 14, 129 to 132, 192 to 195, 252 to 255, and 445 to 448 of SEQ ID NO:39; one tyrosine kinase site (Prosite PS00007) located at about amino acids 144 to 151 of SEQ ID NO:39; five N-glycosylation sites (Prosite PS00001) from about amino acids 294 to 297, 300 to 303, 497 to 500, 546 to 549 and 661 to 664 of SEQ ID NO:39; one amidation site (Prosite PS00009) from about amino acids 44 to 47 of SEQ ID NO:39; and eleven N-myristoylation sites (Prosite PS00008) from about amino acids 37 to 42, 92 to 97, 100 to 105, 120 to 125, 184 to 189, 216 to 221, 264 to 269, 432 to 437, 441 to 446, 531 to 536, and 553 to 558 of SEQ ID NO:39.

A hydropathy plot of human 58324 was performed. Polypeptides of the invention include fragments which include: all or part of a hydrophobic sequence, e.g., the sequence from about amino acid 173 to 193, from about 314 to 335, and from about 667 to 691 of SEQ ID NO:39; all or part of a hydrophilic sequence, e.g., the sequence from about amino acid 40 to 50, from about 194 to 201, and from about 538 to 546 of SEQ ID NO:39; a sequence which includes a Cys, or a glycosylation site.

The 38554, 57301 and 58324 proteins contain a significant number of structural characteristics in common with members of the SLC21 or 22 transporter families. As used herein, the terms “transporter,” “organic ion transporter,” “organic anion transporter,” “SLC21 family, or SLC22 family” include secondary active transport proteins. Secondary active transporters couple the active transport of one molecule, e.g., an ion, e.g., an organic ion (e.g., an organic anion or a cation, a prostaglandin, a steroidal compound (e.g., estrone-3-sulfate), a bile acid, a drug, a neurotransmitter, a sulfated lipophilic metabolite, a glucuronidated lipophilic metabolite, a polyamine, a carnitine, or a choline) against its concentration gradient to the energy gained by concomitant transport of a second molecule, e.g., another ion (e.g., a bicarbonate ion or a dicarboxylate ion) with its concentration gradient.

The SLC21 or SLC22 families of proteins are characterized by at least one, two, three, four, five, six, seven, eight, nine, ten, eleven, preferably twelve transmembrane domains. Typically, the hydrophobic transmembrane domains anchor the transporter within a cell or organelle membrane and through coordinated allosteric movements, affect the transport function across the membrane. The non-transmembrane loops between and beyond the transmembrane domains of the transporter determine the ion binding specificity and provide the ion binding and release activity for the transporter. Some members of these families also have a transporter domain, and/or a kazal domain.

A GAP alignment of 38554 with an SLC21 family member, organic anion transporting protein 14 (OATP-F, accession number 7839587 in GenPept, corresponding to AF260704 in Genbank, SEQ ID NO:44) results in 99.7% identity between the two sequences (as determined from a matrix made by matblas from blosum62.iij). A GAP alignment of 57301 with an SLC22 family member, organic anion transporter 4 (hOAT4, SEQ ID NO:47, accession number 7707622 in GenPept, corresponding to AB026116 in Genbank) results in 51.7% identity between the two sequences (as determined from a matrix made by matblas from blosum62.iij). A GAP alignment of 57301 with an SLC22 family member, renal-specific transporter (mouse RST, SEQ ID NO:48, accession number 2696709 in GenPept, corresponding to BAA23875 in GenBank) results in 71.4% identity between the two sequences (as determined from a matrix made by matblas from blosum62.iij). A GAP alignment of 58324 with an SLC21 family member, organic anion transporter (OATP-E, SEQ ID NO:49, accession number 6683743 in GenPept, corresponding to AB026116 in Genbank) results in 30% identity between the two sequences (as determined from a matrix made by matblas from blosum62.iij).

A 38554, 57301 or 58324 polypeptide can include at least one, two, three, four, five, six, seven, eight, nine, ten, eleven, preferably twelve “transmembrane domains” or regions homologous with a “transmembrane domain”.

As used herein, the term “transmembrane domain” includes an amino acid sequence of about 10 to 40 amino acid residues in length and spans the plasma membrane. Transmembrane domains are rich in hydrophobic residues, e.g., at least 50%, 60%, 70%, 80%, 90%, 95% or more of the amino acids of a transmembrane domain are hydrophobic, e.g., leucines, isoleucines, tyrosines, or tryptophans. Transmembrane domains typically have alpha-helical structures and are described in, for example, Zagotta, W. N. et al., (1996) Annual Rev. Neurosci. 19:235-263, the contents of which are incorporated herein by reference.

In a preferred embodiment, a 38554, 57301 or 58324 polypeptide or protein has at least one, two, three, four, five, six, seven, eight, nine, ten, eleven, preferably twelve “transmembrane domains” or regions which include at least about 12 to 35 more preferably about 14 to 30 or 15 to 25 amino acid residues each and have at least about 60%, 70% 80% 90% 95%, 99%, or 100% homology with a “transmembrane domain,” e.g., the transmembrane domains of human 38554, 57301 or 58324 (e.g., residues 42 to 58, 80 to 102, 111 to 128, 190 to 212, 221 to 245, 274 to 295, 354 to 373, 393 to 414, 427 to 446, 553 to 577, 588 to 612, and 641 to 664 of SEQ ID NO:33 or SEQ ID NO:54; residues 21 to 37, 151 to 167, 174 to 196, 204 to 222, 232 to 255, 263 to 279, 352 to 369, 378 to 400, 409 to 426, 436 to 455, 466 to 486 and 495 to 515 of SEQ ID NO:36; or residues 107 to 126, 150 to 166, 173 to 193, 231 to 254, 265 to 289, 314 to 335, 372 to 391, 420 to 444, 457 to 475, 580 to 603, 614 to 635, and 667 to 691 of SEQ ID NO:39). The transmembrane domains in 38554, 57301 and 58324 can be seen in hydropathy plots as regions of about 15 to 25 amino acids where the hydropathy trace is mostly above the horizontal line.

To identify the presence of a “transmembrane” domain in a 38554, 57301 or 58324 protein sequence, and make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

A 38554, 57301 or 58324 polypeptide can include at least one, two, three, four, five, six, seven, eight, nine, ten, eleven, twelve, preferably thirteen “non-transmembrane regions.” As used herein, the term “non-transmembrane region” includes an amino acid sequence not identified as a transmembrane domain. The non-transmembrane regions in 38554 are located at about amino acids 1 to 41, 59 to 79, 103 to 110, 129 to 189, 213 to 220, 246 to 273, 296 to 353, 374 to 392, 415 to 426, 447 to 552, 578 to 587, 613 to 640, and 665 to 712 of SEQ ID NO:33 or SEQ ID NO:54. The non-transmembrane regions in 57301 are located at about amino acids 1 to 20, 38 to 150, 168 to 173, 197 to 203, 223 to 231, 256 to 262, 280 to 351, 370 to 377, 401 to 408, 427 to 435, 456 to 465, 487 to 494, and 516 to 553 of SEQ ID NO:36. The non-transmembrane regions in 58324 are located at about amino acids 1 to 106, 127 to 149, 167 to 172, 194 to 230, 255 to 264, 290 to 313, 336 to 371, 392 to 419, 445 to 456, 476 to 579, 604 to 613, 636 to 666, and 692 to 719 of SEQ ID NO:39.

The non-transmembrane regions of 38554, 57301 or 58324 include at least one, two, three, four, five, six, preferably seven cytoplasmic regions. When located at the N-terminus, the cytoplasmic region is referred to herein as the “N-terminal cytoplasmic domain.” As used herein, an “N-terminal cytoplasmic domain” includes an amino acid sequence having about 1 to 300, preferably about 1 to 250, preferably about 1 to 200, more preferably about 1 to 150, or even more preferably about 1 to 110 amino acid residues in length and is located inside of a cell or within the cytoplasm of a cell. The C-terminal amino acid residue of an “N-terminal cytoplasmic domain” is adjacent to an N-terminal amino acid residue of a transmembrane domain in a 38554, 57301 or 58324 protein. For example, an N-terminal cytoplasmic domain is located at about amino acid residues 1 to 41 of SEQ ID NO:33 or SEQ ID NO:54, 1 to 20 of SEQ ID NO:36, and 1 to 106 of SEQ ID NO:39.

In a preferred embodiment, a 38554, 57301 or 58324 polypeptide or protein has an N-terminal cytoplasmic domain or a region which includes at least about 5, preferably about 10 to 200, and more preferably about 15 to 110 amino acid residues and has at least about 60%, 70% 80% 90% 95%, 99%, or 100% homology with an “N-terminal cytoplasmic domain,” e.g., the N-terminal cytoplasmic domain of human 38554, 57301 or 58324 (e.g., residues 1 to 41 of SEQ ID NO:33 or SEQ ID NO:54, 1 to 20 of SEQ ID NO:36, and 1 to 106 of SEQ ID NO:39).

In another embodiment, a cytoplasmic region of a 38554, 57301 or 58324 protein can include the C-terminus and can be a “C-terminal cytoplasmic domain,” also referred to herein as a “C-terminal cytoplasmic tail.” As used herein, a “C-terminal cytoplasmic domain” includes an amino acid sequence having a length of at least about 15, preferably about 20 to 60, more preferably about 25 to 55 amino acid residues and is located inside of a cell or within the cytoplasm of a cell. The N-terminal amino acid residue of a “C-terminal cytoplasmic domain” is adjacent to a C-terminal amino acid residue of a transmembrane domain in a 38554, 57301 or 58324 protein. For example, a C-terminal cytoplasmic domain is located at about amino acid residues 665 to 712 of SEQ ID NO:33 or SEQ ID NO:54, 516 to 553 of SEQ ID NO:36, and 692 to 719 of SEQ ID NO:39.

In a preferred embodiment, a 38554, 57301 or 58324 polypeptide or protein has a C-terminal cytoplasmic domain or a region which includes at least about 5, preferably about 15 to 60, and more preferably about 25 to 55 amino acid residues and has at least about 60%, 70% 80% 90% 95%, 99%, or 100% homology with a C-terminal cytoplasmic domain,” e.g., the C-terminal cytoplasmic domain of human 38554, 57301 or 58324 (e.g., residues 665 to 712 of SEQ ID NO:33 or SEQ ID NO:54, 516 to 553 of SEQ ID NO:36, and 692 to 719 of SEQ ID NO:39).

In another embodiment, a 38554, 57301 or 58324 protein includes at least one, two, three, four, preferably five cytoplasmic loops. As used herein, the term “loop” includes an amino acid sequence that resides outside of a phospholipid membrane, having a length of at least about 4, preferably about 5 to 150, more preferably about 6 to 120 amino acid residues, and has an amino acid sequence that connects two transmembrane domains within a protein or polypeptide. Accordingly, the N-terminal amino acid of a loop is adjacent to a C-terminal amino acid of a transmembrane domain in a 38554, 57301 or 58324 molecule, and the C-terminal amino acid of a loop is adjacent to an N-terminal amino acid of a transmembrane domain in a 38554, 57301, or 58324 molecule. As used herein, a “cytoplasmic loop” includes a loop located inside of a cell or within the cytoplasm of a cell. For example, a “cytoplasmic loop” can be found at about amino acid residues 103 to 110, 213 to 220, 296 to 353, 415 to 426, and 578 to 587 of SEQ ID NO:33 or SEQ ID NO:54; 168 to 173, 223 to 231, 280 to 351, 401 to 408, and 456 to 465 of SEQ ID NO:36; 167 to 172, 255 to 264, 336 to 371, 445 to 456, and 604 to 613 of SEQ ID NO:39.

In a preferred embodiment, a 38554, 57301 or 58324 polypeptide or protein has a cytoplasmic loop or a region which includes at least about 4, preferably about 5 to 100, and more preferably about 6 to 80 amino acid residues and has at least about 60%, 70% 80% 90% 95%, 99%, or 100% homology with a cytoplasmic loop,” e.g., a cytoplasmic loop of human 38554, 57301 or 58324 (e.g., residues 103 to 110, 213 to 220, 296 to 353, 415 to 426, and 578 to 587 of SEQ ID NO:33 or SEQ ID NO:54; 168 to 173, 223 to 231, 280 to 351, 401 to 408, and 456 to 465 of SEQ ID NO:36; 167 to 172, 255 to 264, 336 to 371, 445 to 456, and 604 to 613 of SEQ ID NO:39).

In another embodiment, a 38554, 57301 or 58324 protein includes at least one, two, three, four, five, preferably six non-cytoplasmic loops. As used herein, a “non-cytoplasmic loop” includes an amino acid sequence located outside of a cell or within an intracellular organelle. Non-cytoplasmic loops include extracellular domains (i.e., outside of the cell) and intracellular domains (i.e., within the cell). When referring to membrane-bound proteins found in intracellular organelles (e.g., mitochondria, endoplasmic reticulum, peroxisomes microsomes, vesicles, endosomes, and lysosomes), non-cytoplasmic loops include those domains of the protein that reside in the lumen of the organelle or the matrix or the intermembrane space. For example, a “non-cytoplasmic loop” can be found at about amino acid residues 59 to 79, 129 to 189, 246 to 273, 374 to 392, 447 to 452, and 613 to 640 of SEQ ID NO:33 or SEQ ID NO:54; 38 to 150, 197 to 203, 256 to 262, 370 to 377, 427 to 435, and 487 to 494 of SEQ ID NO:36; 127 to 149, 194 to 230, 290 to 313, 392 to 419, 476 to 579, and 636 to 666 of SEQ ID NO:39.

In a preferred embodiment, a 38554, 57301 or 58324 polypeptide or protein has at least one non-cytoplasmic loop or a region which includes at least about 4, preferably about 5 to 150, more preferably about 6 to 120 amino acid residues and has at least about 60%, 70% 80% 90% 95%, 99%, or 100% homology with a “non-cytoplasmic loop,” e.g., at least one non-cytoplasmic loop of human 38554, 57301, or 58324 (e.g., residues 59 to 79, 129 to 189, 246 to 273, 374 to 392, 447 to 452, and 613 to 640 of SEQ ID NO:33 or SEQ ID NO:54; 38 to 150, 197 to 203, 256 to 262, 370 to 377, 427 to 435, and 487 to 494 of SEQ ID NO:36; 127 to 149, 194 to 230, 290 to 313, 392 to 419, 476 to 579, and 636 to 666 of SEQ ID NO:39).

A 38554, 57301 or 58324 protein or polypeptide can include a “transporter domain” or a region homologous to a “transporter domain.” As used herein, the term “transporter domain” includes an amino acid sequence of about 20 to 250 amino acid residues in length, resides in a non-cytoplasmic loop and participates in the transport of a molecule; e.g. an ion, (e.g., an organic anion or cation, a hormone or a metabolite) across a membrane, e.g. a cell or organelle membrane and can have a bit score (PSI-BLAST) for the alignment of the sequence to a transporter domain of at least 80. Preferably, a transporter domain includes at least about 30 to 225 amino acids, more preferably about 35 to 215 amino acid residues, or about 40 to 195 amino acids and has a bit score for the alignment of the sequence to a transporter domain (PSI-BLAST) of at least 100, 120, 135 or greater. The transporter domain of 38554 and 58324 is homologous to ProDom family PD005488 (“Transporter Protein Transmembrane Transport Similar Matrin F/G Organic Anion Sodium-Independent;” SEQ ID NO:43, ProDomain Release 2000.1; see also ProDomain No. PD005488, Release 1999.2). An alignment of this domain of 38554 (amino acids 476 to 667 of SEQ ID NO:33 or SEQ ID NO:54) with PD005488 resulted in 44% identity as determined by PSI-BLAST. An alignment of this domain of 58324 (amino acids 502 to 672 of SEQ ID NO:39) with PD005488 resulted in 32% identity as determined by PSI-BLAST. The transporter domain of 57301 is homologous to ProDom family PD151320 (“Organic Transporter-like Transport Protein Renal Anion Transporter Cationic Kidney-Specific Solute,” SEQ ID NO:46, ProDomain Release 1999.2). An alignment of this region (amino acids 102 to 145 of SEQ ID NO:36) with PD151320 resulted in 56% identity as determined by PSI-BLAST.

In a preferred embodiment a 38554, 57301 or 58324 polypeptide or protein has a “transporter domain” or a region which includes at least about 30 to 225 amino acids, more preferably about 35 to 215 amino acid residues, or about 40 to 195 amino acid residues and has at least about 60%, 70% 80% 90% 95%, 99%, or 100% homology with a “transporter domain,” e.g., the transporter domain of human 38554, 57301 or 58324, (e.g., residues 476 to 667 of SEQ ID NO:33 or SEQ ID NO:54, 102 to 145 of SEQ ID NO:36, or 502 to 672 of SEQ ID NO:39).

For further identification of a transporter domain in a 38554, 57301, or 58324 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul S F et al. (1997) Nucleic Acids Res. 25:3389-3402; Gouzy et al. (1999) Computers and Chemistry 23:333-340) of the SWISS-PROT 38 and TREMBL protein databases. The database automatically generates a consensus sequence for each domain. A BLAST search was performed against the HMM database resulting in the identification of a “Organic Transporter-like Transport Protein Renal Anion Transporter Cationic Kidney-Specific Solute” domain in the amino acid sequence of human 57301 at about residues 102 to 145 of SEQ ID NO:36 and a “Transporter Protein Transmembrane Transport Similar Matrin F/G Organic Anion Sodium-Independent” domain in the amino acid sequence of human 38554 and 58324 at about residues 476 to 667 of SEQ ID NO:33 or SEQ ID NO:54, or 502 to 672 of SEQ ID NO:39.

A 57301 polypeptide can further include a “sugar (and other) transporter domain” or regions homologous with a “sugar (and other) transporter domain” (SEQ ID NO:45, PFAM Accession Number PF00083). As used herein, the term “sugar (and other) transporter domain” includes an amino acid sequence of about 420 to 440 amino acid residues in length and transports molecules, e.g., ions, sugars or metabolites. An alignment of the sugar (and other) transporter domain (amino acids 106 to 530 of SEQ ID NO:36) of human 57301 with a consensus amino acid sequence (SEQ ID NO:45) derived from a hidden Markov model yields a bit score of −15.3.

Sugar (and other) transporter domains can have sequences similar to three Prosite signature sequences (two copies of PS00216 and one copy of PS00217). A sequence similar to copy one of the first Prosite signature sequence (PS00216, [LIVMSTAG]-[LIVMFSAG]-x(2)-[LIVMSA]-[DE]-x-[LIVMFYWA]-G-R-[RK]-x(4,6)-[GSTA], SEQ ID NO:51), with a mismatch at only the first residue of the consensus, is located about between the second and third transmembrane domains of the human 57301 polypeptide and can be found at about amino acids 163 to 179 of SEQ ID NO:36. A sequence similar to copy two of the first Prosite signature sequence, with a mismatch of only an S instead of the [DE], is located about between the eighth and ninth transmembrane domains of the human 57301 polypeptide and can be found at about amino acids 396 to 411 of SEQ ID NO:36. These signature sequences are involved in the conformational change required for transport. A sequence similar to the second Prosite signature sequence (PS00217, [LIVMF]-x-G-[LIVMFA]-x(2)-G-x(8)-[LIFY]-x(2)-[EQ]-x(6)-[RK], SEQ ID NO:52), with a conserved substitution of an A for the first G, a one amino acid insertion after the fourth residue of the consensus, and only one amino acid between the [LIFY] and the [EQ], is located about the end of the fourth and in the loop before the fifth transmembrane domain of the human 57301 polypeptide and can be found at about amino acids 205 to 230 of SEQ ID NO:36. In the above conserved motifs, and other motifs described herein, the standard IUPAC one-letter code for the amino acids is used. Each element in the pattern is separated by a dash (-); square brackets ([ ]) indicate the particular residues that are accepted at that position; x indicates that any residue is accepted at that position; and numbers in parentheses (( )) indicate the number of residues represented by the accompanying amino acid.

A 38554 or 58324 molecule can include a kazal domain or regions homologous with a “kazal domain” (PFAM Accession Number PF00050, SEQ ID NO:41. As used herein, the term “kazal domain” includes an amino acid sequence of about 45 to 55 amino acid residues in length and is characterized by the pattern of cysteine residues, required for disulfide bonding into a specific structure used for contact with the substrate. The kazal domain (HMM) has been assigned the SMART identifier kazal (SEQ ID NO:42). An alignment of the kazal domain (amino acids 476 to 523 of SEQ ID NO:33 or SEQ ID NO:54, or 502 to 549 of SEQ ID NO:39) of human 38554 or 58324, respectively, with a consensus amino acid sequence (SEQ ID NO:41) derived from a hidden Markov model yields a bit score of −7.7 and −13.8, respectively. An alignment of the kazal domain (amino acids 475 to 523 of SEQ ID NO:33 or SEQ ID NO:54) of human 38554 with a SMART consensus amino acid sequence (SEQ ID NO:42) derived from modular architecture analysis yields a bit score of −1.8.

To identify the presence of a “sugar (and other) transporter” domain or a “kazal” domain in a 38554, 57301, or 58324 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a “sugar (and other) transporter” domain in the amino acid sequence of human 57301 at about residues 106 to 530 of SEQ ID NO:36; a “kazal” domain in the amino acid sequence of human 38554 at about residues 476 to 523 of SEQ ID NO:33 or SEQ ID NO:54; and a “kazal” domain in the amino acid sequence of human 58324 at about residues 502 to 549 of SEQ ID NO:39.

An additional method to identify the presence of a “kazal” domain in a 38554 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (2000) Nucl. Acids Res 28:231. The database contains domains identified by profiling with the hidden Markov models of the HMMer2 search program (R. Durbin et al. (1998) Biological sequence analysis: probabilistic models of proteins and nucleic acids. Cambridge University Press). The database also is extensively annotated and monitored by experts to enhance accuracy. A search was performed against the HMM database resulting in the identification of a “kazal” domain in the amino acid sequence of 38554 at about residues 475 to 523 of SEQ ID NO:33 or SEQ ID NO:54.

A 38554, 57301 or 58324 family member can include at least one, two, three, four, five, six, seven, eight, nine, ten, eleven, preferably twelve transmembrane domains; at least one, two, three, four, five, six, preferably seven cytoplasmic regions, including N- and C-terminal cytoplasmic domains and at least one, two, three, four, preferably five cytoplasmic loops; and at least one, two, three, four, five, preferably six non-cytoplasmic loops. Additionally, a 38554 or a 58324 family member can include at least one kazal domain and a 57301 family member can include a sugar (and other) transporter domain. A 38554 family member can further include at least one at least one peroxisomal targeting signal (PSORT PTS2).

Furthermore, a 38554 family member can include at least one, five, ten, preferably thirteen protein kinase C phosphorylation sites (PS00005); at least one, three, six, and preferably eleven casein kinase II phosphorylation sites (PS00006); at least one, two, four, and preferably six N-glycosylation sites (PS00001); at least one tyrosine kinase phosphorylation site (PS00007); and at least one, five, ten, and preferably twelve N-myristoylation sites (PS00008). A 57301 family member can include at least one, two, three, preferably four protein kinase C phosphorylation sites (PS00005); at least one, two, three, and preferably four casein kinase II phosphorylation sites, (PS00006); at least one, two, preferably three N-glycosylation sites (PS00001); at least one, preferably two cAMP- and cGMP-dependent protein kinase phosphorylation sites (PS00004); at least one, preferably two amidation sites (PS00009); and at least one, two, four, and preferably eight N-myristoylation sites (PS00008). Furthermore, a 58324 family member can include at least one, two, four, preferably seven protein kinase C phosphorylation sites (PS00005); at least one, two, three, four, and preferably five casein kinase II phosphorylation sites (PS00006); at least one, two, four, and preferably five N-glycosylation sites (PS00001); at least one tyrosine kinase phosphorylation site (PS00007); at least one amidation site (PS00009); and at least one, three, seven and preferably eleven N-myristoylation sites (PS00008).

As the 38554, 57301 or 58324 polypeptides of the invention can modulate 38554-, 57301- or 58324-mediated activities, they can be useful for developing novel diagnostic and therapeutic agents for transporter-associated or other 38554-, 57301- or 58324-associated disorders, as described below.

The SLC21 and SLC22 families are polyspecific transporters of organic ions. Some members of the SLC21 family transport organic anions, others transport prostaglandins. Some members of the SLC22 family transport organic anions, others transport organic cations, and some may transport either type of ion. They participate in activities as diverse as intestinal or hepatic absorption of metabolites, renal reabsorption of cations or excretion of cations. Members of these families also transport a wide variety of drugs and xenobiotics, many of which are harmful to the body. In addition, organic ion transporters are responsible for the transport of the metabolites of most lipophilic compounds, e.g., sulfate and glucuronide conjugates (Moller, J. V. and Sheikh, M. I. (1982) Pharmacol Rev. 34:315-358; Pritchard, J. B. and Miller, D. S. (1993) Physiol. Rev. 73:765-796; Ullrich, K. J. (1997) J. Membr. Biol. 158:95-107; Ullrich, K. J. and Rumrich, G. (1993) Clin. Investig. 71:843-848; Petzinger, E. (1994) Rev. Physiol. Biochem. Pharmacol. 123:47-211).

Proper function of members of these families is important for many physiological processes. At the cellular level, aberrant or deficient organic ion transporter activity can detrimentally affect functions such as cellular proliferation, growth, differentiation, or migration. At the tissue level, aberrant or deficient organic ion transporter activity can detrimentally affect inter- or intra-cellular communication; or musculoskeletal function. At the organ level, aberrant or deficient organic ion transporter activity can detrimentally affect kidney, liver or cardiac function. At the organism level, aberrant or deficient organic ion transporter activity can detrimentally affect systemic responses, such as nervous system responses, hormonal responses (e.g., insulin response), or immune responses; and protection of cells from toxic compounds (e.g., carcinogens, toxins, or mutagens).

As used herein, a “38554, 57301 or 58324 activity”, “biological activity of 38554, 57301 or 58324” or “functional activity of 38554, 57301 or 58324”, refers to an activity exerted by a 38554, 57301 or 58324 protein, polypeptide or nucleic acid molecule on e.g., a 38554-, 57301- or 58324-responsive cell or on a 38554, 57301 or 58324 substrate, e.g., a protein substrate, as determined in vivo or in vitro. In one embodiment, a 38554, 57301 or 58324 activity is a direct activity, such as an association with a 38554, 57301 or 58324 target molecule. A “target molecule” or “binding partner” is a molecule with which a 38554, 57301 or 58324 protein binds or interacts in nature. In an exemplary embodiment, 38554, 57301 or 58324 is a transporter, e.g., an SLC21 or 22 family organic ion transporter, and thus binds to or interacts in nature with a molecule, e.g., an organic ion.

A 38554, 57301 or 58324 activity can also be an indirect activity, e.g., a cellular signaling activity mediated by interaction of the 38554, 57301 or 58324 protein with a 38554, 57301 or 58324 receptor. Based on the above-described sequence structures and similarities to molecules of known function, the 38554, 57301 or 58324 molecules of the present invention have similar biological activities as SLC21 or 22 family members. For example, the 38554, 57301 or 58324 proteins of the present invention can have one or more of the following activities: (1) the ability to reside within a membrane; (2) the ability to interact with a substrate or target molecule; (3) the ability to transport a substrate or target molecule, e.g., an ion, e.g., an organic ion (e.g., an organic anion, an organic cation, a prostaglandin, a steroidal compound (e.g., estrone-3-sulfate), a bile acid, a drug, a neurotransmitter, a sulfated lipophilic metabolite, a glucuronidated lipophilic metabolite, a polyamine, a carnitine, or a choline) across a membrane; (4) the ability to transport a second substrate or target molecule, e.g., an ion, (e.g., a bicarbonate ion or a dicarboxylate ion), across a membrane; (5) the ability to interact with and/or modulate a second non-transporter protein; (6) the ability to modulate cellular signaling and/or gene transcription (e.g., either directly or indirectly); (7) the ability to protect cells and/or tissues from organic ions; (8) the ability to protect cells and/or tissues from organic anions; (9) the ability to modulate hormonal responses; (10) the ability to modulate metabolism; and (11) the ability to modulate excretion.

The 38554, 57301 or 58324 molecules of the invention can modulate the activities of cells in tissues where they are expressed. For example in TaqMan analysis, 38554 mRNA is expressed at high levels in human brain cortex and hypothalamus tissue and at medium levels in dorsal root ganglion, spinal cord, choroid plexus, and testes. In the neurological tissues, the expression is found on glial cells, with an epithelial cell similarity in choroid plexus. Expression of 38554 mRNA in monkey and rodent neurological tissues confirms the expression found in human neurological tissues. Regulation of expression is found in rodent dorsal root ganglion after axotomy. Also for example, 57301 mRNA is expressed at high levels in kidney and 58324 mRNA is expressed at small levels in hemangioma tissue. Accordingly, the 38554, 57301 or 58324 molecules of the invention can act as therapeutic or diagnostic agents for one or more of a pain disorder, a nervous system disorder, an immune, e.g., inflammatory disorder, a testicular disorder, a kidney disorder, or an angiogenesis disorder, as well as disorders in tissues where 38554 molecules are expressed at lower levels as described below. Small amounts of 38554 expression were found in normal artery, human umbilical vein endothelial cells, hemangioma tissue, tissue from heart undergoing congestive heart failure, and kidney. Trace amounts of 32468 expression were found in salivary glands, normal colon, colon tumor, normal lung, normal tonsil, mammary gland and pancreas.

The 38554, 57301 or 58324 molecules of the invention can play an important role in pain disorders. In addition, the 38554 molecules of the invention can be used to treat and/or diagnose pain disorders in part because 38554 mRNA is expressed in glial cells, cells with important roles in neuropathic pain and/or because 38554 expression is regulated in the dorsal root ganglion after axotomy.

The 38554 molecules can be used to treat neurological disorders in part because the 38554 mRNA is expressed in the brain cortex, hypothalamus tissue, dorsal root ganglion, spinal cord, and choroid plexus.

The 38554 molecules of the invention can be used to treat and/or diagnose a variety of immune, e.g., inflammatory disorders in part because the 38554 mRNA is expressed in the choroid plexus. The choroid plexus is responsible for the secretion of the cerebral fluid and is involved in inflammatory responses

The 38554 molecules can be used to treat testicular disorders in part because the 38554 mRNA is expressed in the testis. The blood-testis barrier is analogous to the blood-brain barrier in the physiology of seminiferous tubules and maturation of spermatozoa as they develop into spermatids. Transporter molecules, such as 38554, can play a role in the maintenance of this barrier and supply of ions to the developing spermatids.

The 57301 molecules can be used to treat renal disorders in part because the 57301 mRNA is expressed in the kidney.

The 38554 and 58324 molecules can be used to treat angiogenic disorders in part because the 38554 mRNA is expressed in normal artery, human umbilical vein endothelial cells, hemangioma tissue, and tissue from heart undergoing congestive heart failure, and 58324 mRNA is expressed in hemangioma tissue.

Thus, the 38554, 57301 or 58324 molecules can act as novel diagnostic targets and therapeutic agents for controlling one or more of a pain disorder, a nervous system disorder, an immune, e.g., inflammatory disorder, a testicular disorder, a kidney disorder, an angiogenesis disorder, as described above, or other organic ion transport, organic ion absorption or excretion, inter- or intra-cellular signaling, and/or hormonal response disorders.

### Gene Expression Analysis

Human 38554, 57301 and 58324 expression was measured by TaqMan® quantitative PCR (Perkin Elmer Applied Biosystems) in cDNA prepared from a variety of normal and diseased (e.g., cancerous) human tissues or cell lines.

This analysis found 38554 mRNA expression at high levels in human brain cortex and hypothalamus tissue, at medium levels in dorsal root ganglion, spinal cord, choroid plexus, and testes, at small levels in normal artery, human umbilical vein endothelial cells, hemangioma tissue, tissue from heart undergoing congestive heart failure, and kidney; and at trace levels in salivary glands, normal colon, colon tumor, normal lung, normal tonsil, mammary gland and pancreas; 57301 mRNA expression at high levels in kidney; and 58324 mRNA expression at small levels in hemangioma tissue.

### Expression of 38554 in Rodent Tissue and Rodent Pain Models

To study the expression of 38554 in animal models of pain, rats were subjected to the following procedures: ligation of the sciatic nerve to produce chronic constriction injury (Bennett G J & Xie Y K, 1988; Pain 33; 87-107), plantar injection of complete Freund's adjuvant (Stein C, Millan M J and Herz A, 1988; Pharmacol Biochem Behav 31; 445-451) to produce inflammatory pain, or axotomy of the sciatic nerve (Curtis et al., 1994; Neuron 12; 191-204) to produce chronic pain. TaqMan® quantitative PCR (PE Applied Biosystems) to measure the expression of the rat ortholog of human 38554 in cDNA prepared from a variety of normal and diseased (e.g., pain models) tissues was performed by the same methods as for the human tissue, as described above, except 18S RNA was used as an internal amplicon reference and reference probe; the integrity of the RNA samples following DNase I treatment was confirmed by 1.2% agarose gel electrophoresis; probes were designed by PrimerExpress software (PE Biosystems) based on the sequence of the rat 38554 gene; 200 nM of forward and reverse primers plus 100 nM probe for 18S and 900 nM forward and reverse primers plus 250 nM probe; and the Ct value of the rat 38554 gene is normalized by subtracting the Ct value of the 18S to obtain a _{Δ}Ct value using the following formula: _{Δ}Ct=averageCt_{rat 38554}−averageCt_{18S}.

The results indicated high levels of rat 38554 expression in brain and spinal cord, with low levels of rat 38554 expression in dorsal root ganglion. There was trace levels of rat 38554 expression in superior cervical ganglion, ovary and uterus. In the analysis of tissues using pain models, up-regulation of rat 38554 expression is found in rodent dorsal root ganglion after axotomy.

### In Situ Hybridization

The human 38554 clone was used to make probes for in situ hybridization experiments. Expression of 38554 mRNA in monkey and rodent neurological tissues confirms the expression found by TaqMan® quantitative PCR in human neurological tissues. In the rat neurological tissues, in situ hybridization found 38554 expression in cortex, hippocampus, spinal cord, and cerebrum. In the monkey neurological tissues, in situ hybridization found 38554 expression in the cortex, choroid plexus and dorsal root ganglion. Glial cells were the specific cell type labeled in these tissues; in choroid plexus, the cells are similar to epithelial cells.

### Human 55063 (NMDA-1)

The present invention is based, at least in part, on the discovery of novel molecules, referred to herein as “human NMDA-1” or “HNMDA-1” or “55063” nucleic acid and polypeptide molecules, which are novel members of the glutamate-gated ion channel family. These novel molecules are capable of, for example, modulating a glutamate-gated ion channel mediated activity (e.g., an NMDA mediated activity) in a neural cell (e.g., in the brain and/or spinal cord). These novel molecules are capable of binding neurotransmitters, e.g., L-glutamate and glycine, and transporting ions, e.g., Ca²⁺, across neural membranes and, thus, play a role in or function in a variety of cellular processes, e.g., mediating excitatory postsynaptic currents (e.g., long term potentiation).

The human NMDA-1 or 55063 sequence (SEQ ID NO:56), which is approximately 4197 nucleotides long including untranslated regions, contains a predicted methionine-initiated coding sequence of about 3345 nucleotides, not including the termination codon (nucleotides 1-3345 of SEQ ID NO:56; 1-3345 of SEQ ID NO:58). The coding sequence encodes a 1115 amino acid protein (SEQ ID NO:57).

The HNMDA-1 amino acid sequence was aligned with the amino acid sequence of the rat NMDA-L (Accession No. 1050330; SEQ ID NO:59) amino acid sequence using the CLUSTAL W (1.74) multiple sequence alignment program to show the homology between the two proteins.

A search using the polypeptide sequence of SEQ ID NO:57 was performed against the HMM database in PFAM resulting in the identification of a potential ligand-gated ion channel family domain in the amino acid sequence of HNMDA-1 at about residues 674-952 of SEQ ID NO:57 (score=198.1).

A search using the polypeptide sequence of SEQ ID NO:57 was also performed against the HMM database in SMART resulting in the identification of a potential glutamate-gated ion channel family domain in the amino acid sequence of HNMDA-1 at about residues 565-910 of SEQ ID NO:57 (score=267.4).

The amino acid sequence of HNMDA-1 was analyzed using the program PSORT to predict the localization of the proteins within the cell. This program assesses the presence of different targeting and localization amino acid sequences within the query sequence. The results of this analysis show that HNMDA-1 may be localized to the endoplasmic reticulum, mitochondria, or nucleus.

Searches of the amino acid sequence of HNMDA-1 were further performed against the Prosite database. These searches resulted in the identification in the amino acid sequence of HNMDA-1 of a number of potential N-glycosylation sites, a potential cAMP- and cGMP-dependent protein kinase phosphorylation site, a number of potential protein kinase C phosphorylation sites, a number of potential casein kinase II phosphorylation sites, a potential tyrosine kinase phosphorylation site, a number of potential N-myristoylation sites, a number of potential amidation sites, and a potential ATP/GTP-binding site motif A (P-loop).

A MEMSAT analysis of the polypeptide sequence of SEQ ID NO:57 was also performed predicting three possible transmembrane domains in the amino acid sequence of HNMDA-1 (SEQ ID NO:57) at about residues 677-695, 748-770, and 931-951. Further analysis of the amino acid sequence of SEQ ID NO:57 (e.g., alignment with, for example, a known rat NMDA protein, SEQ ID NO:59) resulted in the identification of a fourth transmembrane domain at about amino acid residues 713-734 of SEQ ID NO:57.

As used herein, a “glutamate-gated ion channel” includes a protein or polypeptide which is a member of the ligand-gated ion channel family and is involved in binding ligands, (e.g., binding L-glutamate and glycine), and transporting ions (e.g., Ca²⁺) across the plasma membrane of a cell (e.g., a neural cell). Glutamate-gated ion channels regulate long term potentiation in a cell and, typically, have glutamate substrate specificity. Examples of glutamate-gated ion channels include kainate, AMPA, and NMDA receptors.

As used herein, a “glutamate-gated ion channel mediated activity” includes an activity which involves a glutamate-gated ion channel in a cell (e.g., in a neural cell). Glutamate-gated ion channel mediated activities include the binding of a ligand (e.g., L-glutamine and/or glycine); the transporting of Ca²⁺ across a neural membrane; the regulation of long term potentiation; and the regulation of synapse formation underlying memory, learning, and formation of neural networks during development.

As the HNMDA-1 molecules of the present invention are glutamate-gated ion channels, they may be useful for developing novel diagnostic and therapeutic agents for glutamate-gated ion channel associated disorders. As used herein, the term “glutamate-gated ion channel associated disorder” includes a disorder, disease, or condition which is characterized by an aberrant, e.g., upregulated or downregulated, glutamate-gated ion channel mediated activity. Glutamate-gated ion channel associated disorders typically result in, e.g., upregulated or downregulated, Ca²⁺ levels in a cell (e.g., a neural cell). Examples of glutamate-gated ion channel associated disorders include disorders associated with long term synapse potentiation, acute and chronic neurological disorders, psychiatric disorders, and neuropathic pain syndromes. Glutamate-gated ion channel associated disorders can detrimentally affect conveyance of sensory impulses from the periphery to the brain and/or conductance of motor impulses from the brain to the periphery; integration of reflexes; interpretation of sensory impulses; and emotional, intellectual (e.g., learning and memory), or motor processes.

The term “family” when referring to the polypeptide and nucleic acid molecules of the invention is intended to mean two or more polypeptides or nucleic acid molecules having a common structural domain or motif and having sufficient amino acid or nucleotide sequence homology as defined herein. For example, the family of HNMDA-1 polypeptides comprise at least one “transmembrane domain” and preferably four transmembrane domains. As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al, (1996) Annual Rev. Neurosci. 19: 235-263, the contents of which are incorporated herein by reference. A MEMSAT analysis resulted in the identification of four transmembrane domains in the amino acid sequence of HNMDA-1 (SEQ ID NO:57) at about residues 7-28, 677-695, 748-770, and 931-951.

Accordingly, HNMDA-1 polypeptides having at least 50-60% homology, preferably about 60-70%, more preferably about 70-80%, or about 80-90% homology with a transmembrane domain of human HNMDA-1 are within the scope of the invention.

In another embodiment of the invention features HNMDA-1 molecules which contain a signal sequence. As used herein, a “signal peptide” includes a peptide of at least about 20 amino acid residues in length which occurs at the N-terminus of secretory and integral membrane proteins and which contains at least 55% hydrophobic amino acid residues. In a preferred embodiment, a signal sequence contains at least about 15-45 amino acid residues, preferably about 20-42 amino acid residues. Signal sequences of 25-35 amino acid residues and 28-32 amino acid residues are also within the scope of the invention. As used herein, a signal sequence has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline). Such a “signal peptide”, also referred to in the art as a “signal sequence”, serves to direct a protein containing such a sequence to a lipid bilayer. For example, a signal sequence can be found at about amino acids 1-22 of SEQ ID NO:57 (Met1 to Ala22 of the HNMDA-1 amino acid sequence).

Accordingly, HNMDA-1 polypeptides having at least 50-60% homology, preferably about 60-70%, more preferably about 70-80%, or about 80-90% homology with a signal sequence domain of HNMDA-1 are within the scope of the invention.

In another embodiment, an HNMDA-1 molecule of the present invention is identified based on the presence of at least one “ligand-gated ion channel family domain.” As used herein, the term “ligand-gated ion channel family domain” includes a protein domain having at least about 200-400 amino acid residues, having a bit score of at least 100 when compared against a ligand-gated ion channel family domain Hidden Markov Model (HMM), and, preferably, a ligand-gated ion channel mediated activity. Preferably, a ligand-gated ion channel family domain includes a polypeptide having an amino acid sequence of about 250-400, 250-350, or more preferably, about 278 amino acid residues, a bit score of at least 160, 170, 180, 190, or more preferably about 198.1, and, preferably a ligand-gated ion channel mediated activity. To identify the presence of a ligand-gated ion channel family domain in an HNMDA-1 protein, and make the determination that a protein of interest has a particular profile, the amino acid sequence of the protein may be searched against a database of known protein domains (e.g., the PFAM HMM database). A PFAM ligand-gated ion channel family domain has been assigned the PFAM Accession PF00060. A search was performed against the PFAM HMM database resulting in the identification of a ligand-gated ion channel family domain in the amino acid sequence of an HNMDA-1 at about residues 674-952 of SEQ ID NO:57.

Preferably, a “ligand-gated ion channel family domain” has a “ligand-gated ion channel mediated activity” as described herein. For example, a ligand-gated ion channel family domain may have the ability to bind a ligand, e.g., a neurotransmitter (e.g., acetylcholine, serotonin, glycine, glutamate, and/or GABA), on a cell (e.g., a neural cell); and the ability to regulate ion transport in a cell (e.g., Ca²⁺, K⁺, H⁺, Cl⁻, Mg²⁺ and/or Na⁺). Accordingly, identifying the presence of a “ligand-gated ion channel family domain” can include isolating a fragment of an HNMDA-1 molecule (e.g., an HNMDA-1 polypeptide) and assaying for the ability of the fragment to exhibit one of the aforementioned ligand-gated ion channel mediated activities.

In another embodiment, an HNMDA-1 molecule of the present invention is identified based on the presence of at least one “glutamate-gated ion channel family domain.” As used herein, the term “glutamate-gated ion channel family domain,” also known as an “ionotropic glutamate receptor family domain,” includes a protein domain having at least about 200-500 amino acid residues, having a bit score of at least 200 when compared against a glutamate-gated ion channel family domain Hidden Markov Model (HMM), and a glutamate-gated ion channel mediated activity. Preferably, a glutamate-gated ion channel family domain includes a polypeptide having an amino acid sequence of about 250-450, 300-400, 325-375, or more preferably, about 345 amino acid residues, a bit score of at least 210, 220, 230, 240, 250, 260, or more preferably about 267.4, and a glutamate-gated ion channel mediated activity. To identify the presence of a glutamate-gated ion channel family domain in an HNMDA-1 protein, and make the determination that a protein of interest has a particular profile, the amino acid sequence of the protein may be searched against a database of known protein domains (e.g., the PFAM HMM database). A PFAM glutamate-gated ion channel family domain has been assigned the InterPro Accession IPR001320. A search was performed against the PFAM HMM database resulting in the identification of a glutamate-gated ion channel family domain in the amino acid sequence of an HNMDA-1 at about residues 565-910 of SEQ ID NO:57.

Preferably, a “glutamate-gated ion channel family domain” has a “glutamate-gated ion channel mediated activity” as described herein. For example, a glutamate-gated ion channel family domain may have the ability to bind a ligand, e.g., L-glutamate and/or glycine, on a cell (e.g., a neural cell); and the ability to regulate Ca²⁺ transport in a cell. Accordingly, identifying the presence of a “glutamate-gated ion channel family domain” can include isolating a fragment of an HNMDA-1 molecule (e.g., an HNMDA-1 polypeptide) and assaying for the ability of the fragment to exhibit one of the aforementioned glutamate-gated ion channel mediated activities.

A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

In a preferred embodiment, the HNMDA-1 molecules of the invention include at least one, preferably two, more preferably three, and even more preferably four transmembrane domain(s) and at least one of the following domains: a signal peptide, a ligand-gated ion channel family domain, and/or a glutamate-gated ion channel family domain.

Isolated HNMDA-1 polypeptides of the present invention have an amino acid sequence sufficiently identical to the amino acid sequence of SEQ ID NO:57 or are encoded by a nucleotide sequence sufficiently identical to SEQ ID NO:56 or 58. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains having at least 50%, 55%, 60%, 65%, 70%, 75%, 80%, 85%, 85%, 90%, 95%, 96%, 97%, 98%, 99% or more homology or identity across the amino acid sequences of the domains and contain at least one and preferably two structural domains or motifs, are defined herein as sufficiently identical. Furthermore, amino acid or nucleotide sequences which share at least 50%, 55%, 60%, 65%, 70%, 75%, 80%, 85%, 85%, 90%, 95%, 96%, 97%, 98%, 99% or more homology or identity and share a common functional activity are defined herein as sufficiently identical.

In a preferred embodiment, an HNMDA-1 polypeptide includes at least one or more of the following domains: a transmembrane domain, a signal peptide, a ligand-gated ion channel family domain, and/or a glutamate-gated ion channel family domain, and has an amino acid sequence at least about 50%, 55%, 60%, 65%, 70%, 75%, 80%, 85%, 85%, 90%, 95%, 96%, 97%, 98%, 99% or more homologous or identical to the amino acid sequence of SEQ ID NO:57. In yet another preferred embodiment, an HNMDA-1 polypeptide includes at least one or more of the following domains: a transmembrane domain, a signal peptide, a ligand-gated ion channel family domain, and/or a glutamate-gated ion channel family domain, and is encoded by a nucleic acid molecule having a nucleotide sequence which hybridizes under stringent hybridization conditions to a complement of a nucleic acid molecule comprising the nucleotide sequence of SEQ ID NO:56 or SEQ ID NO:58. In another preferred embodiment, an HNMDA-1 polypeptide includes at least one or more of the following domains: a transmembrane domain, a signal peptide, a ligand-gated ion channel family domain, and/or a glutamate-gated ion channel family domain, and has an HNMDA-1 activity.

As used interchangeably herein, an “HNMDA-1 activity”, “biological activity of HNMDA-1” or “functional activity of HNMDA-1,” refers to an activity exerted by an HNMDA-1 polypeptide or nucleic acid molecule on an HNMDA-1 responsive cell or tissue, or on an HNMDA-1 polypeptide substrate, as determined in vivo, or in vitro, according to standard techniques. In one embodiment, an HNMDA-1 activity is a direct activity, such as an association with an HNMDA-1-target molecule. As used herein, a “substrate,” “target molecule,” or “binding partner” is a molecule with which an HNMDA-1 polypeptide binds or interacts in nature, such that HNMDA-1-mediated function is achieved. An HNMDA-1 target molecule can be a non-HNMDA-1 molecule or an HNMDA-1 polypeptide or polypeptide of the present invention. In an exemplary embodiment, an HNMDA-1 target molecule is an HNMDA-1 ligand, e.g., a glutamate-gated ion channel ligand such as L-glutamate or glycine. Alternatively, an HNMDA-1 activity is an indirect activity, such as a cellular signaling activity mediated by interaction of the HNMDA-1 polypeptide with an HNMDA-1 ligand. The biological activities of HNMDA-1 are described herein. For example, the HNMDA-1 polypeptides of the present invention can have one or more of the following activities: (1) modulate Ca²⁺ transport across a cell membrane, (2) modulate intracellular Ca²⁺ concentration, (3) bind a ligand, e.g., L-glutamate, and/or glycine, (4) influence long term synapse potentiation, (5) modulate synapse formation, e.g., synapse formation related to memory or learning, and/or (6) modulate synapse formation related to the formation of neural networks during development.

### Human 52991

Membrane transport molecules (e.g., channels/pores, permeases, and transporters) play important roles in the ability of the cell to regulate homeostasis, to grow and divide, and to communicate with other cells, e.g., to secrete and receive signaling molecules, such as hormones, reactive oxygen species, ions, neurotransmitters, and cytokines. A wide variety of human diseases and disorders are associated with defects in transporter or other membrane transport molecules, including certain types of liver disorders (e.g., due to defects in transport of long-chain fatty acids (Al Odaib et al. (1998) New Eng. J. Med. 339: 1752-1757)), hyperlysinemia (due to a transport defect of lysine into mitochondria (Oyanagi et al. (1986) Inherit. Metab. Dis. 9: 313-316), and cataracts (Wintour (1997) Clin Exp Pharmacol Physiol 24(1):1-9). The present invention is based, in part, on the discovery of a novel human transporter, referred to herein as “52991”

The human 52991 sequence (SEQ ID NO:60), which is approximately 2247 nucleotides long including untranslated regions, contains a predicted methionine-initiated coding sequence of about 1995 nucleotides (nucleotides 51-2045 of SEQ ID NO:60; 1-1995 of SEQ ID NO:62), not including the terminal codon. The coding sequence encodes a 665 amino acid protein (SEQ ID NO:61).

This mature protein form is approximately 665 amino acid residues in length (from about amino acid 1 to amino acid 665 of SEQ ID NO:61). Human 52991 contains the following regions or other structural features: a predicted Na+ dependent nucleoside transporter domain located at about amino acid residues 198-587 of SEQ ID NO:61; thirteen predicted transmembrane domains which extend from about amino acid residues 104-120, 128-144, 175-191, 201-217, 228-244, 262-282, 289-313, 336-354, 363-382, 418-442, 454-473, 528-550 and 568-586 of SEQ ID NO:61; five predicted N-glycosylation sites (PS00001) located at about amino acids 30-33, 34-37, 604-607, 610-613, and 638-641 of SEQ ID NO:61; nine predicted protein kinase C phosphorylation sites (PS00005) located at about amino acids 36-38, 100-102, 193-195, 385-387, 391-393, 523-525, 556-558, 611-613, and 643-645 of SEQ ID NO:61; four predicted casein kinase II phosphorylation sites (PS00006) located at about amino acids 50-53, 300-303, 330-333, and 589-592 of SEQ ID NO:61; one predicted tyrosine kinase phosphorylation site (PS00007) located at about amino acids 80-87 of SEQ ID NO:61; ten predicted N-myristoylation sites (PS00008) located at about amino acids 107-112, 347-352, 357-362, 413-418, 544-549, 564-569, 572-577, 584-589, 636-641, and 660-665 of SEQ ID NO:61; one predicted amidation site (PS00009) located at about amino acids 161-164 of SEQ ID NO:61; and two predicted prokaryotic membrane lipoprotein lipid attachment sites (PS00013) located at about amino acids 111-121 and 571-581 of SEQ ID NO:61.

For general information regarding PFAM identifiers, PS prefix and PF prefix domain identification numbers, refer to Sonnhammer et al. (1997) Protein 28:405-420.

A hydropathy plot of human 52991 was performed. Polypeptides of the invention include fragments which include: all or part of a hydrophobic sequence, e.g., the sequence from about amino acid 105 to 120, from about 420 to 440, and from about 530 to 550 of SEQ ID NO:61; all or part of a hydrophilic sequence, e.g., a sequence below the dashed line, e.g., the sequence from about amino acid 20 to 35, from about 65 to 85, and from about 380 to 390 of SEQ ID NO:61; a sequence which includes a Cys, or a glycosylation site.

The 52991 protein contains a significant number of structural characteristics in common with members of the transporter family. The term “family” when referring to the protein and nucleic acid molecules of the invention means two or more proteins or nucleic acid molecules having a common structural domain or motif and having sufficient amino acid or nucleotide sequence homology as defined herein. As used herein, the term “transporter” includes a molecule which is involved in the movement of an ion or a biochemical molecule from one side of a lipid bilayer to the other, for example, against a preexisting concentration gradient. Transporters are usually involved in the movement of biochemical compounds which would normally not be able to cross a membrane (e.g., a protein, an ion, or other small molecule, such as ATP, signaling molecules, vitamins, and cofactors). Transporter molecules are involved in the growth, development, and differentiation of cells, in the regulation of cellular homeostasis, in the metabolism and catabolism of biochemical molecules necessary for energy production or storage, in intra- or intercellular signaling, in metabolism or catabolism of metabolically important biomolecules, and in the removal of potentially harmful compounds from the interior of the cell. Examples of transporters include GSH transporters, ATP transporters, and fatty acid transporters. As transporters, the transporter molecules of the present invention provide novel diagnostic targets and therapeutic agents to control transporter-associated disorders.

As used herein, a “52991 activity”, “biological activity of 52991” or “functional activity of 52991”, refers to an activity exerted by a 52991 protein, polypeptide or nucleic acid molecule on e.g., a 52991-responsive cell or on a 52991 substrate, e.g., a lipid or protein substrate, as determined in vivo or in vitro. In one embodiment, a 52991 activity is a direct activity, such as an association with a 52991 target molecule. A “target molecule” or “binding partner” is a molecule with which a 52991 protein binds or interacts in nature, e.g., a molecule to which the 52991 protein transports across a biological membrane. A 52991 activity can also be an indirect activity, e.g., a cellular signaling activity mediated by interaction of the 52991 protein with a 52991 ligand. For example, the 52991 proteins of the present invention can have one or more of the following activities: 1) modulate the import and export of molecules from cells, e.g., sugars, amino acids and possibly other metabolites, 2) modulate intra- or intercellular signaling, 3) modulate removal of potentially harmful compounds from the cell, or facilitate the compartmentalization of these molecules into a sequestered intracellular space (e.g., the peroxisome), and 4) modulate transport of molecules across membranes, e.g., the plasma membrane, or the membrane of a mitochondrion, a peroxisome, a lysosome, the endoplasmic reticulum, the nucleus, or a vacuole and 5) the ability to antagonize or inhibit, competitively or non-competitively, any of 1-4. Therefore, the 52991 protein may play a role in the transport of molecules into cells or across membranes in cells or organelles that lack such molecules or alternatively in the transport of molecules across membranes from cells or organelles that have an excess of such molecules.

The 52991 transporter protein has similarities to previously characterized sodium nucleoside cotransporters. (Huang, Q. Q., et al. (1994) J Biol Chem 269(27):17757-60; Ritzel, M. W. et al. (1997) Am J Phiosiol 272(2 Pt 1):C707-14; Pajor, A. M. Biochim Biosphys Acta (1998) 141(1):266-9; Che, M et al. (1995) J Biol Chem 270(23):13596-9; Yao, S. Y., et al. (1996) Mol Pharmacol 50(6):127-35). Thus, the 52991 transporter may play a role similar to that of such known cotransporters in transporting nucleosides or nucleoside analogs. More specifically, the 52991 transporter may be involved in the intestinal absorption and/or the renal handling of pyrimidine nucleosides, such a thymidine and uridine, or pyrimidine analogs, such as AZT, used to treat transporter-associated disorders. Therefore, regulation of 52991 transporter activity may be an important strategy in controlling transporter-associated disorders associated with the inhibition or stimulation of 52991 transporter activity.

A 52991 polypeptide can include a “Na+ dependent nucleoside transporter domain” or regions homologous with a “Na+ dependent nucleoside transporter domain”. As used herein, the term “Na+ dependent nucleoside transporter domain” includes an amino acid sequence of about 200-500 amino acid residues in length and having a bit score for the alignment of the sequence to the transporter domain (HMM) of at least 8. Preferably, a transporter domain includes at least about 300-450 amino acids, more preferably about 350-425 amino acid residues, or about 375-400 amino acids and has a bit score for the alignment of the sequence to the transporter domain (HMM) of at least 16, 50, 100, 200, 300, 400, 500 or greater. The transporter domain (HMM) has been assigned the PFAM Accession PF01773. The transporter domain (amino acids 198 to 587 of SEQ ID NO:61) of human 52991 aligns with a consensus amino acid sequence (SEQ ID NO:63) derived from a hidden Markov model.

In a preferred embodiment 52991 polypeptide or protein has a “Na+ dependent nucleoside transporter domain” or a region which includes at least about 200-500 more preferably about 300-450 or 375-400 amino acid residues and has at least about 60%, 70%, 80%, 90%, 95%, 99%, or 100% homology with a “Na+ dependent nucleoside transporter domain,” e.g., the Na+ dependent nucleoside transporter domain of human 52991 (e.g., amino acid residues 198-587 of SEQ ID NO:61).

To identify the presence of a “transporter” domain in a 52991 protein sequence, and make the determination that a polypeptide or protein of interest has a particular profile, the amino acid sequence of the protein can be searched against a database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al., (1997) Proteins 28(3):405-420 and a detailed description of HMMs can be found, for example, in Gribskov et al., (1990) Meth. Enzymol. 183:146-159; Gribskov et al., (1987) Proc. Natl. Acad. Sci. USA 84:4355-4358; Krogh et al., (1994) J. Mol. Biol. 235:1501-1531; and Stultz et al., (1993) Protein Sci. 2:305-314, the contents of which are incorporated herein by reference.

A search was performed against the HMM database resulting in the identification of a “Na+ dependent nucleoside transporter” domain in the amino acid sequence of human 52991 at about residues 198-587 of SEQ ID NO:61.

For further identification of domains in a 52991 protein sequence, and make the determination that a polypeptide or protein of interest has a particular profile, the amino acid sequence of the protein can be searched against a database of domains, e.g., the ProDom database (Corpet et al. (1999), Nucl. Acids Res. 27:263-267). The ProDom protein domain database consists of an automatic compilation of homologous domains. Current versions of ProDom are built using recursive PSI-BLAST searches (Altschul S F et al. (1997) Nucleic Acids Res. 25:3389-3402; Gouzy et al. (1999) 23:333-340) of the SWISS-PROT 38 and TREMBL protein databases. The database automatically generates a consensus sequence for each domain.

A BLAST search was performed against the HMM database resulting in the identification of regions homologous to ProDom family PD003768, (“nucleoside transporter cotransporter transmembrane concentrative sodium/nucleoside na/nucleoside sodium-coupled” SEQ ID NOs:64 and 65, ProDomain Release 2001.1). The “nucleoside transporter cotransporter transmembrane concentrative sodium/nucleoside na/nucleoside sodium-coupled” domain (amino acids 262-585 and 201-261 of SEQ ID NO:61) of human 52991 aligns with consensus amino acid sequences (SEQ ID NOs:64 and 65) derived from a hidden Markov model. The consensus sequences for SEQ ID NOs:64 and 65 are 53% and 34% identical over amino acids 262 to 585 and 201 to 261 of SEQ ID NO:61, respectively.

A BLAST search was performed against the HMM database resulting in the identification of regions homologous to ProDom family PD351462, (“concentrative cotransporter Na+ nucleoside HCNT3 MCNT3” SEQ ID NO:66, ProDomain Release 2001.1. The “concentrative cotransporter na+ nucleoside HCNT3 MCNT3” domain (amino acids 587-663 of SEQ ID NO:61) of human 52991 aligns with consensus amino acid sequences (SEQ ID NO:66) derived from a hidden Markov model. The consensus sequence for SEQ ID NO:66 is 81% identical over amino acids 587 to 663 of SEQ ID NO:61.

A BLAST search was performed against the HMM database resulting in the identification of regions homologous to ProDom family PD008773, (“nucleoside cotransporter concentrative sodium/nucleoside na/nucleoside sodium-coupled transmembrane” SEQ ID NO:67, ProDomain Release 2001.1). The “nucleoside cotransporter concentrative sodium/nucleoside na/nucleoside sodium-coupled transmembrane” domain (amino acids 93-195 of SEQ ID NO:61) of human 52991 aligns with a consensus amino acid sequence (SEQ ID NO:67) derived from a hidden Markov model. The consensus sequence for SEQ ID NO:67 is 52% identical over amino acids 93 to 195 of SEQ ID NO:61.

A BLAST search was performed against the HMM database resulting in the identification of regions homologous to ProDom family PD353176 (“concentrative cotransporter Na+ nucleoside HCNT3 MCNT3” SEQ ID NO:68, ProDomain Release 2001.1). The “concentrative cotransporter Na+ nucleoside HCNT3 MCNT3” domain (amino acids 1-91 of SEQ ID NO:61) of human 52991 aligns with consensus amino acid sequences (SEQ ID NO:68) derived from a hidden Markov model. The consensus sequence for SEQ ID NO:68 is 61% identical over amino acids 1 to 91 of SEQ ID NO:61.

In one embodiment, a 52991 protein includes at least one transmembrane domain. As used herein, the term “transmembrane domain” includes an amino acid sequence of about 15 amino acid residues in length that spans a phospholipid membrane. More preferably, a transmembrane domain includes about at least 18, 20, 22, 24, 25, 30, 35 or 40 amino acid residues and spans a phospholipid membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al., (1996) Annual Rev. Neuronsci. 19: 235-63, the contents of which are incorporated herein by reference.

In a preferred embodiment, a 52991 polypeptide or protein has at least one transmembrane domain or a region which includes at least 18, 20, 22, 24, 25, 30, 35 or 40 amino acid residues and has at least about 60%, 70% 80% 90% 95%, 99%, or 100% homology with a “transmembrane domain,” e.g., at least one transmembrane domain of human 52991 (e.g., amino acid residues 104-120 of SEQ ID NO:61).

In another embodiment, a 52991 protein includes at least one “non-transmembrane domain.” As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a non-transmembrane domain is adjacent to an N-terminal amino acid residue of a transmembrane domain in a naturally-occurring 52991, or 52991-like protein.

In a preferred embodiment, a 52991 polypeptide or protein has a “non-transmembrane domain” or a region which includes at least about 1-105, preferably about 2-103, more preferably about 3-103, and even more preferably about 5-103 amino acid residues, and has at least about 60%, 70% 80% 90% 95%, 99% or 100% homology with a “non-transmembrane domain”, e.g., a non-transmembrane domain of human 52991 (e.g., residues 1-103 and 587-665 of SEQ ID NO:61). Preferably, a non-transmembrane domain is capable of catalytic activity (e.g., transport of molecules across a lipid bilayer).

A non-transmembrane domain located at the N-terminus of a 52991 protein or polypeptide is referred to herein as an “N-terminal non-transmembrane domain.” As used herein, an “N-terminal non-transmembrane domain” includes an amino acid sequence having about 1-105, preferably about 40-103, more preferably about 80-103, or even more preferably about 90-103 amino acid residues in length and is located outside the boundaries of a membrane. For example, an N-terminal non-transmembrane domain is located at about amino acid residues 1-103 of SEQ ID NO:61.

Similarly, a non-transmembrane domain located at the C-terminus of a 52991 protein or polypeptide is referred to herein as a “C-terminal non-transmembrane domain.” As used herein, a “C-terminal non-transmembrane domain” includes an amino acid sequence having about 1-80, preferably about 30-80, preferably about 40-78, more preferably about 60-78 amino acid residues in length and is located outside the boundaries of a membrane. For example, a C-terminal non-transmembrane domain is located at about amino acid residues 587-665 of SEQ ID NO:61.

In a preferred embodiment, a 52991 family member can include at least one Na+ dependent nucleoside transporter family domain (PFAM Accession Number PF01773). Furthermore, a 52991 family member can include at least one, two, three, four, and preferably five N-glycosylation site (PS00001); at least one, two, three, four, five, six, seven, eight, and preferably nine protein kinase C phosphorylation sites (PS00005); at least one, two, three, and preferably four casein kinase II phosphorylation sites (PS00006); at least one tyrosine kinase phosphorylation site (PS00007); at least one, two, three, four, five, six, seven, eight, nine, and preferably ten N-myristolyation sites (PS00008); at least one amidation site (PS00009); and at least one, preferably two prokaryotic membrane lipoprotein lipid attachment sites (PS00013).

As the 52991 polypeptides of the invention may modulate 52991-mediated activities, they may be useful for developing novel diagnostic and therapeutic agents for 52991-mediated or related disorders, as described below.

As used herein, a “transporter-associated disorder” includes a disorder, disease or condition which is caused or characterized by a misregulation (e.g., downregulation or upregulation) of a transporter-mediated activity. Transporter-associated disorders can detrimentally affect cellular functions such as cellular proliferation, growth, differentiation, or migration, cellular regulation of homeostasis, inter- or intra-cellular communication; tissue function, such as cardiac function or musculoskeletal function; systemic responses in an organism, such as nervous system responses, hormonal responses (e.g., insulin response), or immune responses; and protection of cells from toxic compounds (e.g., carcinogens, toxins, mutagens, and toxic byproducts of metabolic activity (e.g., reactive oxygen species)). Accordingly, 52991 protein may mediate various disorders, including cellular proliferative and/or differentiative disorders, immune disorders, blood vessel disorders, bone metabolism, liver disorders, and pain or metabolism disorders. As the 52991 polypeptides of the invention may modulate 52991-mediated activities, they may be useful for developing novel diagnostic and therapeutic agents for 52991-mediated or related disorders, as described below.

The 52991 nucleic acid and protein of the invention can be used to treat and/or diagnose a variety of proliferative disorders, e.g., such disorders include hematopoietic neoplastic disorders.

Transporter-associated or related disorders also include immune disorders, such as autoimmune disorders or immune deficiency disorders, e.g., congenital X-linked infantile hypogammaglobulinemia, transient hypogammaglobulinemia, common variable immunodeficiency, selective IgA deficiency, chronic mucocutaneous candidiasis, or severe combined immunodeficiency.

Aberrant expression and/or activity of 52991 molecules may mediate disorders associated with bone metabolism.

Disorders which may be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. Additionally, 52991 may play an important role in the regulation of metabolism or pain disorders.

### Gene Expression Analysis of 52991

TaqMan real-time quantitative RT-PCR is used to detect the presence of RNA transcript corresponding to human 52991 relative to a no template control in a panel of human tissues or cells. It is found that the highest expression of 52991 orthologs are expressed in osteoclasts and pancreas tissue, as shown in Table 14. Relatively high expression is also seen in normal breast tissue, ovary tumor tissue, colon tumor tissue, lung tumor tissue, and neutrophils. It is also of note that there is decreased expression of 52991 in breast tumor, and pancreatic tumor compared to normal breast and pancreatic tissue. Furthermore, there is increased expression of 52991 in ovary tumor, colon tumor, lung tumor, fibrotic liver tissue and diseased aorta tissue compared to normal ovary, colon, lung and liver tissue and normal artery tissue.

TABLE 14

| Tissue Type | Mean | β 2 Mean | ∂∂ Ct | Expression |
|---|---|---|---|---|
| Artery normal | 38.56 | 23.51 | 13.75 | 0 |
| Aorta diseased | 34.45 | 23.72 | 9.43 | 1.4497 |
| Vein normal | 38.4 | 21.66 | 15.43 | 0 |
| Coronary SMC | 35.59 | 22.23 | 12.06 | 0 |
| HUVEC | 40 | 22.77 | 15.93 | 0 |
| Hemangioma | 38.57 | 21.02 | 16.25 | 0 |
| Heart normal | 36.29 | 21.9 | 13.09 | 0 |
| Heart CHF | 37.17 | 22.17 | 13.7 | 0 |
| Kidney | 33.94 | 21.73 | 10.91 | 0.5197 |
| Skeletal Muscle | 38.99 | 23.35 | 14.34 | 0 |
| Adipose normal | 36.91 | 22.07 | 13.53 | 0 |
| Pancreas | 30.75 | 23.59 | 5.85 | 17.337 |
| Primary osteoblasts | 33.66 | 22.11 | 10.24 | 0.8269 |
| Osteoclasts (diff) | 25.91 | 18.95 | 5.65 | 19.915 |
| Skin normal | 33.06 | 23.68 | 8.07 | 3.7212 |
| Spinal cord normal | 37.02 | 22.2 | 13.51 | 0 |
| Brain Cortex normal | 39.39 | 23.39 | 14.7 | 0 |
| Brain Hypothalamus normal | 35.68 | 23.86 | 10.52 | 0 |
| Nerve | 38.91 | 23.24 | 14.37 | 0 |
| DRG (Dorsal Root Ganglion) | 40 | 23.27 | 15.43 | 0 |
| Breast normal | 31.11 | 22.46 | 7.34 | 6.1508 |
| Breast tumor | 32.51 | 22.27 | 8.94 | 2.0361 |
| Ovary normal | 33.22 | 21.23 | 10.68 | 0.6095 |
| Ovary Tumor | 29.16 | 20.57 | 7.29 | 6.412 |
| Prostate Normal | 34.99 | 20.88 | 12.81 | 0.1393 |
| Prostate Tumor | 36.73 | 21.61 | 13.82 | 0 |
| Salivary glands | 33.48 | 21.18 | 10.99 | 0.4917 |
| Colon normal | 30.81 | 19.93 | 9.57 | 1.3111 |
| Colon Tumor | 31.1 | 22.77 | 7.03 | 7.6517 |
| Lung normal | 33.22 | 19.54 | 12.38 | 0.1883 |
| Lung tumor | 30.05 | 21.51 | 7.23 | 6.6612 |
| Lung COPD | 31.3 | 19.79 | 10.21 | 0.8443 |
| Colon IBD | 29.48 | 19.06 | 9.12 | 1.8035 |
| Liver normal | 34.72 | 21.63 | 11.79 | 0.2834 |
| Liver fibrosis | 32.23 | 22.04 | 8.88 | 2.1152 |
| Spleen normal | 36.28 | 19.91 | 15.06 | 0 |
| Tonsil normal | 30.25 | 19.33 | 9.62 | 1.2708 |
| Lymph node normal | 33.69 | 21.07 | 11.31 | 0.3939 |
| Small intestine normal | 32.12 | 21.54 | 9.28 | 1.6086 |
| Macrophages | 33.07 | 18.34 | 13.43 | 0.0906 |
| Synovium | 32.27 | 21.02 | 9.95 | 1.011 |
| BM-MNC | 31.01 | 20.34 | 9.37 | 1.5165 |
| Activated PBMC | 36.84 | 19.38 | 16.16 | 0 |
| Neutrophils | 28.73 | 20.36 | 7.06 | 7.4943 |
| Megakaryocytes | 38.08 | 20.26 | 16.51 | 0 |
| Erythroid | 39.47 | 23.13 | 15.03 | 0 |
| positive control | 32.78 | 21.49 | 9.99 | 0.9834 |

### Human 59914 and 59921

The present invention is based, in part, on the discovery of novel genes encoding choline transporters, the genes being referred to herein as “59914 and 59921”.

The human 59914 cDNA sequence (SEQ ID NO:69), which is approximately 2473 nucleotide residues long including non-translated regions, contains a methionine-initiated coding sequence (without the 5′- and 3′-non-translated regions) of about 2151 nucleotide residues, excluding termination codon (i.e., nucleotide residues 88-2238 of SEQ ID NO:69; 1-2151 of SEQ ID NO:71). The coding sequence encodes a 717 amino acid protein having the amino acid sequence SEQ ID NO:70.

The human 59921 cDNA sequence (SEQ ID NO:72), which is approximately 2233 nucleotide residues long including non-translated regions, contains a methionine-initiated coding sequence (without the 5′- and 3′-non-translated regions) of about 1959 nucleotide residues, excluding termination codon (i.e., nucleotide residues 110-2068 of SEQ ID NO:72; 1-1959 of SEQ ID NO:74). The coding sequence encodes a 653 amino acid protein having the amino acid sequence SEQ ID NO:73.

Human 59914 and 59921 contain the following regions or other structural features: 1) a conserved region of sequence which is shared by both 59914 and 59921 proteins, and by other choline transporter (or choline transporter-like) proteins described herein and in O'Regan et al. (2000) PNAS 97(4):1835-1840. This region will henceforth be referred to as “conserved choline transporter domain”, and is located at about amino acid residues 479-598 of SEQ ID NO:70 and about amino acid residues 402-521 of SEQ ID NO:73; 2) transmembrane domains at about amino acid residues 39-61, 242-263, 270-287, 326-346, 371-395, 461-484, 514-536, 591-605, 608-632, and 649-672 of SEQ ID NO:70, and at about 33-57, 215-231, 239-262, 284-305, 328-352, 384-411, 436-458, 514-532, 534-555, and 563-586 of SEQ ID NO:73, 59914 and 59921 proteins therefore have about 10 transmembrane domains, as is characteristic of previously characterized choline transporters; 3) conserved cysteine residues at about amino acid residues 36, 79, 117, 121, 149, 168, 182, 557, 558, 561, and 681 of SEQ ID NO:70 and about amino acid residues 29, 72, 116, 120, 143, 162, 179, 480, 481, 484, and 596 of SEQ ID NO:73, and also found in other choline transporter (and choline transporter-like) proteins described herein and in O'Regan et al. (2000) PNAS 97(4):1835-1840. 59914 and 59921 proteins therefore have about 11 conserved cysteines, as is characteristic of previously characterized choline transporters; 4) and post translational modification sites including: predicted N-glycosylation sites (Pfam accession number PS00001) at about amino acid residues 2-5, 33-36, 88-91, 190-193, 314-317, 416-419, and 425-428 of SEQ ID NO:70 and at about amino acid residues 136-139, 151-154, 412-415, 503-506, and 521-524 of SEQ ID NO:73; predicted protein kinase C phosphorylation sites (Pfam accession number PS00005) at about amino acid residues 4-6, 152-154, 187-189, 587-589, 633-635, 693-695, and 714-716 of SEQ ID NO:70 and at about amino acid residues 90-92, 155-157, 210-212, 232-234, 276-278, 319-321, 510-512, 608-610, 625-627, and 639-641 of SEQ ID NO:73; predicted cAMP- and cGMP-dependent kinase phosphorylation sites (Pfam accession number PS00004) at about amino acid residues 27-30 and 74-77 of SEQ ID NO:73; predicted casein kinase II phosphorylation sites (Pfam accession number PS00006) located at about amino acid residues 10-13, 35-38, 84-87, 127-130, 140-143, 210-213, 305-308, and 587-590 of SEQ ID NO:70 and at about amino acid residues 77-80, 85-88, 127-130, 210-213, 276-279, 414-417, 510-513, 584-587, and 588-591 of SEQ ID NO:73; predicted N-myristoylation sites (Pfam accession number PS00008) at about amino acid residues 204-209, 215-220, 248-253, 285-290, 310-315, 435-440, and 481-486 of SEQ ID NO:70 and at about amino acid residues 69-74, 81-86, 107-112, 139-144, 207-212, 355-360, 386-391, 404-409, 504-509, and 550-555 of SEQ ID NO:73; a predicted tyrosine kinase phosphorylation site (Pfam accession number PS00007) at about amino acid residues 491-499 of SEQ ID NO:70; and a predicted amidation site (Pfam accession number PS00009) at about amino acid residues 72-75 of SEQ ID NO:73.

A hydropathy plot of human 59914 was performed. Polypeptides of the invention include fragments which include: all or part of a hydrophobic sequence, e.g., the sequences of about residues 39-61, 242-263, and 326-346 of SEQ ID NO:70; all or part of a hydrophilic sequence, e.g., the sequences of residues 62-90, 347-370, and 633-648 of SEQ ID NO:70; a sequence which includes a cysteine residue; or a glycosylation site.

A hydropathy plot of human 59921 was performed. Polypeptides of the invention include fragments which include: all or part of a hydrophobic sequence, e.g., the sequences of about residues 33-57, 215-231, and 328-352 of SEQ ID NO:73; all or part of a hydrophilic sequence, e.g., the sequences of residues 58-214, 263-283, and 306-327 of SEQ ID NO:73; a sequence which includes a cysteine residue; or a glycosylation site.

The 59914 and 59921 proteins contain a number of structural characteristics in common with members of the choline transporter family. Choline transporter family members all show several transmembrane domains and can reasonably be thought to traverse the membrane about 10 times. In one embodiment, a first, large and variable loop between transmembrane domains 1 and 2 is potentially extracellular and glycosylated. In one embodiment, a highly conserved region covers the last four transmembrane domains and includes the fourth extracellular loop that contains about three conserved cysteines. Choline transporter family members generally lack a clear signal peptide and are targeted to the plasma membrane via their transmembrane domains.

59914 and 59921 proteins can include a conserved choline transporter domain. As used herein, a “conserved choline transporter domain” refers to a protein domain having an amino acid sequence of about 50-250 amino acid residues in length, preferably about 75-175 amino acid residues in length, more preferably about 100-150 amino acid residues in length, and most preferably about 119-121 amino acid residues in length; and which has about 1-10 conserved cysteine residues, preferably about 2-8 conserved cysteine residues, and more preferably about 3-7 conserved cysteine residues.

In one embodiment, the conserved choline transporter domain can have one, preferably both, of the following consensus sequences: [LVI]-A-G-A-Xaa(2)-[ST]-[CY]-Y-[FW]-Xaa(3)-K-Xaa(n1)-P-Xaa(2)-P-[LI]-Xaa(5)-[IR]-Xaa(3)-Y-H-Xaa-G-Xaa(4)-G-Xaa(2)-[LI]-[LI]-Xaa(4)-[IM]-Xaa(2)-[VMI]-[VI]-[VL] (SEQ ID NO:79) and/or L-K-[ERG]-Xaa(2)-[HN]-Xaa(n2)-C-C-Xaa-W-C-L-[DE]-Xaa(8)-N-A-Y-Xaa(3)-[AS]-I-Xaa(4)-F-C-Xaa-S-A-K-D-A-[FI]-Xaa-[IL]-L-Xaa(2)-N (SEQ ID NO:80)

In these consensus sequence patterns, each element in the pattern is separated by a dash (-); square [ ] brackets indicate the particular residues that are accepted at that position; Xaa indicates any residue is accepted at that position; repetition of a particular element is indicated by following the element with a numerical value or variable enclosed in parentheses (i.e., above, Xaa(2) indicates 2 residues of any type are repeated, and Xaa(n1) indicates that a range of residues of any type are repeated, as described herein); and the standard IUPAC one-letter code for the amino acids is used. n1 in the first consensus sequence (SEQ ID NO:79) can be 1-8, preferably 2-6, more preferably 3-4, and n2 in the second consensus sequence (SEQ ID NO:80) can be 8-15, preferably 10-13, more preferably 11-12.

These consensus sequences are found from about residues 479-533 and 540-598, of the 59914 protein (of SEQ ID NO:70), respectively; and from about residues 402-455 and 462-521 of the 59921 protein (of SEQ ID NO:73), respectively.

A conserved choline transporter domain is found in at least the following choline transporter (or choline transporter-like (CTL)) proteins: human CTL1 (Genbank accession number CAB75541; SEQ ID NO:75); human CTL2 (Genbank accession number CAB75542; SEQ ID NO:76); rat CTL1 (Genbank accession number CAB75555; SEQ ID NO:77); and torpedo CTL1 (Genbank accession number CAB75556; SEQ ID NO:78). For example, in the human CTL1 protein (Genbank accession number CAB75541; SEQ ID NO:75), the conserved choline transporter domain as described herein is found from about amino acids 407-525 (of SEQ ID NO:75), and the consensus sequences as described herein (SEQ ID NO:79 and SEQ ID NO:80) are found from about amino acids 407-460 and 467-525, respectively (of SEQ ID NO:75).

In one embodiment, the 59914 and 59921 polypeptides or proteins have a conserved choline transporter domain which includes at least about 50-250, preferably about 75-175, more preferably about 100-150, and most preferably about 119-121 amino acid residues in length and has at least about 60%, 70%, 80%, 90%, 95%, 99%, or 100% homology with a conserved choline transporter domain, e.g., the conserved choline transporter domain of human 59914 or 59921 (e.g., residues 479-598 of SEQ ID NO:70 or residues 402-521 of SEQ ID NO:73). In another embodiment, the 59914 and 59921 polypeptides or proteins have a conserved choline transporter domain which includes at least about 50-250, preferably about 75-175, more preferably about 100-150, and most preferably about 119-121 amino acid residues in length; has at least about 60%, 70%, 80%, 90%, 95%, 99%, or 100% homology with a conserved choline transporter domain, e.g., the conserved choline transporter domain of human 59914 or 59921 (e.g., residues 479-598 of SEQ ID NO:70 or residues 402-521 of SEQ ID NO:73); and has about 1-10, preferably about 2-8, and more preferably about 3-7 conserved cysteine residues (e.g., at about positions 553, 554, 557, 558, 561, and 585 of SEQ ID NO:70, and at about positions 409, 477, 478, 480, 481, 484, and 508 of SEQ ID NO:73).

In another embodiment, the 59914 and 59921 polypeptides or proteins have a conserved choline transporter domain which includes at least about 50-250, preferably about 75-175, more preferably about 100-150, and most preferably about 119-121 amino acid residues in length; has at least about 60%, 70%, 80%, 90%, 95%, 99%, or 100% homology with a conserved choline transporter domain, e.g., the conserved choline transporter domain of human 59914 or 59921 (e.g., residues 479-598 of SEQ ID NO:70 or residues 402-521 of SEQ ID NO:73); has about 1-10, preferably about 2-8, and more preferably about 3-7 conserved cysteine residues (e.g., at about positions 553, 554, 557, 558, 561, and 585 of SEQ ID NO:70, and at about positions 409, 477, 478, 480, 481, 484, and 508 of SEQ ID NO:73); and has one or more of the conserved choline transporter domain consensus sequences described herein. In still another embodiment, the 59914 and 59921 polypeptides or proteins have a conserved choline transporter domain which includes at least about 50-250, preferably about 75-175, more preferably about 100-150, and most preferably about 119-121 amino acid residues in length; has at least about 60%, 70%, 80%, 90%, 95%, 99%, or 100% homology with a conserved choline transporter domain, e.g., the conserved choline transporter domain of human 59914 or 59921 (e.g., residues 479-598 of SEQ ID NO:70 or residues 402-521 of SEQ ID NO:73); has about 1-10, preferably about 2-8, and more preferably about 3-7 conserved cysteine residues (e.g., at about positions 553, 554, 557, 558, 561, and 585 of SEQ ID NO:70, and at about positions 409, 477, 478, 480, 481, 484, and 508 of SEQ ID NO:73); has one or more of the conserved choline transporter domain consensus sequences described herein; and has at least one 59914 and 59921 biological activity as described herein

In one embodiment, 59914 and 59921 proteins include at least ten transmembrane domains. As used herein, the term “transmembrane domain” includes an amino acid sequence of about 5 amino acid residues in length that spans the plasma membrane. More preferably, a transmembrane domain includes about at least 10, 15, 20 or 22-25 amino acid residues and spans a membrane. Transmembrane domains are rich in hydrophobic residues, and typically have an alpha-helical structure. In a preferred embodiment, at least 50%, 60%, 70%, 80%, 90%, or 95% or more of the amino acids of a transmembrane domain are hydrophobic, e.g., leucines, isoleucines, tyrosines, or tryptophans. Transmembrane domains are described in, for example, Zagotta W. N. et al. (1996) Annu. Rev. Neurosci. 19:235-263, the contents of which are incorporated herein by reference. Transmembrane domains exist at least from about amino acid residues 39-61, 242-263, 270-287, 326-346, 371-395, 461-484, 514-536, 591-605, 608-632, and 649-672 of SEQ ID NO:70, and at least from about amino acid residues 33-57, 215-231, 239-262, 284-305, 328-352, 384-411, 436-458, 514-532, 534-555, and 563-586 of SEQ ID NO:73.

A 59914 and 59921 family member can include at least one conserved choline transporter domain. Furthermore, a 59914 and 59921 family member can include at least one, preferably at least 5, more preferably at least 9, and still more preferably 10 transmembrane domains; at least one, preferably 5-7, N-glycosylation sites; at least one, preferably 7-10, protein kinase C phosphorylation sites; at least one, preferably 8-9 casein kinase II phosphorylation sites; and at least one, preferably 7-10 N-myristoylation sites.

59914 and 59921 are homologous to human CTL1 (Genbank accession number CAB75541; SEQ ID NO:75) and human CTL2 (Genbank accession number CAB75542; SEQ ID NO:76), both human choline transporter-like proteins known in the art. An alignment of hCTL1 with the amino acid sequence of 59914 (SEQ ID NO:70) reveals 39.7% identity and 29.4% homology. An alignment of hCTL2 with the amino acid sequence of 59914 (SEQ ID NO:70) reveals 66.6% identity and 54.8% homology. An alignment of hCTL1 with the amino acid sequence of 59921 (SEQ ID NO:73) reveals 57.3% identity and 47.7% homology. An alignment of hCTL2 with the amino acid sequence of 59921 (SEQ ID NO:73) reveals 41.5% identity and 29.6% homology. [The alignments described in this paragraph were performed using the GAP alignment program with a BLOSUM62 scoring matrix, a gap open penalty of 12, and a gap extend penalty of 4.]

Like the 59914 and 59921 proteins, hCTL1 and hCTL2 contain a conserved choline transporter domain (from about amino acid residues 407-525 of SEQ ID NO:75 and about amino acid residues 468-587 of SEQ ID NO:76, respectively); 10 transmembrane domains, as described in O'Regan, supra; and 10 conserved cysteines, as described in O'Regan, supra.

59914 and 59921 are also homologous to rat and torpedo (marbled electric ray) choline transporter like proteins (rCTL1 (Genbank accession number CAB75555; SEQ ID NO:77) and tCTL1 (Genbank accession number CAB75556; SEQ ID NO:78), respectively). rCTL1 and tCTL1 are described in O'Regan, supra, and were discovered in the context of suppressing a yeast choline transport mutation (the addition of tCTL1 to yeast increased high-affinity choline uptake in mutant yeast). An alignment of rCTL1 with the amino acid sequence of 59914 (SEQ ID NO:70) reveals 39.5% identity and 29.2% homology. An alignment of tCTL1 with the amino acid sequence of 59914 (SEQ ID NO:70) reveals 40.2% identity and 29.3% homology. An alignment of rCTL1 with the amino acid sequence of 59921 (SEQ ID NO:73) reveals 57.5% identity and 47.4% homology. An alignment of tCTL1 with the amino acid sequence of 59921 (SEQ ID NO:73) reveals 56.3% identity and 45.2% homology. [The alignments described in this paragraph were performed using the GAP alignment program with a BLOSUM62 scoring matrix, a gap open penalty of 12, and a gap extend penalty of 4.]

Like the 59914 and 59921 proteins, rCTL1 and tCTL1 contain a conserved choline transporter domain (from about amino acid residues 406-524 of SEQ ID NO:77 and about amino acid residues 399-518 of SEQ ID NO:78, respectively); 10 transmembrane domains, as described in O'Regan, supra; and 10 conserved cysteines, as described in O'Regan, supra.

Based on the above described sequence similarities, the 59914 and 59921 molecules of the present invention belong to the choline transporter family (as described herein). Consequently, the 59914 and 59921 molecules of the invention have similar biological activities as choline transporter family members, and are useful in treating the same disorders as choline transporter family members.

Based on sequence similarities of 59914 and 59921 to sequences of known expression pattern, 59914 and 59921 molecules of the invention can exhibit similar expression patterns, and therefore can be useful in treating disorders associated with tissues in which they are expressed.

Because the 59914 and 59921 polypeptides of the invention can modulate 59914 and 59921-mediated activities, they can be used as novel diagnostic and therapeutic agents or used to develop novel diagnostic and therapeutic agents for 59914 and 59921-mediated or related disorders (e.g., disorders associated with choline transporter family members), as described below.

As used herein, a “59914 and 59921 activity”, “biological activity of 59914 and 59921”, or “functional activity of 59914 and 59921”, refers to an activity of a choline transporter family member, and refers to an activity exerted by 59914 and 59921 proteins, polypeptides or nucleic acid molecules on, for example, 59914 and 59921-responsive cells or on 59914 and 59921 substrates (e.g., protein substrates) as determined in vivo or in vitro. In one embodiment, a 59914 and 59921 activity is a direct activity, such as association with 59914 and 59921 target molecules. “Target molecules” or “binding partners” of 59914 and 59921 proteins are molecules with which the 59914 and 59921 proteins bind or interact in nature. In an exemplary embodiment, such target molecules include choline, its metabolites, and/or compounds of which choline is a component or precursor, e.g., which 59914 and 59921 proteins can transport into cells from the extracellular fluid, e.g., for plasma membrane synthesis.

A 59914 and 59921 activity can also be an indirect activity, such as an activity mediated by interaction of the 59914 and 59921 protein with a 59914 and 59921 target molecule such that the target molecule modulates a downstream cellular activity, e.g., a cellular signaling activity modulated indirectly by interaction of the 59914 and 59921 protein with a 59914 and 59921 target molecule (e.g., choline, its metabolites, and/or compounds of which choline is a component or precursor).

For example, the 59914 and 59921 proteins of the present invention can have one or more of the following activities: (1) the ability to modulate (e.g., promote, catalyze, regulate, initiate, facilitate or inhibit) the manufacture of choline metabolites and/or compounds of which choline is a component or precursor, e.g., phospholipids (e.g., phosphatidylcholine (lecithin), sphingomyelin, sphingophosphorylcholine, and platelet activating factor), acetylcholine, very low density lipoproteins (VLDLs), and betaine, e.g., by transporting choline into or out of cells; (2) the ability to modulate (e.g., promote, catalyze, regulate, initiate, facilitate or inhibit) transport of choline, its metabolites, and/or compounds of which choline is a component or precursor across membranes (e.g., plasma membranes), e.g., from an extracellular medium into a cell, or vice versa; (3) the ability to modulate (e.g., promote, catalyze, regulate, initiate, facilitate or inhibit) transport of choline, its metabolites, and/or compounds of which choline is a component or precursor across barriers between tissues (e.g., the blood-brain barrier).

Other activities of the 59914 and 59921 proteins of the present invention include one or more of the following: (1) the ability to modulate (e.g., promote, regulate, initiate, facilitate or inhibit) the synthesis of, and the structural maintenance and reinforcement of, cellular components (e.g., membranes (e.g., plasma membranes) and microsomes); (2) the ability to modulate (e.g., promote, regulate, initiate, facilitate or inhibit) cellular nutrition; (3) the ability to modulate (e.g., promote, regulate, initiate, facilitate or inhibit) muscle control; (4) the ability to modulate (e.g., promote, regulate, initiate, facilitate or inhibit) memory; and (5) the ability to modulate (e.g., promote, regulate, initiate, facilitate or inhibit) message transmission (e.g., nervous system message transmission).

Still other activities of the 59914 and 59921 proteins of the present invention include one or more of the following: (1) the ability to modulate (e.g., promote, regulate, initiate, facilitate or inhibit) liver homeostasis, e.g., by transporting fat and/or cholesterol from the liver, e.g., by modulating the transport of choline, its metabolites, and/or compounds of which choline is a component or precursor (e.g., VLDLs); and (2) the ability to modulate (e.g., promote, regulate, initiate, facilitate or inhibit) cellular signaling, e.g., by modulating the transport of choline, its metabolites, and/or compounds of which choline is a component or precursor (e.g., sphingophosphorylcholine and platelet activating factor).

Other activities of the 59914 and 59921 proteins of the present invention include one or more of the following: (1) the ability to modulate (e.g., promote, regulate, initiate, facilitate or inhibit) liver disorders (e.g., hepatocyte apoptosis and others described herein), e.g., by maintaining proper choline levels (e.g., by preventing choline deficiency), e.g., by modulating the transport of choline, its metabolites, and/or compounds of which choline is a component or precursor; (2) the ability to modulate (e.g., promote, regulate, initiate, facilitate or inhibit) central nervous system (CNS) disorders (e.g., hepatocyte apoptosis, and others described herein), e.g., by maintaining proper choline levels (e.g., by preventing choline excess), e.g., by modulating the transport of choline, its metabolites, and/or compounds of which choline is a component or precursor; and (3) the ability to modulate (e.g., promote, regulate, initiate, facilitate or inhibit) cardiovascular disorders, e.g., by preventing buildup of homocysteines in the blood (e.g., by converting them to methionine) e.g., by modulating the transport of choline, its metabolites, and/or compounds of which choline is a component or precursor (e.g., betaine).

Other activities, as described below, include the ability to modulate function, survival, morphology, proliferation and/or differentiation of cells of tissues in which 59914 and 59921 molecules are expressed. Thus, the 59914 and 59921 molecules can act as novel diagnostic targets and therapeutic agents for controlling disorders involving aberrant activities of these cells. 59914 and 59921 molecules described herein can act as novel diagnostic targets and therapeutic agents for prognosticating, diagnosing, preventing, inhibiting, alleviating, or curing choline transporter-related disorders. As the 59914 and 59921 molecules of the invention can modulate choline transporter activities, they are useful for developing novel diagnostic and therapeutic agents for 59914 and 59921-mediated or related disorders, as described herein.

As used herein, a “choline transporter disorder” includes a disorder, disease or condition which is caused by, characterized by, or associated with a misregulation (e.g., an aberrant downregulation or upregulation) of an choline transporter activity or an abnormal choline transporter activity. Choline transporter disorders can detrimentally affect cellular functions such as amino acid nutrition, cellular regulation of homeostasis, membrane structural integrity, and inter- or intra-cellular communication.

Accordingly, the 59914 and 59921 molecules of the invention, as choline transporters, can mediate, and can act as novel diagnostic targets and therapeutic agents for controlling, one or more choline transporter-associated disorders, including CNS-related (e.g., neurological) disorders; liver-related (i.e., hepatic) disorders; skeletal muscle-related disorders; lung-related (i.e., pulmonary) disorders, prostate-related disorders, kidney-related (i.e., renal) disorders, pancreas-related disorders, colon-related disorders, cellular proliferative and/or differentiative disorders; hormonal disorders; immune and inflammatory disorders; cardiovascular disorders; blood vessel disorders; and platelet disorders.

Further, polymorphisms associated with particular 59914 and 59921 alleles, such as those associated with risk of choline transporter-associated disorders, can be used as markers to diagnose abnormal function of tissues and/or cells in which 59914 and 59921 are expressed (described herein), and therefore can be used as markers for disorders associated with such tissues. For example, abnormal and/or aberrant 59914 and 59921 expression (e.g., expression of 59914 and 59921 in cells, such as tumor cells, that do not normally express them, or increased expression of 59914 and 59921 in cells that do normally express them) can be used as a marker for the progression, migration and metastasis of cancerous cells. In particular, abnormal and/or aberrant 59914 and 59921 expression can be used as a marker for the progression, migration and metastasis of cancers of the tissues and/or cells in which 59914 and 59921 are expressed (described herein).

Additional choline transporter disorders include CNS-related (e.g., neurological) disorders, hepatic disorders, skeletal muscle-related disorders, pulmonary (lung) disorders, prostate disorders, renal (kidney) disorders, pancreatic disorders and colonic disorders.

The 59914 and 59921 molecules of the invention can be used to monitor, treat and/or diagnose a variety of proliferative disorders. Such disorders include hematopoietic neoplastic disorders.

Choline transporter disorders can include hormonal disorders, such as conditions or diseases in which the production and/or regulation of hormones in an organism is aberrant.

Choline transporter disorders also include immune disorders, such as autoimmune disorders or immune deficiency disorders.

### Gene Expression of 59914 and 59921

TaqMan analysis indicates significant 59914 expression in normal brain cortex; moderate 59914 expression in human umbilical vein endothelial cells (HUVEC), prostate tumor and lung tumor; low levels of 59914 expression in colon tumor, kidney, and hypothalamus. It also indicates significant 59921 expression in kidney, pancreas, and colon tumor; and low to moderate 59921 levels of expression in spinal cord, hypothalamus, nerve, dorsal root ganglia, prostate tumor, lung tumor, salivary glands, and liver fibrosis

### Human 33751

Nucleotide and corresponding amino acid sequences for an ion channel family member, referred to herein as “33751” are disclosed. 33751 protein is a member of a family of voltage-gated potassium channel genes that includes the eag, erg, and elk genes.

The human 33751 sequence (SEQ ID NO:81), which is approximately 4113 nucleotides long including untranslated regions, contains a predicted methionine-initiated coding sequence of about 3588 nucleotides long (nucleotides 101-3688 of SEQ ID NO:81; 1-3588 of SEQ ID NO:83), not including the termination codon. The coding sequence encodes a 1196 amino acid protein (see SEQ ID NO:82).

Human 33751 contains the following regions or other structural features: one predicted ion transport protein domain (PFAM Accession Number PF00520) located at about amino acid residues 450 to 662 of SEQ ID NO:82; one predicted PAS domain (PFAM Accession Number PF00989) located at about amino acids 41 to 60 of SEQ ID NO:82; one predicted PAC domain (PFAM Accession Number PF00785) located at about amino acids 93 to 120 of SEQ ID NO:82; one predicted cyclic nucleotide-binding domain (PFAM Accession Number PF00027) located at about amino acids 760 to 850 of SEQ ID NO:82; six predicted transmembrane segments located at about amino acids 412 to 433, 453 to 470, 495 to 513, 549 to 573, 614 to 630, and 642 to 666 of SEQ ID NO:82; one predicted N-terminal cytoplasmic domain located at about amino acids 1 to 411 of SEQ ID NO:82; one predicted C-terminal cytoplasmic domain located at about amino acids 667 to 1196 of SEQ ID NO:82; two predicted cytoplasmic loops located at about amino acids 471 to 494, and 574 to 613 of SEQ ID NO:82; three predicted extracellular loops located at about amino acids 434 to 452, 514 to 548, and 631 to 641 of SEQ ID NO:82; ten predicted N-glycosylation sites (PS00001) located at about amino acids 102 to 105, 230 to 233, 338 to 341, 369 to 372, 600 to 603, 661 to 664, 736 to 739, 881 to 884, 905 to 908, and 1139 to 1142 of SEQ ID NO:82; four predicted cAMP- and cGMP-dependent protein kinase phosphorylation sites (PS00004) located at about amino acids 171 to 174, 271 to 274, 346 to 349, and 893 to 896 of SEQ ID NO:82; twenty-one predicted Protein Kinase C phosphorylation sites (PS00005) located at about amino acids 74 to 76, 169 to 171, 187 to 189, 239 to 241, 269 to 271, 344 to 346, 354 to 356, 371 to 373, 392 to 394, 528 to 530, 582 to 584, 609 to 611, 639 to 641, 673 to 675, 869 to 871, 916 to 918, 922 to 924, 974 to 976, 985 to 987, 1096 to 1098, and 1099 to 1101 of SEQ ID NO:82; twenty-four predicted Casein Kinase II phosphorylation sites (PS00006) located at about amino acids 55 to 58, 133 to 136, 209 to 212, 275 to 278, 317 to 320, 514 to 517, 609 to 612, 637 to 640, 793 to 796, 821 to 824, 829 to 832, 857 to 860, 879 to 882, 883 to 886, 899 to 902, 906 to 909, 939 to 942, 963 to 966, 985 to 988, 1020 to 1023, 1027 to 1030, 1091 to 1094, 1134 to 1137, and 1170 to 1173 of SEQ ID NO:82; three predicted Tyrosine kinase phosphorylation sites (PS00007) located at about amino acids 92 to 99, 241 to 248, and 440 to 446 of SEQ ID NO:82; ten predicted N-myristylation sites (PS00008) located at about amino acids 515 to 520, 524 to 529, 592 to 597, 660 to 665, 713 to 718, 748 to 753, 951 to 956, 958 to 963, 1018 to 1023, and 1129 to 1134 of SEQ ID NO:82; and one predicted Amidation site (PS00009) located at about amino acids 595 to 598 of SEQ ID NO:82.

For general information regarding PFAM identifiers, PS prefix and PF prefix domain identification numbers, refer to Sonnhammer et al. (1997) Protein 28:405-420.

TABLE 15
Summary of transmembrane domains.

| Transmembrane | | Extracellular |
|---|---|---|
| segment | | sequence |
| located in | Cytoplasmic sequence located | located in SEQ ID |
| SEQ ID NO: 82 | in SEQ ID NO: 82 | NO: 82 |
| about 412 to 433 | about 1 to 411 (N-terminal) | |
| about 453 to 470 | | about 434 to 452 |
| about 495 to 513 | about 471 to 494 | |
| about 549 to 573 | | about 514 to 548 |
| about 614 to 630 | about 574 to 613 | |
| about 642 to 666 | | about 631 to 641 |
| | about 667 to 1196 (C-terminal) | |

A hydropathy plot of human 33751 was performed. Polypeptides of the invention include fragments which include: all or part of a hydrophobic sequence, e.g., the sequence from about amino acid 125 to 132, from about 380 to 388, and from about 782 to 790 of SEQ ID NO:82; all or part of a hydrophilic sequence, e.g., the sequence of from about amino acid 72 to 84, from about 285 to 310, and from about 880 to 902 of SEQ ID NO:82; a sequence which includes a Cys, or a glycosylation site.

The 33751 protein contains a significant number of structural characteristics in common with members of the potassium channel family. As used herein, a “potassium channel” includes a protein or polypeptide that is involved in receiving, conducting, and transmitting signals in an electrically excitable cell, e.g., a neuronal cell or a muscle cell. Potassium channels are potassium ion selective, and can determine membrane excitability (the ability of, for example, a neuron to respond to a stimulus and convert it into an impulse). Potassium channels can also influence the resting potential of membranes, wave forms and frequencies of action potentials, and thresholds of excitation. Potassium channels are typically expressed in electrically excitable cells, e.g., neurons, muscle, endocrine, and egg cells, and may form heteromultimeric structures, e.g., composed of pore-forming a and cytoplasmic b subunits. Potassium channels may also be found in nonexcitable cells (e.g., thymus cells), where they may play a role in, e.g., signal transduction. Potassium channel proteins contain six transmembrane helices, wherein the last two helices flank a loop (a P-loop) which determines potassium ion selectivity. Examples of potassium channels include: (1) the voltage-gated potassium channels, (2) the ligand-gated potassium channels, e.g., neurotransmitter-gated potassium channels, and (3) cyclic-nucleotide-gated potassium channels. Voltage-gated and ligand-gated potassium channels are expressed in the brain, e.g., in brainstem monoaminergic and forebrain cholinergic neurons, where they are involved in the release of neurotransmitters, or in the dendrites of hippocampal and neocortical pyramidal cells, where they are involved in the processes of learning and memory formation. For a detailed description of potassium channels, see Kandel E. R. et al., Principles of Neural Science, second edition, (Elsevier Science Publishing Co., Inc., N.Y. (1985)), the contents of which are incorporated herein by reference.

33751 polypeptides belong to a small gene family of voltage-gated potassium channels that includes the eag, erg, and elk genes (Shi, W. et al. (1997) supra). These genes have been described either Drosophila or mammals (Warmke J, Ganetzky B (1994) Proc Natl Acad Sci USA 91:3438-3442; Ludwig J. et al. (1994) EMBO J. 13:4451-4458; Warmke J, Drysdale R, Ganetzky B (1991) Science 252:1560-1562; Titus S A, Warmke J W, Ganetzky B (1997) J Neurosci 17:875-881). The 33751 polypeptides are highly homologous to the human erg1, and rat erg2 and erg3 previously identified (Shi, W. et al. (1997) supra). These channels share the six membrane-spanning architecture of the Kv class (Shaker-related) of voltage-gated potassium channels, but otherwise are distantly related to the Kv class channels. The channels encoded by the eag-related genes are relatively slowly activating, as compared with Kv class potassium channels (Ludwig J. et al. (1994) supra), and have some similarities to slowly activating potassium currents that are important in determining the threshold firing properties of neurons (Brown D A (1988) M currents. In: Ion channels (Narahashi T, ed), pp 55-94. New York: Plenum; Yamada W M et al. (1989) Multiple channels and calcium dynamics. In: Methods in neutronal modeling (Koch C, Segev I, eds), pp 97-133. Cambridge, Mass.: Bradford; Wang H S, McKinnon D (1996) J Physiol (Lond) 492:467-478). As might be anticipated from the biophysical properties of these channels, mutations in either the eag gene or the erg gene of Drosophila result in a hyperexcitable phenotype (Titus et al. (1997) supra; Wang et al. (1997) supra.

Accordingly, 33751-activity may be involved in neurological processes, including PLC-mediated conductances associated with the propagation of action potentials, synaptic transmission, as well nociceptive responses, and neuropathic pain, as described in more detail below.

A 33751 polypeptide can include an “ion transport protein domain”, or regions homologous with an “ion transport protein domain.”

As used herein, the term “ion transport protein domain” includes an amino acid sequence of about 150 to 280 amino acid residues in length and having a bit score for the alignment of the sequence to the ion transport protein domain profile (Pfam HMM) of at least 50. Preferably, an ion transport protein domain includes at least about 180 to 250 amino acids, more preferably about 200 to 220 amino acid residues, or about 213 amino acids and has a bit score for the alignment of the sequence to the ion transport protein domain (HMM) of at least 60, preferable 80, 95 or greater. The ion transport protein domain (HMM) has been assigned the PFAM Accession Number PF00520. The ion transport protein domain (amino acids 450 to 662 of SEQ ID NO:82) of human 33751 aligns with a consensus amino acid sequence (SEQ ID NO:84) derived from a hidden Markov model.

In a preferred embodiment 33751 polypeptide or protein has an “ion transport protein domain” or a region which includes at least about 150 to 280, more preferably about 180 to 250, or 200 to 220 amino acid residues and has at least about 50%, 60%, 70% 80% 90% 95%, 99%, or 100% homology with an “ion transport protein domain,” e.g., the ion transport protein domain of human 33751 (e.g., residues 450 to 662 of SEQ ID NO:82).

To identify the presence of an “ion transport protein domain” in a 33751 protein sequence, and make the determination that a polypeptide or protein of interest has a particular profile, the amino acid sequence of the protein can be searched against the Pfam database of HMMs (e.g., the Pfam database, release 2.1) using the default parameters. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3): 405-420 and a detailed description of HMMs can be found, for example, in Gribskov et al. (1990) Meth. Enzymol. 183: 146-159; Gribskov et al. (1987) Proc. Natl. Acad. Sci. USA 84: 4355-4358; Krogh et al. (1994) J. Mol. Biol. 235: 1501-1531; and Stultz et al. (1993) Protein Sci. 2: 305-314, the contents of which are incorporated herein by reference. A search was performed against the HMM database resulting in the identification of an “ion transport protein domain” in the amino acid sequence of human 33751 at about residues 450 to 662 SEQ ID NO:82.

A 33751 polypeptide can further include a “PAS domain” or regions homologous with a “PAS domain.” A PAS domain appears in archaea, eubacteria and eukarya. PAS domain have been found in EAG-like K+-channels.

As used herein, a “PAS domain” includes an amino acid sequence of about 10 to 100 amino acid residues in length that is involved in ligand and/or protein-protein interactions. Preferably, the PAS domain interacts with the body of the channel, affecting gating, inactivation, and/or voltage sensitivity. Preferably, the PAS domain is located at the N-terminal cytoplasmic region of the 33751 polypeptide. The term “PAS domain” includes an amino acid sequence of about 10 to 100 amino acid residues in length and having a bit score for the alignment of the sequence to the cyclic nucleotide binding domain (HMM) of at least 5. Preferably, a PAS domain includes at least about 10 to 100 amino acids, more preferably about 13 to 50 amino acid residues, or about 17 to 25 amino acids and has a bit score for the alignment of the sequence to the PAS domain (HMM) of at least 6 or greater. The PAS domain (HMM) has been assigned the PFAM Accession PF00989. The PAS domain (amino acids 41 to 60 of SEQ ID NO:82) of human 33751 aligns with a consensus amino acid sequence (SEQ ID NO:85) derived from a hidden Markov model.

To identify the presence of a “PAS” domain in a 33751 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (200) Nucl. Acids Res 28:231. The database contains domains identified by profiling with the hidden Markov models of the HMMer2 search program (R. Durbin et al. (1998) Biological sequence analysis: probabilistic models of proteins and nucleic acids. Cambridge University Press). The database also is extensively annotated and monitored by experts to enhance accuracy. A search was performed against the HMM database resulting in the identification of a “PAS” domain in the amino acid sequence of human 33751 at about residues 41 to 60 of SEQ ID NO:82.

In a preferred embodiment, a 33751 polypeptide or protein has a “PAS domain” or a region which includes at least about 10 to 100, more preferably about 13 to 50, or 17 to 25 amino acid residues and has at least about 50%, 60%, 70% 80% 90% 95%, 99%, or 100% homology with a “PAS domain,” e.g., the PAS domain of human 33751 (e.g., residues 41 to 60 of SEQ ID NO:82).

A 33751 polypeptide can further include a “PAC domain” or regions homologous with a “PAC domain.” As used herein, a “PAC domain” includes an amino acid sequence of about 10 to 100 amino acid residues in length. Preferably, the PAC domain contributes to the folding of the PAS domain. Preferably, the PAC domain is located at the C-terminal end of the PAS domain in a 33751 polypeptide. The term “PAC domain” includes an amino acid sequence of about 10 to 100 amino acid residues in length and having a bit score for the alignment of the sequence to the cyclic nucleotide binding domain (HMM) of at least 10. Preferably, a PAC domain includes at least about 10 to 100 amino acids, more preferably about 20 to 50 amino acid residues, or about 25 to 30 amino acids and has a bit score for the alignment of the sequence to the PAC domain (HMM) of at least 15 or greater. The PAC domain (HMM) has been assigned the PFAM Accession PF00785. The PAC domain (amino acids 93 to 120 of SEQ ID NO:82) of human 33751 aligns with a consensus amino acid sequence (SEQ ID NO:86) derived from a hidden Markov model.

To identify the presence of a “PAC” domain in a 33751 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (200) Nucl. Acids Res 28:231. The database contains domains identified by profiling with the hidden Markov models of the HMMer2 search program (R. Durbin et al. (1998) Biological sequence analysis: probabilistic models of proteins and nucleic acids. Cambridge University Press). The database also is extensively annotated and monitored by experts to enhance accuracy. A search was performed against the HMM database resulting in the identification of a “PAC” domain in the amino acid sequence of human 33751 at about residues 93 to 120 of SEQ ID NO:82.

In a preferred embodiment, a 33751 polypeptide or protein has a “PAC domain” or a region which includes at least about 10 to 100, 20 to 50, or 25 to 30 amino acid residues and has at least about 50%, 60%, 70% 80% 90% 95%, 99%, or 100% homology with a “PAC domain,” e.g., the PAC domain of human 33751 (e.g., residues 93 to 120 of SEQ ID NO:82).

A 33751 molecule can further include a cyclic nucleotide binding domain or regions homologous with a “cyclic nucleotide binding domain.” As used herein, the term “cyclic nucleotide binding domain” includes an amino acid sequence of about 50 to 200 amino acid residues in length and having a bit score for the alignment of the sequence to the cyclic nucleotide binding domain (HMM) of at least 50. Preferably, a cyclic nucleotide binding domain is capable of binding a cyclic nucleotide (e.g., cAMP or cGMP), and is composed of three I-helices and a distinctive eight-stranded anti-parallel J-barrel structure. Preferably, a cyclic nucleotide binding domain includes at least about 50 to 200 amino acids, more preferably about 70 to 120 amino acid residues, or about 85 to 95 amino acids and has a bit score for the alignment of the sequence to the cyclic nucleotide binding domain (HMM) of at least 75 or greater. The cyclic nucleotide binding domain (HMM) has been assigned the PFAM Accession PF00027. The cyclic nucleotide binding domain (amino acids 760 to 850 of SEQ ID NO:82) of human 33751 aligns with a consensus amino acid sequence (SEQ ID NO:87) derived from a hidden Markov model.

To identify the presence of a “cyclic nucleotide binding” domain in a 33751 protein sequence, and make the determination that a polypeptide or protein of interest has a particular profile, the amino acid sequence of the protein can be searched against a SMART database (Simple Modular Architecture Research Tool) of HMMs as described in Schultz et al. (1998), Proc. Natl. Acad. Sci. USA 95:5857 and Schultz et al. (200) Nucl. Acids Res 28:231. The database contains domains identified by profiling with the hidden Markov models of the HMMer2 search program (R. Durbin et al. (1998) Biological sequence analysis: probabilistic models of proteins and nucleic acids. Cambridge University Press.) The database also is extensively annotated and monitored by experts to enhance accuracy. A search was performed against the HMM database resulting in the identification of a “cyclic nucleotide binding” domain in the amino acid sequence of human 33751 at about residues 760 to 850 of SEQ ID NO:82.

In a preferred embodiment a 33751 polypeptide or protein has a “cyclic nucleotide binding domain” or a region which includes at least about 50 to 200, more preferably about 70 to 120, or 85 to 95 amino acid residues and has at least about 50%, 60%, 70% 80% 90% 95%, 99%, or 100% homology with a “cyclic nucleotide binding domain,” e.g., the cyclic nucleotide binding domain of human 33751 (e.g., residues 760 to 850 of SEQ ID NO:82).

A 33751 protein further includes a predicted N-terminal cytoplasmic domain located at about amino acids 1-411 of SEQ ID NO:82. As used herein, a “N-terminal cytoplasmic domain” includes an amino acid sequence having about 1 to 600, preferably about 1 to 500, or even more preferably about 1 to 420 amino acid residues in length and is located inside of a cell or intracellularly. The C-terminal amino acid residue of a “N-terminal cytoplasmic domain” is adjacent to a N-terminal amino acid residue of a transmembrane domain in a 33751 protein. For example, a N-terminal cytoplasmic domain is located at about amino acid residues 1 to 411 of SEQ ID NO:82.

In a preferred embodiment 33751 polypeptide or protein has an “N-terminal cytoplasmic domain” or a region which includes at least about 1 to 600, preferably about 100 to 420, and even more preferably about 411 amino acid residues and has at least about 60%, 70% 80% 90% 95%, 99%, or 100% homology with an “N-terminal cytoplasmic domain,” e.g., the N-terminal cytoplasmic domain of human 33751 (e.g., residues (1 to 411 of SEQ ID NO:82).

In another embodiment, a 33751 protein includes a “C-terminal cytoplasmic domain,” also referred to herein as a C-terminal cytoplasmic tail, in the sequence of the protein. As used herein, a “C-terminal cytoplasmic domain” includes an amino acid sequence having a length of at least about 200, more preferably 400 or more amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring 33751 protein. For example, a C-terminal cytoplasmic domain is found at about amino acid residues 667 to 1196 of SEQ ID NO:82.

In a preferred embodiment, a 33751 polypeptide or protein has a C-terminal cytoplasmic domain or a region which includes at least about 200, more preferably 400 or more amino acid residues and has at least about 60%, 70% 80% 90% 95%, 99%, or 100% homology with an “C-terminal cytoplasmic domain,” e.g., the C-terminal cytoplasmic domain of human 33751 (e.g., residues 667 to 1196 of SEQ ID NO:82).

33751 proteins can further include at least one, two, three, four, five, and preferably six transmembrane domains. As used herein, the term “transmembrane domain” includes an amino acid sequence of about 10 to 45, preferably 12 to 30, and most preferably 15 to 25, amino acid residues in length that spans the plasma membrane. More preferably, a transmembrane domain includes about at least 17, 18, 19, 22, or 25 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta W. N. et al, (1996) Annual Rev. Neurosci. 19: 235-263, the contents of which are incorporated herein by reference. Amino acid residues 412 to 433, 453 to 470, 495 to 513, 549 to 573, 614 to 630, and 642 to 666 of SEQ ID NO:82 are transmembrane domains. Accordingly, proteins having at least 50-60% homology, preferably about 60-70%, more preferably about 70-80%, about 80-90%, or about 90-100% homology with amino acids 412 to 433, 453 to 470, 495 to 513, 549 to 573, 614 to 630, and 642 to 666 of SEQ ID NO:82 are within the scope of the invention.

In another embodiment, a 33751 protein includes at least one, or two cytoplasmic loop, also referred to herein as a cytoplasmic domain. As used herein, a “cytoplasmic loop” includes an amino acid sequence having a length of at least about 10, preferably about 20, amino acid residues located within a cell or within the cytoplasm of a cell. For example, a cytoplasmic loop is found at about amino acids 471 to 494, or 574 to 613 of SEQ ID NO:82.

In a preferred embodiment 33751 polypeptide or protein has at least one cytoplasmic loop or a region which includes at least about 10, preferably about 20 amino acid residues and has at least about 60%, 70% 80% 90% 95%, 99%, or 100% homology with an “cytoplasmic loop,” e.g., at least one cytoplasmic loop of human 33751 (e.g., residues 471 to 494, or 574 to 613 of SEQ ID NO:82).

In another embodiment, a 33751 protein include at least one, two, or three extracellular loop. As defined herein, the term “loop” includes an amino acid sequence that resides outside of a phospholipid membrane, having a length of at least about 20 to 70, and preferably about 30 to 50 amino acid residues, and has an amino acid sequence that connects two transmembrane domains within a protein or polypeptide. Extracellular domains are located outside of the cell. Accordingly, the N-terminal amino acid of a non-cytoplasmic loop is adjacent to a C-terminal amino acid of a transmembrane domain in a 33751 protein, and the C-terminal amino acid of a non-cytoplasmic loop is adjacent to an N-terminal amino acid of a transmembrane domain in a 33751 protein. For example, an “extracellular loop” can be found at about amino acids 434 to 452, 514 to 548, and 631 to 641 of SEQ ID NO:82.

In a preferred embodiment, a 33751 polypeptide or protein has at least one, two, or three extracellular loops or regions which include at least about 5, preferably about 5 to 80, and more preferably about 20 to 50 amino acid residues and has at least about 60%, 70% 80% 90% 95%, 99%, or 100% homology with an “non-cytoplasmic loop,” e.g., at least one non-cytoplasmic loop of human 33751 (e.g., residues 403 to 433, 493 to 540, and 604 to 645 of SEQ ID NO:82).

Accordingly, in one embodiment of the invention, a 33751 includes at least one, two, three, four, five, preferably six, transmembrane domains, at least one, or two cytoplasmic loops, and/or at least one, two, or three non-cytoplasmic loops. In another embodiment, the 33751 further includes an N-terminal and a C-terminal cytoplasmic domains.

A 33751 family member can include at least one predicted ion transport protein domain, at least one predicted PAS domain, at least one predicted PAC domain, and at least one predicted cyclic nucleotide-binding domain. Furthermore, a 33751 family member can include at least one, two, three, four, five, six, seven, eight, nine, or preferably ten predicted N-glycosylation sites (PS00001); at least one, two, three, or preferably four predicted cAMP- and cGMP-dependent protein kinase phosphorylation sites (PS00004); at least one, two, three, four, five, six, seven, eight, nine, ten, eleven, twelve, thirteen, fourteen, fifteen, sixteen, seventeen, eighteen, nineteen, twenty, or preferably twenty-one predicted protein kinase C phosphorylation sites (PS00005); at least one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or preferably twenty-four predicted casein kinase II phosphorylation sites (PS00006); at least one, two, or preferably three tyrosine kinase phosphorylation sites (PS00007); at least one, two, three, four, five, six, seven, eight, nine, or preferably ten predicted N-myristylation sites (PS00008); and at least one predicted amidation site (PS00009).

As the 33751 polypeptides of the invention may modulate 33751-mediated activities, they may be useful as of for developing novel diagnostic and therapeutic agents for 33751-mediated or related disorders, as described below.

As used herein, a “33751 activity,” “biological activity of 33751,” or “functional activity of 33751,” refers to an activity exerted by a 33751 protein, polypeptide or nucleic acid molecule on e.g., a 33751-responsive cell or on a 33751 substrate, e.g., a protein substrate, as determined in vivo or in vitro. In one embodiment, a 33751 activity is a direct activity, such as an association with a 33751 target molecule. A “target molecule” “substrate” or “binding partner” is a molecule with which a 33751 protein binds or interacts in nature. A 33751 activity can also be an indirect activity, e.g., a cellular signaling activity mediated by interaction of the 33751 protein with a 33751-binding partner. In an exemplary embodiment, 33751 is controlling one or more of membrane excitability, and/or the frequency and pattern of neuronal firing.

Based on the above-described sequence similarities and the tissue distribution described below, the 33751 molecules of the present invention are predicted to have similar biological activities as potassium channel family members. Thus, a 33751 potassium channel or subsequence or variant polypeptide may have one or more of the aforesaid domains and, therefore, one or more activities or functions characteristic of a potassium channel family member, including, but not limited to, (1) controlling neurotransmitter release from neurons; (2) modulating repolarization of the neuronal cell membrane; (3) contributing to the formation of voltage-gated potassium channels; (4) binding to cyclic nucleotides; (5) regulating nociceptive responses; (6) regulating synaptic transmission; (7) modulating pain or inflammation response; or (8) regulating the frequency and pattern of neuronal firing. Thus, the 33751 molecules can act as novel diagnostic targets and therapeutic agents for controlling potassium channel associated disorders.

Activation of K+ channels affects the frequency and the pattern of neuronal, firing. Several voltage-gated K+ channels are expressed in subpopulation of sensory neurons including those involved in nociception. It has been shown that the expression of some voltage-gated K+ channels decreases in dorsal root ganglion neurons after axotomy, and that the peak of K+ currents is reduced in sensory neurons during chronic inflammation. Furthermore, administration of K+ channel openers potentiated the antinociception produced by agonists of 1-2-adrenoreceptors or by morphin.

TaqMan experiments show high levels of 33751 mRNA expression in the human brain, followed by the dorsal root ganglion (DRG) and spinal cord (Table 16). A TaqMan experiment detecting mRNA expression of the rat ortholog of human 33751 revealed a similar pattern of expression to that of the human gene. The results indicate that 33751 gene is a nervous system specific gene. Further, TaqMan experiments in a rat model show down-regulation of 33751 mRNA in the DRG after CCI and after axotomy. TaqMan experiments in the rat model show down-regulation of 33751 mRNA in spinal cord after CFA injection and after axotomy (Tables 17-19). In situ hybridization with a human probe shows the expression of 33751 mRNA in monkey and rat brain, spinal cord, and DRG. In the monkey cord, 33751 mRNA is expressed in lamina V in large size neurons, most likely spinothalamic neurons. In the DRG, a small subpopulation of neurons expressed high levels of 33751 mRNA. Another subpopulation of sensory neurons expressed much lower levels of 33751 mRNA. Down-regulation of 33751 was observed by in situ hybridization 14 and 28 days after axotomy. Accordingly, 33751 may be critical for hypersensitivity in different pain states, and thus may represent a unique target for pain.

Animal models of pain response include, but are not limited to, axotomy, the cutting or severing of an axon; chronic constriction injury (CCI), a model of neuropathic pain which involves ligation of the sciatic nerve in rodents, e.g., rats; or intraplantar Freund's adjuvant injection as a model of arthritic pain. Other animal models of pain response are described in, e.g., ILAR Journal (1999) Volume 40, Number 3 (entire issue). TaqMan experiments in rat animal models show no regulation in DRGs. However, 33751 mRNA is up-regulated in the spinal cord after CCI axotomy, and after CFA intraplantar injection. These experiments indicate a role for the 33751 molecule in pain response.

Therefore, 33751-associated disorders can detrimentally affect regulation and modulation of the pain response; and vasoconstriction response and pain therefrom. Examples of 33751 associated disorders in which the 33751 molecules of the invention may be directly or indirectly involved include pain, pain syndromes, and inflammatory disorders, including inflammatory pain, and therefore, modulators of the activity or expression of 33751 polypeptides may be useful for developing novel diagnostic and therapeutic agents for controlling pain, pain disorders, and inflammatory disorders.

Agents that modulate 33751 polypeptide or nucleic acid activity or expression can be used to treat pain elicited by any medical condition. A subject receiving the treatment can be additionally treated with a second agent, e.g., an anti-inflammatory agent, an antibiotic, or a chemotherapeutic agent, to further ameliorate the condition.

The 33751 molecules can also act as novel diagnostic targets and therapeutic agents controlling pain caused by other disorders, e.g., cancer, e.g., prostate cancer. Accordingly, the 33751 molecules can act as novel diagnostic targets and therapeutic agents for controlling one or more of cellular proliferative and/or differentiative disorders, or pain therefrom.

As the 33751 mRNA is highly expressed in the brain, 33751 molecules can also act as novel diagnostic targets and therapeutic agents for brain disorders.

Tissue Distribution of 33751 mRNA by TaqMan Analysis

A TaqMan experiment shows 33751 mRNA was highly expressed in brain, followed by DRG and spinal cord. The relative tissue distribution of 33751 mRNA is depicted in tabular form in Table 16.

The relative tissue distribution of 33751 mRNA using rat panels is depicted in tabular form in Tables 17-19. Expression of rat 33751 mRNA was observed in DRG, brain, SCG, spinal, optic nerve, and thyroid. A TaqMan experiment in an additional rat panel shows down-regulation of 33751 in DRG after CCI and axotomy. A final TaqMan experiment in a rat panel shows down-regulation of 33751 in spinal cord after CFA injection and after axotomy. The relative tissue distribution of 33751 mRNA is depicted in tabular form in Tables 17-19.

TABLE 16

| Tissue Type | 33751 | β2.803 | ∂Ct | Expression |
|---|---|---|---|---|
| Adrenal Gland | 38.89 | 18.04 | 20.86 | 0.00 |
| Brain | 25.27 | 20.21 | 5.06 | 29.98 |
| Heart | 40.00 | 18.22 | 21.78 | 0.00 |
| Kidney | 37.24 | 18.34 | 18.90 | 0.00 |
| Liver | 39.98 | 18.78 | 21.20 | 0.00 |
| Lung | 40.00 | 16.48 | 23.52 | 0.00 |
| Mammary Gland | 40.00 | 17.60 | 22.41 | 0.00 |
| Pancreas | 38.25 | 20.64 | 17.61 | 0.00 |
| Placenta | 37.57 | 18.09 | 19.48 | 0.00 |
| Prostate | 40.00 | 17.47 | 22.54 | 0.00 |
| Salivary Gland | 40.00 | 18.52 | 21.49 | 0.00 |
| Muscle | 40.00 | 20.29 | 19.71 | 0.00 |
| Sm. Intestine | 38.10 | 18.48 | 19.62 | 0.00 |
| Spleen | 40.00 | 16.29 | 23.72 | 0.00 |
| Stomach | 32.67 | 17.76 | 14.91 | 0.03 |
| Teste | 34.00 | 18.88 | 15.13 | 0.03 |
| Thymus | 35.32 | 17.23 | 18.09 | 0.00 |
| Trachea | 40.00 | 18.32 | 21.68 | 0.00 |
| Uterus | 40.00 | 18.56 | 21.45 | 0.00 |
| Spinal Cord | 30.83 | 18.84 | 11.99 | 0.25 |
| DRG | 29.89 | 19.06 | 10.83 | 0.55 |
| Skin | 40.00 | 18.18 | 21.83 | 0.00 |

TABLE 17

| Tissue | r33751 | 18S | ∂Ct | Expression |
|---|---|---|---|---|
| Brain | 35.23 | 12.59 | 22.64 | 0.01 |
| Spinal Cord | 36.91 | 12.39 | 24.52 | 0.00 |
| DRG | 33.27 | 12.07 | 21.20 | 0.03 |
| SCG | 35.79 | 13.00 | 22.79 | 0.01 |
| Optic Nerve | 37.44 | 13.23 | 24.21 | 0.00 |
| Hairy Skin | 40.00 | 14.06 | 25.94 | 0.00 |
| Gastro Muscle | 39.68 | 12.39 | 27.29 | 0.00 |
| Heart | 40.00 | 13.29 | 26.71 | 0.00 |
| Kidney | 40.00 | 13.12 | 26.88 | 0.00 |
| Liver | 40.00 | 12.61 | 27.39 | 0.00 |
| Lung | 40.00 | 13.48 | 26.53 | 0.00 |
| Spleen | 40.00 | 13.12 | 26.88 | 0.00 |
| Aorta | 40.00 | 13.33 | 26.67 | 0.00 |
| Adrenal Gland | 40.00 | 12.75 | 27.25 | 0.00 |
| Salivary Gland | 40.00 | 12.51 | 27.50 | 0.00 |
| Thyroid | 35.88 | 12.46 | 23.43 | 0.01 |
| Prostate | 40.00 | 13.52 | 26.48 | 0.00 |
| Thymus | 40.00 | 12.69 | 27.32 | 0.00 |
| Trachea | 40.00 | 13.92 | 26.09 | 0.00 |
| Esophagus | 40.00 | 12.86 | 27.14 | 0.00 |
| Duodenum | 39.97 | 13.92 | 26.06 | 0.00 |
| Diaphragm | 40.00 | 14.34 | 25.66 | 0.00 |
| Colon | 40.00 | 15.05 | 24.96 | 0.00 |

TABLE 18

| Tissue | r33751 | 18S | ∂Ct | Expression |
|---|---|---|---|---|
| Naïve DRG | 33.84 | 12.83 | 21.01 | 0.005 |
| I DRG CCI 3 | 33.61 | 13.00 | 20.61 | 0.006 |
| I DRG CCI 7 | 35.42 | 13.08 | 22.34 | 0.002 |
| I DRG CCI 10 | 36.74 | 12.98 | 23.77 | 0.001 |
| I DRG CCI 14 | 36.95 | 12.77 | 24.18 | 0.001 |
| I DRG CCI 28 | 36.64 | 12.76 | 23.88 | 0.001 |
| Naïve DRG | 33.88 | 12.86 | 21.02 | 0.005 |
| I DRG CFA 1 | 33.23 | 12.92 | 20.31 | 0.008 |
| I DRG CFA 3 | 34.59 | 13.01 | 21.58 | 0.003 |
| I DRG CFA 7 | 34.19 | 12.57 | 21.62 | 0.003 |
| I DRG CFA 10 | 33.99 | 12.61 | 21.38 | 0.004 |
| I DRG CFA 14 | 34.05 | 12.79 | 21.27 | 0.004 |
| I DRG CFA 28 | 33.34 | 13.13 | 20.21 | 0.008 |
| Naïve DRG | 33.80 | 12.73 | 21.07 | 0.005 |
| I DRG AXT 1 | 32.98 | 12.45 | 20.53 | 0.007 |
| I DRG AXT 3 | 34.30 | 13.35 | 20.96 | 0.005 |
| I DRG AXT 7 | 36.17 | 12.74 | 23.43 | 0.001 |
| I DRG AXT 14 | 35.32 | 12.54 | 22.78 | 0.001 |

TABLE 19

| Tissue | r33751 | 18S | ∂Ct | Expression |
|---|---|---|---|---|
| Naïve SC | 36.49 | 13.55 | 22.94 | 0.001 |
| I SC CCI 3 | 37.02 | 13.56 | 23.46 | 0.001 |
| I SC CCI 7 | 36.66 | 13.56 | 23.11 | 0.001 |
| I SC CCI 10 | 36.14 | 13.76 | 22.38 | 0.002 |
| I SC CCI 14 | 36.96 | 13.26 | 23.70 | 0.001 |
| I SC CCI 28 | 38.19 | 13.34 | 24.85 | 0.000 |
| Naïve SC | 38.79 | 13.10 | 23.98 | 0.001 |
| I SC CFA 1 | 37.10 | 13.73 | 23.38 | 0.001 |
| I SC CFA 3 | 36.24 | 13.18 | 23.07 | 0.001 |
| I SC CFA 7 | 36.66 | 12.73 | 23.93 | 0.001 |
| I SC CFA 10 | 37.94 | 13.26 | 24.68 | 0.000 |
| I SC CFA 14 | 39.17 | 14.04 | 25.13 | 0.000 |
| I SC CFA 28 | 37.23 | 13.79 | 23.45 | 0.001 |
| Naïve SC | 36.76 | 13.61 | 23.15 | 0.001 |
| I SC AXT 1 | 36.13 | 13.50 | 22.63 | 0.002 |
| I SC AXT 3 | 38.53 | 13.16 | 25.37 | 0.000 |
| I SC AXT 7 | 38.02 | 13.11 | 24.92 | 0.000 |
| I SC AXT 14 | 38.84 | 14.04 | 24.80 | 0.000 |

### Electrophysiological Characterization of 33751

Electrophysiological characterization of the 33751 polypeptide was performed. The characteristic pattern of voltage-dependent inactivation of the human 33751 and its rat ortholog was found (Shi, W. et al. (1997) J. Neurosci. Vol. 17(24):9423). The conductance of 33751-expressed in CHOK1 cells maintained moderate depolarizations below −10 mV. 33751 polypeptide showed sustained currents without inactivation. The effect of dofetillide on the membrane potential of 911 cells transiently transfected with 33751 versus vector alone was also studied. In this experiment, 911 cells were placed at 8,000 cells per well, and loaded with a MP dye for 40 minutes at 37° C. 10×30 mM KCl or 0.3-3 uM dofetilide (made up in assay buffer) was added after 3 minute when baseline was read.

### Definitions

The 18607, 15603, 69318, 12303, 48000, 52920, 5433, 38554, 57301, 58324, 55063, 52991, 59914, 59921 or 33751 protein, fragments thereof, and derivatives and other variants of the sequence in SEQ ID NO:2, 6, 14, 19, 22, 25, 28, 33, 36, 39, 54, 57, 61, 70, 73 or 82 thereof are collectively referred to as “polypeptides or proteins of the invention” or “18607, 15603, 69318, 12303, 48000, 52920, 5433, 38554, 57301, 58324, 55063, 52991, 59914, 59921 or 33751 polypeptides or proteins”. Nucleic acid molecules encoding such polypeptides or proteins are collectively referred to as “nucleic acids of the invention” or “18607, 15603, 69318, 12303, 48000, 52920, 5433, 38554, 57301, 58324, 55063, 52991, 59914, 59921 or 33751 nucleic acids.”

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology (1989) John Wiley & Sons, N.Y., 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a 18607, 15603, 69318, 12303, 48000, 52920, 5433, 38554, 57301, 58324, 55063, 52991, 59914, 59921 or 33751 protein, preferably a mammalian 18607, 15603, 69318, 12303, 48000, 52920, 5433, 38554, 57301, 58324, 55063, 52991, 59914, 59921 or 33751 protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of 18607, 15603, 69318, 12303, 48000, 52920, 5433, 38554, 57301, 58324, 55063, 52991, 59914, 59921 or 33751 protein having less than about 30%, 20%, 10% and more preferably 5% (by dry weight), of non-18607, 15603, 69318, 12303, 48000, 52920, 5433, 38554, 57301, 58324, 55063, 52991, 59914, 59921 or 33751 protein (also referred to herein as a “contaminating protein”), or of chemical precursors or non-18607, 15603, 69318, 12303, 48000, 52920, 5433, 38554, 57301, 58324, 55063, 52991, 59914, 59921 or 33751 chemicals. When the 18607, 15603, 69318, 12303, 48000, 52920, 5433, 38554, 57301, 58324, 55063, 52991, 59914, 59921 or 33751 protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of 18607, 15603, 69318, 12303, 48000, 52920, 5433, 38554, 57301, 58324, 55063, 52991, 59914, 59921 or 33751 (e.g., the sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83) without abolishing or more preferably, without substantially altering a biological activity, whereas an “essential” amino acid residue results in such a change. For example, amino acid residues that are conserved among the polypeptides of the present invention, e.g., those present in the conserved domains, are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 18607, 15603, 69318, 12303, 48000, 52920, 5433, 38554, 57301, 58324, 55063, 52991, 59914, 59921 or 33751 protein is preferably replaced with another amino acid residue from the same side chain family. Alternatively, in another embodiment, mutations can be introduced randomly along all or part of a 18607, 15603, 69318, 12303, 48000, 52920, 5433, 38554, 57301, 58324, 55063, 52991, 59914, 59921 or 33751 coding sequence, such as by saturation mutagenesis, and the resultant mutants can be screened for 18607, 15603, 69318, 12303, 48000, 52920, 5433, 38554, 57301, 58324, 55063, 52991, 59914, 59921 or 33751 biological activity to identify mutants that retain activity. Following mutagenesis of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, the encoded protein can be expressed recombinantly and the activity of the protein can be determined.

As used herein, a “biologically active portion” of a 18607, 15603, 69318, 12303, 48000, 52920, 5433, 38554, 57301, 58324, 55063, 52991, 59914, 59921 or 33751 protein includes a fragment of a 18607, 15603, 69318, 12303, 48000, 52920, 5433, 38554, 57301, 58324, 55063, 52991, 59914, 59921 or 33751 protein which participates in an interaction between a 18607, 15603, 69318, 12303, 48000, 52920, 5433, 38554, 57301, 58324, 55063, 52991, 59914, 59921 or 33751 molecule and a non-18607, 15603, 69318, 12303, 48000, 52920, 5433, 38554, 57301, 58324, 55063, 52991, 59914, 59921 or 33751 molecule. Biologically active portions of a 18607, 15603, 69318, 12303, 48000, 52920, 5433, 38554, 57301, 58324, 55063, 52991, 59914, 59921 or 33751 protein include peptides comprising amino acid sequences sufficiently homologous to or derived from the amino acid sequence of the 18607, 15603, 69318, 12303, 48000, 52920, 5433, 38554, 57301, 58324, 55063, 52991, 59914, 59921 or 33751 protein, e.g., the amino acid sequence shown in SEQ ID NO:2, 6, 14, 19, 22, 25, 28, 33, 36, 39, 54, 57, 61, 70, 73 or 82, which include fewer amino acids than the full length 18607, 15603, 69318, 12303, 48000, 52920, 5433, 38554, 57301, 58324, 55063, 52991, 59914, 59921 or 33751 protein, and exhibit at least one activity of a 18607, 15603, 69318, 12303, 48000, 52920, 5433, 38554, 57301, 58324, 55063, 52991, 59914, 59921 or 33751 protein. Typically, biologically active portions comprise a domain or motif with at least one activity of the 18607, 15603, 69318, 12303, 48000, 52920, 5433, 38554, 57301, 58324, 55063, 52991, 59914, 59921 or 33751 protein. A biologically active portion of a 18607, 15603, 69318, 12303, 48000, 52920, 5433, 38554, 57301, 58324, 55063, 52991, 59914, 59921 or 33751 protein can be a polypeptide which is, for example, 10, 25, 50, 100, 200 or more amino acids in length. Biologically active portions of a 18607, 15603, 69318, 12303, 48000, 52920, 5433, 38554, 57301, 58324, 55063, 52991, 59914, 59921 or 33751 protein can be used as targets for developing agents which modulate a 18607, 15603, 69318, 12303, 48000, 52920, 5433, 38554, 57301, 58324, 55063, 52991, 59914, 59921 or 33751 mediated activity.

Calculations of homology or sequence identity (the terms “homology” and “identity” are used interchangeably herein) between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more, preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to 18607, 15603, 69318, 12303, 48000, 52920, 5433, 38554, 57301, 58324, 55063, 52991, 59914, 59921 or 33751 nucleic acid molecules of the invention. BLAST protein searches can be performed with the XBLAST program, score=50, wordlength=3 to obtain amino acid sequences homologous to 18607, 15603, 69318, 12303, 48000, 52920, 5433, 38554, 57301, 58324, 55063, 52991, 59914, 59921 or 33751 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Particular 18607, 15603, 69318, 12303, 48000, 52920, 5433, 38554, 57301, 58324, 55063, 52991, 59914, 59921 or 33751 polypeptides of the present invention have an amino acid sequence substantially identical to the amino acid sequence of SEQ ID NO:2, 6, 14, 19, 22, 25, 28, 33, 36, 39, 54, 57, 61, 70, 73 or 82. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%, 91%, 92%, 93%, 94%, 95%, 96%, 97%, 98% or 99% identity to SEQ ID NO:2, 6, 14, 19, 22, 25, 28, 33, 36, 39, 54, 57, 61, 70, 73 or 82 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%, 91%, 92%, 93%, 94%, 95%, 96%, 97%, 98% or 99% identity to SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

As used herein, cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, a “cellular proliferation, growth, differentiation, or migration process” is a process by which a cell increases in number, size or content, by which a cell develops a specialized set of characteristics which differ from that of other cells, or by which a cell moves closer to or further from a particular location or stimulus.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, an “angiogenic or angiogenesis disorder” includes a disease or disorder which affects or is caused by aberrant or deficient angiogenesis. Disorders involving angiogenesis include, but are not limited to, aberrant or excess angiogenesis in tumors such as hemangiomas and Kaposi's sarcoma, von Hippel-Lindau disease, as well as the angiogenesis associated with tumor growth; aberrant or excess angiogenesis in diseases such as a Castleman's disease or fibrodysplasia ossificans progressiva; aberrant or deficient angiogenesis associated with aging, complications of healing certain wounds and complications of diseases such as diabetes and rheumatoid arthritis; or aberrant or deficient angiogenesis associated with hereditary hemorrhagic telangiectasia, autosomal dominant polycystic kidney disease, myelodysplastic syndrome or Klippel-Trenaunay-Weber syndrome.

As used herein, disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

As used herein, disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

As used herein, disorders involving the kidney, also referred to herein as renal disorders, include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus., Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypemephroma, adenocarcinoma of kidney), which includes urothelial carcinomas of renal pelvis.

Examples of disorders of the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

As used herein, disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

As used herein, disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

As used herein, hormonal disorders and diseases include type I and type II diabetes mellitus, pituitary disorders (e.g., growth disorders), thyroid disorders (e.g., hypothyroidism or hyperthyroidism), and reproductive or fertility disorders (e.g., disorders which affect the organs of the reproductive system, e.g., the prostate gland, the uterus, or the vagina; disorders which involve an imbalance in the levels of a reproductive hormone in a subject; disorders affecting the ability of a subject to reproduce; and disorders affecting secondary sex characteristic development, e.g., adrenal hyperplasia).

Aberrant expression and/or activity of the molecules of the invention can mediate disorders associated with bone metabolism. “Bone metabolism” refers to direct or indirect effects in the formation or degeneration of bone structures, e.g., bone formation, bone resorption, etc., which can ultimately affect the concentrations in serum of calcium and phosphate. This term also includes activities mediated by the molecules of the invention in bone cells, e.g. osteoclasts and osteoblasts, that can in turn result in bone formation and degeneration. For example, molecules of the invention can support different activities of bone resorbing osteoclasts such as the stimulation of differentiation of monocytes and mononuclear phagocytes into osteoclasts. Accordingly, molecules of the invention that modulate the production of bone cells can influence bone formation and degeneration, and thus can be used to treat bone disorders. Examples of such disorders include, but are not limited to, osteoporosis, osteodystrophy, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, rickets, sarcoidosis, glucocorticoid antagonism, malabsorption syndrome, steatorrhea, tropical sprue, idiopathic hypercalcemia and milk fever.

As used herein, “a prostate disorder” refers to an abnormal condition occurring in the male pelvic region characterized by, e.g., male sexual dysfunction and/or urinary symptoms. This disorder may be manifested in the form of genitourinary inflammation (e.g., inflammation of smooth muscle cells) as in several common diseases of the prostate including prostatitis, benign prostatic hyperplasia and cancer, e.g., adenocarcinoma or carcinoma, of the prostate.

Examples of immune, e.g., inflammatory, (e.g. respiratory inflammatory) disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, inflammatory bowel disease, e.g. Crohn's disease and ulcerative colitis, aphthous ulcer, iritis, conjunctivitis, keratoconjunctivitis, asthma, allergic asthma, chronic obstructive pulmonary disease, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

As used herein, disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degenration, multiple system atrophy, including striatonigral degenration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

As used herein, disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

As used herein, disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, Leydig (interstitial) cell tumors and sertoli cell tumors (androblastoma), and testicular lymphoma, and miscellaneous lesions of tunica vaginalis.

As used herein, skeletal muscle, or musculoskeletal, disorders include, but are not limited to, muscular dystrophy (e.g., Duchenne muscular dystrophy, Becker muscular dystrophy, Emery-Dreifuss muscular dystrophy, limb-girdle muscular dystrophy, facioscapulohumeral muscular dystrophy, myotonic dystrophy, oculopharyngeal muscular dystrophy, distal muscular dystrophy, and congenital muscular dystrophy), motor neuron diseases (e.g., amyotrophic lateral sclerosis, infantile progressive spinal muscular atrophy, intermediate spinal muscular atrophy, spinal bulbar muscular atrophy, and adult spinal muscular atrophy), myopathies (e.g., inflammatory myopathies (e.g., dermatomyositis and polymyositis), myotonia congenita, paramyotonia congenita, central core disease, nemaline myopathy, myotubular myopathy, and periodic paralysis), tumors such as rhabdomyosarcoma, and metabolic diseases of muscle (e.g., phosphorylase deficiency, acid maltase deficiency, phosphofructokinase deficiency, debrancher enzyme deficiency, mitochondrial myopathy, carnitine deficiency, carnitine palmityl transferase deficiency, phosphoglycerate kinase deficiency, phosphoglycerate mutase deficiency, lactate dehydrogenase deficiency, and myoadenylate deaminase deficiency).

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders which can be treated or diagnosed by methods described herein include, but are not limited to, disorders associated with an accumulation in the liver of fibrous tissue, such as that resulting from an imbalance between production and degradation of the extracellular matrix accompanied by the collapse and condensation of preexisting fibers. The methods described herein can be used to diagnose or treat hepatocellular necrosis or injury induced by a wide variety of agents including processes which disturb homeostasis, such as an inflammatory process, tissue damage resulting from toxic injury or altered hepatic blood flow, and infections (e.g., bacterial, viral and parasitic). For example, the methods can be used for the early detection of hepatic injury, such as portal hypertension or hepatic fibrosis. In addition, the methods can be employed to detect liver fibrosis attributed to inborn errors of metabolism, for example, fibrosis resulting from a storage disorder such as Gaucher's disease (lipid abnormalities) or a glycogen storage disease, A1-antitrypsin deficiency; a disorder mediating the accumulation (e.g., storage) of an exogenous substance, for example, hemochromatosis (iron-overload syndrome) and copper storage diseases (Wilson's disease), disorders resulting in the accumulation of a toxic metabolite (e.g., tyrosinemia, fructosemia and galactosemia) and peroxisomal disorders (e.g., Zellweger syndrome). Additionally, the methods described herein can be used for the early detection and treatment of liver injury associated with the administration of various chemicals or drugs, such as for example, methotrexate, isonizaid, oxyphenisatin, methyldopa, chlorpromazine, tolbutamide or alcohol, or which represents a hepatic manifestation of a vascular disorder such as obstruction of either the intrahepatic or extrahepatic bile flow or an alteration in hepatic circulation resulting, for example, from chronic heart failure, veno-occlusive disease, portal vein thrombosis or Budd-Chiari syndrome.

As used herein, a “hepatic disorder” includes a disorder, disease or condition which affects the liver. The term hepatic disorder includes a disorder caused by the over- or under-production of hepatic enzymes, e.g., alanine aminotransferase, aspartate aminotransferase, or γ-glutammyl transferase, in the liver. For example, a hepatic disorder includes hepatic fibrosis, a hepatic disorder caused by a drug, a hepatic disorder caused by prolonged ethanol uptake, a hepatic injury caused by carbon tetrachloride exposure, hepatitis, liver tumors, cirrhosis of the liver, hemochromatosis, liver parasite induced disorders, alpha-1 antitrypsin deficiency, or autoimmune hepatitis. Hepatic disorders are disclosed at, for example, the American Liver Foundation website.

A hepatic disorder also includes a hepatic cell disorder. As used herein a “hepatic cell disorder” includes a disorder characterized by aberrant or unwanted hepatic cell activity, e.g., proliferation, migration, angiogenesis, or aberrant expression of cell surface adhesion molecules.

Additionally, the molecules of the invention can play an important role in the etiology of certain viral diseases, including but not limited to Hepatitis B, Hepatitis C and Herpes Simplex Virus (HSV). Modulators of the activity of the molecules of the invention could be used to control viral diseases. The modulators can be used in the treatment and/or diagnosis of viral infected tissue or virus-associated tissue fibrosis, especially liver and liver fibrosis. Also, such modulators can be used in the treatment and/or diagnosis of virus-associated carcinoma, especially hepatocellular cancer.

As used herein, a “viral pathogen” or “viral pathogen disorder” includes respiratory viral pathogens and their associated disorders include, for example, adenovirus, resulting in upper and lower respiratory tract infections; conjuctivitis and diarrhea; echovirus, resulting in upper respiratory tract infections, pharyngitis and rash; rhinovirus, resulting in upper respiratory tract infections; cosackievirus, resulting in Pleurodynia, herpangia, hand-foot-mouth disease; coronavirus, resulting in upper respiratory tract infections; influenza A and B viruses, resulting in influenza; parainfluenza virus 1-4, resulting in upper and lower respiratory tract infections and croup; respiratory syncytial virus, resulting in bronchiolitis and pneumonia. Digestive viral pathogens and their associated disorders include, for example, mumps virus, resulting in mumps, pancreatitis, and orchitis; rotavirus, resulting in childhood diarrhea; Norwalk Agent, resulting in gastroenteritis; hepatitis A virus, resulting in acute viral hepatitis; hepatitis B virus, hepatitis D virus and hepatitis C virus, resulting in acute or chronic hepatitis; hepatitis E virus, resulting in enterically transmitted hepatitis. Systemic viral pathogens associated with disorders involving skin eruptions include, for example, measles virus, resulting in measles (rubeola); rubella virus, resulting in German measles (rubella); parvovirus, resulting in erythema infectiosum and aplastic anemia; varicella-zoster virus, resulting in chicken pox and shingles; herpes simplex virus 1-associated, resulting in cold sores; and herpes simplex virus 2, resulting in genital herpes. Systemic viral pathogens associated with hematopoietic disorders include, for example, cytomegalovirus, resulting in cytomegalic inclusion disease; Epstein-Barr virus, resulting in mononucleosis; HTLV-1, resulting in adult T-cell leukemia and tropical spastic paraparesis; HTLV-II; and HIV 1 and HIV 2, resulting in AIDS. Arboviral pathogens associated with hemorrhagic fevers include, for example, dengue virus 1-4, resulting in dengue and hemorrhagic fever; yellow fever virus, resulting in yellow fever; Colorado tick fever virus, resulting in Colorado tick fever; and regional hemorrhagic fever viruses, resulting in Bolivian, Argentinian, Lassa fever. Viral pathogens associated with warty growths and other hyperplasias include, for example, papillomavirus, resulting in condyloma and cervical carcinoma; and molluscum virus, resulting in molluscum contagiosum. Viral pathogens associated with central nervous system disorders include, for example, poliovirus, resulting in poliomyelitis; rabiesvirus, associated with rabies; JC virus, associated with progressive multifocal leukoencephalophathy; and arboviral encephalitis viruses, resulting in Eastern, Western, Venezuelan, St. Louis, or California group encephalitis. Viral pathogens associated with cancer include, for example, human papillomaviruses, implicated in the genesis of several cancers including squamous cell carcinoma of the cervix and anogenital region, oral cancer and laryngeal cancers; Epstein-Barr virus, implicated in pathogenesis of the African form of Burkitt lymphoma, B-cell lymphomas, Hodgkin disease, and nasopharyngeal carcinomas; hepatitis B virus, implicated in liver cancer; human T-cell leukemia virus type 1 (HTLV-1), associated with T-cell leukemia/lymphoma; and the Kaposi sarcoma herpesvirus (KSHV).

“Blood platelet disorders” include, but are not limited to, thrombocytopenia due to a reduced number of megakaryocytes in the bone marrow, for example, as a result of chemotherapy; invasive disorders, such as leukemia, idiopathic or drug- or toxin-induced aplasia of the marrow, or rare hereditary amegakaryocytic thrombocytopenias; ineffective thrombopoiesis, for example, as a result of megaloblastic anemia, alcohol toxicity, vitamin B12 or folate deficiency, myelodysplastic disorders, or rare hereditary disorders (e.g., Wiskott-Aldrich syndrome and May-hegglin anomaly); a reduction in platelet distribution, for example, as a result of cirrhosis, a splenic invasive disease (e.g., Gaucher's disease), or myelofibrosis with extramedullary myeloid metaplasia; increased platelet destruction, for example, as a result of removal of IgG-coated platelets by the mononuclear phagocytic system (e.g., idiopathic thrombocytopenic purpura (ITP), secondary immune thrombocytopenia (e.g., systemic lupus erythematosus, lymphoma, or chronic lymphocytic leukemia), drug-related immune thrombocytopenias (e.g., as with quinidine, aspirin, and heparin), post-transfusion purpura, and neonatal thrombocytopenia as a result of maternal platelet autoantibodies or maternal platelet alloantibodies). Also included are thrombocytopenia secondary to intravascular clotting and thrombin induced damage to platelets as a result of, for example, obstetric complications, metastatic tumors, severe gram-negative bacteremia, thrombotic thrombocytopenic purpura, or severe illness. Also included is dilutional thrombocytopenia, for example, due to massive hemorrhage. Blood platelet disorders also include, but are not limited to, essential thrombocytosis and thrombocytosis associated with, for example, splenectomy, acute or chronic inflammatory diseases, hemolytic anemia, carcinoma, Hodgkin's disease, lymphoproliferative disorders, and malignant lymphomas.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

As used herein, neurological, central nervous system or neurodegenerative disorders include disorders of the central nervous system (CNS) and the peripheral nervous system, e.g., cognitive and neurodegenerative disorders, Examples of neurological disorders include, but are not limited to, autonomic function disorders such as hypertension and sleep disorders, and neuropsychiatric disorders, such as depression, schizophrenia, schizoaffective disorder, Korsakoff's psychosis, alcoholism, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity. Such neurological disorders include, for example, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer's disease and Pick's disease, degenerative diseases of basal ganglia and brain stem, including Parkinsonism, idiopathic Parkinson's disease (paralysis agitans) and other Lewy diffuse body diseases, progressive supranuclear palsy, corticobasal degeneration, multiple system atrophy, including striatonigral degeneration, Shy-Drager syndrome, and olivopontocerebellar atrophy, and Huntington's disease, senile dementia, Gilles de la Tourette's syndrome, epilepsy, and Jakob-Creutzfieldt disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease. Further CNS-related disorders include, for example, those listed in the American Psychiatric Association's Diagnostic and Statistical manual of Mental Disorders (DSM), the most current version of which is incorporated herein by reference in its entirety.

Disorders involving the eye and vision include, but are not limited to, granulomatous uveitis, cataracts, trachoma, corneal dystrophies, e.g., granular dystrophy or lattice dystrophy, glaucomas, retrolental fibroplasia, diabetes mellitus, hypertensive and arteriosclerotic retinopathy, retinitis pigmentosa, macular degeneration, retinoblastoma, papillaedema, and optic neuritis.

Additionally, molecules of the invention can play an important role in the regulation of metabolism or pain disorders. Diseases of metabolic imbalance include, but are not limited to, obesity, anorexia nervosa, cachexia, lipid disorders, and diabetes. Examples of pain disorders include headache posttherapeutic neuralgia, diabetic neuropathy, postmastectomy pain syndrome, stump pain, reflex sympathetic dystrophy, trigeminal neuralgia, neuropathic pain, orofacial neuropathic pain, osteoarthritis, arthritis, e.g., rheumatoid arthritis, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, back pain, chronic nonspecific pain, pain associated with surgery, psychogenic pain, tooth pain, musculoskeletal pain disorder, chronic pelvic pain, nonorganic chronic headache, tension-type headache, cluster headache, migraine, complex regional pain syndrome, vaginismus, nerve trunk pain, somatoform pain disorder, cyclical mastalgia, chronic fatigue syndrome, multiple somatization syndrome, chronic pain disorder, cancer pain, somatization disorder, Syndrome X, facial pain, idiopathic pain disorder, posttraumatic rheumatic pain modulation disorder (fibrositis syndrome), hyperalgesia, and Tangier disease.

As used herein, the term “pain signaling mechanisms” includes the cellular mechanisms involved in the development and regulation of pain, e.g., pain elicited by noxious chemical, mechanical, or thermal stimuli, in a subject, e.g., a mammal such as a human. In mammals, the initial detection of noxious chemical, mechanical, or thermal stimuli, a process referred to as “nociception”, occurs predominantly at the peripheral terminals of specialized, small diameter sensory neurons. These sensory neurons transmit the information to the central nervous system, evoking a perception of pain or discomfort and initiating appropriate protective reflexes.

Various aspects of the invention are described in further detail below.

### Isolated Nucleic Acid Molecules

In one aspect, the invention provides, an isolated or purified, nucleic acid molecule that encodes a 18607, 15603, 69318, 12303, 48000, 52920, 5433, 38554, 57301, 58324, 55063, 52991, 59914, 59921 or 33751 polypeptide described herein, e.g., a full length 18607, 15603, 69318, 12303, 48000, 52920, 5433, 38554, 57301, 58324, 55063, 52991, 59914, 59921 or 33751 protein or a fragment thereof, e.g., a biologically active portion of 18607, 15603, 69318, 12303, 48000, 52920, 5433, 38554, 57301, 58324, 55063, 52991, 59914, 59921 or 33751 protein. Also included is a nucleic acid fragment suitable for use as a hybridization probe, which can be used, e.g., to identify a nucleic acid molecule encoding a polypeptide of the invention, 18607, 15603, 69318, 12303, 48000, 52920, 5433, 38554, 57301, 58324, 55063, 52991, 59914, 59921 or 33751 mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or a portion of any of this nucleotide sequence. In one embodiment, the nucleic acid molecule includes sequences encoding the human 18607, 15603, 69318, 12303, 48000, 52920, 5433, 38554, 57301, 58324, 55063, 52991, 59914, 59921 or 33751 protein (i.e., “the coding region” of SEQ ID NO:1, 5, 13, 18, 21, 24, 27, 32, 35, 38, 53, 56, 60, 69, 72 or 81, as shown in SEQ ID NO:3, 7, 15, 20, 23, 26, 29, 34, 37, 40, 55, 58, 62, 71, 74 or 83, respectively), as well as 5′ untranslated sequences and 3′ untranslated sequences. Alternatively, the nucleic acid molecule can include only the coding region of SEQ ID NO:1, 5, 13, 18, 21, 24, 27, 32, 35, 38, 53, 56, 60, 69, 72 or 81 (e.g., SEQ ID NO:3, 7, 15, 20, 23, 26, 29, 34, 37, 40, 55, 58, 62, 71, 74 or 83) and, e.g., no flanking sequences which normally accompany the subject sequence. In another embodiment, the nucleic acid molecule encodes a sequence corresponding to a fragment of the protein corresponding to domains within SEQ ID NO:2, 6, 14, 19, 22, 25, 28, 33, 36, 39, 54, 57, 61, 70, 73 or 82.

In another embodiment, an isolated nucleic acid molecule of the invention includes a nucleic acid molecule which is a complement of the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or a portion of any of these nucleotide sequences. In other embodiments, the nucleic acid molecule of the invention is sufficiently complementary to the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83 such that it can hybridize to the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, thereby forming a stable duplex.

In one embodiment, an isolated nucleic acid molecule of the present invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or a portion, preferably of the same length, of any of these nucleotide sequences.

### 18607, 15603, 69318, 12303, 48000, 52920, 5433, 38554, 57301, 58324, 55063, 52991, 59914, 59921 or 33751 Nucleic Acid Fragments

A nucleic acid molecule of the invention can include only a portion of the nucleic acid sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83. For example, such a nucleic acid molecule can include a fragment which can be used as a probe or primer or a fragment encoding a portion of a 18607, 15603, 69318, 12303, 48000, 52920, 5433, 38554, 57301, 58324, 55063, 52991, 59914, 59921 or 33751 protein, e.g., an immunogenic or biologically active portion of a 18607, 15603, 69318, 12303, 48000, 52920, 5433, 38554, 57301, 58324, 55063, 52991, 59914, 59921 or 33751 protein. A fragment can comprise those nucleotides of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, which encode a domain of human 18607, 15603, 69318, 12303, 48000, 52920, 5433, 38554, 57301, 58324, 55063, 52991, 59914, 59921 or 33751. The nucleotide sequence determined from the cloning of the 18607, 15603, 69318, 12303, 48000, 52920, 5433, 38554, 57301, 58324, 55063, 52991, 59914, 59921 or 33751 gene allows for the generation of probes and primers designed for use in identifying and/or cloning other 18607, 15603, 69318, 12303, 48000, 52920, 5433, 38554, 57301, 58324, 55063, 52991, 59914, 59921 or 33751 family members, or fragments thereof, as well as 18607, 15603, 69318, 12303, 48000, 52920, 5433, 38554, 57301, 58324, 55063, 52991, 59914, 59921 or 33751 homologs, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 100 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a 18607, 15603, 69318, 12303, 48000, 52920, 5433, 38554, 57301, 58324, 55063, 52991, 59914, 59921 or 33751 nucleic acid fragment can include a sequence corresponding to a domain, as described herein.

18607, 15603, 69318, 12303, 48000, 52920, 5433, 38554, 57301, 58324, 55063, 52991, 59914, 59921 or 33751 probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or of a naturally occurring allelic variant or mutant of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83.

In a preferred embodiment the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a domain identified in the 18607, 15603, 69318, 12303, 48000, 52920, 5433, 38554, 57301, 58324, 55063, 52991, 59914, 59921 or 33751 sequences, as disclosed herein.

In another embodiment a set of primers is provided, e.g., primers suitable for use in a PCR, which can be used to amplify a selected region of a 18607, 15603, 69318, 12303, 48000, 52920, 5433, 38554, 57301, 58324, 55063, 52991, 59914, 59921 or 33751 sequence, e.g., a domain, region, site or other sequence described herein. The primers should be at least 5, 10, or 50 base pairs in length and less than 100, or less than 200, base pairs in length. The primers should be identical, or differ by one base from a sequence disclosed herein or from a naturally occurring variant.

A nucleic acid fragment can encode an epitope bearing region of a polypeptide described herein.

A nucleic acid fragment encoding a “biologically active portion of a 18607, 15603, 69318, 12303, 48000, 52920, 5433, 38554, 57301, 58324, 55063, 52991, 59914, 59921 or 33751 polypeptide” can be prepared by isolating a portion of the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, which encodes a polypeptide having a 18607, 15603, 69318, 12303, 48000, 52920, 5433, 38554, 57301, 58324, 55063, 52991, 59914, 59921 or 33751 biological activity (e.g., the biological activities of the 18607, 15603, 69318, 12303, 48000, 52920, 5433, 38554, 57301, 58324, 55063, 52991, 59914, 59921 or 33751 proteins are described herein), expressing the encoded portion of the 18607, 15603, 69318, 12303, 48000, 52920, 5433, 38554, 57301, 58324, 55063, 52991, 59914, 59921 or 33751 protein (e.g., by recombinant expression in vitro) and assessing the activity of the encoded portion of the 18607, 15603, 69318, 12303, 48000, 52920, 5433, 38554, 57301, 58324, 55063, 52991, 59914, 59921 or 33751 protein. A nucleic acid fragment encoding a biologically active portion of a 18607, 15603, 69318, 12303, 48000, 52920, 5433, 38554, 57301, 58324, 55063, 52991, 59914, 59921 or 33751 polypeptide, can comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500 or more nucleotides in length and hybridizes under stringent hybridization conditions to a nucleic acid molecule of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83.

### 18607, 15603, 69318, 12303, 48000, 52920, 5433, 38554, 57301, 58324, 55063, 52991, 59914, 59921 or 33751 Nucleic Acid Variants

The invention further encompasses nucleic acid molecules that differ from the nucleotide sequence shown in SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83. Such differences can be due to degeneracy of the genetic code (and result in a nucleic acid which encodes the same 18607, 15603, 69318, 12303, 48000, 52920, 5433, 38554, 57301, 58324, 55063, 52991, 59914, 59921 or 33751 proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2, 6, 14, 19, 22, 25, 28, 33, 36, 39, 54, 57, 61, 70, 73 or 82. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2, 6, 14, 19, 22, 25, 28, 33, 36, 39, 54, 57, 61, 70, 73 or 82 or a fragment of this sequence. Such nucleic acid molecules can readily be identified as being able to hybridize under stringent conditions, to the nucleotide sequence shown in SEQ ID NO:2, 6, 14, 19, 22, 25, 28, 33, 36, 39, 54, 57, 61, 70, 73 or 82 or a fragment of the sequence. Nucleic acid molecules corresponding to orthologs, homologs, and allelic variants of the 18607, 15603, 69318, 12303, 48000, 52920, 5433, 38554, 57301, 58324, 55063, 52991, 59914, 59921 or 33751 cDNAs of the invention can further be isolated by mapping to the same chromosome or locus as the 18607, 15603, 69318, 12303, 48000, 52920, 5433, 38554, 57301, 58324, 55063, 52991, 59914, 59921 or 33751 gene.

Preferred variants include those that are correlated with activities specific to the molecules of the invention, i.e. calcium channel activity, calcium/sodium antiporter activity, potassium channel activity, organic ion transporter activity, choline transporter activity, or other.

Allelic variants of 18607, 15603, 69318, 12303, 48000, 52920, 5433, 38554, 57301, 58324, 55063, 52991, 59914, 59921 or 33751, e.g., human 18607, 15603, 69318, 12303, 48000, 52920, 5433, 38554, 57301, 58324, 55063, 52991, 59914, 59921 or 33751, include both functional and non-functional proteins. Functional allelic variants are naturally occurring amino acid sequence variants of the 18607, 15603, 69318, 12303, 48000, 52920, 5433, 38554, 57301, 58324, 55063, 52991, 59914, 59921 or 33751 protein within a population that maintain the ability to bind a 18607, 15603, 69318, 12303, 48000, 52920, 5433, 38554, 57301, 58324, 55063, 52991, 59914, 59921 or 33751 ligand or substrate and/or modulate calcium channel activity, calcium/sodium antiporter activity, potassium channel activity, organic ion transporter activity or choline transporter activity. Functional allelic variants will typically contain only conservative substitution of one or more amino acids of SEQ ID NO:2, 6, 14, 19, 22, 25, 28, 33, 36, 39, 54, 57, 61, 70, 73 or 82, or substitution, deletion or insertion of non-critical residues in non-critical regions of the protein. Non-functional allelic variants are naturally-occurring amino acid sequence variants of the 18607, 15603, 69318, 12303, 48000, 52920, 5433, 38554, 57301, 58324, 55063, 52991, 59914, 59921 or 33751, e.g., human 18607, 15603, 69318, 12303, 48000, 52920, 5433, 38554, 57301, 58324, 55063, 52991, 59914, 59921 or 33751, protein within a population that do not have the ability to bind a 18607, 15603, 69318, 12303, 48000, 52920, 5433, 38554, 57301, 58324, 55063, 52991, 59914, 59921 or 33751 ligand or substrate and/or modulate calcium channel activity, calcium/sodium antiporter activity, potassium channel activity, organic ion transporter activity or choline transporter activity. Non-functional allelic variants will typically contain a non-conservative substitution, a deletion, or insertion, or premature truncation of the amino acid sequence of SEQ ID NO:2, 6, 14, 19, 22, 25, 28, 33, 36, 39, 54, 57, 61, 70, 73 or 82, or a substitution, insertion, or deletion in critical residues or critical regions of the protein.

Moreover, nucleic acid molecules encoding other 18607, 15603, 69318, 12303, 48000, 52920, 5433, 38554, 57301, 58324, 55063, 52991, 59914, 59921 or 33751 family members and, thus, which have a nucleotide sequence which differs from the 18607, 15603, 69318, 12303, 48000, 52920, 5433, 38554, 57301, 58324, 55063, 52991, 59914, 59921 or 33751 sequences of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83 are intended to be within the scope of the invention.

### Antisense Nucleic Acid Molecules, Ribozymes and Modified 18607, 15603, 69318, 12303, 48000, 52920, 5433, 38554, 57301, 58324, 55063, 52991, 59914, 59921 or 33751 Nucleic Acid Molecules

In another aspect, the invention features, an isolated nucleic acid molecule which is antisense to 18607, 15603, 69318, 12303, 48000, 52920, 5433, 38554, 57301, 58324, 55063, 52991, 59914, 59921 or 33751. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire 18607, 15603, 69318, 12303, 48000, 52920, 5433, 38554, 57301, 58324, 55063, 52991, 59914, 59921 or 33751 coding strand, or to only a portion thereof (e.g., the coding region of human 18607, 15603, 69318, 12303, 48000, 52920, 5433, 38554, 57301, 58324, 55063, 52991, 59914, 59921 or 33751 corresponding to SEQ ID NO:3, 7, 15, 20, 23, 26, 29, 34, 37, 40, 55, 58, 62, 71, 74 or 83, respectively). In another embodiment, the antisense nucleic acid molecule is antisense to a “noncoding region” of the coding strand of a nucleotide sequence encoding 18607, 15603, 69318, 12303, 48000, 52920, 5433, 38554, 57301, 58324, 55063, 52991, 59914, 59921 or 33751 (e.g., the 5′ and 3′ untranslated regions).

An antisense nucleic acid can be designed such that it is complementary to the entire coding region of 18607, 15603, 69318, 12303, 48000, 52920, 5433, 38554, 57301, 58324, 55063, 52991, 59914, 59921 or 33751 mRNA, but more preferably is an oligonucleotide which is antisense to only a portion of the coding or noncoding region of 18607, 15603, 69318, 12303, 48000, 52920, 5433, 38554, 57301, 58324, 55063, 52991, 59914, 59921 or 33751 mRNA. For example, the antisense oligonucleotide can be complementary to the region surrounding the translation start site of 18607, 15603, 69318, 12303, 48000, 52920, 5433, 38554, 57301, 58324, 55063, 52991, 59914, 59921 or 33751 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a 18607, 15603, 69318, 12303, 48000, 52920, 5433, 38554, 57301, 58324, 55063, 52991, 59914, 59921 or 33751 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically or selectively bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a 18607, 15603, 69318, 12303, 48000, 52920, 5433, 38554, 57301, 58324, 55063, 52991, 59914, 59921 or 33751-encoding nucleic acid can include one or more sequences complementary to the nucleotide sequence of a 18607, 15603, 69318, 12303, 48000, 52920, 5433, 38554, 57301, 58324, 55063, 52991, 59914, 59921 or 33751 cDNA disclosed herein (i.e., SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a 18607, 15603, 69318, 12303, 48000, 52920, 5433, 38554, 57301, 58324, 55063, 52991, 59914, 59921 or 33751-encoding mRNA. See, e.g., Cech et al. U.S. Pat. No. 4,987,071; and Cech et al. U.S. Pat. No. 5,116,742. Alternatively, 18607, 15603, 69318, 12303, 48000, 52920, 5433, 38554, 57301, 58324, 55063, 52991, 59914, 59921 or 33751 mRNA can be used to select a catalytic RNA having a specific ribonuclease activity from a pool of RNA molecules. See, e.g., Bartel and Szostak (1993) Science 261:1411-1418.

18607, 15603, 69318, 12303, 48000, 52920, 5433, 38554, 57301, 58324, 55063, 52991, 59914, 59921 or 33751 gene expression can be inhibited by targeting nucleotide sequences complementary to the regulatory region of the 18607, 15603, 69318, 12303, 48000, 52920, 5433, 38554, 57301, 58324, 55063, 52991, 59914, 59921 or 33751 (e.g., the 18607, 15603, 69318, 12303, 48000, 52920, 5433, 38554, 57301, 58324, 55063, 52991, 59914, 59921 or 33751 promoter and/or enhancers) to form triple helical structures that prevent transcription of the 18607, 15603, 69318, 12303, 48000, 52920, 5433, 38554, 57301, 58324, 55063, 52991, 59914, 59921 or 33751 gene in target cells. See generally, Helene (1991) Anticancer Drug Des. 6:569-84; Helene (1992) Ann. N.Y. Acad. Sci. 660:27-36; and Maher (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A 18607, 15603, 69318, 12303, 48000, 52920, 5433, 38554, 57301, 58324, 55063, 52991, 59914, 59921 or 33751 nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4: 5-23).

As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. 93: 14670-675.

PNAs of 18607, 15603, 69318, 12303, 48000, 52920, 5433, 38554, 57301, 58324, 55063, 52991, 59914, 59921 or 33751 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of 18607, 15603, 69318, 12303, 48000, 52920, 5433, 38554, 57301, 58324, 55063, 52991, 59914, 59921 or 33751 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. (1996) supra)); or as probes or primers for DNA sequencing or hybridization (Hyrup et al. (1996) supra; Perry-O'Keefe supra).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide can be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a 18607, 15603, 69318, 12303, 48000, 52920, 5433, 38554, 57301, 58324, 55063, 52991, 59914, 59921 or 33751 nucleic acid of the invention, two complementary regions one having a fluorophore and one a quencher such that the molecular beacon is useful for quantitating the presence of the 18607, 15603, 69318, 12303, 48000, 52920, 5433, 38554, 57301, 58324, 55063, 52991, 59914, 59921 or 33751 nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### Isolated 18607, 15603, 69318, 12303, 48000, 52920, 5433, 38554, 57301, 58324, 55063, 52991, 59914, 59921 or 33751 Polypeptides

In another aspect, the invention features, an isolated 18607, 15603, 69318, 12303, 48000, 52920, 5433, 38554, 57301, 58324, 55063, 52991, 59914, 59921 or 33751 protein, or fragment, e.g., a biologically active portion, for use as immunogens or antigens to raise or test (or more generally to bind) anti-18607, 15603, 69318, 12303, 48000, 52920, 5433, 38554, 57301, 58324, 55063, 52991, 59914, 59921 or 33751 antibodies. 18607, 15603, 69318, 12303, 48000, 52920, 5433, 38554, 57301, 58324, 55063, 52991, 59914, 59921 or 33751 protein can be isolated from cells or tissue sources using standard protein purification techniques. 18607, 15603, 69318, 12303, 48000, 52920, 5433, 38554, 57301, 58324, 55063, 52991, 59914, 59921 or 33751 protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present in a native cell.

In a preferred embodiment, a 18607, 15603, 69318, 12303, 48000, 52920, 5433, 38554, 57301, 58324, 55063, 52991, 59914, 59921 or 33751 polypeptide has one or more of the following characteristics: 1) it has the ability to modulate membrane excitability; 2) it has the ability to influence the resting potential of membranes; 3) it has the ability to modulate wave forms and frequencies of action potentials; 4) it has the ability to modulate thresholds of excitation; 5) it has the ability to modulate neurite outgrowth and synaptogenesis; 6) it had the ability to modulate signal transduction, 7) it has the ability to bind a second messenger; 8) it has the ability to bind diacylglycerol; 9) it has the ability to regulate the flow of cations through a membrane; 10) it has the ability to transport a substrate or target molecule, e.g., an ion (e.g., a calcium ion) across a membrane; 11) it has the ability to transport a second substrate or target molecule, e.g., another ion (e.g., a sodium ion) across a membrane; 12) it has the ability to transport a third substrate or target molecule, e.g., another ion (e.g., a potassium ion) across a membrane; 13) it has the ability to interact with and/or modulate the activity of a second non-transporter protein; 14) it has the ability to modulate cellular signaling and/or gene transcription (e.g., either directly or indirectly; 15) it has the ability to interact with a non-TWIK protein molecule; 16) it has the ability to activate a TWIK-dependent signal transduction pathway; 17) it has the ability to modulate the release of neurotransmitters; 18) it has the ability to protect cells and/or tissues from organic ions; 19) it has the ability to modulate intracellular Ca²⁺ concentration; 20) it has the ability to bind a ligand, e.g., L-glutamate, and/or glycine; 21) it has the ability to modulate (e.g., promote, catalyze, regulate, initiate, facilitate or inhibit) the manufacture of choline metabolites and/or compounds of which choline is a component or precursor, e.g., phospholipids (e.g., phosphatidylcholine (lecithin), sphingomyelin, sphingophosphorylcholine, and platelet activating factor), acetylcholine, very low density lipoproteins (VLDLs), and betaine, e.g., by transporting choline into or out of cells; 22) it has the ability to modulate (e.g., promote, catalyze, regulate, initiate, facilitate or inhibit) transport of choline, its metabolites, and/or compounds of which choline is a component or precursor across membranes (e.g., plasma membranes), e.g., from an extracellular medium into a cell, or vice versa; 23) it has the ability to modulate (e.g., promote, catalyze, regulate, initiate, facilitate or inhibit) transport of choline, its metabolites, and/or compounds of which choline is a component or precursor across barriers between tissues (e.g., the blood-brain barrier); 24) it has a molecular weight, e.g., a deduced molecular weight, preferably ignoring any contribution of post translational modifications, amino acid composition or other physical characteristic of a 18607, 15603, 69318, 12303, 48000, 52920, 5433, 38554, 57301, 58324, 55063, 52991, 59914, 59921 or 33751 polypeptide, e.g., a polypeptide of SEQ ID NO:2, 6, 14, 19, 22, 25, 28, 33, 36, 39, 54, 57, 61, 70, 73 or 82; 25) it has an overall sequence similarity of at least 60%, preferably at least 70%, more preferably at least 80, 90, or 95%, with a polypeptide of SEQ ID NO:2, 6, 14, 19, 22, 25, 28, 33, 36, 39, 54, 57, 61, 70, 73 or 82; 26) it is expressed in a multitude of human tissues and cell lines (refer to section for each molecule of the invention); and 27) it has specific domains which are preferably about 70%, 80%, 90% or 95% identical to the identified amino acid residues of SEQ ID NO:2, 6, 14, 19, 22, 25, 28, 33, 36, 39, 54, 57, 61, 70, 73 or 82 (refer to section for each molecule of the invention for domain names and locations within amino acid sequence).

In a preferred embodiment the 18607, 15603, 69318, 12303, 48000, 52920, 5433, 38554, 57301, 58324, 55063, 52991, 59914, 59921 or 33751 protein, or fragment thereof, differs from the corresponding sequence in SEQ ID NO:2, 6, 14, 19, 22, 25, 28, 33, 36, 39, 54, 57, 61, 70, 73 or 82. In one embodiment it differs by at least one but by less than 15, 10 or 5 amino acid residues. In another it differs from the corresponding sequence in SEQ ID NO:2, 6, 14, 19, 22, 25, 28, 33, 36, 39, 54, 57, 61, 70, 73 or 82 by at least one residue but less than 20%, 15%, 10% or 5% of the residues in it differ from the corresponding sequence in SEQ ID NO:2, 6, 14, 19, 22, 25, 28, 33, 36, 39, 54, 57, 61, 70, 73 or 82. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the identified or conserved domain(s) within SEQ ID NO:2, 6, 14, 19, 22, 25, 28, 33, 36, 39, 54, 57, 61, 70, 73 or 82. In another embodiment one or more differences are in the cidentified or conserved domain(s) within SEQ ID NO:2, 6, 14, 19, 22, 25, 28, 33, 36, 39, 54, 57, 61, 70, 73 or 82.

Other embodiments include a protein that contains one or more changes in amino acid sequence, e.g., a change in an amino acid residue which is not essential for activity. Such 18607, 15603, 69318, 12303, 48000, 52920, 5433, 38554, 57301, 58324, 55063, 52991, 59914, 59921 or 33751 proteins differ in amino acid sequence from SEQ ID NO:2, 6, 14, 19, 22, 25, 28, 33, 36, 39, 54, 57, 61, 70, 73 or 82, yet retain biological activity.

In one embodiment, the protein includes an amino acid sequence at least about 60%, 65%, 70%, 75%, 80%, 85%, 90%, 95%, 98% or more homologous to SEQ ID NO:2, 6, 14, 19, 22, 25, 28, 33, 36, 39, 54, 57, 61, 70, 73 or 82.

A 18607, 15603, 69318, 12303, 48000, 52920, 5433, 38554, 57301, 58324, 55063, 52991, 59914, 59921 or 33751 protein or fragment is provided which varies from the sequence of SEQ ID NO:2, 6, 14, 19, 22, 25, 28, 33, 36, 39, 54, 57, 61, 70, 73 or 82 in regions defined by amino acids that are not within identified or conserved domains or regions by at least one but by less than 15, 10 or 5 amino acid residues in the protein or fragment but which does not differ from SEQ ID NO:2, 6, 14, 19, 22, 25, 28, 33, 36, 39, 54, 57, 61, 70, 73 or 82 in regions defined by amino acids that are within identified or conserved domains or regions. (If this comparison requires alignment the sequences should be aligned for maximum homology. “Looped” out sequences from deletions or insertions, or mismatches, are considered differences.) In some embodiments the difference is at a non-essential residue or is a conservative substitution, while in others the difference is at an essential residue or is a non-conservative substitution.

In one embodiment, a biologically active portion of a 18607, 15603, 69318, 12303, 48000, 52920, 5433, 38554, 57301, 58324, 55063, 52991, 59914, 59921 or 33751 protein includes an identified domain (refer to section for each molecule of the invention). Moreover, other biologically active portions, in which other regions of the protein are deleted, can be prepared by recombinant techniques and evaluated for one or more of the functional activities of a native 18607, 15603, 69318, 12303, 48000, 52920, 5433, 38554, 57301, 58324, 55063, 52991, 59914, 59921 or 33751 protein.

In a preferred embodiment, the 18607, 15603, 69318, 12303, 48000, 52920, 5433, 38554, 57301, 58324, 55063, 52991, 59914, 59921 or 33751 protein has an amino acid sequence shown in SEQ ID NO:2, 6, 14, 19, 22, 25, 28, 33, 36, 39, 54, 57, 61, 70, 73 or 82. In other embodiments, the 18607, 15603, 69318, 12303, 48000, 52920, 5433, 38554, 57301, 58324, 55063, 52991, 59914, 59921 or 33751 protein is sufficiently or substantially identical to SEQ ID NO:2, 6, 14, 19, 22, 25, 28, 33, 36, 39, 54, 57, 61, 70, 73 or 82. In yet another embodiment, the 18607, 15603, 69318, 12303, 48000, 52920, 5433, 38554, 57301, 58324, 55063, 52991, 59914, 59921 or 33751 protein is sufficiently or substantially identical to SEQ ID NO:2, 6, 14, 19, 22, 25, 28, 33, 36, 39, 54, 57, 61, 70, 73 or 82 and retains the functional activity of the protein of SEQ ID NO:2, 6, 14, 19, 22, 25, 28, 33, 36, 39, 54, 57, 61, 70, 73 or 82, as described in detail in the subsections above.

### 18607, 15603, 69318, 12303, 48000, 52920, 5433, 38554, 57301, 58324, 55063, 52991, 59914, 59921 or 33751 Chimeric or Fusion Proteins

In another aspect, the invention provides 18607, 15603, 69318, 12303, 48000, 52920, 5433, 38554, 57301, 58324, 55063, 52991, 59914, 59921 or 33751 chimeric or fusion proteins. As used herein, a 18607, 15603, 69318, 12303, 48000, 52920, 5433, 38554, 57301, 58324, 55063, 52991, 59914, 59921 or 33751 “chimeric protein” or “fusion protein” includes a 18607, 15603, 69318, 12303, 48000, 52920, 5433, 38554, 57301, 58324, 55063, 52991, 59914, 59921 or 33751 polypeptide linked to a non-18607, 15603, 69318, 12303, 48000, 52920, 5433, 38554, 57301, 58324, 55063, 52991, 59914, 59921 or 33751 polypeptide. A “non-18607, 15603, 69318, 12303, 48000, 52920, 5433, 38554, 57301, 58324, 55063, 52991, 59914, 59921 or 33751 polypeptide” refers to a polypeptide having an amino acid sequence corresponding to a protein which is not substantially homologous to the 18607, 15603, 69318, 12303, 48000, 52920, 5433, 38554, 57301, 58324, 55063, 52991, 59914, 59921 or 33751 protein, e.g., a protein which is different from the 18607, 15603, 69318, 12303, 48000, 52920, 5433, 38554, 57301, 58324, 55063, 52991, 59914, 59921 or 33751 protein and which is derived from the same or a different organism. The 18607, 15603, 69318, 12303, 48000, 52920, 5433, 38554, 57301, 58324, 55063, 52991, 59914, 59921 or 33751 polypeptide of the fusion protein can correspond to all or a portion e.g., a fragment described herein of a 18607, 15603, 69318, 12303, 48000, 52920, 5433, 38554, 57301, 58324, 55063, 52991, 59914, 59921 or 33751 amino acid sequence. In a preferred embodiment, a 18607, 15603, 69318, 12303, 48000, 52920, 5433, 38554, 57301, 58324, 55063, 52991, 59914, 59921 or 33751 fusion protein includes at least one (or two) biologically active portion of a 18607, 15603, 69318, 12303, 48000, 52920, 5433, 38554, 57301, 58324, 55063, 52991, 59914, 59921 or 33751 protein. The non-18607, 15603, 69318, 12303, 48000, 52920, 5433, 38554, 57301, 58324, 55063, 52991, 59914, 59921 or 33751 polypeptide can be fused to the N-terminus or C-terminus of the 18607, 15603, 69318, 12303, 48000, 52920, 5433, 38554, 57301, 58324, 55063, 52991, 59914, 59921 or 33751 polypeptide.

The fusion protein can include a moiety which has a high affinity for a ligand. For example, the fusion protein can be a GST-18607, 15603, 69318, 12303, 48000, 52920, 5433, 38554, 57301, 58324, 55063, 52991, 59914, 59921 or 33751 fusion protein in which the 18607, 15603, 69318, 12303, 48000, 52920, 5433, 38554, 57301, 58324, 55063, 52991, 59914, 59921 or 33751 sequences are fused to the C-terminus of the GST sequences. Such fusion proteins can facilitate the purification of recombinant 18607, 15603, 69318, 12303, 48000, 52920, 5433, 38554, 57301, 58324, 55063, 52991, 59914, 59921 or 33751. Alternatively, the fusion protein can be a 18607, 15603, 69318, 12303, 48000, 52920, 5433, 38554, 57301, 58324, 55063, 52991, 59914, 59921 or 33751 protein containing a heterologous signal sequence at its N-terminus. In certain host cells (e.g., mammalian host cells), expression and/or secretion of 18607, 15603, 69318, 12303, 48000, 52920, 5433, 38554, 57301, 58324, 55063, 52991, 59914, 59921 or 33751 can be increased through use of a heterologous signal sequence.

Fusion proteins can include all or a part of a serum protein, e.g., a portion of an immunoglobulin (e.g., IgG, IgA, or IgE), e.g., an Fc region and/or the hinge C1 and C2 sequences of an immunoglobulin or human serum albumin.

The 18607, 15603, 69318, 12303, 48000, 52920, 5433, 38554, 57301, 58324, 55063, 52991, 59914, 59921 or 33751 fusion proteins of the invention can be incorporated into pharmaceutical compositions and administered to a subject in vivo. The 18607, 15603, 69318, 12303, 48000, 52920, 5433, 38554, 57301, 58324, 55063, 52991, 59914, 59921 or 33751 fusion proteins can be used to affect the bioavailability of a 18607, 15603, 69318, 12303, 48000, 52920, 5433, 38554, 57301, 58324, 55063, 52991, 59914, 59921 or 33751 substrate. 18607, 15603, 69318, 12303, 48000, 52920, 5433, 38554, 57301, 58324, 55063, 52991, 59914, 59921 or 33751 fusion proteins can be useful therapeutically for the treatment of disorders caused by, for example, (i) aberrant modification or mutation of a gene encoding a 18607, 15603, 69318, 12303, 48000, 52920, 5433, 38554, 57301, 58324, 55063, 52991, 59914, 59921 or 33751 protein; (ii) mis-regulation of the 18607, 15603, 69318, 12303, 48000, 52920, 5433, 38554, 57301, 58324, 55063, 52991, 59914, 59921 or 33751 gene; and (iii) aberrant post-translational modification of a 18607, 15603, 69318, 12303, 48000, 52920, 5433, 38554, 57301, 58324, 55063, 52991, 59914, 59921 or 33751 protein.

Moreover, the 18607, 15603, 69318, 12303, 48000, 52920, 5433, 38554, 57301, 58324, 55063, 52991, 59914, 59921 or 33751-fusion proteins of the invention can be used as immunogens to produce anti-18607, 15603, 69318, 12303, 48000, 52920, 5433, 38554, 57301, 58324, 55063, 52991, 59914, 59921 or 33751 antibodies in a subject, to purify 18607, 15603, 69318, 12303, 48000, 52920, 5433, 38554, 57301, 58324, 55063, 52991, 59914, 59921 or 33751 ligands and in screening assays to identify molecules which inhibit the interaction of 18607, 15603, 69318, 12303, 48000, 52920, 5433, 38554, 57301, 58324, 55063, 52991, 59914, 59921 or 33751 with a 18607, 15603, 69318, 12303, 48000, 52920, 5433, 38554, 57301, 58324, 55063, 52991, 59914, 59921 or 33751 substrate.

Expression vectors are commercially available that already encode a fusion moiety (e.g., a GST polypeptide). A 18607, 15603, 69318, 12303, 48000, 52920, 5433, 38554, 57301, 58324, 55063, 52991, 59914, 59921 or 33751-encoding nucleic acid can be cloned into such an expression vector such that the fusion moiety is linked in-frame to the 18607, 15603, 69318, 12303, 48000, 52920, 5433, 38554, 57301, 58324, 55063, 52991, 59914, 59921 or 33751 protein.

### Variants of 18607, 15603, 69318, 12303, 48000, 52920, 5433, 38554, 57301, 58324, 55063, 52991, 59914, 59921 or 33751 Proteins

In another aspect, the invention also features a variant of a 18607, 15603, 69318, 12303, 48000, 52920, 5433, 38554, 57301, 58324, 55063, 52991, 59914, 59921 or 33751 polypeptide, e.g., which functions as an agonist (mimetics) or as an antagonist. Variants of the 18607, 15603, 69318, 12303, 48000, 52920, 5433, 38554, 57301, 58324, 55063, 52991, 59914, 59921 or 33751 proteins can be generated by mutagenesis, e.g., discrete point mutation, the insertion or deletion of sequences or the truncation of a 18607, 15603, 69318, 12303, 48000, 52920, 5433, 38554, 57301, 58324, 55063, 52991, 59914, 59921 or 33751 protein. An agonist of the 18607, 15603, 69318, 12303, 48000, 52920, 5433, 38554, 57301, 58324, 55063, 52991, 59914, 59921 or 33751 proteins can retain substantially the same, or a subset, of the biological activities of the naturally occurring form of a 18607, 15603, 69318, 12303, 48000, 52920, 5433, 38554, 57301, 58324, 55063, 52991, 59914, 59921 or 33751 protein. An antagonist of a 18607, 15603, 69318, 12303, 48000, 52920, 5433, 38554, 57301, 58324, 55063, 52991, 59914, 59921 or 33751 protein can inhibit one or more of the activities of the naturally occurring form of the 18607, 15603, 69318, 12303, 48000, 52920, 5433, 38554, 57301, 58324, 55063, 52991, 59914, 59921 or 33751 protein by, for example, competitively modulating a 18607, 15603, 69318, 12303, 48000, 52920, 5433, 38554, 57301, 58324, 55063, 52991, 59914, 59921 or 33751-mediated activity of a 18607, 15603, 69318, 12303, 48000, 52920, 5433, 38554, 57301, 58324, 55063, 52991, 59914, 59921 or 33751 protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the 18607, 15603, 69318, 12303, 48000, 52920, 5433, 38554, 57301, 58324, 55063, 52991, 59914, 59921 or 33751 protein.

Variants of a 18607, 15603, 69318, 12303, 48000, 52920, 5433, 38554, 57301, 58324, 55063, 52991, 59914, 59921 or 33751 protein can be identified by screening combinatorial libraries of mutants, e.g., truncation mutants, of a 18607, 15603, 69318, 12303, 48000, 52920, 5433, 38554, 57301, 58324, 55063, 52991, 59914, 59921 or 33751 protein for agonist or antagonist activity.

Libraries of fragments e.g., N terminal, C terminal, or internal fragments, of a 18607, 15603, 69318, 12303, 48000, 52920, 5433, 38554, 57301, 58324, 55063, 52991, 59914, 59921 or 33751 protein coding sequence can be used to generate a variegated population of fragments for screening and subsequent selection of variants of a 18607, 15603, 69318, 12303, 48000, 52920, 5433, 38554, 57301, 58324, 55063, 52991, 59914, 59921 or 33751 protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Recursive ensemble mutagenesis (REM), a new technique which enhances the frequency of functional mutants in the libraries, can be used in combination with the screening assays to identify 18607, 15603, 69318, 12303, 48000, 52920, 5433, 38554, 57301, 58324, 55063, 52991, 59914, 59921 or 33751 variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated 18607, 15603, 69318, 12303, 48000, 52920, 5433, 38554, 57301, 58324, 55063, 52991, 59914, 59921 or 33751 library. For example, a library of expression vectors can be transfected into a cell line, e.g., a cell line, which ordinarily responds to 18607, 15603, 69318, 12303, 48000, 52920, 5433, 38554, 57301, 58324, 55063, 52991, 59914, 59921 or 33751 in a substrate-dependent manner. The transfected cells are then contacted with 18607, 15603, 69318, 12303, 48000, 52920, 5433, 38554, 57301, 58324, 55063, 52991, 59914, 59921 or 33751 and the effect of the expression of the mutant on signaling by the 18607, 15603, 69318, 12303, 48000, 52920, 5433, 38554, 57301, 58324, 55063, 52991, 59914, 59921 or 33751 substrate can be detected, e.g., by measuring either calcium channel activity, calcium/sodium antiporter activity, potassium channel activity, organic ion transporter activity, choline transporter activity, or other activity. Plasmid DNA can then be recovered from the cells which score for inhibition, or alternatively, potentiation of signaling by the 18607, 15603, 69318, 12303, 48000, 52920, 5433, 38554, 57301, 58324, 55063, 52991, 59914, 59921 or 33751 substrate, and the individual clones further characterized.

In another aspect, the invention features a method of making a 18607, 15603, 69318, 12303, 48000, 52920, 5433, 38554, 57301, 58324, 55063, 52991, 59914, 59921 or 33751 polypeptide, e.g., a peptide having a non-wild type activity, e.g., an antagonist, agonist, or super agonist of a naturally occurring 18607, 15603, 69318, 12303, 48000, 52920, 5433, 38554, 57301, 58324, 55063, 52991, 59914, 59921 or 33751 polypeptide, e.g., a naturally occurring 18607, 15603, 69318, 12303, 48000, 52920, 5433, 38554, 57301, 58324, 55063, 52991, 59914, 59921 or 33751 polypeptide. The method includes altering the sequence of a 18607, 15603, 69318, 12303, 48000, 52920, 5433, 38554, 57301, 58324, 55063, 52991, 59914, 59921 or 33751 polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a 18607, 15603, 69318, 12303, 48000, 52920, 5433, 38554, 57301, 58324, 55063, 52991, 59914, 59921 or 33751 polypeptide a biological activity of a naturally occurring 18607, 15603, 69318, 12303, 48000, 52920, 5433, 38554, 57301, 58324, 55063, 52991, 59914, 59921 or 33751 polypeptide. The method includes altering the sequence, e.g., by substitution or deletion of one or more residues, of a 18607, 15603, 69318, 12303, 48000, 52920, 5433, 38554, 57301, 58324, 55063, 52991, 59914, 59921 or 33751 polypeptide, e.g., altering the sequence of a non-conserved region, or a domain or residue described herein, and testing the altered polypeptide for the desired activity.

### Anti-18607, 15603, 69318, 12303, 48000, 52920, 5433, 38554, 57301, 58324, 55063, 52991, 59914, 59921 or 33751 Antibodies

In another aspect, the invention provides an anti-18607, 15603, 69318, 12303, 48000, 52920, 5433, 38554, 57301, 58324, 55063, 52991, 59914, 59921 or 33751 antibody. The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab′)₂ fragments which can be generated by treating the antibody with an enzyme such as papain or pepsin, respectively.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length 18607, 15603, 69318, 12303, 48000, 52920, 5433, 38554, 57301, 58324, 55063, 52991, 59914, 59921 or 33751 protein or, antigenic peptide fragment of 18607, 15603, 69318, 12303, 48000, 52920, 5433, 38554, 57301, 58324, 55063, 52991, 59914, 59921 or 33751 can be used as an immunogen or can be used to identify anti-18607, 15603, 69318, 12303, 48000, 52920, 5433, 38554, 57301, 58324, 55063, 52991, 59914, 59921 or 33751 antibodies made with other immunogens, e.g., cells, membrane preparations, and the like. The antigenic peptide of 18607, 15603, 69318, 12303, 48000, 52920, 5433, 38554, 57301, 58324, 55063, 52991, 59914, 59921 or 33751 should include at least 8 amino acid residues of the amino acid sequence shown in SEQ ID NO:2, 6, 14, 19, 22, 25, 28, 33, 36, 39, 54, 57, 61, 70, 73 or 82 and encompasses an epitope of 18607, 15603, 69318, 12303, 48000, 52920, 5433, 38554, 57301, 58324, 55063, 52991, 59914, 59921 or 33751. Preferably, the antigenic peptide includes at least 10 amino acid residues, more preferably at least 15 amino acid residues, even more preferably at least 20 amino acid residues, and most preferably at least 30 amino acid residues.

Fragments of 18607, 15603, 69318, 12303, 48000, 52920, 5433, 38554, 57301, 58324, 55063, 52991, 59914, 59921 or 33751 which include hydrophilic regions of SEQ ID NO:2, 6, 14, 19, 22, 25, 28, 33, 36, 39, 54, 57, 61, 70, 73 or 82 can be used to make, e.g., used as immunogens or used to characterize the specificity of an antibody, antibodies against hydrophilic regions of the 18607, 15603, 69318, 12303, 48000, 52920, 5433, 38554, 57301, 58324, 55063, 52991, 59914, 59921 or 33751 protein. Similarly, fragments of 18607, 15603, 69318, 12303, 48000, 52920, 5433, 38554, 57301, 58324, 55063, 52991, 59914, 59921 or 33751 which include hydrophobic regions of SEQ ID NO:2, 6, 14, 19, 22, 25, 28, 33, 36, 39, 54, 57, 61, 70, 73 or 82 can be used to make an antibody against a hydrophobic region of the 18607, 15603, 69318, 12303, 48000, 52920, 5433, 38554, 57301, 58324, 55063, 52991, 59914, 59921 or 33751 protein; fragments of 18607, 15603, 69318, 12303, 48000, 52920, 5433, 38554, 57301, 58324, 55063, 52991, 59914, 59921 or 33751 which include residues within extra cellular domain(s) of SEQ ID NO:2, 6, 14, 19, 22, 25, 28, 33, 36, 39, 54, 57, 61, 70, 73 or 82 can be used to make an antibody against an extracellular or non-cytoplasmic region of the 18607, 15603, 69318, 12303, 48000, 52920, 5433, 38554, 57301, 58324, 55063, 52991, 59914, 59921 or 33751 protein; fragments of 18607, 15603, 69318, 12303, 48000, 52920, 5433, 38554, 57301, 58324, 55063, 52991, 59914, 59921 or 33751 which include residues within intracellular regions of SEQ ID NO:2, 6, 14, 19, 22, 25, 28, 33, 36, 39, 54, 57, 61, 70, 73 or 82 can be used to make an antibody against an intracellular region of the 18607, 15603, 69318, 12303, 48000, 52920, 5433, 38554, 57301, 58324, 55063, 52991, 59914, 59921 or 33751 protein; a fragment of 18607, 15603, 69318, 12303, 48000, 52920, 5433, 38554, 57301, 58324, 55063, 52991, 59914, 59921 or 33751 which include residues within identified or conserved domains of SEQ ID NO:2, 6, 14, 19, 22, 25, 28, 33, 36, 39, 54, 57, 61, 70, 73 or 82 can be used to make an antibody against the identified or conserved domain of the 18607, 15603, 69318, 12303, 48000, 52920, 5433, 38554, 57301, 58324, 55063, 52991, 59914, 59921 or 33751 protein.

Antibodies reactive with, or specific or selective for, any of these regions, or other regions or domains described herein are provided.

Preferred epitopes encompassed by the antigenic peptide are regions of 18607, 15603, 69318, 12303, 48000, 52920, 5433, 38554, 57301, 58324, 55063, 52991, 59914, 59921 or 33751 located on the surface of the protein, e.g., hydrophilic regions, as well as regions with high antigenicity. For example, an Emini surface probability analysis of the human 18607, 15603, 69318, 12303, 48000, 52920, 5433, 38554, 57301, 58324, 55063, 52991, 59914, 59921 or 33751 protein sequence can be used to indicate the regions that have a particularly high probability of being localized to the surface of the 18607, 15603, 69318, 12303, 48000, 52920, 5433, 38554, 57301, 58324, 55063, 52991, 59914, 59921 or 33751 protein and are thus likely to constitute surface residues useful for targeting antibody production.

In a preferred embodiment the antibody can bind to the extracellular portion of the 18607, 15603, 69318, 12303, 48000, 52920, 5433, 38554, 57301, 58324, 55063, 52991, 59914, 59921 or 33751 protein, e.g., it can bind to a whole cell which expresses the 18607, 15603, 69318, 12303, 48000, 52920, 5433, 38554, 57301, 58324, 55063, 52991, 59914, 59921 or 33751 protein. In another embodiment, the antibody binds an intracellular portion of the 18607, 15603, 69318, 12303, 48000, 52920, 5433, 38554, 57301, 58324, 55063, 52991, 59914, 59921 or 33751 protein.

In a preferred embodiment the antibody binds an epitope on any domain or region on 18607, 15603, 69318, 12303, 48000, 52920, 5433, 38554, 57301, 58324, 55063, 52991, 59914, 59921 or 33751 proteins described herein.

Additionally, chimeric, humanized, and completely human antibodies are also within the scope of the invention. Chimeric, humanized, but most preferably, completely human antibodies are desirable for applications which include repeated administration, e.g., therapeutic treatment of human patients, and some diagnostic applications.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559).

A humanized or complementarity determining region (CDR)-grafted antibody will have at least one or two, but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a 18607, 15603, 69318, 12303, 48000, 52920, 5433, 38554, 57301, 58324, 55063, 52991, 59914, 59921 or 33751 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison (1985) Science 229:1202-1207, by Oi et al. (1986) BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 18607, 15603, 69318, 12303, 48000, 52920, 5433, 38554, 57301, 58324, 55063, 52991, 59914, 59921 or 33751 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. (1986) Nature 321:552-525; Verhoeyan et al. (1988) Science 239:1534; Beidler et al. (1988) J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-18607, 15603, 69318, 12303, 48000, 52920, 5433, 38554, 57301, 58324, 55063, 52991, 59914, 59921 or 33751 antibody can be a single chain antibody. A single-chain antibody (scFV) can be engineered as described in, for example, Colcher et al. (1999) Ann. N Y Acad. Sci. 880:263-80; and Reiter (1996) Clin. Cancer Res. 2:245-52. The single chain antibody can be dimerized or multimerized to generate multivalent antibodies having specificities for different epitopes of the same target 18607, 15603, 69318, 12303, 48000, 52920, 5433, 38554, 57301, 58324, 55063, 52991, 59914, 59921 or 33751 protein.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-18607, 15603, 69318, 12303, 48000, 52920, 5433, 38554, 57301, 58324, 55063, 52991, 59914, 59921 or 33751 antibody (e.g., monoclonal antibody) can be used to isolate 18607, 15603, 69318, 12303, 48000, 52920, 5433, 38554, 57301, 58324, 55063, 52991, 59914, 59921 or 33751 by standard techniques, such as affinity chromatography or immunoprecipitation. Moreover, an anti-18607, 15603, 69318, 12303, 48000, 52920, 5433, 38554, 57301, 58324, 55063, 52991, 59914, 59921 or 33751 antibody can be used to detect 18607, 15603, 69318, 12303, 48000, 52920, 5433, 38554, 57301, 58324, 55063, 52991, 59914, 59921 or 33751 protein (e.g., in a cellular lysate or cell supernatant) in order to evaluate the abundance and pattern of expression of the protein. Anti-18607, 15603, 69318, 12303, 48000, 52920, 5433, 38554, 57301, 58324, 55063, 52991, 59914, 59921 or 33751 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹U, ³⁵S or ³H.

In preferred embodiments, an antibody can be made by immunizing with a purified 18607, 15603, 69318, 12303, 48000, 52920, 5433, 38554, 57301, 58324, 55063, 52991, 59914, 59921 or 33751 antigen, or a fragment thereof, e.g., a fragment described herein, a membrane associated antigen, tissues, e.g., crude tissue preparations, whole cells, preferably living cells, lysed cells, or cell fractions, e.g., membrane fractions.

Antibodies which bind only a native 18607, 15603, 69318, 12303, 48000, 52920, 5433, 38554, 57301, 58324, 55063, 52991, 59914, 59921 or 33751 protein, only denatured or otherwise non-native 18607, 15603, 69318, 12303, 48000, 52920, 5433, 38554, 57301, 58324, 55063, 52991, 59914, 59921 or 33751 protein, or which bind both, are within the invention. Antibodies with linear or conformational epitopes are within the invention. Conformational epitopes sometimes can be identified by identifying antibodies which bind to native but not denatured 18607, 15603, 69318, 12303, 48000, 52920, 5433, 38554, 57301, 58324, 55063, 52991, 59914, 59921 or 33751 protein.

### Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a 18607, 15603, 69318, 12303, 48000, 52920, 5433, 38554, 57301, 58324, 55063, 52991, 59914, 59921 or 33751 nucleic acid in a form suitable for expression of the nucleic acid in a host cell.

Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., 18607, 15603, 69318, 12303, 48000, 52920, 5433, 38554, 57301, 58324, 55063, 52991, 59914, 59921 or 33751 proteins, mutant forms of 18607, 15603, 69318, 12303, 48000, 52920, 5433, 38554, 57301, 58324, 55063, 52991, 59914, 59921 or 33751 proteins, fusion proteins, and the like).

The recombinant expression vectors of the invention can be designed for expression of 18607, 15603, 69318, 12303, 48000, 52920, 5433, 38554, 57301, 58324, 55063, 52991, 59914, 59921 or 33751 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in 18607, 15603, 69318, 12303, 48000, 52920, 5433, 38554, 57301, 58324, 55063, 52991, 59914, 59921 or 33751 activity assays, (e.g., direct assays or competitive assays described in detail below), or to generate antibodies specific or selective for 18607, 15603, 69318, 12303, 48000, 52920, 5433, 38554, 57301, 58324, 55063, 52991, 59914, 59921 or 33751 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The 18607, 15603, 69318, 12303, 48000, 52920, 5433, 38554, 57301, 58324, 55063, 52991, 59914, 59921 or 33751 expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub et al., (1986) Reviews—Trends in Genetics 1:1.

Another aspect the invention provides a host cell which includes a nucleic acid molecule described herein, e.g., a 18607, 15603, 69318, 12303, 48000, 52920, 5433, 38554, 57301, 58324, 55063, 52991, 59914, 59921 or 33751 nucleic acid molecule within a recombinant expression vector or a 18607, 15603, 69318, 12303, 48000, 52920, 5433, 38554, 57301, 58324, 55063, 52991, 59914, 59921 or 33751 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications can occur in succeeding generations due to either mutation or environmental influences, such progeny may not, in fact, be identical to the parent cell, but are still included within the scope of the term as used herein.

A host cell can be any prokaryotic or eukaryotic cell. For example, a 18607, 15603, 69318, 12303, 48000, 52920, 5433, 38554, 57301, 58324, 55063, 52991, 59914, 59921 or 33751 protein can be expressed in bacterial cells such as E. coli, insect cells, yeast or mammalian cells (such as Chinese hamster ovary (CHO) cells or CV-1 origin, SV-40 (COS) cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a 18607, 15603, 69318, 12303, 48000, 52920, 5433, 38554, 57301, 58324, 55063, 52991, 59914, 59921 or 33751 protein. Accordingly, the invention further provides methods for producing a 18607, 15603, 69318, 12303, 48000, 52920, 5433, 38554, 57301, 58324, 55063, 52991, 59914, 59921 or 33751 protein using the host cells of the invention. In one embodiment, the method includes culturing the host cell of the invention (into which a recombinant expression vector encoding a 18607, 15603, 69318, 12303, 48000, 52920, 5433, 38554, 57301, 58324, 55063, 52991, 59914, 59921 or 33751 protein has been introduced) in a suitable medium such that a 18607, 15603, 69318, 12303, 48000, 52920, 5433, 38554, 57301, 58324, 55063, 52991, 59914, 59921 or 33751 protein is produced. In another embodiment, the method further includes isolating a 18607, 15603, 69318, 12303, 48000, 52920, 5433, 38554, 57301, 58324, 55063, 52991, 59914, 59921 or 33751 protein from the medium or the host cell.

In another aspect, the invention features, a cell or purified preparation of cells which include a 18607, 15603, 69318, 12303, 48000, 52920, 5433, 38554, 57301, 58324, 55063, 52991, 59914, 59921 or 33751 transgene, or which otherwise misexpress 18607, 15603, 69318, 12303, 48000, 52920, 5433, 38554, 57301, 58324, 55063, 52991, 59914, 59921 or 33751. The cell preparation can consist of human or non-human cells, e.g., rodent cells, e.g., mouse or rat cells, rabbit cells, or pig cells. In preferred embodiments, the cell or cells include a 18607, 15603, 69318, 12303, 48000, 52920, 5433, 38554, 57301, 58324, 55063, 52991, 59914, 59921 or 33751 transgene, e.g., a heterologous form of a 18607, 15603, 69318, 12303, 48000, 52920, 5433, 38554, 57301, 58324, 55063, 52991, 59914, 59921 or 33751, e.g., a gene derived from humans (in the case of a non-human cell). The 18607, 15603, 69318, 12303, 48000, 52920, 5433, 38554, 57301, 58324, 55063, 52991, 59914, 59921 or 33751 transgene can be misexpressed, e.g., overexpressed or underexpressed. In other preferred embodiments, the cell or cells include a gene which misexpresses an endogenous 18607, 15603, 69318, 12303, 48000, 52920, 5433, 38554, 57301, 58324, 55063, 52991, 59914, 59921 or 33751, e.g., a gene the expression of which is disrupted, e.g., a knockout. Such cells can serve as a model for studying disorders which are related to mutated or misexpressed 18607, 15603, 69318, 12303, 48000, 52920, 5433, 38554, 57301, 58324, 55063, 52991, 59914, 59921 or 33751 alleles or for use in drug screening.

In another aspect, the invention features, a human cell, e.g., a hematopoietic stem cell, transformed with nucleic acid which encodes a subject 18607, 15603, 69318, 12303, 48000, 52920, 5433, 38554, 57301, 58324, 55063, 52991, 59914, 59921 or 33751 polypeptide.

Also provided are cells, preferably human cells, e.g., human hematopoietic or fibroblast cells, in which an endogenous 18607, 15603, 69318, 12303, 48000, 52920, 5433, 38554, 57301, 58324, 55063, 52991, 59914, 59921 or 33751 is under the control of a regulatory sequence that does not normally control the expression of the endogenous 18607, 15603, 69318, 12303, 48000, 52920, 5433, 38554, 57301, 58324, 55063, 52991, 59914, 59921 or 33751 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 18607, 15603, 69318, 12303, 48000, 52920, 5433, 38554, 57301, 58324, 55063, 52991, 59914, 59921 or 33751 gene. For example, an endogenous 18607, 15603, 69318, 12303, 48000, 52920, 5433, 38554, 57301, 58324, 55063, 52991, 59914, 59921 or 33751 gene which is “transcriptionally silent,” e.g., not normally expressed, or expressed only at very low levels, can be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

### Transgenic Animals

The invention provides non-human transgenic animals. Such animals are useful for studying the function and/or activity of a 18607, 15603, 69318, 12303, 48000, 52920, 5433, 38554, 57301, 58324, 55063, 52991, 59914, 59921 or 33751 protein and for identifying and/or evaluating modulators of 18607, 15603, 69318, 12303, 48000, 52920, 5433, 38554, 57301, 58324, 55063, 52991, 59914, 59921 or 33751 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous 18607, 15603, 69318, 12303, 48000, 52920, 5433, 38554, 57301, 58324, 55063, 52991, 59914, 59921 or 33751 gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a 18607, 15603, 69318, 12303, 48000, 52920, 5433, 38554, 57301, 58324, 55063, 52991, 59914, 59921 or 33751 protein to particular cells. A transgenic founder animal can be identified based upon the presence of a 18607, 15603, 69318, 12303, 48000, 52920, 5433, 38554, 57301, 58324, 55063, 52991, 59914, 59921 or 33751 transgene in its genome and/or expression of 18607, 15603, 69318, 12303, 48000, 52920, 5433, 38554, 57301, 58324, 55063, 52991, 59914, 59921 or 33751 mRNA in tissues or cells of the animals. A transgenic founder animal can then be used to breed additional animals carrying the transgene. Moreover, transgenic animals carrying a transgene encoding a 18607, 15603, 69318, 12303, 48000, 52920, 5433, 38554, 57301, 58324, 55063, 52991, 59914, 59921 or 33751 protein can further be bred to other transgenic animals carrying other transgenes.

18607, 15603, 69318, 12303, 48000, 52920, 5433, 38554, 57301, 58324, 55063, 52991, 59914, 59921 or 33751 proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

### Uses

The nucleic acid molecules, proteins, protein homologs, and antibodies described herein can be used in one or more of the following methods: a) screening assays;

b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a 18607, 15603, 69318, 12303, 48000, 52920, 5433, 38554, 57301, 58324, 55063, 52991, 59914, 59921 or 33751 protein (e.g., via a recombinant expression vector in a host cell in gene therapy applications), to detect a 18607, 15603, 69318, 12303, 48000, 52920, 5433, 38554, 57301, 58324, 55063, 52991, 59914, 59921 or 33751 mRNA (e.g., in a biological sample) or a genetic alteration in a 18607, 15603, 69318, 12303, 48000, 52920, 5433, 38554, 57301, 58324, 55063, 52991, 59914, 59921 or 33751 gene, and to modulate 18607, 15603, 69318, 12303, 48000, 52920, 5433, 38554, 57301, 58324, 55063, 52991, 59914, 59921 or 33751 activity, as described further below. The 18607, 15603, 69318, 12303, 48000, 52920, 5433, 38554, 57301, 58324, 55063, 52991, 59914, 59921 or 33751 proteins can be used to treat disorders characterized by insufficient or excessive production of a 18607, 15603, 69318, 12303, 48000, 52920, 5433, 38554, 57301, 58324, 55063, 52991, 59914, 59921 or 33751 substrate or production of 18607, 15603, 69318, 12303, 48000, 52920, 5433, 38554, 57301, 58324, 55063, 52991, 59914, 59921 or 33751 inhibitors. In addition, the 18607, 15603, 69318, 12303, 48000, 52920, 5433, 38554, 57301, 58324, 55063, 52991, 59914, 59921 or 33751 proteins can be used to screen for naturally occurring 18607, 15603, 69318, 12303, 48000, 52920, 5433, 38554, 57301, 58324, 55063, 52991, 59914, 59921 or 33751 substrates, to screen for drugs or compounds which modulate 18607, 15603, 69318, 12303, 48000, 52920, 5433, 38554, 57301, 58324, 55063, 52991, 59914, 59921 or 33751 activity, as well as to treat disorders characterized by insufficient or excessive production of 18607, 15603, 69318, 12303, 48000, 52920, 5433, 38554, 57301, 58324, 55063, 52991, 59914, 59921 or 33751 protein or production of 18607, 15603, 69318, 12303, 48000, 52920, 5433, 38554, 57301, 58324, 55063, 52991, 59914, 59921 or 33751 protein forms which have decreased, aberrant or unwanted activity compared to 18607, 15603, 69318, 12303, 48000, 52920, 5433, 38554, 57301, 58324, 55063, 52991, 59914, 59921 or 33751 wild type protein (e.g., aberrant or deficient calcium channel activity, calcium/sodium antiporter activity, potassium channel activity, organic ion transporter activity, choline transporter activity, or other activity). Moreover, the anti-18607, 15603, 69318, 12303, 48000, 52920, 5433, 38554, 57301, 58324, 55063, 52991, 59914, 59921 or 33751 antibodies of the invention can be used to detect and isolate 18607, 15603, 69318, 12303, 48000, 52920, 5433, 38554, 57301, 58324, 55063, 52991, 59914, 59921 or 33751 proteins, regulate the bioavailability of 18607, 15603, 69318, 12303, 48000, 52920, 5433, 38554, 57301, 58324, 55063, 52991, 59914, 59921 or 33751 proteins, and modulate 18607, 15603, 69318, 12303, 48000, 52920, 5433, 38554, 57301, 58324, 55063, 52991, 59914, 59921 or 33751 activity.

A method of evaluating a compound for the ability to interact with, e.g., bind, a subject 18607, 15603, 69318, 12303, 48000, 52920, 5433, 38554, 57301, 58324, 55063, 52991, 59914, 59921 or 33751 polypeptide is provided. The method includes: contacting the compound with the subject 18607, 15603, 69318, 12303, 48000, 52920, 5433, 38554, 57301, 58324, 55063, 52991, 59914, 59921 or 33751 polypeptide; and evaluating ability of the compound to interact with, e.g., to bind or form a complex with the subject 18607, 15603, 69318, 12303, 48000, 52920, 5433, 38554, 57301, 58324, 55063, 52991, 59914, 59921 or 33751 polypeptide. This method can be performed in vitro, e.g., in a cell free system, or in vivo, e.g., in a two-hybrid interaction trap assay. This method can be used to identify naturally occurring molecules which interact with subject 18607, 15603, 69318, 12303, 48000, 52920, 5433, 38554, 57301, 58324, 55063, 52991, 59914, 59921 or 33751 polypeptide. It can also be used to find natural or synthetic inhibitors of subject 18607, 15603, 69318, 12303, 48000, 52920, 5433, 38554, 57301, 58324, 55063, 52991, 59914, 59921 or 33751 polypeptide. Screening methods are discussed in more detail below.

### Screening Assays:

The invention provides methods (also referred to herein as “screening assays”) for identifying modulators, i.e., candidate or test compounds or agents (e.g., proteins, peptides, peptidomimetics, peptoids, small molecules or other drugs) which bind to 18607, 15603, 69318, 12303, 48000, 52920, 5433, 38554, 57301, 58324, 55063, 52991, 59914, 59921 or 33751 proteins, have a stimulatory or inhibitory effect on, for example, 18607, 15603, 69318, 12303, 48000, 52920, 5433, 38554, 57301, 58324, 55063, 52991, 59914, 59921 or 33751 expression or 18607, 15603, 69318, 12303, 48000, 52920, 5433, 38554, 57301, 58324, 55063, 52991, 59914, 59921 or 33751 activity, or have a stimulatory or inhibitory effect on, for example, the expression or activity of a 18607, 15603, 69318, 12303, 48000, 52920, 5433, 38554, 57301, 58324, 55063, 52991, 59914, 59921 or 33751 substrate. Compounds thus identified can be used to modulate the activity of target gene products (e.g., 18607, 15603, 69318, 12303, 48000, 52920, 5433, 38554, 57301, 58324, 55063, 52991, 59914, 59921 or 33751 genes) in a therapeutic protocol, to elaborate the biological function of the target gene product, or to identify compounds that disrupt normal target gene interactions.

In one embodiment, the invention provides assays for screening candidate or test compounds which are substrates of a 18607, 15603, 69318, 12303, 48000, 52920, 5433, 38554, 57301, 58324, 55063, 52991, 59914, 59921 or 33751 protein or polypeptide or a biologically active portion thereof. In another embodiment, the invention provides assays for screening candidate or test compounds which bind to or modulate the activity of a 18607, 15603, 69318, 12303, 48000, 52920, 5433, 38554, 57301, 58324, 55063, 52991, 59914, 59921 or 33751 protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909-13; Erb et al. (1994) Proc. Natl. Acad. Sci. USA 91:11422-426; Zuckermann et al. (1994). J. Med. Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a 18607, 15603, 69318, 12303, 48000, 52920, 5433, 38554, 57301, 58324, 55063, 52991, 59914, 59921 or 33751 protein or biologically active portion thereof is contacted with a test compound, and the ability of the test compound to modulate 18607, 15603, 69318, 12303, 48000, 52920, 5433, 38554, 57301, 58324, 55063, 52991, 59914, 59921 or 33751 activity is determined. Determining the ability of the test compound to modulate 18607, 15603, 69318, 12303, 48000, 52920, 5433, 38554, 57301, 58324, 55063, 52991, 59914, 59921 or 33751 activity can be accomplished by monitoring, for example, calcium channel activity, calcium/sodium antiporter activity, potassium channel activity, organic ion transporter activity, choline transporter activity, or other activity. The cell, for example, can be of mammalian origin, e.g., human.

The ability of the test compound to modulate 18607, 15603, 69318, 12303, 48000, 52920, 5433, 38554, 57301, 58324, 55063, 52991, 59914, 59921 or 33751 binding to a compound, e.g., a 18607, 15603, 69318, 12303, 48000, 52920, 5433, 38554, 57301, 58324, 55063, 52991, 59914, 59921 or 33751 substrate, or to bind to 18607, 15603, 69318, 12303, 48000, 52920, 5433, 38554, 57301, 58324, 55063, 52991, 59914, 59921 or 33751 can also be evaluated. This can be accomplished, for example, by coupling the compound, e.g., the substrate, with a radioisotope or enzymatic label such that binding of the compound, e.g., the substrate, to 18607, 15603, 69318, 12303, 48000, 52920, 5433, 38554, 57301, 58324, 55063, 52991, 59914, 59921 or 33751 can be determined by detecting the labeled compound, e.g., substrate, in a complex. Alternatively, 18607, 15603, 69318, 12303, 48000, 52920, 5433, 38554, 57301, 58324, 55063, 52991, 59914, 59921 or 33751 could be coupled with a radioisotope or enzymatic label to monitor the ability of a test compound to modulate 18607, 15603, 69318, 12303, 48000, 52920, 5433, 38554, 57301, 58324, 55063, 52991, 59914, 59921 or 33751 binding to a 18607, 15603, 69318, 12303, 48000, 52920, 5433, 38554, 57301, 58324, 55063, 52991, 59914, 59921 or 33751 substrate in a complex. For example, compounds (e.g., 18607, 15603, 69318, 12303, 48000, 52920, 5433, 38554, 57301, 58324, 55063, 52991, 59914, 59921 or 33751 substrates) can be labeled with ¹²⁵I, ¹⁴C, ³⁵S or ³H., either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a 18607, 15603, 69318, 12303, 48000, 52920, 5433, 38554, 57301, 58324, 55063, 52991, 59914, 59921 or 33751 substrate) to interact with 18607, 15603, 69318, 12303, 48000, 52920, 5433, 38554, 57301, 58324, 55063, 52991, 59914, 59921 or 33751 with or without the labeling of any of the interactants can be evaluated. For example, a microphysiometer can be used to detect the interaction of a compound with 18607, 15603, 69318, 12303, 48000, 52920, 5433, 38554, 57301, 58324, 55063, 52991, 59914, 59921 or 33751 without the labeling of either the compound or the 18607, 15603, 69318, 12303, 48000, 52920, 5433, 38554, 57301, 58324, 55063, 52991, 59914, 59921 or 33751. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 18607, 15603, 69318, 12303, 48000, 52920, 5433, 38554, 57301, 58324, 55063, 52991, 59914, 59921 or 33751.

In yet another embodiment, a cell-free assay is provided in which a 18607, 15603, 69318, 12303, 48000, 52920, 5433, 38554, 57301, 58324, 55063, 52991, 59914, 59921 or 33751 protein or biologically active portion thereof is contacted with a test compound and the ability of the test compound to bind to the 18607, 15603, 69318, 12303, 48000, 52920, 5433, 38554, 57301, 58324, 55063, 52991, 59914, 59921 or 33751 protein or biologically active portion thereof is evaluated. Preferred biologically active portions of the 18607, 15603, 69318, 12303, 48000, 52920, 5433, 38554, 57301, 58324, 55063, 52991, 59914, 59921 or 33751 proteins to be used in assays of the present invention include fragments which participate in interactions with non-18607, 15603, 69318, 12303, 48000, 52920, 5433, 38554, 57301, 58324, 55063, 52991, 59914, 59921 or 33751 molecules, e.g., fragments with high surface probability scores.

Soluble and/or membrane-bound forms of isolated proteins (e.g., 18607, 15603, 69318, 12303, 48000, 52920, 5433, 38554, 57301, 58324, 55063, 52991, 59914, 59921 or 33751 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the 18607, 15603, 69318, 12303, 48000, 52920, 5433, 38554, 57301, 58324, 55063, 52991, 59914, 59921 or 33751 protein to bind to a target molecule can be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either 18607, 15603, 69318, 12303, 48000, 52920, 5433, 38554, 57301, 58324, 55063, 52991, 59914, 59921 or 33751, an anti-18607, 15603, 69318, 12303, 48000, 52920, 5433, 38554, 57301, 58324, 55063, 52991, 59914, 59921 or 33751 antibody or its target molecule to facilitate separation of complexed from uncomplexed forms of one or both of the proteins, as well as to accommodate automation of the assay. Binding of a test compound to a 18607, 15603, 69318, 12303, 48000, 52920, 5433, 38554, 57301, 58324, 55063, 52991, 59914, 59921 or 33751 protein, or interaction of a 18607, 15603, 69318, 12303, 48000, 52920, 5433, 38554, 57301, 58324, 55063, 52991, 59914, 59921 or 33751 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/18607, 15603, 69318, 12303, 48000, 52920, 5433, 38554, 57301, 58324, 55063, 52991, 59914, 59921 or 33751 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or 18607, 15603, 69318, 12303, 48000, 52920, 5433, 38554, 57301, 58324, 55063, 52991, 59914, 59921 or 33751 protein, and the mixture incubated under conditions conducive to complex formation (e.g., at physiological conditions for salt and pH).

Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of 18607, 15603, 69318, 12303, 48000, 52920, 5433, 38554, 57301, 58324, 55063, 52991, 59914, 59921 or 33751 binding or activity determined using standard techniques.

Other techniques for immobilizing either a 18607, 15603, 69318, 12303, 48000, 52920, 5433, 38554, 57301, 58324, 55063, 52991, 59914, 59921 or 33751 protein or a target molecule on matrices include using conjugation of biotin and streptavidin. Biotinylated 18607, 15603, 69318, 12303, 48000, 52920, 5433, 38554, 57301, 58324, 55063, 52991, 59914, 59921 or 33751 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

In one embodiment, this assay is performed utilizing antibodies reactive with 18607, 15603, 69318, 12303, 48000, 52920, 5433, 38554, 57301, 58324, 55063, 52991, 59914, 59921 or 33751 protein or target molecules but which do not interfere with binding of the 18607, 15603, 69318, 12303, 48000, 52920, 5433, 38554, 57301, 58324, 55063, 52991, 59914, 59921 or 33751 protein to its target molecule. Such antibodies can be derivatized to the wells of the plate, and unbound target or 18607, 15603, 69318, 12303, 48000, 52920, 5433, 38554, 57301, 58324, 55063, 52991, 59914, 59921 or 33751 protein trapped in the wells by antibody conjugation. Methods for detecting such complexes, in addition to those described above for the GST-immobilized complexes, include immunodetection of complexes using antibodies reactive with the 18607, 15603, 69318, 12303, 48000, 52920, 5433, 38554, 57301, 58324, 55063, 52991, 59914, 59921 or 33751 protein or target molecule, as well as enzyme-linked assays which rely on detecting an enzymatic activity associated with the 18607, 15603, 69318, 12303, 48000, 52920, 5433, 38554, 57301, 58324, 55063, 52991, 59914, 59921 or 33751 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. (1999) Current Protocols in Molecular Biology, J. Wiley, New York); and immunoprecipitation (see, for example, Ausubel et al., eds. (1999) Current Protocols in Molecular Biology, J. Wiley, New York). Such resins and chromatographic techniques are known to one skilled in the art (see, e.g., Heegaard (1998) J Mol Recognit 11:141-8; Hage and Tweed (1997) J Chromatogr B Biomed Sci Appl. 699:499-525). Further, fluorescence energy transfer can also be conveniently utilized, as described herein, to detect binding without further purification of the complex from solution.

In a preferred embodiment, the assay includes contacting the 18607, 15603, 69318, 12303, 48000, 52920, 5433, 38554, 57301, 58324, 55063, 52991, 59914, 59921 or 33751 protein or biologically active portion thereof with a known compound which binds 18607, 15603, 69318, 12303, 48000, 52920, 5433, 38554, 57301, 58324, 55063, 52991, 59914, 59921 or 33751 to form an assay mixture, contacting the assay mixture with a test compound, and determining the ability of the test compound to interact with a 18607, 15603, 69318, 12303, 48000, 52920, 5433, 38554, 57301, 58324, 55063, 52991, 59914, 59921 or 33751 protein, wherein determining the ability of the test compound to interact with a 18607, 15603, 69318, 12303, 48000, 52920, 5433, 38554, 57301, 58324, 55063, 52991, 59914, 59921 or 33751 protein includes determining the ability of the test compound to preferentially bind to 18607, 15603, 69318, 12303, 48000, 52920, 5433, 38554, 57301, 58324, 55063, 52991, 59914, 59921 or 33751 or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the 18607, 15603, 69318, 12303, 48000, 52920, 5433, 38554, 57301, 58324, 55063, 52991, 59914, 59921 or 33751 genes herein identified. In an alternative embodiment, the invention provides methods for determining the ability of the test compound to modulate the activity of a 18607, 15603, 69318, 12303, 48000, 52920, 5433, 38554, 57301, 58324, 55063, 52991, 59914, 59921 or 33751 protein through modulation of the activity of a downstream effector of a 18607, 15603, 69318, 12303, 48000, 52920, 5433, 38554, 57301, 58324, 55063, 52991, 59914, 59921 or 33751 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner.

Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the 18607, 15603, 69318, 12303, 48000, 52920, 5433, 38554, 57301, 58324, 55063, 52991, 59914, 59921 or 33751 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 18607, 15603, 69318, 12303, 48000, 52920, 5433, 38554, 57301, 58324, 55063, 52991, 59914, 59921 or 33751 (“18607, 15603, 69318, 12303, 48000, 52920, 5433, 38554, 57301, 58324, 55063, 52991, 59914, 59921 or 33751-binding proteins” or “18607, 15603, 69318, 12303, 48000, 52920, 5433, 38554, 57301, 58324, 55063, 52991, 59914, 59921 or 33751-bp”) and are involved in 18607, 15603, 69318, 12303, 48000, 52920, 5433, 38554, 57301, 58324, 55063, 52991, 59914, 59921 or 33751 activity. Such 18607, 15603, 69318, 12303, 48000, 52920, 5433, 38554, 57301, 58324, 55063, 52991, 59914, 59921 or 33751-bps can be activators or inhibitors of signals by the 18607, 15603, 69318, 12303, 48000, 52920, 5433, 38554, 57301, 58324, 55063, 52991, 59914, 59921 or 33751 proteins or 18607, 15603, 69318, 12303, 48000, 52920, 5433, 38554, 57301, 58324, 55063, 52991, 59914, 59921 or 33751 targets as, for example, downstream elements of a 18607, 15603, 69318, 12303, 48000, 52920, 5433, 38554, 57301, 58324, 55063, 52991, 59914, 59921 or 33751-mediated signaling pathway.

The two-hybrid system is based on the modular nature of most transcription factors, which consist of separable DNA-binding and activation domains. Briefly, the assay utilizes two different DNA constructs. In one construct, the gene that codes for a 18607, 15603, 69318, 12303, 48000, 52920, 5433, 38554, 57301, 58324, 55063, 52991, 59914, 59921 or 33751 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: 18607, 15603, 69318, 12303, 48000, 52920, 5433, 38554, 57301, 58324, 55063, 52991, 59914, 59921 or 33751 protein can be the fused to the activator domain.) If the “bait” and the “prey” proteins are able to interact, in vivo, forming a 18607, 15603, 69318, 12303, 48000, 52920, 5433, 38554, 57301, 58324, 55063, 52991, 59914, 59921 or 33751-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 18607, 15603, 69318, 12303, 48000, 52920, 5433, 38554, 57301, 58324, 55063, 52991, 59914, 59921 or 33751 protein.

In another embodiment, modulators of 18607, 15603, 69318, 12303, 48000, 52920, 5433, 38554, 57301, 58324, 55063, 52991, 59914, 59921 or 33751 expression are identified. For example, a cell or cell free mixture is contacted with a candidate compound and the expression of 18607, 15603, 69318, 12303, 48000, 52920, 5433, 38554, 57301, 58324, 55063, 52991, 59914, 59921 or 33751 mRNA or protein evaluated relative to the level of expression of 18607, 15603, 69318, 12303, 48000, 52920, 5433, 38554, 57301, 58324, 55063, 52991, 59914, 59921 or 33751 mRNA or protein in the absence of the candidate compound. When expression of 18607, 15603, 69318, 12303, 48000, 52920, 5433, 38554, 57301, 58324, 55063, 52991, 59914, 59921 or 33751 mRNA or protein is greater in the presence of the candidate compound than in its absence, the candidate compound is identified as a stimulator of 18607, 15603, 69318, 12303, 48000, 52920, 5433, 38554, 57301, 58324, 55063, 52991, 59914, 59921 or 33751 mRNA or protein expression. Alternatively, when expression of 18607, 15603, 69318, 12303, 48000, 52920, 5433, 38554, 57301, 58324, 55063, 52991, 59914, 59921 or 33751 mRNA or protein is less (statistically significantly less) in the presence of the candidate compound than in its absence, the candidate compound is identified as an inhibitor of 18607, 15603, 69318, 12303, 48000, 52920, 5433, 38554, 57301, 58324, 55063, 52991, 59914, 59921 or 33751 mRNA or protein expression. The level of 18607, 15603, 69318, 12303, 48000, 52920, 5433, 38554, 57301, 58324, 55063, 52991, 59914, 59921 or 33751 mRNA or protein expression can be determined by methods described herein for detecting 18607, 15603, 69318, 12303, 48000, 52920, 5433, 38554, 57301, 58324, 55063, 52991, 59914, 59921 or 33751 mRNA or protein.

In another aspect, the invention pertains to a combination of two or more of the assays described herein. For example, a modulating agent can be identified using a cell-based or a cell free assay, and the ability of the agent to modulate the activity of a 18607, 15603, 69318, 12303, 48000, 52920, 5433, 38554, 57301, 58324, 55063, 52991, 59914, 59921 or 33751 protein can be confirmed in vivo, e.g., in an animal such as an animal model for aberrant or deficient calcium channel activity, calcium/sodium antiporter activity, potassium channel activity, organic ion transporter activity, or choline transporter activity.

This invention further pertains to novel agents identified by the above-described screening assays. Accordingly, it is within the scope of this invention to further use an agent identified as described herein (e.g., a 18607, 15603, 69318, 12303, 48000, 52920, 5433, 38554, 57301, 58324, 55063, 52991, 59914, 59921 or 33751 modulating agent, an antisense 18607, 15603, 69318, 12303, 48000, 52920, 5433, 38554, 57301, 58324, 55063, 52991, 59914, 59921 or 33751 nucleic acid molecule, a 18607, 15603, 69318, 12303, 48000, 52920, 5433, 38554, 57301, 58324, 55063, 52991, 59914, 59921 or 33751-specific antibody, or a 18607, 15603, 69318, 12303, 48000, 52920, 5433, 38554, 57301, 58324, 55063, 52991, 59914, 59921 or 33751-binding partner) in an appropriate animal model to determine the efficacy, toxicity, side effects, or mechanism of action, of treatment with such an agent. Furthermore, novel agents identified by the above-described screening assays can be used for treatments as described herein.

### Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate 18607, 15603, 69318, 12303, 48000, 52920, 5433, 38554, 57301, 58324, 55063, 52991, 59914, 59921 or 33751 with a disease; (ii) identify an individual from a minute biological sample (tissue typing); and (iii) aid in forensic identification of a biological sample. These applications are described in the subsections below.

### Chromosome Mapping

The 18607, 15603, 69318, 12303, 48000, 52920, 5433, 38554, 57301, 58324, 55063, 52991, 59914, 59921 or 33751 nucleotide sequences or portions thereof can be used to map the location of the 18607, 15603, 69318, 12303, 48000, 52920, 5433, 38554, 57301, 58324, 55063, 52991, 59914, 59921 or 33751 genes on a chromosome. This process is called chromosome mapping. Chromosome mapping is useful in correlating the 18607, 15603, 69318, 12303, 48000, 52920, 5433, 38554, 57301, 58324, 55063, 52991, 59914, 59921 or 33751 sequences with genes associated with disease.

Briefly, 18607, 15603, 69318, 12303, 48000, 52920, 5433, 38554, 57301, 58324, 55063, 52991, 59914, 59921 or 33751 genes can be mapped to chromosomes by preparing PCR primers (preferably 15-25 bp in length) from the 18607, 15603, 69318, 12303, 48000, 52920, 5433, 38554, 57301, 58324, 55063, 52991, 59914, 59921 or 33751 nucleotide sequences. These primers can then be used for PCR screening of somatic cell hybrids containing individual human chromosomes. Only those hybrids containing the human gene corresponding to the 18607, 15603, 69318, 12303, 48000, 52920, 5433, 38554, 57301, 58324, 55063, 52991, 59914, 59921 or 33751 sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio et al. (1983) Science 220:919-924).

Other mapping strategies e.g., in situ hybridization (described in Fan et al. (1990) Proc. Natl. Acad. Sci. USA, 87:6223-27), pre-screening with labeled flow-sorted chromosomes, and pre-selection by hybridization to chromosome specific cDNA libraries can be used to map 18607, 15603, 69318, 12303, 48000, 52920, 5433, 38554, 57301, 58324, 55063, 52991, 59914, 59921 or 33751 to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al. (1988) Human Chromosomes: A Manual of Basic Techniques, Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the 18607, 15603, 69318, 12303, 48000, 52920, 5433, 38554, 57301, 58324, 55063, 52991, 59914, 59921 or 33751 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

18607, 15603, 69318, 12303, 48000, 52920, 5433, 38554, 57301, 58324, 55063, 52991, 59914, 59921 or 33751 sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the present invention are useful as additional DNA markers for RFLP (described in U.S. Pat. No. 5,272,057).

Furthermore, the sequences of the present invention can also be used to determine the actual base-by-base DNA sequence of selected portions of an individual's genome. Thus, the 18607, 15603, 69318, 12303, 48000, 52920, 5433, 38554, 57301, 58324, 55063, 52991, 59914, 59921 or 33751 nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 5, 13, 18, 21, 24, 27, 32, 35, 38, 53, 56, 60, 69, 72 or 81 can provide positive individual identification with a panel of perhaps 10 to 1,000 primers which each yield a noncoding amplified sequence of 100 bases. If predicted coding sequences, such as those in 0 are used, a more appropriate number of primers for positive individual identification would be 500-2,000.

If a panel of reagents from 18607, 15603, 69318, 12303, 48000, 52920, 5433, 38554, 57301, 58324, 55063, 52991, 59914, 59921 or 33751 nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial 18607, 15603, 69318, 12303, 48000, 52920, 5433, 38554, 57301, 58324, 55063, 52991, 59914, 59921 or 33751 sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another, “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, 5, 13, 18, 21, 24, 27, 32, 35, 38, 53, 56, 60, 69, 72 or 81 (e.g., fragments derived from the noncoding regions of SEQ ID NO:1, 5, 13, 18, 21, 24, 27, 32, 35, 38, 53, 56, 60, 69, 72 or 81 having a length of at least 20 bases, preferably at least 30 bases) are particularly appropriate for this use.

The 18607, 15603, 69318, 12303, 48000, 52920, 5433, 38554, 57301, 58324, 55063, 52991, 59914, 59921 or 33751 nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such 18607, 15603, 69318, 12303, 48000, 52920, 5433, 38554, 57301, 58324, 55063, 52991, 59914, 59921 or 33751 probes can be used to identify tissue by species and/or by organ type.

In a similar fashion, these reagents, e.g., 18607, 15603, 69318, 12303, 48000, 52920, 5433, 38554, 57301, 58324, 55063, 52991, 59914, 59921 or 33751 primers or probes can be used to screen tissue culture for contamination (i.e. screen for the presence of a mixture of different types of cells in a culture).

### Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes 18607, 15603, 69318, 12303, 48000, 52920, 5433, 38554, 57301, 58324, 55063, 52991, 59914, 59921 or 33751.

Such disorders include, e.g., a disorder associated with the misexpression of 18607, 15603, 69318, 12303, 48000, 52920, 5433, 38554, 57301, 58324, 55063, 52991, 59914, 59921 or 33751 gene; a cellular proliferation or differentiation disorder, a cardiovascular, endothelial, breast, lung, colon, prostate, pancreas, brain, blood vessel, platelet, bone, immune, metabolic, kidney, ovarian, viral, pain, liver, skeletal muscle testicular, eye, hormonal, neurological, neurodegenerative, or angiogenic disorder.

The method includes one or more of the following: detecting, in a tissue of the subject, the presence or absence of a mutation which affects the expression of the 18607, 15603, 69318, 12303, 48000, 52920, 5433, 38554, 57301, 58324, 55063, 52991, 59914, 59921 or 33751 gene, or detecting the presence or absence of a mutation in a region which controls the expression of the gene, e.g., a mutation in the 5′ control region; detecting, in a tissue of the subject, the presence or absence of a mutation which alters the structure of the 18607, 15603, 69318, 12303, 48000, 52920, 5433, 38554, 57301, 58324, 55063, 52991, 59914, 59921 or 33751 gene; detecting, in a tissue of the subject, the misexpression of the 18607, 15603, 69318, 12303, 48000, 52920, 5433, 38554, 57301, 58324, 55063, 52991, 59914, 59921 or 33751 gene, at the mRNA level, e.g., detecting a non-wild type level of an mRNA; or detecting, in a tissue of the subject, the misexpression of the gene, at the protein level, e.g., detecting a non-wild type level of a 18607, 15603, 69318, 12303, 48000, 52920, 5433, 38554, 57301, 58324, 55063, 52991, 59914, 59921 or 33751 polypeptide.

In preferred embodiments the method includes: ascertaining the existence of at least one of: a deletion of one or more nucleotides from the 18607, 15603, 69318, 12303, 48000, 52920, 5433, 38554, 57301, 58324, 55063, 52991, 59914, 59921 or 33751 gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1, 5, 13, 18, 21, 24, 27, 32, 35, 38, 53, 56, 60, 69, 72 or 81, or naturally occurring mutants thereof or 5′ or 3′ flanking sequences naturally associated with the 18607, 15603, 69318, 12303, 48000, 52920, 5433, 38554, 57301, 58324, 55063, 52991, 59914, 59921 or 33751 gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the 18607, 15603, 69318, 12303, 48000, 52920, 5433, 38554, 57301, 58324, 55063, 52991, 59914, 59921 or 33751 gene; the presence of a non-wild type splicing pattern of a messenger RNA transcript of the gene; or a non-wild type level of 18607, 15603, 69318, 12303, 48000, 52920, 5433, 38554, 57301, 58324, 55063, 52991, 59914, 59921 or 33751.

Methods of the invention can be used prenatally or to determine if a subject's offspring will be at risk for a disorder.

In preferred embodiments the method includes determining the structure of a 18607, 15603, 69318, 12303, 48000, 52920, 5433, 38554, 57301, 58324, 55063, 52991, 59914, 59921 or 33751 gene, an abnormal structure being indicative of risk for the disorder.

In preferred embodiments the method includes contacting a sample from the subject with an antibody to the 18607, 15603, 69318, 12303, 48000, 52920, 5433, 38554, 57301, 58324, 55063, 52991, 59914, 59921 or 33751 protein or a nucleic acid, which hybridizes specifically with the gene. These and other embodiments are discussed below.

### Diagnostic and Prognostic Assays

The presence, level, or absence of 18607, 15603, 69318, 12303, 48000, 52920, 5433, 38554, 57301, 58324, 55063, 52991, 59914, 59921 or 33751 protein or nucleic acid in a biological sample can be evaluated by obtaining a biological sample from a test subject and contacting the biological sample with a compound or an agent capable of detecting 18607, 15603, 69318, 12303, 48000, 52920, 5433, 38554, 57301, 58324, 55063, 52991, 59914, 59921 or 33751 protein or nucleic acid (e.g., mRNA, genomic DNA) that encodes 18607, 15603, 69318, 12303, 48000, 52920, 5433, 38554, 57301, 58324, 55063, 52991, 59914, 59921 or 33751 protein such that the presence of 18607, 15603, 69318, 12303, 48000, 52920, 5433, 38554, 57301, 58324, 55063, 52991, 59914, 59921 or 33751 protein or nucleic acid is detected in the biological sample. The term “biological sample” includes tissues, cells and biological fluids isolated from a subject, as well as tissues, cells and fluids present within a subject. A preferred biological sample is serum. The level of expression of the 18607, 15603, 69318, 12303, 48000, 52920, 5433, 38554, 57301, 58324, 55063, 52991, 59914, 59921 or 33751 gene can be measured in a number of ways, including, but not limited to: measuring the mRNA encoded by the 18607, 15603, 69318, 12303, 48000, 52920, 5433, 38554, 57301, 58324, 55063, 52991, 59914, 59921 or 33751 genes; measuring the amount of protein encoded by the 18607, 15603, 69318, 12303, 48000, 52920, 5433, 38554, 57301, 58324, 55063, 52991, 59914, 59921 or 33751 genes; or measuring the activity of the protein encoded by the 18607, 15603, 69318, 12303, 48000, 52920, 5433, 38554, 57301, 58324, 55063, 52991, 59914, 59921 or 33751 genes.

The level of mRNA corresponding to the 18607, 15603, 69318, 12303, 48000, 52920, 5433, 38554, 57301, 58324, 55063, 52991, 59914, 59921 or 33751 gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length 18607, 15603, 69318, 12303, 48000, 52920, 5433, 38554, 57301, 58324, 55063, 52991, 59914, 59921 or 33751 nucleic acid, such as the nucleic acid of SEQ ID NO:1, 5, 13, 18, 21, 24, 27, 32, 35, 38, 53, 56, 60, 69, 72 or 81, or a portion thereof, such as an oligonucleotide of at least 7, 15, 30, 50, 100, 250 or 500 nucleotides in length and sufficient to specifically hybridize under stringent conditions to 18607, 15603, 69318, 12303, 48000, 52920, 5433, 38554, 57301, 58324, 55063, 52991, 59914, 59921 or 33751 mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the 18607, 15603, 69318, 12303, 48000, 52920, 5433, 38554, 57301, 58324, 55063, 52991, 59914, 59921 or 33751 genes.

The level of mRNA in a sample that is encoded by one of 18607, 15603, 69318, 12303, 48000, 52920, 5433, 38554, 57301, 58324, 55063, 52991, 59914, 59921 or 33751 can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the 18607, 15603, 69318, 12303, 48000, 52920, 5433, 38554, 57301, 58324, 55063, 52991, 59914, 59921 or 33751 gene being analyzed.

In another embodiment, the methods further contacting a control sample with a compound or agent capable of detecting 18607, 15603, 69318, 12303, 48000, 52920, 5433, 38554, 57301, 58324, 55063, 52991, 59914, 59921 or 33751 mRNA, or genomic DNA, and comparing the presence of 18607, 15603, 69318, 12303, 48000, 52920, 5433, 38554, 57301, 58324, 55063, 52991, 59914, 59921 or 33751 mRNA or genomic DNA in the control sample with the presence of 18607, 15603, 69318, 12303, 48000, 52920, 5433, 38554, 57301, 58324, 55063, 52991, 59914, 59921 or 33751 mRNA or genomic DNA in the test sample.

A variety of methods can be used to determine the level of protein encoded by 18607, 15603, 69318, 12303, 48000, 52920, 5433, 38554, 57301, 58324, 55063, 52991, 59914, 59921 or 33751. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect 18607, 15603, 69318, 12303, 48000, 52920, 5433, 38554, 57301, 58324, 55063, 52991, 59914, 59921 or 33751 protein in a biological sample in vitro as well as in vivo. In vitro techniques for detection of 18607, 15603, 69318, 12303, 48000, 52920, 5433, 38554, 57301, 58324, 55063, 52991, 59914, 59921 or 33751 protein include enzyme linked immunosorbent assays (ELISAs), immunoprecipitations, immunofluorescence, enzyme immunoassay (EIA), radioimmunoassay (RIA), and Western blot analysis. In vivo techniques for detection of 18607, 15603, 69318, 12303, 48000, 52920, 5433, 38554, 57301, 58324, 55063, 52991, 59914, 59921 or 33751 protein include introducing into a subject a labeled anti-18607, 15603, 69318, 12303, 48000, 52920, 5433, 38554, 57301, 58324, 55063, 52991, 59914, 59921 or 33751 antibody. For example, the antibody can be labeled with a radioactive marker whose presence and location in a subject can be detected by standard imaging techniques.

In another embodiment, the methods further include contacting the control sample with a compound or agent capable of detecting 18607, 15603, 69318, 12303, 48000, 52920, 5433, 38554, 57301, 58324, 55063, 52991, 59914, 59921 or 33751 protein, and comparing the presence of 18607, 15603, 69318, 12303, 48000, 52920, 5433, 38554, 57301, 58324, 55063, 52991, 59914, 59921 or 33751 protein in the control sample with the presence of 18607, 15603, 69318, 12303, 48000, 52920, 5433, 38554, 57301, 58324, 55063, 52991, 59914, 59921 or 33751 protein in the test sample.

The invention also includes kits for detecting the presence of 18607, 15603, 69318, 12303, 48000, 52920, 5433, 38554, 57301, 58324, 55063, 52991, 59914, 59921 or 33751 in a biological sample. For example, the kit can include a compound or agent capable of detecting 18607, 15603, 69318, 12303, 48000, 52920, 5433, 38554, 57301, 58324, 55063, 52991, 59914, 59921 or 33751 protein or mRNA in a biological sample; and a standard. The compound or agent can be packaged in a suitable container. The kit can further comprise instructions for using the kit to detect 18607, 15603, 69318, 12303, 48000, 52920, 5433, 38554, 57301, 58324, 55063, 52991, 59914, 59921 or 33751 protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted 18607, 15603, 69318, 12303, 48000, 52920, 5433, 38554, 57301, 58324, 55063, 52991, 59914, 59921 or 33751 expression or activity. As used herein, the term “unwanted” includes an unwanted phenomenon involved in a biological response such as pain or deregulated cell proliferation.

In one embodiment, a disease or disorder associated with aberrant or unwanted 18607, 15603, 69318, 12303, 48000, 52920, 5433, 38554, 57301, 58324, 55063, 52991, 59914, 59921 or 33751 expression or activity is identified. A test sample is obtained from a subject and 18607, 15603, 69318, 12303, 48000, 52920, 5433, 38554, 57301, 58324, 55063, 52991, 59914, 59921 or 33751 protein or nucleic acid (e.g., mRNA or genomic DNA) is evaluated, wherein the level, e.g., the presence or absence, of 18607, 15603, 69318, 12303, 48000, 52920, 5433, 38554, 57301, 58324, 55063, 52991, 59914, 59921 or 33751 protein or nucleic acid is diagnostic for a subject having or at risk of developing a disease or disorder associated with aberrant or unwanted 18607, 15603, 69318, 12303, 48000, 52920, 5433, 38554, 57301, 58324, 55063, 52991, 59914, 59921 or 33751 expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted 18607, 15603, 69318, 12303, 48000, 52920, 5433, 38554, 57301, 58324, 55063, 52991, 59914, 59921 or 33751 expression or activity. For example, such methods can be used to determine whether a subject can be effectively treated with an agent for a cellular proliferation or differentiation disorder, a cardiovascular, endothelial, breast, lung, colon, prostate, pancreas, brain, blood vessel, platelet, bone, immune, metabolic, kidney, ovarian, viral, pain, liver, skeletal muscle testicular, eye, hormonal, neurological, neurodegenerative or angiogenic disorder.

The methods of the invention can also be used to detect genetic alterations in a 18607, 15603, 69318, 12303, 48000, 52920, 5433, 38554, 57301, 58324, 55063, 52991, 59914, 59921 or 33751 gene, thereby determining if a subject with the altered gene is at risk for a disorder characterized by misregulation in 18607, 15603, 69318, 12303, 48000, 52920, 5433, 38554, 57301, 58324, 55063, 52991, 59914, 59921 or 33751 protein activity or nucleic acid expression, such as a a cellular proliferation or differentiation disorder, a cardiovascular, endothelial, breast, lung, colon, prostate, pancreas, brain, blood vessel, platelet, bone, immune, metabolic, kidney, ovarian, viral, pain, liver, skeletal muscle testicular, eye, hormonal neurological, neurodegenerative or angiogenic disorder. In preferred embodiments, the methods include detecting, in a sample from the subject, the presence or absence of a genetic alteration characterized by at least one of an alteration affecting the integrity of a gene encoding a 18607, 15603, 69318, 12303, 48000, 52920, 5433, 38554, 57301, 58324, 55063, 52991, 59914, 59921 or 33751-protein, or the mis-expression of the 18607, 15603, 69318, 12303, 48000, 52920, 5433, 38554, 57301, 58324, 55063, 52991, 59914, 59921 or 33751 gene. For example, such genetic alterations can be detected by ascertaining the existence of at least one of 1) a deletion of one or more nucleotides from a 18607, 15603, 69318, 12303, 48000, 52920, 5433, 38554, 57301, 58324, 55063, 52991, 59914, 59921 or 33751 gene; 2) an addition of one or more nucleotides to a 18607, 15603, 69318, 12303, 48000, 52920, 5433, 38554, 57301, 58324, 55063, 52991, 59914, 59921 or 33751 gene; 3) a substitution of one or more nucleotides of a 18607, 15603, 69318, 12303, 48000, 52920, 5433, 38554, 57301, 58324, 55063, 52991, 59914, 59921 or 33751 gene, 4) a chromosomal rearrangement of a 18607, 15603, 69318, 12303, 48000, 52920, 5433, 38554, 57301, 58324, 55063, 52991, 59914, 59921 or 33751 gene; 5) an alteration in the level of a messenger RNA transcript of a 18607, 15603, 69318, 12303, 48000, 52920, 5433, 38554, 57301, 58324, 55063, 52991, 59914, 59921 or 33751 gene, 6) aberrant modification of a 18607, 15603, 69318, 12303, 48000, 52920, 5433, 38554, 57301, 58324, 55063, 52991, 59914, 59921 or 33751 gene, such as of the methylation pattern of the genomic DNA, 7) the presence of a non-wild type splicing pattern of a messenger RNA transcript of a 18607, 15603, 69318, 12303, 48000, 52920, 5433, 38554, 57301, 58324, 55063, 52991, 59914, 59921 or 33751 gene, 8) a non-wild type level of a 18607, 15603, 69318, 12303, 48000, 52920, 5433, 38554, 57301, 58324, 55063, 52991, 59914, 59921 or 33751-protein, 9) allelic loss of a 18607, 15603, 69318, 12303, 48000, 52920, 5433, 38554, 57301, 58324, 55063, 52991, 59914, 59921 or 33751 gene, and 10) inappropriate post-translational modification of a 18607, 15603, 69318, 12303, 48000, 52920, 5433, 38554, 57301, 58324, 55063, 52991, 59914, 59921 or 33751-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the 18607, 15603, 69318, 12303, 48000, 52920, 5433, 38554, 57301, 58324, 55063, 52991, 59914, 59921 or 33751-gene. This method can include the steps of collecting a sample of cells from a subject, isolating nucleic acid (e.g., genomic, mRNA or both) from the sample, contacting the nucleic acid sample with one or more primers which specifically hybridize to a 18607, 15603, 69318, 12303, 48000, 52920, 5433, 38554, 57301, 58324, 55063, 52991, 59914, 59921 or 33751 gene under conditions such that hybridization and amplification of the 18607, 15603, 69318, 12303, 48000, 52920, 5433, 38554, 57301, 58324, 55063, 52991, 59914, 59921 or 33751 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a 18607, 15603, 69318, 12303, 48000, 52920, 5433, 38554, 57301, 58324, 55063, 52991, 59914, 59921 or 33751 gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 18607, 15603, 69318, 12303, 48000, 52920, 5433, 38554, 57301, 58324, 55063, 52991, 59914, 59921 or 33751 can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al. (1996) Human Mutation 7: 244-255; Kozal et al. (1996) Nature Medicine 2: 753-759). For example, genetic mutations in 18607, 15603, 69318, 12303, 48000, 52920, 5433, 38554, 57301, 58324, 55063, 52991, 59914, 59921 or 33751 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 18607, 15603, 69318, 12303, 48000, 52920, 5433, 38554, 57301, 58324, 55063, 52991, 59914, 59921 or 33751 gene and detect mutations by comparing the sequence of the sample 18607, 15603, 69318, 12303, 48000, 52920, 5433, 38554, 57301, 58324, 55063, 52991, 59914, 59921 or 33751 with the corresponding wild-type (control) sequence. Automated sequencing procedures can be utilized when performing the diagnostic assays (Naeve et al. (1995) Biotechniques 19:448-53), including sequencing by mass spectrometry.

Other methods for detecting mutations in the 18607, 15603, 69318, 12303, 48000, 52920, 5433, 38554, 57301, 58324, 55063, 52991, 59914, 59921 or 33751 gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in 18607, 15603, 69318, 12303, 48000, 52920, 5433, 38554, 57301, 58324, 55063, 52991, 59914, 59921 or 33751 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in 18607, 15603, 69318, 12303, 48000, 52920, 5433, 38554, 57301, 58324, 55063, 52991, 59914, 59921 or 33751 genes. For example, single strand conformation polymorphism (SSCP) can be used to detect differences in electrophoretic mobility between mutant and wild type nucleic acids (Orita et al. (1989) Proc Natl. Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control 18607, 15603, 69318, 12303, 48000, 52920, 5433, 38554, 57301, 58324, 55063, 52991, 59914, 59921 or 33751 nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci. USA 86:6230).

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell. Probes 6: 1). It is anticipated that in certain embodiments amplification can also be performed using Taq ligase for amplification (Barany (1991) Proc. Natl. Acad. Sci. USA 88:189-93). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a 18607, 15603, 69318, 12303, 48000, 52920, 5433, 38554, 57301, 58324, 55063, 52991, 59914, 59921 or 33751 gene.

### Use of 18607, 15603, 69318, 12303, 48000, 52920, 5433, 38554, 57301, 58324, 55063, 52991, 59914, 59921 or 33751 Molecules as Surrogate Markers

The 18607, 15603, 69318, 12303, 48000, 52920, 5433, 38554, 57301, 58324, 55063, 52991, 59914, 59921 or 33751 molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the 18607, 15603, 69318, 12303, 48000, 52920, 5433, 38554, 57301, 58324, 55063, 52991, 59914, 59921 or 33751 molecules of the invention can be detected, and can be correlated with one or more biological states in vivo. For example, the 18607, 15603, 69318, 12303, 48000, 52920, 5433, 38554, 57301, 58324, 55063, 52991, 59914, 59921 or 33751 molecules of the invention can serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers can serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease can be made using cholesterol levels as a surrogate marker, and an analysis of HIV infection can be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The 18607, 15603, 69318, 12303, 48000, 52920, 5433, 38554, 57301, 58324, 55063, 52991, 59914, 59921 or 33751 molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a 18607, 15603, 69318, 12303, 48000, 52920, 5433, 38554, 57301, 58324, 55063, 52991, 59914, 59921 or 33751 marker) transcription or expression, the amplified marker can be in a quantity which is more readily detectable than the drug itself. Also, the marker can be more easily detected due to the nature of the marker itself; for example, using the methods described herein, anti-18607, 15603, 69318, 12303, 48000, 52920, 5433, 38554, 57301, 58324, 55063, 52991, 59914, 59921 or 33751 antibodies can be employed in an immune-based detection system for a 18607, 15603, 69318, 12303, 48000, 52920, 5433, 38554, 57301, 58324, 55063, 52991, 59914, 59921 or 33751 protein marker, or 18607, 15603, 69318, 12303, 48000, 52920, 5433, 38554, 57301, 58324, 55063, 52991, 59914, 59921 or 33751-specific radiolabeled probes can be used to detect a 18607, 15603, 69318, 12303, 48000, 52920, 5433, 38554, 57301, 58324, 55063, 52991, 59914, 59921 or 33751 mRNA marker. Furthermore, the use of a pharmacodynamic marker can offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The 18607, 15603, 69318, 12303, 48000, 52920, 5433, 38554, 57301, 58324, 55063, 52991, 59914, 59921 or 33751 molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., 18607, 15603, 69318, 12303, 48000, 52920, 5433, 38554, 57301, 58324, 55063, 52991, 59914, 59921 or 33751 protein or RNA) for specific tumor markers in a subject, a drug or course of treatment can be selected that is optimized for the treatment of the specific tumor likely to be present in the subject. Similarly, the presence or absence of a specific sequence mutation in 18607, 15603, 69318, 12303, 48000, 52920, 5433, 38554, 57301, 58324, 55063, 52991, 59914, 59921 or 33751 dNA can correlate with a 18607, 15603, 69318, 12303, 48000, 52920, 5433, 38554, 57301, 58324, 55063, 52991, 59914, 59921 or 33751 drug response. The use of pharmacogenomic markers therefore permits the application of the most appropriate treatment for each subject without having to administer the therapy.

### Pharmaceutical Compositions

The nucleic acid and polypeptides, fragments thereof, as well as anti-18607, 15603, 69318, 12303, 48000, 52920, 5433, 38554, 57301, 58324, 55063, 52991, 59914, 59921 or 33751 antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody, unconjugated or conjugated as described herein, can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent can, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### Methods of Treatment:

The present invention provides for both prophylactic and therapeutic methods of treating a subject at risk of (or susceptible to) a disorder or having a disorder associated with aberrant or unwanted 18607, 15603, 69318, 12303, 48000, 52920, 5433, 38554, 57301, 58324, 55063, 52991, 59914, 59921 or 33751 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments can be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the 18607, 15603, 69318, 12303, 48000, 52920, 5433, 38554, 57301, 58324, 55063, 52991, 59914, 59921 or 33751 molecules of the present invention or 18607, 15603, 69318, 12303, 48000, 52920, 5433, 38554, 57301, 58324, 55063, 52991, 59914, 59921 or 33751 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted 18607, 15603, 69318, 12303, 48000, 52920, 5433, 38554, 57301, 58324, 55063, 52991, 59914, 59921 or 33751 expression or activity, by administering to the subject a 18607, 15603, 69318, 12303, 48000, 52920, 5433, 38554, 57301, 58324, 55063, 52991, 59914, 59921 or 33751 or an agent which modulates 18607, 15603, 69318, 12303, 48000, 52920, 5433, 38554, 57301, 58324, 55063, 52991, 59914, 59921 or 33751 expression or at least one 18607, 15603, 69318, 12303, 48000, 52920, 5433, 38554, 57301, 58324, 55063, 52991, 59914, 59921 or 33751 activity. Subjects at risk for a disease which is caused or contributed to by aberrant or unwanted 18607, 15603, 69318, 12303, 48000, 52920, 5433, 38554, 57301, 58324, 55063, 52991, 59914, 59921 or 33751 expression or activity can be identified by, for example, any or a combination of diagnostic or prognostic assays as described herein. Administration of a prophylactic agent can occur prior to the manifestation of symptoms characteristic of the 18607, 15603, 69318, 12303, 48000, 52920, 5433, 38554, 57301, 58324, 55063, 52991, 59914, 59921 or 33751 aberrance, such that a disease or disorder is prevented or, alternatively, delayed in its progression. Depending on the type of 18607, 15603, 69318, 12303, 48000, 52920, 5433, 38554, 57301, 58324, 55063, 52991, 59914, 59921 or 33751 aberrance, for example, a 18607, 15603, 69318, 12303, 48000, 52920, 5433, 38554, 57301, 58324, 55063, 52991, 59914, 59921 or 33751, 18607, 15603, 69318, 12303, 48000, 52920, 5433, 38554, 57301, 58324, 55063, 52991, 59914, 59921 or 33751 agonist or 18607, 15603, 69318, 12303, 48000, 52920, 5433, 38554, 57301, 58324, 55063, 52991, 59914, 59921 or 33751 antagonist agent can be used for treating the subject. The appropriate agent can be determined based on screening assays described herein.

It is possible that some 18607, 15603, 69318, 12303, 48000, 52920, 5433, 38554, 57301, 58324, 55063, 52991, 59914, 59921 or 33751 disorders can be caused, at least in part, by an abnormal level of gene product, or by the presence of a gene product exhibiting abnormal activity. As such, the reduction in the level and/or activity of such gene products would bring about the amelioration of disorder symptoms.

The 18607, 15603, 69318, 12303, 48000, 52920, 5433, 38554, 57301, 58324, 55063, 52991, 59914, 59921 or 33751 molecules can act as novel diagnostic targets and therapeutic agents for controlling one or more of a cellular proliferation and/or differentiation disorder, a cardiovascular, endothelial, breast, lung, colon, prostate, pancreas, brain, blood vessel, platelet, bone, immune, metabolic, kidney, ovarian, viral, pain, liver, skeletal muscle testicular, eye, hormonalneurological, neurodegenerative or angiogenic disorder, all of which are described above.

As discussed, successful treatment of 18607, 15603, 69318, 12303, 48000, 52920, 5433, 38554, 57301, 58324, 55063, 52991, 59914, 59921 or 33751 disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of 18607, 15603, 69318, 12303, 48000, 52920, 5433, 38554, 57301, 58324, 55063, 52991, 59914, 59921 or 33751 disorders. Such molecules can include, but are not limited to peptides, phosphopeptides, small organic or inorganic molecules, or antibodies (including, for example, polyclonal, monoclonal, humanized, human, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules can be utilized in treating or preventing a disease characterized by 18607, 15603, 69318, 12303, 48000, 52920, 5433, 38554, 57301, 58324, 55063, 52991, 59914, 59921 or 33751 expression is through the use of aptamer molecules specific for 18607, 15603, 69318, 12303, 48000, 52920, 5433, 38554, 57301, 58324, 55063, 52991, 59914, 59921 or 33751 protein. Aptamers are nucleic acid molecules having a tertiary structure which permits them to specifically or selectively bind to protein ligands (see, e.g., Osborne et al. (1997) Curr. Opin. Chem. Biol. 1: 5-9; and Patel (1997) Curr Opin Chem Biol 1:32-46). Since nucleic acid molecules can in many cases be more conveniently introduced into target cells than therapeutic protein molecules can be, aptamers offer a method by which 18607, 15603, 69318, 12303, 48000, 52920, 5433, 38554, 57301, 58324, 55063, 52991, 59914, 59921 or 33751 protein activity can be specifically decreased without the introduction of drugs or other molecules which can have pluripotent effects.

Antibodies can be generated that are both specific for target gene product and that reduce target gene product activity. Such antibodies can, therefore, by administered in instances whereby negative modulatory techniques are appropriate for the treatment of 18607, 15603, 69318, 12303, 48000, 52920, 5433, 38554, 57301, 58324, 55063, 52991, 59914, 59921 or 33751 disorders. For a description of antibodies, see the Antibody section above.

In circumstances wherein injection of an animal or a human subject with a 18607, 15603, 69318, 12303, 48000, 52920, 5433, 38554, 57301, 58324, 55063, 52991, 59914, 59921 or 33751 protein or epitope for stimulating antibody production is harmful to the subject, it is possible to generate an immune response against 18607, 15603, 69318, 12303, 48000, 52920, 5433, 38554, 57301, 58324, 55063, 52991, 59914, 59921 or 33751 through the use of anti-idiotypic antibodies (see, for example, Herlyn (1999) Ann Med 31:66-78; and Bhattacharya-Chatterjee and Foon (1998) Cancer Treat Res. 94:51-68). If an anti-idiotypic antibody is introduced into a mammal or human subject, it should stimulate the production of anti-anti-idiotypic antibodies, which should be specific to the 18607, 15603, 69318, 12303, 48000, 52920, 5433, 38554, 57301, 58324, 55063, 52991, 59914, 59921 or 33751 protein.

Vaccines directed to a disease characterized by 18607, 15603, 69318, 12303, 48000, 52920, 5433, 38554, 57301, 58324, 55063, 52991, 59914, 59921 or 33751 expression can also be generated in this fashion.

In instances where the target antigen is intracellular and whole antibodies are used, internalizing antibodies can be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate 18607, 15603, 69318, 12303, 48000, 52920, 5433, 38554, 57301, 58324, 55063, 52991, 59914, 59921 or 33751 disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate 18607, 15603, 69318, 12303, 48000, 52920, 5433, 38554, 57301, 58324, 55063, 52991, 59914, 59921 or 33751 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647. Through the use of isotope-labeling, the “free” concentration of compound which modulates the expression or activity of 18607, 15603, 69318, 12303, 48000, 52920, 5433, 38554, 57301, 58324, 55063, 52991, 59914, 59921 or 33751 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz et al (1995) Analytical Chemistry 67:2142-2144.

Another aspect of the invention pertains to methods of modulating 18607, 15603, 69318, 12303, 48000, 52920, 5433, 38554, 57301, 58324, 55063, 52991, 59914, 59921 or 33751 expression or activity for therapeutic purposes. Accordingly, in an exemplary embodiment, the modulatory method of the invention involves contacting a cell with a 18607, 15603, 69318, 12303, 48000, 52920, 5433, 38554, 57301, 58324, 55063, 52991, 59914, 59921 or 33751 or agent that modulates one or more of the activities of 18607, 15603, 69318, 12303, 48000, 52920, 5433, 38554, 57301, 58324, 55063, 52991, 59914, 59921 or 33751 protein activity associated with the cell. An agent that modulates 18607, 15603, 69318, 12303, 48000, 52920, 5433, 38554, 57301, 58324, 55063, 52991, 59914, 59921 or 33751 protein activity can be an agent as described herein, such as a nucleic acid or a protein, a naturally-occurring target molecule of a 18607, 15603, 69318, 12303, 48000, 52920, 5433, 38554, 57301, 58324, 55063, 52991, 59914, 59921 or 33751 protein (e.g., a 18607, 15603, 69318, 12303, 48000, 52920, 5433, 38554, 57301, 58324, 55063, 52991, 59914, 59921 or 33751 substrate or receptor), a 18607, 15603, 69318, 12303, 48000, 52920, 5433, 38554, 57301, 58324, 55063, 52991, 59914, 59921 or 33751 antibody, a 18607, 15603, 69318, 12303, 48000, 52920, 5433, 38554, 57301, 58324, 55063, 52991, 59914, 59921 or 33751 agonist or antagonist, a peptidomimetic of a 18607, 15603, 69318, 12303, 48000, 52920, 5433, 38554, 57301, 58324, 55063, 52991, 59914, 59921 or 33751 agonist or antagonist, or other small molecule.

In one embodiment, the agent stimulates one or 18607, 15603, 69318, 12303, 48000, 52920, 5433, 38554, 57301, 58324, 55063, 52991, 59914, 59921 or 33751 activities. Examples of such stimulatory agents include active 18607, 15603, 69318, 12303, 48000, 52920, 5433, 38554, 57301, 58324, 55063, 52991, 59914, 59921 or 33751 protein and a nucleic acid molecule encoding 18607, 15603, 69318, 12303, 48000, 52920, 5433, 38554, 57301, 58324, 55063, 52991, 59914, 59921 or 33751. In another embodiment, the agent inhibits one or more 18607, 15603, 69318, 12303, 48000, 52920, 5433, 38554, 57301, 58324, 55063, 52991, 59914, 59921 or 33751 activities. Examples of such inhibitory agents include antisense 18607, 15603, 69318, 12303, 48000, 52920, 5433, 38554, 57301, 58324, 55063, 52991, 59914, 59921 or 33751 nucleic acid molecules, anti-18607, 15603, 69318, 12303, 48000, 52920, 5433, 38554, 57301, 58324, 55063, 52991, 59914, 59921 or 33751 antibodies, and 18607, 15603, 69318, 12303, 48000, 52920, 5433, 38554, 57301, 58324, 55063, 52991, 59914, 59921 or 33751 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 18607, 15603, 69318, 12303, 48000, 52920, 5433, 38554, 57301, 58324, 55063, 52991, 59914, 59921 or 33751 protein or nucleic acid molecule. In one embodiment, the method involves administering an agent (e.g., an agent identified by a screening assay described herein), or combination of agents that modulates (e.g., up regulates or down regulates) 18607, 15603, 69318, 12303, 48000, 52920, 5433, 38554, 57301, 58324, 55063, 52991, 59914, 59921 or 33751 expression or activity. In another embodiment, the method involves administering a 18607, 15603, 69318, 12303, 48000, 52920, 5433, 38554, 57301, 58324, 55063, 52991, 59914, 59921 or 33751 protein or nucleic acid molecule as therapy to compensate for reduced, aberrant, or unwanted 18607, 15603, 69318, 12303, 48000, 52920, 5433, 38554, 57301, 58324, 55063, 52991, 59914, 59921 or 33751 expression or activity.

Stimulation of 18607, 15603, 69318, 12303, 48000, 52920, 5433, 38554, 57301, 58324, 55063, 52991, 59914, 59921 or 33751 activity is desirable in situations in which 18607, 15603, 69318, 12303, 48000, 52920, 5433, 38554, 57301, 58324, 55063, 52991, 59914, 59921 or 33751 is abnormally downregulated and/or in which increased 18607, 15603, 69318, 12303, 48000, 52920, 5433, 38554, 57301, 58324, 55063, 52991, 59914, 59921 or 33751 activity is likely to have a beneficial effect. For example, stimulation of 18607, 15603, 69318, 12303, 48000, 52920, 5433, 38554, 57301, 58324, 55063, 52991, 59914, 59921 or 33751 activity is desirable in situations in which a 18607, 15603, 69318, 12303, 48000, 52920, 5433, 38554, 57301, 58324, 55063, 52991, 59914, 59921 or 33751 is downregulated and/or in which increased 18607, 15603, 69318, 12303, 48000, 52920, 5433, 38554, 57301, 58324, 55063, 52991, 59914, 59921 or 33751 activity is likely to have a beneficial effect. Likewise, inhibition of 18607, 15603, 69318, 12303, 48000, 52920, 5433, 38554, 57301, 58324, 55063, 52991, 59914, 59921 or 33751 activity is desirable in situations in which 18607, 15603, 69318, 12303, 48000, 52920, 5433, 38554, 57301, 58324, 55063, 52991, 59914, 59921 or 33751 is abnormally upregulated and/or in which decreased 18607, 15603, 69318, 12303, 48000, 52920, 5433, 38554, 57301, 58324, 55063, 52991, 59914, 59921 or 33751 activity is likely to have a beneficial effect.

### Pharmacogenomics

The 18607, 15603, 69318, 12303, 48000, 52920, 5433, 38554, 57301, 58324, 55063, 52991, 59914, 59921 or 33751 molecules of the present invention, as well as agents, or modulators which have a stimulatory or inhibitory effect on 18607, 15603, 69318, 12303, 48000, 52920, 5433, 38554, 57301, 58324, 55063, 52991, 59914, 59921 or 33751 activity (e.g., 18607, 15603, 69318, 12303, 48000, 52920, 5433, 38554, 57301, 58324, 55063, 52991, 59914, 59921 or 33751 gene expression) as identified by a screening assay described herein can be administered to individuals to treat (prophylactically or therapeutically) 18607, 15603, 69318, 12303, 48000, 52920, 5433, 38554, 57301, 58324, 55063, 52991, 59914, 59921 or 33751-associated disorders (e.g., aberrant or deficient calcium channel activity, calcium/sodium antiporter activity, potassium channel activity, organic ion transporter activity, or choline transporter activity) associated with aberrant or unwanted 18607, 15603, 69318, 12303, 48000, 52920, 5433, 38554, 57301, 58324, 55063, 52991, 59914, 59921 or 33751 activity.

In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a 18607, 15603, 69318, 12303, 48000, 52920, 5433, 38554, 57301, 58324, 55063, 52991, 59914, 59921 or 33751 molecule or 18607, 15603, 69318, 12303, 48000, 52920, 5433, 38554, 57301, 58324, 55063, 52991, 59914, 59921 or 33751 modulator as well as tailoring the dosage and/or therapeutic regimen of treatment with a 18607, 15603, 69318, 12303, 48000, 52920, 5433, 38554, 57301, 58324, 55063, 52991, 59914, 59921 or 33751 molecule or 18607, 15603, 69318, 12303, 48000, 52920, 5433, 38554, 57301, 58324, 55063, 52991, 59914, 59921 or 33751 modulator.

Pharmacogenomics deals with clinically significant hereditary variations in the response to drugs due to altered drug disposition and abnormal action in affected persons. See, for example, Eichelbaum et al. (1996) Clin. Exp. Pharmacol. Physiol. 23:983-985 and Linder et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP can occur once per every 1000 bases of DNA. A SNP can be involved in a disease process, however, the vast majority can not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach”, can be utilized to identify genes that predict drug response. According to this method, if a gene that encodes a drug's target is known (e.g., a 18607, 15603, 69318, 12303, 48000, 52920, 5433, 38554, 57301, 58324, 55063, 52991, 59914, 59921 or 33751 protein of the present invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a 18607, 15603, 69318, 12303, 48000, 52920, 5433, 38554, 57301, 58324, 55063, 52991, 59914, 59921 or 33751 molecule or 18607, 15603, 69318, 12303, 48000, 52920, 5433, 38554, 57301, 58324, 55063, 52991, 59914, 59921 or 33751 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a 18607, 15603, 69318, 12303, 48000, 52920, 5433, 38554, 57301, 58324, 55063, 52991, 59914, 59921 or 33751 molecule or 18607, 15603, 69318, 12303, 48000, 52920, 5433, 38554, 57301, 58324, 55063, 52991, 59914, 59921 or 33751 modulator, such as a modulator identified by one of the exemplary screening assays described herein.

The present invention further provides methods for identifying new agents, or combinations, that are based on identifying agents that modulate the activity of one or more of the gene products encoded by one or more of the 18607, 15603, 69318, 12303, 48000, 52920, 5433, 38554, 57301, 58324, 55063, 52991, 59914, 59921 or 33751 genes of the present invention, wherein these products can be associated with resistance of the cells to a therapeutic agent. Specifically, the activity of the proteins encoded by the 18607, 15603, 69318, 12303, 48000, 52920, 5433, 38554, 57301, 58324, 55063, 52991, 59914, 59921 or 33751 genes of the present invention can be used as a basis for identifying agents for overcoming agent resistance. By blocking the activity of one or more of the resistance proteins, target cells, e.g., human cells, will become sensitive to treatment with an agent to which the unmodified target cells were resistant.

Monitoring the influence of agents (e.g., drugs) on the expression or activity of a 18607, 15603, 69318, 12303, 48000, 52920, 5433, 38554, 57301, 58324, 55063, 52991, 59914, 59921 or 33751 protein can be applied in clinical trials. For example, the effectiveness of an agent determined by a screening assay as described herein to increase 18607, 15603, 69318, 12303, 48000, 52920, 5433, 38554, 57301, 58324, 55063, 52991, 59914, 59921 or 33751 gene expression, protein levels, or upregulate 18607, 15603, 69318, 12303, 48000, 52920, 5433, 38554, 57301, 58324, 55063, 52991, 59914, 59921 or 33751 activity, can be monitored in clinical trials of subjects exhibiting decreased 18607, 15603, 69318, 12303, 48000, 52920, 5433, 38554, 57301, 58324, 55063, 52991, 59914, 59921 or 33751 gene expression, protein levels, or downregulated 18607, 15603, 69318, 12303, 48000, 52920, 5433, 38554, 57301, 58324, 55063, 52991, 59914, 59921 or 33751 activity. Alternatively, the effectiveness of an agent determined by a screening assay to decrease 18607, 15603, 69318, 12303, 48000, 52920, 5433, 38554, 57301, 58324, 55063, 52991, 59914, 59921 or 33751 gene expression, protein levels, or downregulate 18607, 15603, 69318, 12303, 48000, 52920, 5433, 38554, 57301, 58324, 55063, 52991, 59914, 59921 or 33751 activity, can be monitored in clinical trials of subjects exhibiting increased 18607, 15603, 69318, 12303, 48000, 52920, 5433, 38554, 57301, 58324, 55063, 52991, 59914, 59921 or 33751 gene expression, protein levels, or upregulated 18607, 15603, 69318, 12303, 48000, 52920, 5433, 38554, 57301, 58324, 55063, 52991, 59914, 59921 or 33751 activity. In such clinical trials, the expression or activity of a 18607, 15603, 69318, 12303, 48000, 52920, 5433, 38554, 57301, 58324, 55063, 52991, 59914, 59921 or 33751 gene, and preferably, other genes that have been implicated in, for example, a ion channel-associated or another 18607, 15603, 69318, 12303, 48000, 52920, 5433, 38554, 57301, 58324, 55063, 52991, 59914, 59921 or 33751-associated disorder can be used as a “read out” or markers of the phenotype of a particular cell.

### Other Embodiments

In another aspect, the invention features a method of analyzing a plurality of capture probes. The method is useful, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence, wherein the capture probes are from a cell or subject which expresses 18607, 15603, 69318, 12303, 48000, 52920, 5433, 38554, 57301, 58324, 55063, 52991, 59914, 59921 or 33751 or from a cell or subject in which a 18607, 15603, 69318, 12303, 48000, 52920, 5433, 38554, 57301, 58324, 55063, 52991, 59914, 59921 or 33751 mediated response has been elicited; contacting the array with a 18607, 15603, 69318, 12303, 48000, 52920, 5433, 38554, 57301, 58324, 55063, 52991, 59914, 59921 or 33751 nucleic acid (preferably purified), a 18607, 15603, 69318, 12303, 48000, 52920, 5433, 38554, 57301, 58324, 55063, 52991, 59914, 59921 or 33751 polypeptide (preferably purified), or an anti-18607, 15603, 69318, 12303, 48000, 52920, 5433, 38554, 57301, 58324, 55063, 52991, 59914, 59921 or 33751 antibody, and thereby evaluating the plurality of capture probes. Binding, e.g., in the case of a nucleic acid, hybridization with a capture probe at an address of the plurality, is detected, e.g., by a signal generated from a label attached to the 18607, 15603, 69318, 12303, 48000, 52920, 5433, 38554, 57301, 58324, 55063, 52991, 59914, 59921 or 33751 nucleic acid, polypeptide, or antibody.

The capture probes can be a set of nucleic acids from a selected sample, e.g., a sample of nucleic acids derived from a control or non-stimulated tissue or cell.

The method can include contacting the 18607, 15603, 69318, 12303, 48000, 52920, 5433, 38554, 57301, 58324, 55063, 52991, 59914, 59921 or 33751 nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of 18607, 15603, 69318, 12303, 48000, 52920, 5433, 38554, 57301, 58324, 55063, 52991, 59914, 59921 or 33751. Such methods can be used to diagnose a subject, e.g., to evaluate risk for a disease or disorder, to evaluate suitability of a selected treatment for a subject, to evaluate whether a subject has a disease or disorder.

The method can be used to detect SNPs, as described above.

In another aspect, the invention features, a method of analyzing 18607, 15603, 69318, 12303, 48000, 52920, 5433, 38554, 57301, 58324, 55063, 52991, 59914, 59921 or 33751, e.g., analyzing structure, function, or relatedness to other nucleic acid or amino acid sequences. The method includes: providing a 18607, 15603, 69318, 12303, 48000, 52920, 5433, 38554, 57301, 58324, 55063, 52991, 59914, 59921 or 33751 nucleic acid or amino acid sequence; comparing the 18607, 15603, 69318, 12303, 48000, 52920, 5433, 38554, 57301, 58324, 55063, 52991, 59914, 59921 or 33751 sequence with one or more preferably a plurality of sequences from a collection of sequences, e.g., a nucleic acid or protein sequence database; to thereby analyze 18607, 15603, 69318, 12303, 48000, 52920, 5433, 38554, 57301, 58324, 55063, 52991, 59914, 59921 or 33751.

The method can include evaluating the sequence identity between a 18607, 15603, 69318, 12303, 48000, 52920, 5433, 38554, 57301, 58324, 55063, 52991, 59914, 59921 or 33751 sequence and a database sequence. The method can be performed by accessing the database at a second site, e.g., over the internet. Preferred databases include GenBank™ and SwissProt.

In another aspect, the invention features, a set of oligonucleotides, useful, e.g., for identifying SNP's, or identifying specific alleles of 18607, 15603, 69318, 12303, 48000, 52920, 5433, 38554, 57301, 58324, 55063, 52991, 59914, 59921 or 33751. The set includes a plurality of oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotide which hybridizes to one allele provides a signal that is distinguishable from an oligonucleotides which hybridizes to a second allele.

The sequences of 18607, 15603, 69318, 12303, 48000, 52920, 5433, 38554, 57301, 58324, 55063, 52991, 59914, 59921 or 33751 molecules are provided in a variety of mediums to facilitate use thereof. A sequence can be provided as a manufacture, other than an isolated nucleic acid or amino acid molecule, which contains a 18607, 15603, 69318, 12303, 48000, 52920, 5433, 38554, 57301, 58324, 55063, 52991, 59914, 59921 or 33751 molecule. Such a manufacture can provide a nucleotide or amino acid sequence, e.g., an open reading frame, in a form which allows examination of the manufacture using means not directly applicable to examining the nucleotide or amino acid sequences, or a subset thereof, as they exist in nature or in purified form.

A 18607, 15603, 69318, 12303, 48000, 52920, 5433, 38554, 57301, 58324, 55063, 52991, 59914, 59921 or 33751 nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as compact disc and CD-ROM; electrical storage media such as RAM, ROM, EPROM, EEPROM, and the like; and general hard disks and hybrids of these categories such as magnetic/optical storage media. The medium is adapted or configured for having thereon 18607, 15603, 69318, 12303, 48000, 52920, 5433, 38554, 57301, 58324, 55063, 52991, 59914, 59921 or 33751 sequence information of the present invention.

As used herein, the term “electronic apparatus” is intended to include any suitable computing or processing apparatus of other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phones, pagers, and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 18607, 15603, 69318, 12303, 48000, 52920, 5433, 38554, 57301, 58324, 55063, 52991, 59914, 59921 or 33751 sequence information.

A variety of data storage structures are available to a skilled artisan for creating a computer readable medium having recorded thereon a 18607, 15603, 69318, 12303, 48000, 52920, 5433, 38554, 57301, 58324, 55063, 52991, 59914, 59921 or 33751 nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

By providing the 18607, 15603, 69318, 12303, 48000, 52920, 5433, 38554, 57301, 58324, 55063, 52991, 59914, 59921 or 33751 nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif.

The present invention therefore provides a medium for holding instructions for performing a method for determining whether a subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, wherein the method comprises the steps of determining 18607, 15603, 69318, 12303, 48000, 52920, 5433, 38554, 57301, 58324, 55063, 52991, 59914, 59921 or 33751 sequence information associated with the subject and based on the 18607, 15603, 69318, 12303, 48000, 52920, 5433, 38554, 57301, 58324, 55063, 52991, 59914, 59921 or 33751 sequence information, determining whether the subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder and/or recommending a particular treatment for the disease, disorder, or pre-disease condition.

The present invention further provides in an electronic system and/or in a network, a method for determining whether a subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a disease associated with 18607, 15603, 69318, 12303, 48000, 52920, 5433, 38554, 57301, 58324, 55063, 52991, 59914, 59921 or 33751, wherein the method comprises the steps of determining 18607, 15603, 69318, 12303, 48000, 52920, 5433, 38554, 57301, 58324, 55063, 52991, 59914, 59921 or 33751 sequence information associated with the subject, and based on the 18607, 15603, 69318, 12303, 48000, 52920, 5433, 38554, 57301, 58324, 55063, 52991, 59914, 59921 or 33751 sequence information, determining whether the subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, and/or recommending a particular treatment for the disease, disorder, or pre-disease condition. The method may further comprise the step of receiving phenotypic information associated with the subject and/or acquiring from a network phenotypic information associated with the subject.

The present invention also provides in a network, a method for determining whether a subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, said method comprising the steps of receiving 18607, 15603, 69318, 12303, 48000, 52920, 5433, 38554, 57301, 58324, 55063, 52991, 59914, 59921 or 33751 sequence information from the subject and/or information related thereto, receiving phenotypic information associated with the subject, acquiring information from the network corresponding to 18607, 15603, 69318, 12303, 48000, 52920, 5433, 38554, 57301, 58324, 55063, 52991, 59914, 59921 or 33751 and/or corresponding to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, and based on one or more of the phenotypic information, the 18607, 15603, 69318, 12303, 48000, 52920, 5433, 38554, 57301, 58324, 55063, 52991, 59914, 59921 or 33751 information (e.g., sequence information and/or information related thereto), and the acquired information, determining whether the subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder. The method may further comprise the step of recommending a particular treatment for the disease, disorder, or pre-disease condition.

The present invention also provides a business method for determining whether a subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, said method comprising the steps of receiving information related to 18607, 15603, 69318, 12303, 48000, 52920, 5433, 38554, 57301, 58324, 55063, 52991, 59914, 59921 or 33751 (e.g., sequence information and/or information related thereto), receiving phenotypic information associated with the subject, acquiring information from the network related to 18607, 15603, 69318, 12303, 48000, 52920, 5433, 38554, 57301, 58324, 55063, 52991, 59914, 59921 or 33751 and/or related to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, and based on one or more of the phenotypic information, the 18607, 15603, 69318, 12303, 48000, 52920, 5433, 38554, 57301, 58324, 55063, 52991, 59914, 59921 or 33751 information, and the acquired information, determining whether the subject has a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder or a pre-disposition to a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder. The method may further comprise the step of recommending a particular treatment for the disease, disorder, or pre-disease condition.

The invention also includes an array comprising a 18607, 15603, 69318, 12303, 48000, 52920, 5433, 38554, 57301, 58324, 55063, 52991, 59914, 59921 or 33751 sequence of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be 18607, 15603, 69318, 12303, 48000, 52920, 5433, 38554, 57301, 58324, 55063, 52991, 59914, 59921 or 33751. This allows a profile to be developed showing a battery of genes specifically expressed in one or more tissues.

In addition to such qualitative information, the invention allows the quantitation of gene expression. Thus, not only tissue specificity, but also the level of expression of a battery of genes in the tissue if ascertainable. Thus, genes can be grouped on the basis of their tissue expression per se and level of expression in that tissue. This is useful, for example, in ascertaining the relationship of gene expression in that tissue. Thus, one tissue can be perturbed and the effect on gene expression in a second tissue can be determined. In this context, the effect of one cell type on another cell type in response to a biological stimulus can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, progression of calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder, and processes, such a cellular transformation associated with the calcium channel, calcium/sodium antiporter, potassium channel, organic ion transporter or choline transporter-associated or another 18607, 15603, 69318, 12303, 48000, 52920, 5433, 38554, 57301, 58324, 55063, 52991, 59914, 59921 or 33751-associated disease or disorder.

The array is also useful for ascertaining the effect of the expression of a gene on the expression of other genes in the same cell or in different cells (e.g., acertaining the effect of 18607, 15603, 69318, 12303, 48000, 52920, 5433, 38554, 57301, 58324, 55063, 52991, 59914, 59921 or 33751 expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including 18607, 15603, 69318, 12303, 48000, 52920, 5433, 38554, 57301, 58324, 55063, 52991, 59914, 59921 or 33751) that could serve as a molecular target for diagnosis or therapeutic intervention.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBI).

Thus, the invention features a method of making a computer readable record of a sequence of a 18607, 15603, 69318, 12303, 48000, 52920, 5433, 38554, 57301, 58324, 55063, 52991, 59914, 59921 or 33751 sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features a method of analyzing a sequence. The method includes: providing a 18607, 15603, 69318, 12303, 48000, 52920, 5433, 38554, 57301, 58324, 55063, 52991, 59914, 59921 or 33751 sequence, or record, in computer readable form; comparing a second sequence to the 18607, 15603, 69318, 12303, 48000, 52920, 5433, 38554, 57301, 58324, 55063, 52991, 59914, 59921 or 33751 sequence; thereby analyzing a sequence. Comparison can include comparing to sequences for sequence identity or determining if one sequence is included within the other, e.g., determining if the 18607, 15603, 69318, 12303, 48000, 52920, 5433, 38554, 57301, 58324, 55063, 52991, 59914, 59921 or 33751 sequence includes a sequence being compared. In a preferred embodiment the 18607, 15603, 69318, 12303, 48000, 52920, 5433, 38554, 57301, 58324, 55063, 52991, 59914, 59921 or 33751 or second sequence is stored on a first computer, e.g., at a first site and the comparison is performed, read, or recorded on a second computer, e.g., at a second site. E.g., the 18607, 15603, 69318, 12303, 48000, 52920, 5433, 38554, 57301, 58324, 55063, 52991, 59914, 59921 or 33751 or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

### EXEMPLIFICATION

### Example 1

### Tissue Distribution of 18607, 15603, 69318, 12303, 48000, 52920, 5433, 38554, 57301, 58324, 55063, 52991, 59914, 59921 or 33751 mRNA

Northern blot hybridizations with various RNA samples can be performed under standard conditions and washed under stringent conditions, i.e., 0.2×SSC at 65° C. A DNA probe corresponding to all or a portion of the 18607, 15603, 69318, 12303, 48000, 52920, 5433, 38554, 57301, 58324, 55063, 52991, 59914, 59921 or 33751 cDNA (SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83) or 18607, 15603, 69318, 12303, 48000, 52920, 5433, 38554, 57301, 58324, 55063, 52991, 59914, 59921 or 33751 cDNA can be used. The DNA was radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) can be probed in ExpressHyb hybridization solution (Clontech) and washed at high stringency according to manufacturer's recommendations.

### Example 2

### Recombinant Expression of 18607, 15603, 69318, 12303, 48000, 52920, 5433, 38554, 57301, 58324, 55063, 52991, 59914, 59921 or 33751 in Bacterial Cells

In this example, 18607, 15603, 69318, 12303, 48000, 52920, 5433, 38554, 57301, 58324, 55063, 52991, 59914, 59921 or 33751 is expressed as a recombinant glutathione-S-transferase (GST) fusion polypeptide in E. coli and the fusion polypeptide is isolated and characterized. Specifically, 18607, 15603, 69318, 12303, 48000, 52920, 5433, 38554, 57301, 58324, 55063, 52991, 59914, 59921 or 33751 is fused to GST and this fusion polypeptide is expressed in E. coli, e.g., strain PEB199. Expression of the GST-18607, -15603, -69318, -12303, -48000, -52920, -5433, -38554, -57301, -58324, -55063, -52991, -59914, -59921 or -33751 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant 18607, 15603, 69318, 12303, 48000, 52920, 5433, 38554, 57301, 58324, 55063, 52991, 59914, 59921 or 33751 Protein in COS Cells

To express the 18607, 15603, 69318, 12303, 48000, 52920, 5433, 38554, 57301, 58324, 55063, 52991, 59914, 59921 or 33751 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 18607, 15603, 69318, 12303, 48000, 52920, 5433, 38554, 57301, 58324, 55063, 52991, 59914, 59921 or 33751 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 18607, 15603, 69318, 12303, 48000, 52920, 5433, 38554, 57301, 58324, 55063, 52991, 59914, 59921 or 33751 dNA sequence is amplified by PCR using two primers. The 5′ primer contains the restriction site of interest followed by approximately twenty nucleotides of the 18607, 15603, 69318, 12303, 48000, 52920, 5433, 38554, 57301, 58324, 55063, 52991, 59914, 59921 or 33751 coding sequence starting from the initiation codon; the 3′ end sequence contains complementary sequences to the other restriction site of interest, a translation stop codon, the HA tag or FLAG tag and the last 20 nucleotides of the 18607, 15603, 69318, 12303, 48000, 52920, 5433, 38554, 57301, 58324, 55063, 52991, 59914, 59921 or 33751 coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the 18607, 15603, 69318, 12303, 48000, 52920, 5433, 38554, 57301, 58324, 55063, 52991, 59914, 59921 or 33751 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 18607-, 15603-, 69318-, 12303-, 48000-, 52920-, 5433-, 38554-, 57301-, 58324-, 55063-, 52991-, 59914-, 59921- or 33751-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 18607, 15603, 69318, 12303, 48000, 52920, 5433, 38554, 57301, 58324, 55063, 52991, 59914, 59921 or 33751 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the 18607, 15603, 69318, 12303, 48000, 52920, 5433, 38554, 57301, 58324, 55063, 52991, 59914, 59921 or 33751 coding sequence is cloned directly into the polylinker of the pcDNA/Amp vector using the appropriate restriction sites. The resulting plasmid is transfected into COS cells in the manner described above, and the expression of the 18607, 15603, 69318, 12303, 48000, 52920, 5433, 38554, 57301, 58324, 55063, 52991, 59914, 59921 or 33751 polypeptide is detected by radiolabelling and immunoprecipitation using a 18607, 15603, 69318, 12303, 48000, 52920, 5433, 38554, 57301, 58324, 55063, 52991, 59914, 59921 or 33751 specific monoclonal antibody.

### Example 4

### TaqMan Analysis of 18607, 15603, 69318, 12303, 48000, 52920, 5433, 38554, 57301, 58324, 55063, 52991, 59914, 59921 or 33751

Human 18607, 15603, 69318, 12303, 48000, 52920, 5433, 38554, 57301, 58324, 55063, 52991, 59914, 59921 or 33751 expression was measured by TaqMan® quantitative PCR (Perkin Elmer Applied Biosystems) in cDNA prepared from a variety of normal and diseased (e.g., cancerous) human tissues or cell lines.

Probes were designed by PrimerExpress software (PE Biosystems) based on the sequence of the human 18607, 15603, 69318, 12303, 48000, 52920, 5433, 38554, 57301, 58324, 55063, 52991, 59914, 59921 or 33751 gene. Each human 18607, 15603, 69318, 12303, 48000, 52920, 5433, 38554, 57301, 58324, 55063, 52991, 59914, 59921 or 33751 gene probe was labeled using FAM (6-carboxyfluorescein), and the β2-microglobulin reference probe was labeled with a different fluorescent dye, VIC. The differential labeling of the target gene and internal reference gene thus enabled measurement in same well. Forward and reverse primers and the probes for both β2-microglobulin and target gene were added to the TaqMan® Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target gene. TaqMan matrix experiments were carried out on an ABI PRISM 7700 Sequence Detection System (PE Applied Biosystems). The thermal cycler conditions were as follows: hold for 2 min at 50° C. and 10 min at 95° C., followed by two-step PCR for 40 cycles of 95° C. for 15 sec followed by 60° C. for 1 min.

The following method was used to quantitatively calculate human 18607, 15603, 69318, 12303, 48000, 52920, 5433, 38554, 57301, 58324, 55063, 52991, 59914, 59921 or 33751 gene expression in the various tissues relative to β-2 microglobulin expression in the same tissue. The threshold cycle (Ct) value is defined as the cycle at which a statistically significant increase in fluorescence is detected. A lower Ct value is indicative of a higher mRNA concentration. The Ct value of the human 18607, 15603, 69318, 12303, 48000, 52920, 5433, 38554, 57301, 58324, 55063, 52991, 59914, 59921 or 33751 gene is normalized by subtracting the Ct value of the β-2 microglobulin gene to obtain a _{Δ}Ct value using the following formula: _{Δ}Ct=Ctₛₐₘₚₗₑ−Ct_{β-2 microglobulin}. Expression is then calibrated against a cDNA sample showing a comparatively low level of expression of the human 18607, 15603, 69318, 12303, 48000, 52920, 5433, 38554, 57301, 58324, 55063, 52991, 59914, 59921 or 33751 gene. The _{Δ}Ct value for the calibrator sample is then subtracted from _{Δ}Ct for each tissue sample according to the following formula: _{ΔΔ}Ct=_{Δ}Ct-ₛₐₘₚₗₑ−_{Δ}Ct-_{calibrator}. Relative expression is then calculated using the arithmetic formula given by 2^{-ΔΔCt}.

### Example 5

### In Situ Hybridization of 18607, 15603, 69318, 12303, 48000, 52920, 5433, 38554, 57301, 58324, 55063, 52991, 59914, 59921 or 33751

The following describes the tissue distribution of 18607, 15603, 69318, 12303, 48000, 52920, 5433, 38554, 57301, 58324, 55063, 52991, 59914, 59921 or 33751 mRNA, as may be determined by in situ hybridization analysis using oligonucleotide probes based on the human G2RF sequence.

For in situ analysis, various tissues, e.g. tissues obtained from brain, are first frozen on dry ice. Ten-micrometer-thick sections of the tissues are postfixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections are rinsed in DEPC 2×SSC (1×SSC is 0.15M NaCl plus 0.015M sodium citrate). Tissue is then dehydrated through a series of ethanol washes, incubated in 100% chloroform for 5 minutes, and then rinsed in 100% ethanol for 1 minute and 95% ethanol for 1 minute and allowed to air dry.

Hybridizations are performed with ³⁵S-radiolabeled (5×10⁷ cpm/ml) cRNA probes. Probes are incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides are washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides are then rinsed with 2×SSC at room temperature, washed with 2×SSC at 50° C. for 1 hour, washed with 0.2×SSC at 55° C. for 1 hour, and 0.2×SSC at 60° C. for 1 hour. Sections are then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

The contents of all references, patents and published patent applications cited throughout this application are incorporated herein by reference.

### EQUIVALENTS

Those skilled in the art will recognize, or be able to ascertain using no more than routine experimentation, many equivalents to the specific embodiments of the invention described herein.

What is claimed is:

1. An isolated 18607, 15603, 69318, 12303, 48000, 52920, 5433, 38554, 57301, 58324, 55063, 52991, 59914, 59921 or 33751 nucleic acid molecule selected from the group consisting of:
a) a nucleic acid molecule comprising a nucleotide sequence which is at least 60% identical to the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83;
b) a nucleic acid molecule comprising a fragment of at least 15 nucleotides of the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83;
c) a nucleic acid molecule which encodes a polypeptide comprising the amino acid sequence of SEQ ID NO:2, 6, 14, 19, 22, 25, 28, 33, 36, 39, 54, 57, 61, 70, 73 or 82;
d) a nucleic acid molecule which encodes a fragment of a polypeptide comprising the amino acid sequence of SEQ ID NO:2, 6, 14, 19, 22, 25, 28, 33, 36, 39, 54, 57, 61, 70, 73 or 82, wherein the fragment comprises at least 15 contiguous amino acids of SEQ ID NO:2, 6, 14, 19, 22, 25, 28, 33, 36, 39, 54, 57, 61, 70, 73 or 82;
e) a nucleic acid molecule which encodes a naturally occurring allelic variant of a polypeptide comprising the amino acid sequence of SEQ ID NO:2, 6, 14, 19, 22, 25, 28, 33, 36, 39, 54, 57, 61, 70, 73 or 82, wherein the nucleic acid molecule hybridizes to a nucleic acid molecule comprising SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or a complement thereof, under stringent conditions;
f) a nucleic acid molecule comprising the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83; and
g) a nucleic acid molecule which encodes a polypeptide comprising the amino acid sequence of SEQ ID NO:2, 6, 14, 19, 22, 25, 28, 33, 36, 39, 54, 57, 61, 70, 73 or 82.

2. The isolated nucleic acid molecule of claim 1, which is the nucleotide sequence SEQ ID NO:1, 5, 13, 18, 21, 24, 27, 32, 35, 38, 53, 56, 60, 69, 72 or 81.

3. A host cell which contains the nucleic acid molecule of claim 1.

4. An isolated 18607, 15603, 69318, 12303, 48000, 52920, 5433, 38554, 57301, 58324, 55063, 52991, 59914, 59921 or 33751 polypeptide selected from the group consisting of:
a) a polypeptide which is encoded by a nucleic acid molecule comprising a nucleotide sequence which is at least 60% identical to a nucleic acid comprising the nucleotide sequence of SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or a complement thereof;
b) a naturally occurring allelic variant of a polypeptide comprising the amino acid sequence of SEQ ID NO:2, 6, 14, 19, 22, 25, 28, 33, 36, 39, 54, 57, 61, 70, 73 or 82, wherein the polypeptide is encoded by a nucleic acid molecule which hybridizes to a nucleic acid molecule comprising SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83, or a complement thereof under stringent conditions;
c) a fragment of a polypeptide comprising the amino acid sequence of SEQ ID NO:2, 6, 14, 19, 22, 25, 28, 33, 36, 39, 54, 57, 61, 70, 73 or 82, wherein the fragment comprises at least 15 contiguous amino acids of SEQ ID NO:2, 6, 14, 19, 22, 25, 28, 33, 36, 39, 54, 57, 61, 70, 73 or 82; and
d) the amino acid sequence of SEQ ID NO:2, 6, 14, 19, 22, 25, 28, 33, 36, 39, 54, 57, 61, 70, 73 or 82.

5. An antibody which selectively binds to a polypeptide of claim 4.

6. The polypeptide of claim 4, further comprising heterologous amino acid sequences.

7. A method for producing a polypeptide selected from the group consisting of:
a) a polypeptide comprising the amino acid sequence of SEQ ID NO:2, 6, 14, 19, 22, 25, 28, 33, 36, 39, 54, 57, 61, 70, 73 or 82;
b) a polypeptide comprising a fragment of the amino acid sequence of SEQ ID NO:2, 6, 14, 19, 22, 25, 28, 33, 36, 39, 54, 57, 61, 70, 73 or 82, wherein the fragment comprises at least 15 contiguous amino acids of SEQ ID NO:2, 6, 14, 19, 22, 25, 28, 33, 36, 39, 54, 57, 61, 70, 73 or 82;
c) a naturally occurring allelic variant of a polypeptide comprising the amino acid sequence of SEQ ID NO:2, 6, 14, 19, 22, 25, 28, 33, 36, 39, 54, 57, 61, 70, 73 or 82, wherein the polypeptide is encoded by a nucleic acid molecule which hybridizes to a nucleic acid molecule comprising SEQ ID NO:1, 3, 5, 7, 13, 15, 18, 20, 21, 23, 24, 26, 27, 29, 32, 34, 35, 37, 38, 40, 53, 55, 56, 58, 60, 62, 69, 71, 72, 74, 81 or 83; and
d) the amino acid sequence of SEQ ID NO:2, 6, 14, 19, 22, 25, 28, 33, 36, 39, 54, 57, 61, 70, 73 or 82;
comprising culturing the host cell of claim 3 under conditions in which the nucleic acid molecule is expressed.

8. A method for detecting the presence of a nucleic acid molecule of claim 1 or a polypeptide encoded by the nucleic acid molecule in a sample, comprising:
a) contacting the sample with a compound which selectively hybridizes to the nucleic acid molecule of claim 1 or binds to the polypeptide encoded by the nucleic acid molecule; and
b) determining whether the compound hybridizes to the nucleic acid or binds to the polypeptide in the sample.

9. A kit comprising a compound which selectively hybridizes to a nucleic acid molecule of claim 1 or binds to a polypeptide encoded by the nucleic acid molecule and instructions for use.

10. A method for identifying a compound which binds to a polypeptide or modulates the activity of the polypeptide of claim 4 comprising the steps of:
a) contacting a polypeptide, or a cell expressing a polypeptide of claim 4 with a test compound; and
b) determining whether the polypeptide binds to the test compound or determining the effect of the test compound on the activity of the polypeptide.

11. A method for modulating the activity of a polypeptide of claim 4 comprising contacting the polypeptide or a cell expressing the polypeptide with a compound which binds to the polypeptide in a sufficient concentration to modulate the activity of the polypeptide.

12. A method for identifying a compound capable of treating a disorder characterized by aberrant 18607, 15603, 69318, 12303, 48000, 52920, 5433, 38554, 57301, 58324, 55063, 52991, 59914, 59921 or 33751 activity, comprising assaying the ability of the compound to modulate 18607, 15603, 69318, 12303, 48000, 52920, 5433, 38554, 57301, 58324, 55063, 52991, 59914, 59921 or 33751 nucleic acid expression or 18607, 15603, 69318, 12303, 48000, 52920, 5433, 38554, 57301, 58324, 55063, 52991, 59914, 59921 or 33751 polypeptide activity, thereby identifying a compound capable of treating a disorder characterized by aberrant 18607, 15603, 69318, 12303, 48000, 52920, 5433, 38554, 57301, 58324, 55063, 52991, 59914, 59921 or 33751 activity.

13. A method of identifying a nucleic acid molecule associated with a disorder characterized by aberrant 18607, 15603, 69318, 12303, 48000, 52920, 5433, 38554, 57301, 58324, 55063, 52991, 59914, 59921 or 33751 activity, comprising:
a) contacting a sample from a subject with a disorder characterized by aberrant 18607, 15603, 69318, 12303, 48000, 52920, 5433, 38554, 57301, 58324, 55063, 52991, 59914, 59921 or 33751 activity, comprising nucleic acid molecules with a hybridization probe comprising at least 25 contiguous nucleotides of SEQ ID NO:1, 5, 13, 18, 21, 24, 27, 32, 35, 38, 53, 56, 60, 69, 72 or 81 defined in claim 2; and
b) detecting the presence of a nucleic acid molecule in the sample that hybridizes to the probe, thereby identifying a nucleic acid molecule associated with a disorder characterized by aberrant 18607, 15603, 69318, 12303, 48000, 52920, 5433, 38554, 57301, 58324, 55063, 52991, 59914, 59921 or 33751 activity.

14. A method of identifying a polypeptide associated with a disorder characterized by aberrant 18607, 15603, 69318, 12303, 48000, 52920, 5433, 38554, 57301, 58324, 55063, 52991, 59914, 59921 or 33751 activity, comprising:
a) contacting a sample comprising polypeptides with a 18607, 15603, 69318, 12303, 48000, 52920, 5433, 38554, 57301, 58324, 55063, 52991, 59914, 59921 or 33751 polypeptide defined in claim 4; and
b) detecting the presence of a polypeptide in the sample that binds to the 18607, 15603, 69318, 12303, 48000, 52920, 5433, 38554, 57301, 58324, 55063, 52991, 59914, 59921 or 33751 binding partner, thereby identifying the polypeptide associated with a disorder characterized by aberrant 18607, 15603, 69318, 12303, 48000, 52920, 5433, 38554, 57301, 58324, 55063, 52991, 59914, 59921 or 33751 activity.

15. A method of identifying a subject having a disorder characterized by aberrant 18607, 15603, 69318, 12303, 48000, 52920, 5433, 38554, 57301, 58324, 55063, 52991, 59914, 59921 or 33751 activity, comprising:
a) contacting a sample obtained from the subject comprising nucleic acid molecules with a hybridization probe comprising at least 25 contiguous nucleotides of SEQ ID NO:1, 5, 13, 18, 21, 24, 27, 32, 35, 38, 53, 56, 60, 69, 72 or 81 defined in claim 2; and
b) detecting the presence of a nucleic acid molecule in the sample that hybridizes to the probe, thereby identifying a subject having a disorder characterized by aberrant 18607, 15603, 69318, 12303, 48000, 52920, 5433, 38554, 57301, 58324, 55063, 52991, 59914, 59921 or 33751 activity.

16. A method for treating a subject having a disorder characterized by aberrant 18607, 15603, 69318, 12303, 48000, 52920, 5433, 38554, 57301, 58324, 55063, 52991, 59914, 59921 or 33751 activity, or a subject at risk of developing a disorder characterized by aberrant 18607, 15603, 69318, 12303, 48000, 52920, 5433, 38554, 57301, 58324, 55063, 52991, 59914, 59921 or 33751 activity, comprising administering to the subject a 18607, 15603, 69318, 12303, 48000, 52920, 5433, 38554, 57301, 58324, 55063, 52991, 59914, 59921 or 33751 modulator of the nucleic acid molecule defined in claim 1 or the polypeptide encoded by the nucleic acid molecule or contacting a cell with a 18607, 15603, 69318, 12303, 48000, 52920, 5433, 38554, 57301, 58324, 55063, 52991, 59914, 59921 or 33751 modulator.

17. The method defined in claim 16 wherein said disorder is a cellular proliferative and/or differentiative disorder, brain disorder, blood vessel disorder, platelet disorder, breast disorder, colon disorder, kidney disorder, lung disorder, ovarian disorder, prostate disorder, pancreatic disorder, skeletal muscle disorder, testicular disorder, hormonal disorder, disorder associated with bone metabolism, immune e.g., inflammatory disorder, cardiovascular disorder, endothelial cell disorder, liver disorder, viral disease, pain, metabolic disorder, anemia, angiogenesic disorder, neoplastic disorder, endocrine disorder, neurological disorder or heart disorder.

18. The method of claim 16, wherein the 18607, 15603, 69318, 12303, 48000, 52920, 5433, 38554, 57301, 58324, 55063, 52991, 59914, 59921 or 33751 modulator is
a) a small molecule;
b) peptide;
c) phosphopeptide;
d) anti-18607, 15603, 69318, 12303, 48000, 52920, 5433, 38554, 57301, 58324, 55063, 52991, 59914, 59921 or 33751 antibody;
e) a 18607, 15603, 69318, 12303, 48000, 52920, 5433, 38554, 57301, 58324, 55063, 52991, 59914, 59921 or 33751 polypeptide comprising the amino acid sequence of SEQ ID NO:2, 6, 14, 19, 22, 25, 28, 33, 36, 39, 54, 57, 61, 70, 73 or 82, or a fragment thereof;
f) a 18607, 15603, 69318, 12303, 48000, 52920, 5433, 38554, 57301, 58324, 55063, 52991, 59914, 59921 or 33751 polypeptide comprising an amino acid sequence which is at least 90 percent identical to the amino acid sequence of SEQ ID NO:2, 6, 14, 19, 22, 25, 28, 33, 36, 39, 54, 57, 61, 70, 73 or 82, wherein the percent identity is calculated using the ALIGN program for comparing amino acid sequences, a PAM120 weight residue table, a gap length penalty of 12, and a gap penalty of 4; or
g) an isolated naturally occurring allelic variant of a polypeptide consisting of the amino acid sequence of SEQ ID NO:2, 6, 14, 19, 22, 25, 28, 33, 36, 39, 54, 57, 61, 70, 73 or 82, wherein the polypeptide is encoded by a nucleic acid molecule which hybridizes to a complement of a nucleic acid molecule consisting of SEQ ID NO:1, 5, 13, 18, 21, 24, 27, 32, 35, 38, 53, 56, 60, 69, 72 or 81 at 6×SSC at 45° C., followed by one or more washes in 0.2×SSC, 0.1% SDS at 65° C.

19. The method of claim 16, wherein the 18607, 15603, 69318, 12303, 48000, 52920, 5433, 38554, 57301, 58324, 55063, 52991, 59914, 59921 or 33751 modulator is
a) an antisense 18607, 15603, 69318, 12303, 48000, 52920, 5433, 38554, 57301, 58324, 55063, 52991, 59914, 59921 or 33751 nucleic acid molecule;
b) is a ribozyme;
c) the nucleotide sequence of SEQ ID NO:1, 5, 13, 18, 21, 24, 27, 32, 35, 38, 53, 56, 60, 69, 72 or 81 or a fragment thereof;
d) a nucleic acid molecule encoding a polypeptide comprising an amino acid sequence which is at least 90 percent identical to the amino acid sequence of SEQ ID NO:2, 6, 14, 19, 22, 25, 28, 33, 36, 39, 54, 57, 61, 70, 73 or 82, wherein the percent identity is calculated using the ALIGN program for comparing amino acid sequences, a PAM120 weight residue table, a gap length penalty of 12, and a gap penalty of 4;
e) a nucleic acid molecule encoding a naturally occurring allelic variant of a polypeptide comprising the amino acid sequence of SEQ ID NO:2, 6, 14, 19, 22, 25, 28, 33, 36, 39, 54, 57, 61, 70, 73 or 82, wherein the nucleic acid molecule which hybridizes to a complement of a nucleic acid molecule consisting of SEQ ID NO:1, 5, 13, 18, 21, 24, 27, 32, 35, 38, 53, 56, 60, 69, 72 or 81 at 6×SSC at 45° C., followed by one or more washes in 0.2×SSC, 0.1% SDS at 65° C.; or
f) a gene therapy vector.
